(19) United States
(12) Patent Application Publication
(10) Pub. No.: US 2008/0249137 A1
(43) Pub. Date: Oct. 9, 2008

(54) PPAR active compounds

(76) Inventors: Jack Lin, Hercules, CA (US); Patrick Womack, Berkeley, CA (US); Byunghun Lee, Marina, CA (US); Shenghua Shi, San Diego, CA (US); Chao Zhang, Moraga, CA (US); Dean R. Artis, Kensington, CA (US); Prabha N. Ibrahim, Mountain View, CA (US); Weiru Wang, Lafayette, CA (US); Rebecca Zuckerman, Alameda, CA (US)

Correspondence Address: FOLEY & LARDNER LLP, P.O. BOX 80278, SAN DIEGO, CA, 92138-0278, US

(21) Appl. No.: 11/517,572

(22) Filed: Sep. 6, 2006

Related U.S. Application Data
(60) Provisional application No. 60/789,387, filed on Apr. 5, 2006.
(60) Provisional application No. 60/715,214, filed on Sep. 7, 2005.

(51) Int. Cl. A61K 31/44; C07C 235/34; A61K 31/165; A61K 31/192; A61P 3/04; C07C 57/58; C07D 213/64

(52) U.S. Cl. 514/347; 564/169; 514/621; 546/294; 562/429; 514/568

(57) ABSTRACT

Compounds are described that are active on at least one of PPARα, PPARδ, and PPARγ, which are useful for therapeutic and/or prophylactic methods involving modulation of at least one of PPARα, PPARδ, and PPARγ.

### RELATED PATENT APPLICATIONS

This application claims the benefit of U.S. Prov. App. No. 60/715,214, filed Sep. 7, 2005, and U.S. Prov. App. No. 60/789,387, filed Apr. 5, 2006, both of which are incorporated herein by reference in their entireties and for all purposes.

### FIELD OF THE INVENTION

The present invention relates to the field of modulators for members of the family of nuclear receptors identified as peroxisome proliferator-activated receptors.

### BACKGROUND OF THE INVENTION

The following description is provided solely to assist the understanding of the reader. None of the references cited or information provided is admitted to be prior art to the present invention. Each of the references cited herein is incorporated by reference in its entirety, to the same extent as if each reference were individually indicated to be incorporated by reference herein in its entirety.

The peroxisome proliferator-activated receptors (PPARs) form a subfamily in the nuclear receptor superfamily. Three isoforms, encoded by separate genes, have been identified thusfar: PPARγ, PPARα, and PPARδ.

There are two PPARγ isoforms expressed at the protein level in mouse and human, γ1 and γ2. They differ only in that the latter has 30 additional amino acids at its N terminus due to differential promoter usage within the same gene, and subsequent alternative RNA processing. PPARγ2 is expressed primarily in adipose tissue, while PPARγ1 is expressed in a broad range of tissues.

Murine PPARα was the first member of this nuclear receptor subclass to be cloned; it has since been cloned from humans. PPARα is expressed in numerous metabolically active tissues, including liver, kidney, heart, skeletal muscle, and brown fat. It is also present in monocytes, vascular endothelium, and vascular smooth muscle cells. Activation of PPARα induces hepatic peroxisome proliferation, hepatomegaly, and hepatocarcinogenesis in rodents. These toxic effects are not observed in humans, although the same compounds activate PPARα across species.

Human PPARδ was cloned in the early 1990s and subsequently cloned from rodents. PPARδ is expressed in a wide range of tissues and cells; with the highest levels of expression found in the digestive tract, heart, kidney, liver, adipose, and brain.

The PPARs are ligand-dependent transcription factors that regulate target gene expression by binding to specific peroxisome proliferator response elements (PPREs) in enhancer sites of regulated genes. PPARs possess a modular structure composed of functional domains that include a DNA binding domain (DBD) and a ligand binding domain (LBD). The DBD specifically binds PPREs in the regulatory region of PPAR-responsive genes. The DBD, located in the C-terminal half of the receptor, contains the ligand-dependent activation domain, AF-2. Each receptor binds to its PPRE as a heterodimer with a retinoid X receptor (RXR). Upon binding an agonist, the conformation of a PPAR is altered and stabilized such that a binding cleft, made up in part of the AF-2 domain, is created and recruitment of transcriptional coactivators occurs. Coactivators augment the ability of nuclear receptors to initiate the transcription process. The result of the agonist-induced PPAR-coactivator interaction at the PPRE is an increase in gene transcription. Downregulation of gene expression by PPARs appears to occur through indirect mechanisms. (Bergen, et al., Diabetes Tech. & Ther., 2002, 4:163-174).

The first cloning of a PPAR (PPARα) occurred in the course of the search for the molecular target of rodent hepatic peroxisome proliferating agents. Since then, numerous fatty acids and their derivatives, including a variety of eicosanoids and prostaglandins, have been shown to serve as ligands of the PPARs. Thus, these receptors may play a central role in the sensing of nutrient levels and in the modulation of their metabolism. In addition, PPARs are the primary targets of selected classes of synthetic compounds that have been used in the successful treatment of diabetes and dyslipidemia. As such, an understanding of the molecular and physiological characteristics of these receptors has become extremely important to the development and utilization of drugs used to treat metabolic disorders.

Kota, et al., Pharmacological Research, 2005, 51:85-94, provides a review of biological mechanisms involving PPARs that includes a discussion of the possibility of using PPAR modulators for treating a variety of conditions, including chronic inflammatory disorders such as atherosclerosis, arthritis and inflammatory bowel syndrome, retinal disorders associated with angiogenesis, increased fertility, and neurodegenerative diseases.

Yousef, et al., Journal of Biomedicine and Biotechnology, 2004(3): 156-166, discusses the anti-inflammatory effects of PPARα, PPARγ and PPARδ agonists, suggesting that PPAR agonists may have a role in treating neuronal diseases such as Alzheimer's disease, and autoimmune diseases such as inflammatory bowel disease and multiple sclerosis. A potential role for PPAR agonists in the treatment of Alzheimer's disease has been described in Combs, et al., Journal of Neuroscience 2000, 20(2):558, and such a role for PPAR agonists in Parkinson's disease is discussed in Breidert, et al., Journal of Neurochemistry, 2002, 82:615. A potential related function of PPAR agonists in treatment of Alzheimer's disease, that of regulation of the APP-processing enzyme BACE, has been discussed in Sastre, et al., Journal of Neuroscience, 2003, 23(30):9796. These studies collectively indicate PPAR agonists may provide advantages in treating a variety of neurodegenerative diseases by acting through complementary mechanisms.

Discussion of the anti-inflammatory effects of PPAR agonists is also available in Feinstein, Drug Discovery Today: Therapeutic Strategies, 2004, 1(1):29-34, in relation to multiple sclerosis and Alzheimer's disease; Patel, et al., Journal of Immunology, 2003, 170:2663-2669 in relation to chronic obstructive pulmonary disease and asthma (COPD); Lovett-Racke, et al., Journal of Immunology, 2004, 172:5790-5798 in relation to autoimmune disease; Malhotra, et al., Expert Opinions in Pharmacotherapy, 2005, 6(9):1455-1461, in relation to psoriasis; and Storer, et al., Journal of Neuroimmunology, 2005, 161:113-122, in relation to multiple sclerosis.

This wide range of roles for the PPARs that have been discovered suggest that PPARα, PPARγ and PPARδ may play a role in a wide range of events involving the vasculature, including atherosclerotic plaque formation and stability, thrombosis, vascular tone, angiogenesis, cancer, pregnancy, pulmonary disease, autoimmune disease, and neurological disorders.

Among the synthetic ligands identified for PPARs are thiazolidinediones (TZDs). These compounds were originally developed on the basis of their insulin-sensitizing effects in animal pharmacology studies. Subsequently, it was found that TZDs induced adipocyte differentiation and increased expression of adipocyte genes, including the adipocyte fatty acid-binding protein aP2. Independently, it was discovered that PPARγ interacted with a regulatory element of the aP2 gene that controlled its adipocyte-specific expression. On the basis of these seminal observations, experiments were performed that determined that TZDs were PPARγ ligands and agonists and demonstrate a definite correlation between their in vitro PPARγ activities and their in vivo insulin-sensitizing actions. (Bergen, et al., supra).

Several TZDs, including troglitazone, rosiglitazone, and pioglitazone, have insulin-sensitizing and anti-diabetic activity in humans with type 2 diabetes and impaired glucose tolerance. Farglitazar is a very potent non-TZD PPAR-γ-selective agonist that was recently shown to have anti-diabetic as well as lipid-altering efficacy in humans. In addition to these potent PPARγ ligands, a subset of the non-steroidal anti-inflammatory drugs (NSAIDs), including indomethacin, fenoprofen, and ibuprofen, have displayed weak PPARγ and PPARα activities. (Bergen, et al., supra).

The fibrates, amphipathic carboxylic acids that have been proven useful in the treatment of hypertriglyceridemia, are PPARα ligands. The prototypical member of this compound class, clofibrate, was developed prior to the identification of PPARs, using in vivo assays in rodents to assess lipid-lowering efficacy. (Bergen, et al., supra).

Fu et al., Nature, 2003, 425:9093, demonstrated that the PPARα binding compound, oleylethanolamide, produces satiety and reduces body weight gain in mice.

Clofibrate and fenofibrate have been shown to activate PPARα with a 10-fold selectivity over PPARγ. Bezafibrate acts as a pan-agonist that shows similar potency on all three PPAR isoforms. Wy-14643, the 2-arylthioacetic acid analogue of clofibrate, is a potent murine PPARα agonist as well as a weak PPARγ agonist. In humans, all of the fibrates must be used at high doses (200-1,200 mg/day) to achieve efficacious lipid-lowering activity.

TZDs and non-TZDs have also been identified that are dual PPARγ/α agonists. By virtue of the additional PPARα agonist activity, this class of compounds has potent lipid-altering efficacy in addition to anti-hyperglycemic activity in animal models of diabetes and lipid disorders. KRP-297 is an example of a TZD dual PPARγ/α agonist (Fajas, J. Biol. Chem., 1997, 272:18779-18789); furthermore, DRF-2725 and AZ-242 are non-TZD dual PPARγ/α agonists. (Lohray, et al., J. Med. Chem., 2001, 44:2675-2678; Cronet, et al., Structure (Camb.), 2001, 9:699-706).

In order to define the physiological role of PPARδ, efforts have been made to develop novel compounds that activate this receptor in a selective manner. Amongst the α-substituted carboxylic acids previously described, the potent PPARδ ligand L-165041 demonstrated approximately 30-fold agonist selectivity for this receptor over PPARγ, and it was inactive on murine PPARα (Liebowitz, et al., 2000, FEBS Lett., 473:333-336). This compound was found to increase high-density lipoprotein levels in rodents. It was also reported that GW501516 was a potent, highly-selective PPARδ agonist that produced beneficial changes in serum lipid parameters in obese, insulin-resistant rhesus monkeys. (Oliver et al., Proc. Natl. Acad. Sci., 2001, 98:5306-5311).

In addition to the compounds discussed above, certain thiazole derivatives active on PPARs have been described. (Cadilla, et al., Internat. Appl. PCT/US01/149320, Internat. Publ. WO 02/062774, incorporated herein by reference in its entirety.)

Some tricyclic-α-alkyloxyphenylpropionic acids have been described as dual PPARα/γ agonists in Sauerberg, et al., J. Med. Chem. 2002, 45:789-804.

A group of compounds that are stated to have equal activity on PPARα/γ/δ is described in Morgensen, et al., Bioorg. & Med. Chem. Lett., 2002, 13:257-260.

Oliver et al., describes a selective PPARδ agonist that promotes reverse cholesterol transport. (Oliver, et al., supra)

Yamamoto et al., U.S. Pat. No. 3,489,767 describes “1-(phenylsulfonyl)-indolyl aliphatic acid derivatives” that are stated to have “antiphlogistic, analgesic and antipyretic actions.” (Col. 1, lines 16-19.)

Kato, et al., European patent application 94101551.3, Publication No. 0 610 793 A1, describes the use of 3-(5-methoxy-1-p-toluenesulfonylindol-3-yl)propionic acid (page 6) and 1-(2,3,6-triisopropylphenylsulfonyl)-indole-3-propionic acid (page 9) as intermediates in the synthesis of particular tetracyclic morpholine derivatives useful as analgesics.

### SUMMARY OF THE INVENTION

The present invention relates to compounds active on PPARs, which are useful for a variety of applications including, for example, therapeutic and/or prophylactic methods involving modulation of at least one of PPARα, PPARδ, and PPARγ. Included are compounds that have pan-activity across the PPAR family (i.e., PPARα, PPARδ, and PPARγ), as well as compounds that have significant specificity (at least 5-, 10-, 20-, 50-, or 100-fold greater activity) on a single PPAR, or on two of the three PPARs.

In one aspect, the invention provides compounds of Formula I as follows:

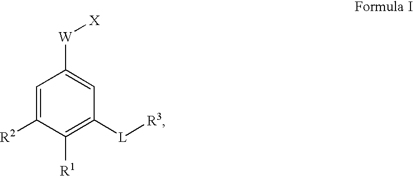

all salts, prodrugs, tautomers, and isomers thereof,
wherein:
X is selected from the group consisting of —C(O)OR¹⁶, —C(O)NR¹⁷R¹⁸, and a carboxylic acid isostere;
W is selected from the group consisting of a covalent bond, —NR⁵¹(CR⁴R⁵)₁₋₂—, —O—(CR⁴R⁵)₁₋₂—, —S—(CR⁴R⁵)₁₋₂—, —(CR⁴R⁵)₁₋₃—, and —CR⁶═CR⁷—;
R¹ and R² are independently selected from the group consisting of hydrogen, halogen, lower alkyl, lower alkenyl, lower alkynyl, —SR⁹, and —OR⁹, wherein lower alkyl, lower alkenyl and lower alkynyl are optionally substituted with one or more substituents selected from the group consisting of fluoro, —OH, —NH₂, lower alkoxy, fluoro substituted lower alkoxy, lower alkylthio, fluoro substituted lower alkylthio, cycloalkyl, heterocycloalkyl, aryl and heteroaryl, wherein cycloalkyl, heterocycloalkyl, aryl and heteroaryl are optionally substituted with one or more substituents selected from the group consisting of halogen, —OH, —NH₂, lower alkyl, fluoro substituted lower alkyl, lower alkenyl, fluoro substituted lower alkenyl, lower alkynyl, fluoro substituted lower alkynyl, lower alkoxy, fluoro substituted lower alkoxy, lower alkylthio, and fluoro substituted lower alkylthio;
R³ is selected from the group consisting of —[(CR⁴R⁵)ₘ—(Y)ₚ]ᵣ—R¹⁰ and —[(CR⁴R⁵)ₘ—(Y)ₚ]ᵣ—Ar₁-M-Ar₂;
L is selected from the group consisting of —O—, —S—, —NR⁵²—, —C(Z)-, —S(O)ₙ—, —C(Z)NR⁵²—, —NR⁵²C(Z)-, —NR⁵²S(O)₂—, —S(O)₂NR⁵²—, —NR⁵²C(Z)NR⁵²—, and —NR⁵²S(O)₂NR⁵²—;
Y is selected from the group consisting of —O—, —S—, —NR⁵³—, —C(Z)-, —S(O)ₙ—, —C(Z)NR⁵⁴—, —NR⁵⁴C(Z)-, —NR⁵⁴S(O)₂—, —S(O)₂NR⁵⁴—, —NR⁵⁴C(Z)NR⁵⁴—, and —NR⁵⁴S(O)₂NR⁵⁴—;
Ar₁ is selected from the group consisting of optionally substituted arylene and optionally substituted heteroarylene;
M is selected from the group consisting of a covalent bond, —CR¹⁹R²⁰—, —O—, —S—, —NR⁵³—, —C(Z)-, and —S(O)ₙ—;
Ar₂ is selected from the group consisting of optionally substituted aryl and optionally substituted heteroaryl;
R⁴ and R⁵ at each occurrence are independently selected from the group consisting of hydrogen, fluoro and lower alkyl, wherein lower alkyl is optionally substituted with one or more substituents selected from the group consisting of fluoro, —OH, —NH₂, lower alkoxy, fluoro substituted lower alkoxy, lower alkylthio, and fluoro substituted lower alkylthio; or
one R⁴ or R⁵ is selected from the group consisting of phenyl, 5-7 membered monocyclic heteroaryl, 3-7 membered monocyclic cycloalkyl, and 5-7 membered monocyclic heterocycloalkyl and any others of R⁴ and R⁵ are independently selected from the group consisting of hydrogen, fluoro and lower alkyl, wherein lower alkyl is optionally substituted with one or more substituents selected from the group consisting of fluoro, —OH, —NH₂, lower alkoxy, fluoro substituted lower alkoxy, lower alkylthio, and fluoro substituted lower alkylthio, and wherein phenyl, monocyclic heteroaryl, monocyclic cycloalkyl and monocyclic heterocycloalkyl are optionally substituted with one or more substituents selected from the group consisting of halogen, —OH, —NH₂, lower alkyl, fluoro substituted lower alkyl, lower alkoxy, fluoro substituted lower alkoxy, lower alkylthio, and fluoro substituted lower alkylthio; or
any two of R⁴ and R⁵ on the same or different carbons combine to form a 3-7 membered monocyclic cycloalkyl or 5-7 membered monocyclic heterocycloalkyl and any others of R⁴ and R⁵ are independently selected from the group consisting of hydrogen, fluoro and lower alkyl, wherein lower alkyl is optionally substituted with one or more substituents selected from the group consisting of fluoro, —OH, —NH₂, lower alkoxy, fluoro substituted lower alkoxy, lower alkylthio, and fluoro substituted lower alkylthio, and wherein the monocyclic cycloalkyl or monocyclic heterocycloalkyl are optionally substituted with one or more substituents selected from the group consisting of halogen, —OH, —NH₂, lower alkyl, fluoro substituted lower alkyl, lower alkoxy, fluoro substituted lower alkoxy, lower alkylthio, and fluoro substituted lower alkylthio;
R⁶ and R⁷ are independently hydrogen or lower alkyl wherein lower alkyl is optionally substituted with one or more substituents selected from the group consisting of fluoro, —OH, —NH₂, lower alkoxy, fluoro substituted lower alkoxy, lower alkylthio, and fluoro substituted lower alkylthio; or
one of R⁶ and R⁷ is selected from the group consisting of phenyl, 5-7 membered monocyclic heteroaryl, 3-7 membered monocyclic cycloalkyl, and 5-7 membered monocyclic heterocycloalkyl and the other of R⁶ and R⁷ is hydrogen or lower alkyl, wherein lower alkyl is optionally substituted with one or more substituents selected from the group consisting of fluoro, —OH, —NH₂, lower alkoxy, fluoro substituted lower alkoxy, lower alkylthio, and fluoro substituted lower alkylthio, and wherein phenyl, monocyclic heteroaryl, monocyclic cycloalkyl and monocyclic heterocycloalkyl are optionally substituted with one or more substituents selected from the group consisting of halogen, —OH, —NH₂, lower alkyl, fluoro substituted lower alkyl, lower alkoxy, fluoro substituted lower alkoxy, lower alkylthio, and fluoro substituted lower alkylthio; or
R⁶ and R⁷ combine to form a 5-7 membered monocyclic cycloalkyl or 5-7 membered monocyclic heterocycloalkyl, wherein the monocyclic cycloalkyl or monocyclic heterocycloalkyl are optionally substituted with one or more substituents selected from the group consisting of halogen, —OH, —NH₂, lower alkyl, fluoro substituted lower alkyl, lower alkoxy, fluoro substituted lower alkoxy, lower alkylthio, and fluoro substituted lower alkylthio;
R⁹ at each occurrence is independently selected from the group consisting of lower alkyl, C₃₋₆ alkenyl, provided, however, that when R⁹ is C₃₋₆ alkenyl, no alkene carbon thereof is bound to the O of —OR⁹ or the S of —SR⁹, C₃₋₆ alkynyl, provided, however, that when R⁹ is C₃₋₆ alkynyl, no alkyne carbon thereof is bound to the O of —OR⁹ or the S of —SR⁹, cycloalkyl, heterocycloalkyl, aryl, and heteroaryl, wherein cycloalkyl, heterocycloalkyl, aryl and heteroaryl are optionally substituted with one or more substituents selected from the group consisting of halogen, —OH, —NH₂, lower alkyl, fluoro substituted lower alkyl, lower alkenyl, fluoro substituted lower alkenyl, lower alkynyl, fluoro substituted lower alkynyl, lower alkoxy, fluoro substituted lower alkoxy, lower alkylthio, and fluoro substituted lower alkylthio, and wherein lower alkyl, C₃₋₆ alkenyl and C₃₋₆ alkynyl are optionally substituted with one or more substituents selected from the group consisting of fluoro, —OH, —NH₂, lower alkoxy, fluoro substituted lower alkoxy, lower alkylthio, fluoro substituted lower alkylthio, cycloalkyl, heterocycloalkyl, aryl and heteroaryl, provided, however, that any substitution on the alkyl, C₃₋₆ alkenyl or C₃₋₆ alkynyl carbon bound to the O of —OR⁹ or the S of —SR⁹ is selected from the group consisting of fluoro, cycloalkyl, heterocycloalkyl, aryl and heteroaryl, wherein cycloalkyl, heterocycloalkyl, aryl, and heteroaryl substituents of alkyl, C₃₋₆ alkenyl and C₃₋₆ alkynyl are optionally substituted with one or more substituents selected from the group consisting of halogen, —OH, —NH₂, lower alkyl, fluoro substituted lower alkyl, lower alkenyl, fluoro substituted lower alkenyl, lower alkynyl, fluoro substituted lower alkynyl, lower alkoxy, fluoro substituted lower alkoxy, lower alkylthio, and fluoro substituted lower alkylthio;
R¹⁰ is selected from the group consisting of optionally substituted cycloalkyl, optionally substituted heterocycloalkyl, optionally substituted aryl, and optionally substituted heteroaryl;
R⁵¹ and R⁵² at each occurrence are independently selected from the group consisting of hydrogen, lower alkyl, phenyl, 5-7 membered monocyclic heteroaryl, 3-7 membered monocyclic cycloalkyl, and 5-7 membered monocyclic heterocycloalkyl, wherein lower alkyl is optionally substituted with one or more substituents selected from the group consisting of fluoro, —OH, —NH₂, lower alkoxy, fluoro substituted lower alkoxy, lower alkylthio, and fluoro substituted lower alkylthio, provided, however, that any substitution on the alkyl carbon bound to the N of —NR⁵¹— or —NR⁵²— is fluoro, and wherein phenyl, monocyclic heteroaryl, monocyclic cycloalkyl and monocyclic heterocycloalkyl are optionally substituted with one or more substituents selected from the group consisting of halogen, —OH, —NH₂, lower alkyl, fluoro substituted lower alkyl, lower alkoxy, fluoro substituted lower alkoxy, lower alkylthio, and fluoro substituted lower alkylthio;
R⁵³ at each occurrence is independently selected from the group consisting of hydrogen, lower alkyl, C₃₋₆ alkenyl, provided, however, that when R⁵³ is C₃₋₆ alkenyl, no alkene carbon thereof is bound to the N of —NR⁵³—, C₃₋₆ alkynyl, provided, however, that when R⁵³ is C₃₋₆ alkynyl, no alkyne carbon thereof is bound to the N of —NR⁵³—, cycloalkyl, heterocycloalkyl, aryl, heteroaryl, —C(Z)NR¹¹R¹², —S(O)₂NR¹¹R¹², —S(O)₂R¹³, —C(Z)R¹³, and —C(Z)OR¹⁵, wherein lower alkyl, C₃₋₆ alkenyl, and C₃₋₆ alkynyl, are optionally substituted with one or more substituents selected from the group consisting of fluoro, —OR²¹, —SR²¹, —NR²²R²³, cycloalkyl, heterocycloalkyl, aryl and heteroaryl, provided, however, that any substitution on the alkyl, C₃₋₆ alkenyl or C₃₋₆ alkynyl carbon bound to the N of any —NR⁵³— is selected from the group consisting of fluoro, cycloalkyl, heterocycloalkyl, aryl, and heteroaryl, and wherein any cycloalkyl, heterocycloalkyl, aryl or heteroaryl is optionally substituted with one or more substituents selected from the group consisting of halogen, —NO₂, —CN, —OR²¹, —SR²¹, —S(O)R²¹—S(O)₂R²¹, —C(Z)R²¹, —C(Z)OR²¹, —NR²²R²³, —C(Z)NR²²R²³, —S(O)₂NR²²R²³, —C(NH)NR²²R²³, —NR²¹C(Z)R²¹, —NR²¹S(O)₂R²¹, —NR²¹C(Z)NR²²R²³, —NR²¹S(O)₂NR²²R²³, lower alkyl, lower alkenyl, and lower alkynyl, wherein the lower alkyl, lower alkenyl, and lower alkynyl optional substituents of cycloalkyl, heterocycloalkyl, aryl or heteroaryl are further optionally substituted with one or more substituents selected from the group consisting of fluoro, —OR²¹, —SR²¹, and —NR²²R²³;
R⁵⁴ at each occurrence is independently selected from the group consisting of hydrogen, lower alkyl, C₃₋₆ alkenyl, provided, however, that when R⁵⁴ is C₃₋₆ alkenyl, no alkene carbon thereof is bound to the N of any —NR⁵⁴—, C₃₋₆ alkynyl, provided, however, that when R⁵⁴ is C₃₋₆ alkynyl, no alkyne carbon thereof is bound to the N of any —NR⁵⁴—, cycloalkyl, heterocycloalkyl, aryl, and heteroaryl, wherein lower alkyl, C₃₋₆ alkenyl, and C₃₋₆ alkynyl, are optionally substituted with one or more substituents selected from the group consisting of fluoro, —OR²¹, —SR²¹, —NR²²R²³, cycloalkyl, heterocycloalkyl, aryl and heteroaryl, provided, however, that any substitution on the alkyl, C₃₋₆ alkenyl or C₃₋₆ alkynyl carbon bound to the N of any —NR⁵⁴— is selected from the group consisting of fluoro, cycloalkyl, heterocycloalkyl, aryl, and heteroaryl, and wherein any cycloalkyl, heterocycloalkyl, aryl or heteroaryl is optionally substituted with one or more substituents selected from the group consisting of halogen, —NO₂, —CN, —OR²¹, —SR²¹, —S(O)R²¹, —S(O)₂R²¹, —C(Z)R²¹, —C(Z)OR²¹, —NR²²R²³, —C(Z)NR²²R²³, —S(O)₂NR²²R²³, —C(NH)NR²²R²³, —NR²¹C(Z)R²¹, —NR²¹S(O)R²¹, —NR²¹C(Z)NR²²R²³, —NR²¹S(O)₂NR²²R²³, lower alkyl, lower alkenyl, and lower alkynyl, wherein the lower alkyl, lower alkenyl, and lower alkynyl optional substituents of cycloalkyl, heterocycloalkyl, aryl or heteroaryl are further optionally substituted with one or more substituents selected from the group consisting of fluoro, —OR²¹, —SR²¹, and —NR²²R²³;
R¹¹ and R¹² at each occurrence are independently selected from the group consisting of hydrogen, lower alkyl, C₃₋₆ alkenyl, provided, however, that when R¹¹ and/or R¹² is C₃₋₆ alkenyl, no alkene carbon thereof is bound to the N of any —C(Z)NR¹¹R¹² or —S(O)₂NR¹¹R¹², C₃₋₆ alkynyl, provided, however, that when R¹¹ and/or R¹² is C₃₋₆ alkynyl, no alkyne carbon thereof is bound to the N of any —C(Z)NR¹¹R¹² or —S(O)₂NR¹¹R¹², cycloalkyl, heterocycloalkyl, aryl, and heteroaryl, wherein lower alkyl, C₃₋₆ alkenyl, and C₃₋₆ alkynyl, are optionally substituted with one or more substituents selected from the group consisting of fluoro, —OR²¹, —SR²¹, —NR²²R²³, cycloalkyl, heterocycloalkyl, aryl and heteroaryl, provided, however, that any substitution on the alkyl, C₃₋₆ alkenyl or C₃₋₆ alkynyl carbon bound to the N of any —C(Z)NR¹¹R¹² or —S(O)₂NR¹¹R¹² is selected from the group consisting of fluoro, cycloalkyl, heterocycloalkyl, aryl, and heteroaryl, and wherein any cycloalkyl, heterocycloalkyl, aryl or heteroaryl is optionally substituted with one or more substituents selected from the group consisting of halogen, —NO₂, —CN, —OR²¹, —SR²¹, —S(O)R²¹, —S(O)₂R²¹, —C(Z)R²¹, —C(Z)OR²¹, —NR²²R²³, —C(Z)NR²²R²³, —S(O)₂NR²²R²³, —C(NH)NR²²R²³, —NR²¹C(Z)R²¹, —NR²¹(O)₂R²¹, —NR²¹C(Z)NR²²R²³, —NR²¹S(O)₂NR²²R²³, lower alkyl, lower alkenyl, and lower alkynyl, wherein the lower alkyl, lower alkenyl, and lower alkynyl optional substituents of cycloalkyl, heterocycloalkyl, aryl or heteroaryl are further optionally substituted with one or more substituents selected from the group consisting of fluoro, —OR²¹, —SR²¹, and —NR²²R²³; or
R¹¹ and R¹² together with the nitrogen to which they are attached form a 5-7 membered monocyclic heterocycloalkyl or a 5 or 7 membered monocyclic nitrogen containing heteroaryl, wherein the monocyclic heterocycloalkyl or monocyclic nitrogen containing heteroaryl is optionally substituted with one or more substituents selected from the group consisting of halogen, —OH, —NH₂, —NO₂, —CN, lower alkyl, fluoro substituted lower alkyl, lower alkoxy, fluoro substituted lower alkoxy, lower alkylthio, fluoro substituted lower alkylthio, mono-alkylamino, di-alkylamino, and cycloalkylamino;
R¹³ at each occurrence is independently selected from the group consisting of lower alkyl, C₃₋₆ alkenyl, provided, however, that when R¹³ is C₃₋₆ alkenyl, no alkene carbon thereof is bound to C(Z) of —C(Z)R¹³, or S(O)₂ of —S(O)₂R¹³, C₃₋₆ alkynyl, provided, however, that when R¹³ is C₃₋₆ alkynyl, no alkyne carbon thereof is bound to C(Z) of —C(Z)R¹³, or S(O)₂ of —S(O)₂R¹³, cycloalkyl, heterocycloalkyl, aryl, and heteroaryl, wherein lower alkyl, C₃₋₆ alkenyl, and C₃₋₆ alkynyl, are optionally substituted with one or more substituents selected from the group consisting of fluoro, —OR²¹, —SR²¹, —NR²²R²³, cycloalkyl, heterocycloalkyl, aryl and heteroaryl, and wherein any cycloalkyl, heterocycloalkyl, aryl or heteroaryl is optionally substituted with one or more substituents selected from the group consisting of halogen, —NO₂, —CN, —OR²¹, —SR²¹, —S(O)R²¹, —S(O)₂R²¹, —C(Z)R²¹, —C(Z)OR²¹, —NR²²R²³, —C(Z)NR²²R²³, —S(O)₂NR²²R²³, —C(NH)NR²²R²³, —NR²¹C(Z)R²¹, —NR²¹S(O)₂R²¹, —NR²²C(Z)NR²²R²³, —NR²¹S(O)₂NR²²R²³, lower alkyl, lower alkenyl, and lower alkynyl, wherein the lower alkyl, lower alkenyl, and lower alkynyl, optional substituents of cycloalkyl, heterocycloalkyl, aryl or heteroaryl are further optionally substituted with one or more substituents selected from the group consisting of fluoro, —OR²¹, —SR²¹, and —NR²²R²³;
R¹⁵ at each occurrence is independently selected from the group consisting of hydrogen, lower alkyl, C₃₋₆ alkenyl, provided, however, that when R¹⁵ is C₃₋₆ alkenyl, no alkene carbon thereof is bound to O of OR¹⁵, C₃₋₆ alkynyl, provided, however, that when R¹⁵ is C₃₋₆ alkynyl, no alkyne carbon thereof is bound to O of OR¹⁵, cycloalkyl, heterocycloalkyl, aryl, and heteroaryl, wherein lower alkyl, C₃₋₆ alkenyl, and C₃₋₆ alkynyl, are optionally substituted with one or more substituents selected from the group consisting of fluoro, —OR²¹, —SR²¹, —NR²²R²³, cycloalkyl, heterocycloalkyl, aryl and heteroaryl, provided, however, that any substitution on the alkyl, C₃₋₆ alkenyl or C₃₋₆ alkynyl carbon bound to the O of any OR¹⁵ is selected from the group consisting of fluoro, cycloalkyl, heterocycloalkyl, aryl, and heteroaryl, and wherein any cycloalkyl, heterocycloalkyl, aryl or heteroaryl is optionally substituted with one or more substituents selected from the group consisting of halogen, —NO₂, —CN, OR²¹, —SR²¹, —S(O)R²¹, —S(O)₂R²¹, —C(Z)R²¹, —C(Z)OR²¹—NR²²R²³, —C(Z)NR²²R²³, —S(O)₂NR²²R²³, —C(NH)NR²²R²³, —NR²¹C(Z)R²¹, —NR²¹S(O)₂R²¹, —NR²¹C(Z)NR²²R²³, —NR²¹S(O)₂NR²²R²³, lower alkyl, lower alkenyl, and lower alkynyl, wherein the lower alkyl, lower alkenyl, and lower alkynyl optional substituents of cycloalkyl, heterocycloalkyl, aryl or heteroaryl are further optionally substituted with one or more substituents selected from the group consisting of fluoro, —OR²¹, —SR²¹, and —NR²²R²³;
R¹⁶ is selected from the group consisting of hydrogen, lower alkyl, phenyl, 5-7 membered monocyclic heteroaryl, 3-7 membered monocyclic cycloalkyl, and 5-7 membered monocyclic heterocycloalkyl, wherein phenyl, monocyclic heteroaryl, monocyclic cycloalkyl and monocyclic heterocycloalkyl are optionally substituted with one or more substituents selected from the group consisting of halogen, —OH, —NH₂, lower alkyl, fluoro substituted lower alkyl, lower alkoxy, fluoro substituted lower alkoxy, lower alkylthio, and fluoro substituted lower alkylthio, and wherein lower alkyl is optionally substituted with one or more substituents selected from the group consisting of fluoro, —OH, —NH₂, lower alkoxy, fluoro substituted lower alkoxy, lower alkylthio and fluoro substituted lower alkylthio, provided, however, that when R¹⁶ is lower alkyl, any substitution on the alkyl carbon bound to the O of OR¹⁶ is fluoro;
R¹⁷ and R¹⁸ are independently selected from the group consisting of hydrogen, lower alkyl, phenyl, 5-7 membered monocyclic heteroaryl, 3-7 membered monocyclic cycloalkyl, and 5-7 membered monocyclic heterocycloalkyl, wherein phenyl, monocyclic heteroaryl, monocyclic cycloalkyl and monocyclic heterocycloalkyl are optionally substituted with one or more substituents selected from the group consisting of halogen, —OH, —NH₂, lower alkyl, fluoro substituted lower alkyl, lower alkoxy, fluoro substituted lower alkoxy, lower alkylthio, and fluoro substituted lower alkylthio, and wherein lower alkyl is optionally substituted with one or more substituents selected from the group consisting of fluoro, —OH, —NH₂, lower alkoxy, fluoro substituted lower alkoxy, lower alkylthio and fluoro substituted lower alkylthio, provided, however, that when R¹⁷ and/or R¹⁸ is lower alkyl, any substitution on the alkyl carbon bound to the N of NR¹⁷R¹⁸ is fluoro; or
R¹⁷ and R¹⁸ together with the nitrogen to which they are attached form a 5-7 membered monocyclic heterocycloalkyl or a 5 or 7 membered nitrogen containing monocyclic heteroaryl, wherein the monocyclic heterocycloalkyl or monocyclic nitrogen containing heteroaryl is optionally substituted with one or more substituents selected from the group consisting of halogen, —OH, —NH₂, lower alkyl, fluoro substituted lower alkyl, lower alkoxy, fluoro substituted lower alkoxy, lower alkylthio, and fluoro substituted lower alkylthio;
R¹⁹ and R²⁰ are independently selected from the group consisting of hydrogen, lower alkyl, lower alkenyl, lower alkynyl, cycloalkyl, heterocycloalkyl, aryl, and heteroaryl, wherein lower alkyl, lower alkenyl, and lower alkynyl, are optionally substituted with one or more substituents selected from the group consisting of fluoro, —OR²¹, —SR²², —NR²²R²³, cycloalkyl, heterocycloalkyl, aryl and heteroaryl, and wherein any cycloalkyl, heterocycloalkyl, aryl or heteroaryl is optionally substituted with one or more substituents selected from the group consisting of halogen, —NO₂, —CN, —OR²¹, —SR²¹, —S(O)R²¹, —S(O)₂R²¹, —C(Z)R²¹, —C(Z)OR²¹, —NR²²R²³, —C(Z)NR²²R²³, —S(O)₂NR²²R²³, —C(NH)NR²²R²³, —NR²¹C(Z)R²¹, —NR²¹S(O)₂R²¹, —NR²¹C(Z)NR²²R²³, —NR²¹S(O)₂NR²²R²³, lower alkyl, lower alkenyl, and lower alkynyl, wherein the lower alkyl, lower alkenyl, and lower alkynyl optional substituents of cycloalkyl, heterocycloalkyl, aryl or heteroaryl are further optionally substituted with substituents selected from the group consisting of fluoro, —OR²¹, —SR²¹, and —NR²²R²³; or
R¹⁹ and R²⁰ combine to form a 3-7 membered monocyclic cycloalkyl or 5-7 membered monocyclic heterocycloalkyl, wherein the monocyclic cycloalkyl or monocyclic heterocycloalkyl are optionally substituted with one or more substituents selected from the group consisting of halogen, —OH, —NH₂, lower alkyl, fluoro substituted lower alkyl, lower alkoxy, fluoro substituted lower alkoxy, lower alkylthio, and fluoro substituted lower alkylthio;
R²¹, R²², and R²³ at each occurrence are independently hydrogen or lower alkyl optionally substituted with one or more substituents selected from the group consisting of fluoro, lower alkoxy, fluoro substituted lower alkoxy, lower alkylthio, fluoro substituted lower alkylthio, mono-alkylamino, di-alkylamino, and cycloalkylamino provided, however, that any substitution on the lower alkyl carbon bound to O, S, or N of any of OR²¹, SR²¹, NR²¹, NR²² or NR²³ is fluoro, and further provided, however, that R²¹ bound to S, S(O), S(O)₂ or C(Z) is not hydrogen; or
R²² and R²³ together with the nitrogen to which they are attached form a 5-7 membered monocyclic heterocycloalkyl or a 5 or 7 membered monocyclic nitrogen containing heteroaryl, wherein the monocyclic heterocycloalkyl or monocyclic nitrogen containing heteroaryl is optionally substituted with one or more substituents selected from the group consisting of halogen, —OH, —NH₂, —NO₂, —CN, lower alkyl, fluoro substituted lower alkyl, lower alkoxy, fluoro substituted lower alkoxy, lower alkylthio, fluoro substituted lower alkylthio, mono-alkylamino, di-alkylamino, and cycloalkylamino;
Z is O or S;
m is 1, 2, 3, or 4;
n is 1 or 2;
p is 0 or 1, provided, however, that when p is 1, m is 1, and L is —O—, —S—, —NR⁵²—, —C(Z)NR⁵²—, —S(O)₂NR⁵²—, —NR⁵²C(Z)NR⁵²—, or NR⁵²S(O)₂NR⁵²—, then Y is not —O—, —S—, —NR⁵³—, —NR⁵⁴C(Z)-, —NR⁵⁴S(O)₂—, —NR⁵⁴C(Z)NR⁵⁴—, or NR⁵⁴S(O)₂NR⁵⁴—; and
r is 0 or 1.

In one embodiment of the compounds of Formula I, at least one of R¹ and R² is other than hydrogen. In one embodiment, one of R¹ and R² is other than hydrogen and the other of R¹ and R² is hydrogen or halogen. In one embodiment, one of R¹ and R² is other than hydrogen and the other of R¹ and R² is hydrogen. In one embodiment, at least one of R¹ and R² is —SR⁹ or —OR⁹, preferably —OR⁹. In one embodiment, one of R¹ and R² is —SR⁹ or —OR⁹, preferably —OR⁹, and the other of R¹ and R² is hydrogen or halogen. In one embodiment, one of R¹ and R² is —SR⁹ or —OR⁹, preferably —OR⁹, and the other of R¹ and R² is hydrogen. In one embodiment, R¹ and R² are both hydrogen.

In one embodiment of the compounds of Formula I, at least one of R¹ and R² is —SR⁹ or —OR⁹, preferably —OR⁹, wherein R⁹ is selected from the group consisting of lower alkyl, C₃₋₆ alkenyl and C₃₋₆ alkynyl, wherein lower alkyl, C₃₋₆ alkenyl and C₃₋₆ alkynyl are optionally substituted with one or more substituents selected from the group consisting of fluoro, —OH, —NH₂, lower alkoxy, fluoro substituted lower alkoxy, lower alkylthio, and fluoro substituted lower alkylthio. In one embodiment, at least one of R¹ and R² is —SR⁹ or —OR⁹, preferably —OR⁹, wherein R⁹ is lower alkyl optionally substituted with one or more substituents selected from the group consisting of fluoro, —OH, lower alkoxy, and lower alkylthio.

In one embodiment of the compounds of Formula I, at least one of R¹ and R² is halogen, lower alkyl, or C₃₋₆ cycloalkyl, wherein lower alkyl is optionally substituted with one or more substituents selected from the group consisting of fluoro, —OH, —NH₂, lower alkoxy, fluoro substituted lower alkoxy, lower alkylthio, fluoro substituted lower alkylthio, and C₃₋₆ cycloalkyl, wherein C₃₋₆ cycloalkyl, as R¹, R² or a substituent of lower alkyl, is optionally substituted with one or more substituents selected from the group consisting of halogen, —OH, —NH₂, lower alkyl, fluoro substituted lower alkyl, lower alkoxy, fluoro substituted lower alkoxy, lower alkylthio, and fluoro substituted lower alkylthio, preferably one of R¹ and R² is hydrogen, preferably R¹ is hydrogen and R² is fluoro, chloro, lower alkyl, fluoro substituted lower alkyl, C₃₋₆ cycloalkyl, or fluoro substituted C₃₋₆ cycloalkyl.

In one embodiment of compounds of Formula I, W is —(CR⁴R⁵)₁₋₃— or —CR⁶═CR⁷—. In a preferred embodiment, W is —CH₂CH₂— or —CH₂—, more preferably —CH₂—, further wherein X is —COOH. In one embodiment, W is —(CH₂)₁₋₃— and at least one of R¹ and R² is —OR⁹, wherein R⁹ is lower alkyl optionally substituted with one or more substituents selected from the group consisting of fluoro, —OH, lower alkoxy, and lower alkylthio. In one embodiment, W is —CH₂CH₂— or —CH₂—, more preferably —CH₂—, X is —COOH and at least one of R¹ and R² is —OR⁹, wherein R⁹ is lower alkyl optionally substituted with one or more substituents selected from the group consisting of fluoro, —OH, lower alkoxy, and lower alkylthio.

In one embodiment of the compounds of Formula I, L is selected from the group consisting of —O—, —S—, —NR⁵²—, —C(Z)-, —S(O)ₙ—, —C(Z)NR⁵²—, —NR⁵²C(Z)-, —NR⁵²S(O)₂—, and —S(O)₂NR⁵²—, where L is preferably —O— or —S(O)₂—, more preferably —S(O)₂—.

In one embodiment of the compounds of Formula I, L is selected from the group consisting of —O—, —S—, —NR⁵²—, —C(Z)-, —S(O)ₙ—, —C(Z)NR⁵²—, —NR⁵²C(Z)-, —NR⁵²S(O)₂—, and —S(O)₂NR⁵²—, preferably —O— or —S(O)₂—, more preferably —S(O)₂—, and at least one of R¹ and R² is —SR⁹ or —OR⁹, preferably —OR⁹, wherein R⁹ is lower alkyl optionally substituted with one or more substituents selected from the group consisting of fluoro, —OH, lower alkoxy, and lower alkylthio.

In one embodiment of the compounds of Formula I, L is selected from the group consisting of —O—, —S—, —NR⁵²—, —C(Z)-, —S(O)ₙ—, —C(Z)NR⁵²—, —NR⁵²C(Z)-, —NR⁵²S(O)₂—, and —S(O)₂NR⁵²—, preferably —O— or —S(O)₂—, more preferably —S(O)₂—, and W is —(CR⁴R⁵)₁₋₃— or —CR⁶═CR⁷—, preferably —CH₂CH₂— or —CH₂—, more preferably —CH₂—.

In one embodiment of the compounds of Formula I, L is selected from the group consisting of —O—, —S—, —NR⁵²—, —C(Z)-, —S(O)ₙ—, —C(Z)NR⁵²—, —NR⁵²C(Z)-, —NR⁵²S(O)₂—, and —S(O)₂NR⁵²—, preferably —O— or —S(O)₂—, more preferably —S(O)₂—, and —R³ is —R¹⁰ or —Ar₁-M-Ar₂.

In one embodiment of the compounds of Formula I, L is selected from the group consisting of —O—, —S—, —NR⁵²—, —C(Z)-, —S(O)ₙ—, —C(Z)NR⁵²—, —NR⁵²C(Z)-, —NR⁵²S(O)₂—, and —S(O)₂NR⁵²—, preferably —O— or —S(O)₂—, more preferably —S(O)₂—, W is —(CH₂)₁₋₃—, preferably —CH₂CH₂— or —CH₂—, more preferably —CH₂—, and at least one of R¹ and R² is —SR⁹ or —OR⁹, preferably —OR⁹, wherein R⁹ is lower alkyl optionally substituted with one or more substituents selected from the group consisting of fluoro, —OH, lower alkoxy, and lower alkylthio, further wherein X is preferably —C(O)OR¹⁶ or a carboxylic acid isostere, more preferably wherein X is —C(O)OH.

In one embodiment of the compounds of Formula I, L is selected from the group consisting of —O—, —S—, —NR⁵²—, —C(Z)-, —S(O)ₙ—, —C(Z)NR⁵²—, —NR⁵²C(Z)-, —NR⁵²S(O)₂—, and —S(O)₂NR⁵²—, preferably —O— or —S(O)₂—, more preferably —S(O)₂—; W is —(CH₂)₁₋₃—, preferably —CH₂CH₂— or —CH₂—, more preferably —CH₂—, and at least one of R¹ and R² is halogen, lower alkyl, or C₃₋₆ cycloalkyl, wherein lower alkyl is optionally substituted with one or more substituents selected from the group consisting of fluoro, —OH, —NH₂, lower alkoxy, fluoro substituted lower alkoxy, lower alkylthio, fluoro substituted lower alkylthio, and C₃₋₆ cycloalkyl, wherein C₃₋₆ cycloalkyl, as R¹, R² or a substituent of lower alkyl, is optionally substituted with one or more substituents selected from the group consisting of halogen, —OH, —NH₂, lower alkyl, fluoro substituted lower alkyl, lower alkoxy, fluoro substituted lower alkoxy, lower alkylthio, and fluoro substituted lower alkylthio, preferably one of R¹ and R² is hydrogen, preferably R¹ is hydrogen and R² is fluoro, chloro, lower alkyl, fluoro substituted lower alkyl, C₃₋₆ cycloalkyl, or fluoro substituted C₃₋₆ cycloalkyl; further wherein X is preferably —C(O)OR¹⁶ or a carboxylic acid isostere, more preferably wherein X is —C(O)OH.

In one embodiment of the compounds of Formula I, L is selected from the group consisting of —O—, —S—, —NR⁵²—, —C(Z)-, —S(O)ₙ—, —C(Z)NR⁵²—, —NR⁵²C(Z)-, —NR⁵²S(O)₂—, and —S(O)₂NR⁵²—, preferably —O— or —S(O)₂—, more preferably —S(O)₂—, W is —(CH₂)₁₋₃—, preferably —CH₂CH₂— or —CH₂—, more preferably —CH₂—, —R³ is —R¹⁰ or —Ar₁-M-Ar₂, and at least one of R¹ and R² is —SR⁹ or —OR⁹, preferably —OR⁹, wherein R⁹ is lower alkyl optionally substituted with one or more substituents selected from the group consisting of fluoro, —OH, lower alkoxy, and lower alkylthio, further wherein X is preferably —C(O)OR¹⁶ or a carboxylic acid isostere, more preferably wherein X is —C(O)OH.

In one embodiment of the compounds of Formula I, L is selected from the group consisting of —O—, —S—, —NR⁵²—, —C(Z)-, —S(O)ₙ—, —C(Z)NR⁵²—, —NR⁵²C(Z)-, —NR⁵²S(O)₂—, and —S(O)₂NR⁵²—, preferably —O— or —S(O)₂—, more preferably —S(O)₂—; W is —(CH₂)₁₋₃—, preferably —CH₂CH₂— or —CH₂—, more preferably —CH₂—, —R³ is —R¹⁰ or —Ar₁-M-Ar₂, and at least one of R¹ and R² is halogen, lower alkyl, or C₃₋₆ cycloalkyl, wherein lower alkyl is optionally substituted with one or more substituents selected from the group consisting of fluoro, —OH, —NH₂, lower alkoxy, fluoro substituted lower alkoxy, lower alkylthio, fluoro substituted lower alkylthio, and C₃₋₆ cycloalkyl, wherein C₃₋₆ cycloalkyl, as R¹, R² or a substituent of lower alkyl, is optionally substituted with one or more substituents selected from the group consisting of halogen, —OH, —NH₂, lower alkyl, fluoro substituted lower alkyl, lower alkoxy, fluoro substituted lower alkoxy, lower alkylthio, and fluoro substituted lower alkylthio, preferably one of R¹ and R² is hydrogen, preferably R¹ is hydrogen and R² is fluoro, chloro, lower alkyl, fluoro substituted lower alkyl, C₃₋₆ cycloalkyl, or fluoro substituted C₃₋₆ cycloalkyl, further wherein X is preferably —C(O)OR¹⁶ or a carboxylic acid isostere, more preferably wherein X is —C(O)OH.

In one embodiment of the compounds of Formula I, L is selected from —S(O)₂—, —NR⁵²S(O)₂—, and —S(O)₂NR⁵²—, preferably —S(O)₂—; W is —(CH₂)₁₋₃—, preferably —CH₂CH₂— or —CH₂—, more preferably —CH₂—, and at least one of R¹ and R² is —SR⁹ or —OR⁹, preferably —OR⁹, wherein R⁹ is lower alkyl optionally substituted with one or more substituents selected from the group consisting of fluoro, —OH, lower alkoxy, and lower alkylthio, further wherein X is preferably —C(O)OR¹⁶ or a carboxylic acid isostere, more preferably wherein X is —C(O)OH.

In one embodiment of the compounds of Formula I, L is selected from —S(O)₂—, —NR⁵²S(O)₂—, and —S(O)₂NR⁵²—, preferably —S(O)₂—, W is —(CH₂)₁₋₃—, preferably —CH₂CH₂— or —CH₂—, more preferably —CH₂—, —R³ is —R¹⁰ or —Ar₁-M-Ar₂, and at least one of R¹ and R² is —SR⁹ or —OR⁹, preferably —OR⁹, wherein R⁹ is lower alkyl optionally substituted with one or more substituents selected from the group consisting of fluoro, —OH, lower alkoxy, and lower alkylthio, further wherein X is preferably —C(O)OR¹⁶ or a carboxylic acid isostere, more preferably wherein X is —C(O)OH.

In one embodiment of compounds of Formula I, L is —O— and —R³ is —[(CR⁴R⁵)ₘ—(Y)ₚ]ᵣ—Ar₁-M-Ar₂. In one embodiment of compounds of Formula I, L is —O—, and —R³ is R¹⁰, wherein R¹⁰ is optionally substituted phenyl. In one embodiment of compounds of Formula I, L is —O—, and —R³ is R¹⁰, wherein R¹⁰ is phenyl optionally substituted with one or more substituents selected from the group consisting of fluoro, —OH, —NH₂, lower alkyl, fluoro substituted lower alkyl (e.g., CF₃ or CF₂CF₃), lower alkoxy, fluoro substituted lower alkoxy (e.g., OCF₃ or OCF₂CF₃), lower alkylthio, and fluoro substituted lower alkylthio (e.g., SCF₃ or SCF₂CF₃).

In one embodiment of compounds of Formula I, L is —S(O)₂— and —R³ is —[(CR⁴R⁵)ₘ—(Y)ₚ]ᵣ—Ar₁-M-Ar₂. In one embodiment of compounds of Formula I, L is —S(O)₂—, and —R³ is R¹⁰, wherein R¹⁰ is optionally substituted phenyl. In one embodiment of compounds of Formula I, L is —S(O)₂—, and —R³ is R¹⁰, wherein R¹⁰ is phenyl optionally substituted with one or more substituents selected from the group consisting of fluoro, —OH, —NH₂, lower alkyl, fluoro substituted lower alkyl (e.g., CF₃ or CF₂CF₃), lower alkoxy, fluoro substituted lower alkoxy (e.g., OCF₃ or OCF₂CF₃), lower alkylthio, and fluoro substituted lower alkylthio (e.g., SCF₃ or SCF₂CF₃).

In one embodiment of compounds of Formula I, L is —S(O)₂—, and —R³ is R¹⁰, wherein R¹⁰ is optionally substituted phenyl, W is —(CH₂)₁₋₃—, preferably —CH₂CH₂— or —CH₂—, more preferably —CH₂—, and at least one of R¹ and R² is —SR⁹ or —OR⁹, preferably —OR⁹, wherein R⁹ is lower alkyl optionally substituted with one or more substituents selected from the group consisting of fluoro, —OH, lower alkoxy, and lower alkylthio, further wherein X is preferably —C(O)OR¹⁶ or a carboxylic acid isostere, more preferably wherein X is —C(O)OH.

In one embodiment, relative to any of the above embodiments, when L is —S(O)₂NR⁵²—, R⁵² is hydrogen, and R² is hydrogen, R¹ is other than —OCH₃. In one embodiment, relative to any of the above embodiments, when L is —S(O)₂NR⁵²—, R¹ is hydrogen.

In one embodiment, relative to any of the above embodiments, compounds are excluded wherein L is —O— or —S—, r=1, p=0, m is 1, 2, 3 or 4, and —R¹⁰ or —Ar₁— is optionally substituted pyrazolyl, optionally substituted imidazolyl, optionally substituted isoxazolyl, optionally substituted oxazolyl, optionally substituted thiazolyl, or optionally substituted isothiazolyl; compounds are also excluded wherein L is —O—, R³ is —R¹⁰ or —(CR⁴R⁵)ₘ—R¹⁰, and —R¹⁰ has a structure of

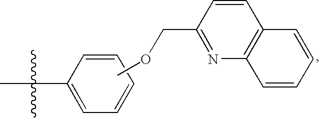

wherein

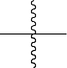

indicates the attachment point to L or —(CR⁴R⁵)ₘ— and wherein the phenyl or quinolinyl rings of R¹⁰ are optionally substituted; compounds are also excluded wherein L is —O— and R³ has a structure of

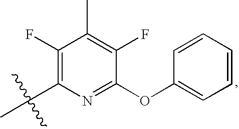

wherein the phenyl ring is optionally substituted and wherein

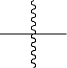

indicates the attachment point to L; the following compounds are also excluded:

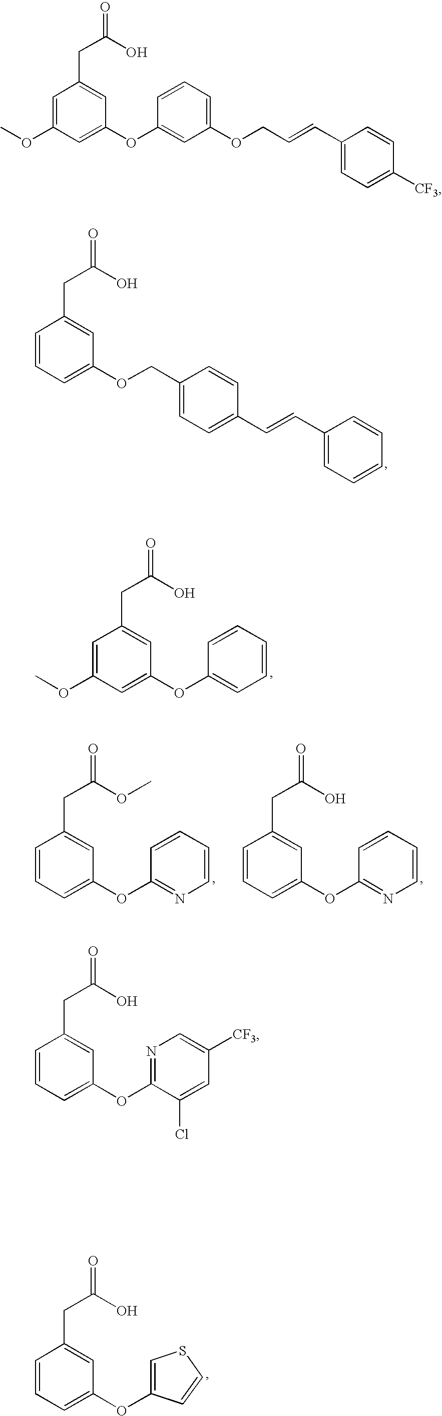

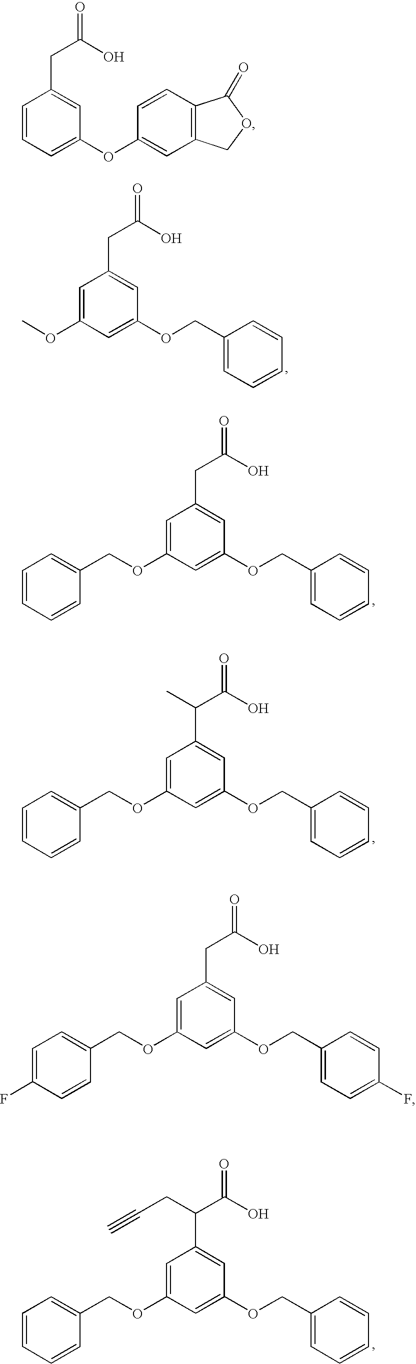

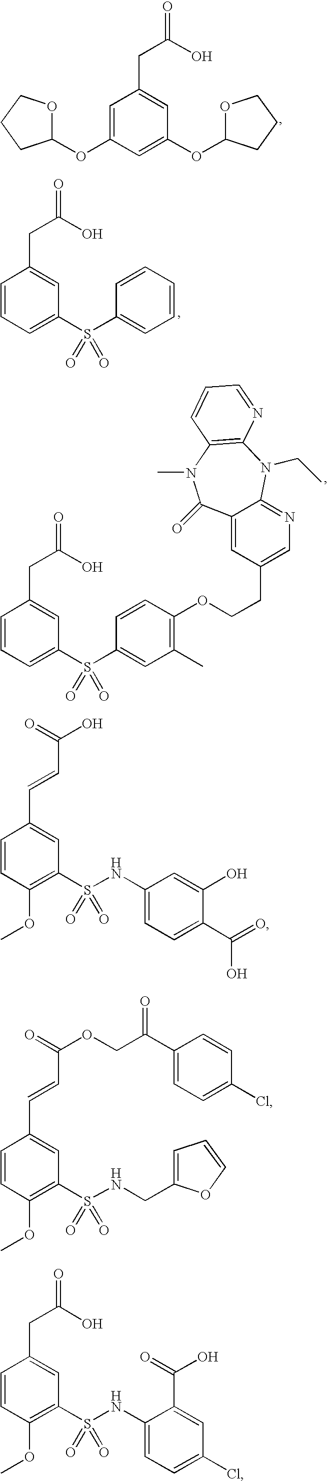

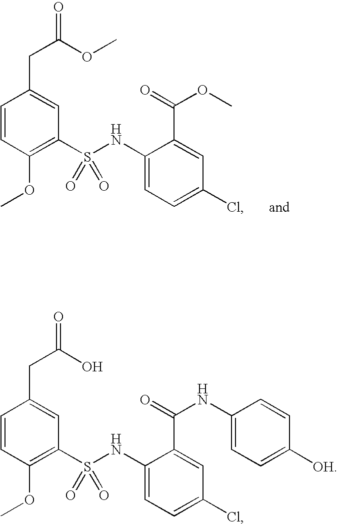

In another embodiment, relative to any of the above embodiments, compounds are excluded where LR³ is any of the following, wherein

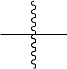

indicates the point of attachment of L to the benzene ring of Formula I:

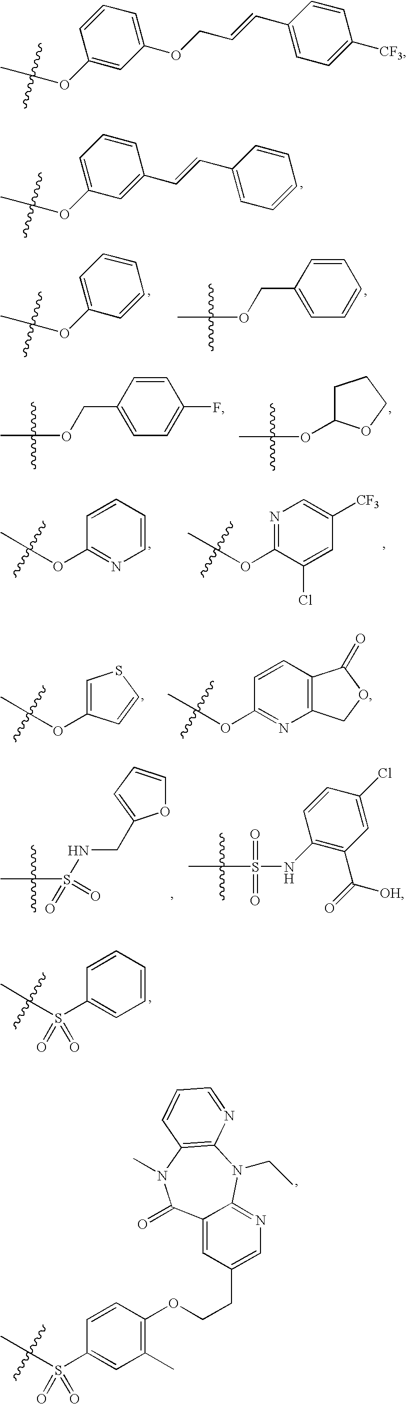

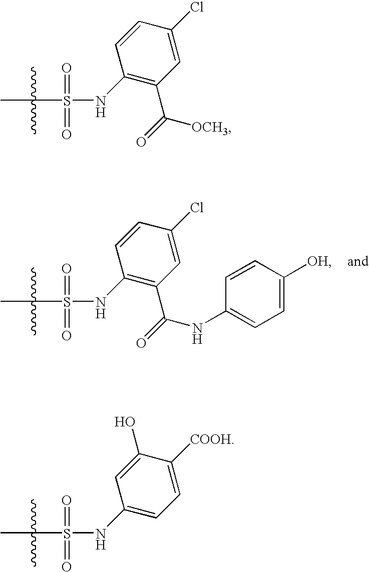

In one embodiment, compounds of Formula I have the following sub-generic structure (Formula Ia):

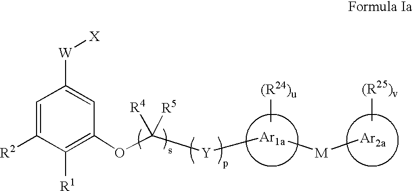

all salts, prodrugs, tautomers, and isomers thereof,
wherein:
W, X, R¹, R², R⁴, R⁵, Y, M, and p are as defined for Formula I;
Ar₁ₐ is selected from the group consisting of arylene and heteroarylene;
Ar₂ₐ is selected from the group consisting of aryl and heteroaryl;
R²⁴ at each occurrence is independently selected from the group consisting of halogen, lower alkyl, lower alkenyl, lower alkynyl, —NO₂, —CN, —OR²⁶, —SR²⁶, —OC(O)R²⁶, —OC(S)R²⁶—C(O)R²⁶, —C(S)R²⁶, —C(O)OR²⁶, —C(S)OR²⁶, —S(O)R²⁶, —S(O)₂R²⁶, —C(O)NR²⁷R²⁸, —C(S)NR²⁷R²⁸, —S(O)₂NR²⁷R²⁸, —C(NH)NR²⁷R²⁸, —NR²⁶C(O)R²⁶, —NR²⁶C(S)R²⁶, —NR²⁶S(O)₂R²⁶, NR²⁶C(O)NR²⁷R²⁸, NR²⁶C(s)NR²⁷R²⁸, —NR²⁶S(O)₂NR²⁷R²⁸, and —NR²⁷R²⁸, wherein lower alkyl is optionally substituted with one or more substituents selected from the group consisting of fluoro, —OR³⁶, —SR³⁶, and —NR³⁷R³⁸, and wherein lower alkenyl and lower alkynyl are optionally substituted with one or more substituents selected from the group consisting of fluoro, —OR³⁶, —SR³⁶, —NR³⁷R³⁸, and —R³⁵;
R²⁵ at each occurrence is independently selected from the group consisting of halogen, lower alkyl, lower alkenyl, lower alkynyl, cycloalkyl, heterocycloalkyl, aryl, heteroaryl, —NO₂, —CN, —OR²⁹, —SR²⁹, —OC(O)R²⁹, —OC(S)R²⁹, —C(O)R²⁹, —C(S)R²⁹, —C(O)OR²⁹, —C(S)OR²⁹, —S(O)R²⁹, —S(O)₂R²⁹, —C(O)NR²⁹R²⁹, —C(S)NR²⁹R²⁹, —S(O)₂NR²⁹R²⁹, —C(NH)NR³⁰R³¹, —NR²⁹C(O)R²⁹, —NR²⁹C(S)R²⁹, —NR²⁹S(O)₂R²⁹, —NR²⁹C(O)NR²⁹R²⁹, —NR²⁹C(S)NR²⁹R²⁹, —NR²⁹S(O)₂NR²⁹R²⁹, and —NR²⁹R²⁹, wherein lower alkyl is optionally substituted with one or more substituents selected from the group consisting of fluoro, —OR³⁶, —SR³⁶, —NR³⁷R³⁸, and —R³², and wherein lower alkenyl and lower alkynyl are optionally substituted with one or more substituents selected from the group consisting of fluoro, —OR³⁶, —SR³⁶, —NR³⁷R³⁸, —R³⁵ and —R³², and wherein cycloalkyl, heterocycloalkyl, aryl, and heteroaryl are optionally substituted with one or more substituents selected from the group consisting of halogen, —NO₂, —CN, —OR³⁶, —SR³⁶, —NR³⁷R³⁸, —R³⁵, —R³³ and —R³⁴;
R²⁶, R²⁷ and R²⁸ at each occurrence are independently selected from the group consisting of hydrogen, lower alkyl, C₃₋₆ alkenyl, provided, however, that no alkene carbon thereof is bound to any O, S, N, C(O), C(S), S(O) or S(O)₂ of R²⁴, and C₃₋₆ alkynyl, provided, however, that no alkyne carbon thereof is bound to any O, S, N, C(O), C(S), S(O) or S(O)₂ of R²⁴, wherein lower alkyl is optionally substituted with one or more substituents selected from the group consisting of fluoro, —OR³⁶, —SR³⁶, and —NR³⁷R³⁸, and wherein lower alkenyl and lower alkynyl are optionally substituted with one or more substituents selected from the group consisting of fluoro, —OR³⁶, —SR³⁶, —NR³⁷R³⁸, and —R³⁵, further provided, however, that R²⁶ bound to S, C(O), C(S), S(O), or S(O)₂ is not hydrogen, or
R²⁷ and R²⁸ combine with the nitrogen to which they are attached to form cycloalkylamino;
R²⁹, R³⁰ and R³¹ at each occurrence are independently selected from the group consisting of hydrogen, lower alkyl, C₃₋₆ alkenyl, provided, however, that no alkene carbon thereof is bound to any O, S, N, C(O), C(S), S(O) or S(O)₂ of R²⁵, C₃₋₆ alkynyl, provided, however, that no alkyne carbon thereof is bound to any O, S, N, C(O), C(S), S(O) or S(O)₂ of R²⁵, cycloalkyl, heterocycloalkyl, aryl and heteroaryl, or
R³⁰ and R³¹ combine with the nitrogen to which they are attached to form a 5-7 membered heterocycloalkyl or a 5 or 7 membered nitrogen containing heteroaryl, wherein lower alkyl is optionally substituted with one or more substituents selected from the group consisting of fluoro, —OR³⁶, —SR³⁶, —NR³⁷R³⁸, and R³², and wherein lower alkenyl and lower alkynyl are optionally substituted with one or more substituents selected from the group consisting of fluoro, —OR³⁶, —SR³⁶, —NR³⁷R³⁸, —R³⁵ and —R³², and wherein cycloalkyl, heterocycloalkyl, aryl, heteroaryl, 5-7 membered heterocycloalkyl, and 5 or 7 membered nitrogen containing heteroaryl are optionally substituted with one or more substituents selected from the group consisting of halogen, —NO₂, —CN, —OH, —NH₂, —OR³⁶, —SR³⁶, —NHR³⁶, —NR³⁷R³⁸, —R³³, —R³⁴, and —R³⁵, further provided, however, that R²⁹ bound to S, C(O), C(S), S(O), or S(O)₂ is not hydrogen;
R³² at each occurrence is independently selected from the group consisting of cycloalkyl, heterocycloalkyl, aryl and heteroaryl, wherein cycloalkyl, heterocycloalkyl, aryl and heteroaryl are optionally substituted with one or more substituents selected from the group consisting of halogen, —NO₂, —CN, —OR³⁶, —R³⁶, —NR³⁷R³⁸, —R³³, —R³⁴, and —R³⁵;
R³³ at each occurrence is independently lower alkenyl optionally substituted with one or more substituents selected from the group consisting of fluoro, —OR³⁶, —SR³⁶, —NR³⁷R³⁸ and —R³⁵;
R³⁴ at each occurrence is independently lower alkynyl optionally substituted with one or more substituents selected from the group consisting of fluoro, —OR³⁶, —SR³⁶, —NR³⁷R³⁸, and —R³⁵;
R³⁵ at each occurrence is independently lower alkyl optionally substituted with one or more substituents selected from the group consisting of fluoro, —OR³⁶, —SR³⁶, and —NR³⁷R³⁸;
R³⁶, R³⁷ and R³⁸ at each occurrence is independently hydrogen or lower alkyl optionally substituted with one or more substituents selected from the group consisting of fluoro, lower alkoxy, fluoro substituted lower alkoxy, lower alkylthio, fluoro substituted lower alkylthio, mono-alkylamino, di-alkylamino, and cycloalkylamino, or —NR³⁷R³⁸ is cycloalkylamino, provided, however, that any substitution on the lower alkyl carbon bound to the O, S, or N of any of OR³⁶, SR³⁶, NR³⁶, NR³⁷ or NR³⁸ is fluoro, and further provided, however, that R³⁶ bound to S is not hydrogen;
u is 0, 1, 2, 3 or 4;
v is 0, 1, 2, 3, 4, or 5;
s is 0, 1, 2, 3 or 4, provided, however, that when s=0, then p=0 and when s is 1, 2, 3, or 4 and p=0, then Ar₁ₐ is not pyrazolyl, imidazolyl, isoxazolyl, oxazolyl, thiazolyl, or isothiazolyl, and when s=0, p=0, and Ar₂ₐ is phenyl,

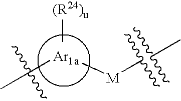

is not

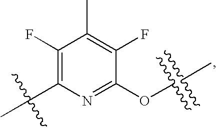

wherein

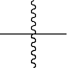

indicates the attachment point to O and

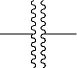

indicates the attachment point to Ar₂ₐ.

In one embodiment, compounds of Formula I have the following sub-generic structure (Formula Ib):

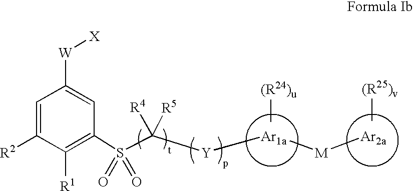

all salts, prodrugs, tautomers, and isomers thereof,
wherein:
W, X, R¹, R², R⁴, R⁵, Y, M, and p are as defined for Formula I;
Ar₁ₐ, Ar₂ₐ, R²⁴, R²⁵, u and v are as defined for Formula Ia; and
t is 0, 1, 2, 3 or 4, provided, however, that when t=0, then p=0.

In one embodiment of the compounds of Formulae Ia or Ib, at least one of R¹ and R² is other than hydrogen. In one embodiment, one of R¹ and R² is other than hydrogen and the other of R¹ and R² is hydrogen or halogen. In one embodiment, one of R¹ and R² is other than hydrogen and the other of R¹ and R² is hydrogen. In one embodiment, at least one of R¹ and R² is —SR⁹ or —OR⁹, preferably —OR⁹. In one embodiment, one of R¹ and R² is —SR⁹ or —OR⁹, preferably —OR⁹, and the other of R¹ and R² is hydrogen or halogen. In one embodiment, one of R¹ and R² is —SR⁹ or —OR⁹, preferably —OR⁹, and the other of R¹ and R² is hydrogen. In one embodiment, R² is —SR⁹ or —OR⁹, preferably —OR⁹, and R² is hydrogen. In one embodiment, R² is —SR⁹ or —OR⁹, preferably —OR⁹, and R¹ is hydrogen. In one embodiment, both R¹ and R² are hydrogen.

In one embodiment of the compounds of Formulae Ia or Ib, at least one of R¹ and R² is halogen, lower alkyl, or C₃₋₆ cycloalkyl, wherein lower alkyl is optionally substituted with one or more substituents selected from the group consisting of fluoro, —OH, —NH₂, lower alkoxy, fluoro substituted lower alkoxy, lower alkylthio, fluoro substituted lower alkylthio, and C₃₋₆ cycloalkyl, wherein C₃₋₆ cycloalkyl, as R¹, R² or a substituent of lower alkyl, is optionally substituted with one or more substituents selected from the group consisting of halogen, —OH, —NH₂, lower alkyl, fluoro substituted lower alkyl, lower alkoxy, fluoro substituted lower alkoxy, lower alkylthio, and fluoro substituted lower alkylthio, preferably one of R¹ and R² is hydrogen, preferably R¹ is hydrogen and R² is fluoro, chloro, lower alkyl, fluoro substituted lower alkyl, C₃₋₆ cycloalkyl, or fluoro substituted C₃₋₆ cycloalkyl.

In one embodiment of the compounds of Formulae Ia or Ib, one of R¹ and R², preferably R², is —SR⁹ or —OR⁹, preferably —OR⁹, the other of R¹ and R², preferably R¹, is hydrogen, and R⁹ is selected from the group consisting of lower alkyl, C₃₋₆ alkenyl, C₃₋₆ alkynyl, and cycloalkyl, wherein lower alkyl, C₃₋₆ alkenyl, C₃₋₆ alkynyl, and cycloalkyl are optionally substituted as described for R⁹ in Formula I. In one embodiment, one of R¹ and R², preferably R², is —SR⁹ or —OR⁹, preferably —OR⁹, the other of R¹ and R², preferably R¹, is hydrogen, and R⁹ is selected from the group consisting lower alkyl, C₃₋₆ alkenyl, C₃₋₆ alkynyl, and cycloalkyl, wherein cycloalkyl is optionally substituted with one or more substituents selected from the group consisting of fluoro, —OH, lower alkyl, fluoro substituted lower alkyl, lower alkoxy, fluoro substituted lower alkoxy, lower alkylthio, and fluoro substituted lower alkylthio, wherein lower alkyl, C₃₋₆ alkenyl, and C₃₋₆ alkynyl are optionally substituted with one or more substituents selected from the group consisting of fluoro, —OH, lower alkoxy, fluoro substituted lower alkoxy, lower alkylthio, fluoro substituted lower alkylthio, and cycloalkyl, wherein the cycloalkyl substituent of alkyl, C₃₋₆ alkenyl, or C₃₋₆ alkynyl is optionally substituted with one or more substituents selected from the group consisting of fluoro, —OH, lower alkyl, fluoro substituted lower alkyl, lower alkoxy, fluoro substituted lower alkoxy, lower alkylthio, and fluoro substituted lower alkylthio. In one embodiment, one of R¹ and R², preferably R², is —SR⁹ or —OR⁹, preferably —OR⁹, the other of R¹ and R², preferably R¹, is hydrogen, and R⁹ is selected from the group consisting of lower alkyl, C₃₋₆ alkenyl, C₃₋₆ alkynyl, and cycloalkyl, wherein the lower alkyl, C₃₋₆ alkenyl, C₃₋₆ alkynyl, and cycloalkyl are optionally substituted with one or more substituents selected from the group consisting of fluoro, lower alkoxy, and lower alkylthio. In one embodiment, one of R¹ and R², preferably R², is —SR⁹ or —OR⁹, preferably —OR⁹, the other of R¹ and R², preferably R¹, is hydrogen, and R⁹ is lower alkyl optionally substituted with one or more substituents selected from the group consisting of fluoro, lower alkoxy, fluoro substituted lower alkoxy, lower alkylthio, fluoro substituted lower alkylthio, cycloalkyl, and fluoro substituted cycloalkyl. In one embodiment, one of R¹ and R², preferably R², is —SR⁹ or —OR⁹, preferably —OR⁹, the other of R¹ and R², preferably R¹, is hydrogen, and R⁹ is lower alkyl optionally substituted with one or more substituents selected from the group consisting of fluoro, lower alkoxy, and lower alkylthio.

In one embodiment of compounds of Formulae Ia or Ib, W is selected from the group consisting of —NR⁵¹(CR⁴R⁵)₁₋₂—, —O—(CR⁴R⁵)₁₋₂—, —S—(CR⁴R⁵)₁₋₂—, —(CR⁴R⁵)₁₋₃—, and —CR⁶═CR⁷—, wherein R⁵¹ is hydrogen or lower alkyl optionally substituted with one or more substituents selected from the group consisting of fluoro, lower alkoxy, fluoro substituted lower alkoxy, lower alkylthio, and fluoro substituted lower alkylthio, and wherein R⁴, R⁵, R⁶ and R⁷ are independently hydrogen or lower alkyl optionally substituted with one or more substituents selected from the group consisting of fluoro, lower alkoxy, fluoro substituted lower alkoxy, lower alkylthio, and fluoro substituted lower alkylthio. In one embodiment W is selected from the group consisting of —(CR⁴R⁵)₁₋₃—, and —CR⁶═CR⁷—. In one embodiment, W is —(CR⁴R⁵)₁₋₂—. In one embodiment, W is —(CR⁴R⁵)—. In one embodiment, W is selected from the group consisting of —(CR⁴R⁵)₁₋₃— and —CR⁶═CR⁷—, wherein R⁴, R⁵, R⁶ and R⁷ are independently hydrogen or lower alkyl optionally substituted with one or more substituents selected from the group consisting of fluoro, lower alkoxy, fluoro substituted lower alkoxy, lower alkylthio, and fluoro substituted lower alkylthio. In one embodiment, W is —(CR⁴R⁵)₁₋₂—, preferably —(CR⁴R⁵)—, wherein R⁴ and R⁵ are independently hydrogen or lower alkyl optionally substituted with one or more substituents selected from the group consisting of fluoro, lower alkoxy, fluoro substituted lower alkoxy, lower alkylthio, and fluoro substituted lower alkylthio. In one embodiment, W is —CH₂CH₂— or —CH₂—, preferably —CH₂—.

In one embodiment of compounds of Formulae Ia or Ib, X is —C(O)OR¹⁶ or a carboxylic acid isostere, preferably X is —COOH. In one embodiment, W is —(CR⁴R⁵)₁₋₂— and X is —C(O)OR¹⁶ or a carboxylic acid isostere, preferably W is —CH₂CH₂— or —CH₂— and X is —COOH.

In one embodiment of compounds of Formulae Ia or Ib, p is 0. In one embodiment of compounds of Formula Ia, Ar₁ₐ is selected from the group consisting of phenyl, pyridinyl, pyrimidinyl, and thiophenyl. In one embodiment of compounds of Formula Ib, Ar₁ₐ is selected from the group consisting of phenyl, pyridinyl, pyrimidinyl, thiophenyl, oxazolyl, isoxazolyl, thiazolyl, isothiazolyl, imidazolyl, and pyrazolyl. In one embodiment of compounds of Formulae Ia or Ib, Ar₁ₐ is selected from the group consisting of phenyl, pyridinyl, oxazolyl, isoxazolyl, thiazolyl, isothiazolyl, imidazolyl, and pyrazolyl, preferably phenyl, pyridinyl, oxazolyl, and thiazolyl.

In one embodiment of compounds of Formulae Ia or Ib, R²⁴ is selected from the group consisting of halogen, lower alkyl, lower alkenyl, lower alkynyl, lower alkoxy and lower alkylthio, wherein lower alkyl, lower alkenyl, lower alkynyl, lower alkoxy or lower alkylthio are optionally substituted with one or more substituents selected from the group consisting of fluoro, —OR³⁶, —SR³⁶, and —NR³⁷R³⁸, where R³⁶, R³⁷ and R³⁸ are as defined in Formulae Ia and Ib. In one embodiment, R²⁴ is selected from the group consisting of halogen, lower alkyl, lower alkoxy, and lower alkylthio, wherein lower alkyl, lower alkoxy and lower alkylthio are optionally substituted with one or more substituents selected from the group consisting of fluoro, lower alkoxy, fluoro substituted lower alkoxy, lower alkylthio, and fluoro substituted lower alkylthio. In one embodiment, R²⁴ is selected from the group consisting of halogen, lower alkoxy, fluoro substituted lower alkoxy, lower alkylthio, fluoro substituted lower alkylthio and lower alkyl, wherein lower alkyl is optionally substituted with one or more substituents selected from the group consisting of fluoro, lower alkoxy, fluoro substituted lower alkoxy, lower alkylthio, and fluoro substituted lower alkylthio.

In one embodiment of compounds of Formulae Ia or Ib, Ar₂ₐ is selected from the group consisting of phenyl, pyridinyl, pyrimidinyl, thiophenyl, oxazolyl, isoxazolyl, thiazolyl, isothiazolyl, imidazolyl, and pyrazolyl. In one embodiment, Ar₂ₐ is selected from the group consisting of phenyl, pyridinyl, and thiophenyl, preferably phenyl and thiophenyl.

In one embodiment of compounds of Formulae Ia or Ib, R²⁵ is selected from the group consisting of halogen, —CN, lower alkyl, lower alkenyl, lower alkynyl, lower alkoxy, lower alkylthio, cycloalkyl, heterocycloalkyl, aryl and heteroaryl, wherein lower alkyl, lower alkenyl, lower alkynyl, cycloalkyl, heterocycloalkyl, aryl and heteroaryl are optionally substituted as described for R²⁵ in Formulae Ia or Ib, wherein lower alkoxy and lower alkylthio are optionally substituted with one or more substituents selected from the group consisting fluoro —R³², —OR³⁶, —SR³⁶, and —NR³⁷R³⁸, where R³², R³⁶, R³⁷ and R³⁸ are as defined in Formulae Ia and Ib. In one embodiment, R²⁵ is selected from the group consisting of halogen, —CN, lower alkyl, lower alkoxy, lower alkylthio, cycloalkyl, heterocycloalkyl, aryl and heteroaryl, wherein lower alkyl, lower alkoxy, and lower alkylthio are optionally substituted with one or more substituents selected from the group consisting of fluoro, lower alkoxy, fluoro substituted lower alkoxy, lower alkylthio, and fluoro substituted lower alkylthio, wherein cycloalkyl, heterocycloalkyl, aryl and heteroaryl are optionally substituted with one or more substituents selected from the group consisting of fluoro, —CN, lower alkyl, fluoro substituted lower alkyl, lower alkoxy, fluoro substituted lower alkoxy, lower alkylthio, and fluoro substituted lower alkylthio. In one embodiment, R²⁵ is selected from the group consisting of halogen, lower alkyl, lower alkoxy, and lower alkylthio, wherein lower alkyl, lower alkoxy, and lower alkylthio are optionally substituted with one or more substituents selected from the group consisting of fluoro, lower alkoxy, fluoro substituted lower alkoxy, lower alkylthio, and fluoro substituted lower alkylthio.

In one embodiment of compounds of Formulae Ia or Ib, M is selected from the group consisting of a covalent bond, —CR¹⁹R²⁰—, —O—, —S—, and —NR⁵³—, preferably M is a covalent bond or —O—.

In one embodiment of compounds of Formulae Ia or Ib, one of R¹ and R², preferably R², is —OR⁹ and the other of R¹ and R², preferably R¹, is hydrogen, W is selected from the group consisting of —(CR⁴R⁵)₁₋₃—, and —CR⁶═CR⁷—, preferably —CH₂CH₂— or —CH₂—, and p is 0.

In one embodiment of compounds of Formulae Ia or Ib, one of R¹ and R², preferably R², is —OR⁹ and the other of R¹ and R², preferably R¹, is hydrogen, W is selected from the group consisting of —(CR⁴R⁵)₁₋₃—, and —CR⁶═CR⁷—, preferably —CH₂CH₂— or —CH₂—, p is 0, Ar₁ₐ is selected from the group consisting of phenyl, pyridinyl, oxazolyl, and thiazolyl and Ar₂ₐ is selected from the group consisting of phenyl, pyridinyl, pyrimidinyl, thiophenyl, oxazolyl, isoxazolyl, thiazolyl, isothiazolyl, imidazolyl, and pyrazolyl.

In one embodiment of compounds of Formulae Ia or Ib, one of R¹ and R², preferably R², is halogen, lower alkyl, or C₃₋₆ cycloalkyl, wherein lower alkyl is optionally substituted with one or more substituents selected from the group consisting of fluoro, —OH, —NH₂, lower alkoxy, fluoro substituted lower alkoxy, lower alkylthio, fluoro substituted lower alkylthio, and C₃₋₆ cycloalkyl, wherein C₃₋₆ cycloalkyl, as R¹, R² or a substituent of lower alkyl, is optionally substituted with one or more substituents selected from the group consisting of halogen, —OH, —NH₂, lower alkyl, fluoro substituted lower alkyl, lower alkoxy, fluoro substituted lower alkoxy, lower alkylthio, and fluoro substituted lower alkylthio, preferably fluoro, chloro, lower alkyl, fluoro substituted lower alkyl, C₃₋₆ cycloalkyl, or fluoro substituted C₃₋₆ cycloalkyl and the other of R¹ and R², preferably R¹, is hydrogen, W is selected from the group consisting of —(CR⁴R⁵)₁₋₃—, and —CR⁶═CR⁷—, preferably —CH₂CH₂— or —CH₂—, p is 0, Ar₁ₐ is selected from the group consisting of phenyl, pyridinyl, oxazolyl, and thiazolyl and Ar₂ₐ is selected from the group consisting of phenyl, pyridinyl, pyrimidinyl, thiophenyl, oxazolyl, isoxazolyl, thiazolyl, isothiazolyl, imidazolyl, and pyrazolyl.

In one embodiment of compounds of Formulae Ia or Ib, one of R¹ and R², preferably R², is —OR⁹ and the other of R¹ and R², preferably R¹, is hydrogen, W is selected from the group consisting of —(CR⁴R⁵)₁₋₃—, and —CR⁶═CR⁷—, preferably —CH₂CH₂— or —CH₂—, p is 0, Ar₁ₐ is phenyl, pyridinyl, oxazolyl, or thiazolyl, Ar₂ₐ is selected from the group consisting of phenyl, pyridinyl, pyrimidinyl, thiophenyl, oxazolyl, isoxazolyl, thiazolyl, isothiazolyl, imidazolyl, and pyrazolyl, and M is selected from the group consisting of a covalent bond, —CR¹⁹R²⁰—, —O—, —S—, and —NR⁵³—.

In one embodiment of compounds of Formulae Ia or Ib, one of R¹ and R², preferably R², is halogen, lower alkyl, or C₃₋₆ cycloalkyl, wherein lower alkyl is optionally substituted with one or more substituents selected from the group consisting of fluoro, —OH, —NH₂, lower alkoxy, fluoro substituted lower alkoxy, lower alkylthio, fluoro substituted lower alkylthio, and C₃₋₆ cycloalkyl, wherein C₃₋₆ cycloalkyl, as R¹, R² or a substituent of lower alkyl, is optionally substituted with one or more substituents selected from the group consisting of halogen, —OH, —NH₂, lower alkyl, fluoro substituted lower alkyl, lower alkoxy, fluoro substituted lower alkoxy, lower alkylthio, and fluoro substituted lower alkylthio, preferably fluoro, chloro, lower alkyl, fluoro substituted lower alkyl, C₃₋₆ cycloalkyl, or fluoro substituted C₃₋₆ cycloalkyl and the other of R¹ and R², preferably R¹, is hydrogen, W is selected from the group consisting of —(CR⁴R⁵)₁₋₃—, and —CR⁶═CR⁷—, preferably —CH₂CH₂— or —CH₂—, p is 0, Ar₁ₐ is phenyl, pyridinyl, oxazolyl, or thiazolyl, Ar₂ₐ is selected from the group consisting of phenyl, pyridinyl, pyrimidinyl, thiophenyl, oxazolyl, isoxazolyl, thiazolyl, isothiazolyl, imidazolyl, and pyrazolyl, and M is selected from the group consisting of a covalent bond, —CR¹⁹R²⁰—, —O—, —S—, and —NR⁵³—.

In one embodiment of compounds of Formulae Ia or Ib, R² is —OR⁹, R¹ is hydrogen, W is —CR⁴R⁵—, X is —C(O)OR¹⁶ or a carboxylic acid isostere, p is 0, t is 0, 1, 2, 3, or 4, s is 0, M is a covalent bond or —O—, Ar₁ₐ is phenyl, pyridinyl, oxazolyl, or thiazolyl, and Ar₂ₐ is phenyl or thiophenyl.

In one embodiment of compounds of Formulae Ia or Ib, R² is fluoro, chloro, lower alkyl, fluoro substituted lower alkyl, C₃₋₆ cycloalkyl, or fluoro substituted C₃₋₆ cycloalkyl, R¹ is hydrogen, W is —CR⁴R⁵—, X is —C(O)OR¹⁶ or a carboxylic acid isostere, p is 0, t is 0, 1, 2, 3, or 4, s is 0, M is a covalent bond or —O—, Ar₁ₐ is phenyl, pyridinyl, oxazolyl, or thiazolyl, and Ar₂ₐ is phenyl or thiophenyl.

In one embodiment of compounds of Formulae Ia or Ib, R² is —OR⁹, wherein R⁹ is lower alkyl optionally substituted as described for R⁹ in Formula I, R¹ is hydrogen, W is —CR⁴R⁵—, X is —C(O)OR¹⁶ or a carboxylic acid isostere, p is 0, t is 0, 1, 2, 3, or 4, s is 0, M is a covalent bond or —O—, Ar₁ₐ is phenyl, pyridinyl, oxazolyl, or thiazolyl, preferably phenyl, R²⁴ is selected from the group consisting of halogen, lower alkyl, lower alkenyl, lower alkynyl, lower alkoxy and lower alkylthio, wherein lower alkyl, lower alkenyl, lower alkynyl, lower alkoxy or lower alkylthio are optionally substituted with one or more substituents selected from the group consisting of fluoro, —OR³⁶, —SR³⁶, and —NR³⁷R³⁸, where R³⁶, R³⁷ and R³⁸ are as defined in Formulae Ia and Ib, Ar₂ₐ is phenyl or thiophenyl, preferably phenyl, and R²⁵ is selected from the group consisting of halogen, —CN, lower alkyl, lower alkenyl, lower alkynyl, lower alkoxy, lower alkylthio, cycloalkyl, heterocycloalkyl, aryl and heteroaryl, wherein lower alkyl, lower alkenyl, lower alkynyl, cycloalkyl, heterocycloalkyl, aryl and heteroaryl are optionally substituted as described for R²⁵ in Formulae Ia or Ib, and lower alkoxy and lower alkylthio are optionally substituted with one or more substituents selected from the group consisting of fluoro, —R³², —OR³⁶, —SR³⁶, and —NR³⁷R³⁸, where R³², R³⁶, R³⁷ and R³⁸ are as defined in Formulae Ia and Ib.

In one embodiment of compounds of Formulae Ia or Ib, R² is —OR⁹, wherein R⁹ is lower alkyl optionally substituted with one or more substituents selected from the group consisting of fluoro, lower alkoxy, and lower alkylthio, R¹ is hydrogen, W is —CH₂—, X is —COOH, p is 0, t is 0, 1, 2, 3, or 4, s is 0, M is a covalent bond or —O—, Ar₁ₐ is phenyl, pyridinyl, oxazolyl, or thiazolyl, R²⁴ is selected from the group consisting of halogen, lower alkyl, lower alkoxy, and lower alkylthio, wherein lower alkyl, lower alkoxy and lower alkylthio are optionally substituted with one or more substituents selected from the group consisting of fluoro, lower alkoxy, fluoro substituted lower alkoxy, lower alkylthio, and fluoro substituted lower alkylthio, Ar₂ₐ is phenyl or thiophenyl, preferably phenyl, R²⁵ is selected from the group consisting of halogen, —CN, lower alkyl, lower alkoxy, lower alkylthio, cycloalkyl, heterocycloalkyl, aryl and heteroaryl, wherein lower alkyl, lower alkoxy, and lower alkylthio are optionally substituted with one or more substituents selected from the group consisting of fluoro, lower alkoxy, fluoro substituted lower alkoxy, lower alkylthio, and fluoro substituted lower alkylthio, and wherein cycloalkyl, heterocycloalkyl, aryl and heteroaryl are optionally substituted with one or more substituents selected from the group consisting of fluoro, —CN, lower alkyl, fluoro substituted lower alkyl, lower alkoxy, fluoro substituted lower alkoxy, lower alkylthio, and fluoro substituted lower alkylthio.

In one embodiment of compounds of Formulae Ia or Ib, R² is fluoro, chloro, lower alkyl, fluoro substituted lower alkyl, C₃₋₆ cycloalkyl, or fluoro substituted C₃₋₆ cycloalkyl, R¹ is hydrogen, W is —CR⁴R⁵—, X is —C(O)OR¹⁶ or a carboxylic acid isostere, p is 0, t is 0, 1, 2, 3, or 4, s is 0, M is a covalent bond or —O—, Ar₁ₐ is phenyl, pyridinyl, oxazolyl, or thiazolyl, preferably phenyl, R²⁴ is selected from the group consisting of halogen, lower alkyl, lower alkenyl, lower alkynyl, lower alkoxy and lower alkylthio, wherein lower alkyl, lower alkenyl, lower alkynyl, lower alkoxy or lower alkylthio are optionally substituted with one or more substituents selected from the group consisting of fluoro, —OR³⁶, —SR³⁶, and —NR³⁷R³⁸, where R³⁶, R³⁷ and R³⁸ are as defined in Formulae Ia and Ib, Ar₂ₐ is phenyl or thiophenyl, preferably phenyl, and R²⁵ is selected from the group consisting of halogen, —CN, lower alkyl, lower alkenyl, lower alkynyl, lower alkoxy, lower alkylthio, cycloalkyl, heterocycloalkyl, aryl and heteroaryl, wherein lower alkyl, lower alkenyl, lower alkynyl, cycloalkyl, heterocycloalkyl, aryl and heteroaryl are optionally substituted as described for R²⁵ in Formulae Ia or Ib, and lower alkoxy and lower alkylthio are optionally substituted with one or more substituents selected from the group consisting of fluoro, —R³², —OR³⁶, —SR³⁶, and —NR³⁷R³⁸, where R³², R³⁶, R³⁷ and R³⁸ are as defined in Formulae Ia and Ib.

In one embodiment of compounds of Formulae Ia or Ib, R² is fluoro, chloro, lower alkyl, fluoro substituted lower alkyl, C₃₋₆ cycloalkyl, or fluoro substituted C₃₋₆ cycloalkyl, R¹ is hydrogen, W is —CH₂—, X is —COOH, p is 0, t is 0, 1, 2, 3, or 4, s is 0, M is a covalent bond or —O—, Ar₁ₐ is phenyl, pyridinyl, oxazolyl, or thiazolyl, R²⁴ is selected from the group consisting of halogen, lower alkyl, lower alkoxy, and lower alkylthio, wherein lower alkyl, lower alkoxy and lower alkylthio are optionally substituted with one or more substituents selected from the group consisting of fluoro, lower alkoxy, fluoro substituted lower alkoxy, lower alkylthio, and fluoro substituted lower alkylthio, Ar₂ₐ is phenyl or thiophenyl, preferably phenyl, R²⁵ is selected from the group consisting of halogen, —CN, lower alkyl, lower alkoxy, lower alkylthio, cycloalkyl, heterocycloalkyl, aryl and heteroaryl, wherein lower alkyl, lower alkoxy, and lower alkylthio are optionally substituted with one or more substituents selected from the group consisting of fluoro, lower alkoxy, fluoro substituted lower alkoxy, lower alkylthio, and fluoro substituted lower alkylthio, and wherein cycloalkyl, heterocycloalkyl, aryl and heteroaryl are optionally substituted with one or more substituents selected from the group consisting of fluoro, —CN, lower alkyl, fluoro substituted lower alkyl, lower alkoxy, fluoro substituted lower alkoxy, lower alkylthio, and fluoro substituted lower alkylthio.

In one embodiment, compounds of Formula I have the following sub-generic structure (Formula Ic):

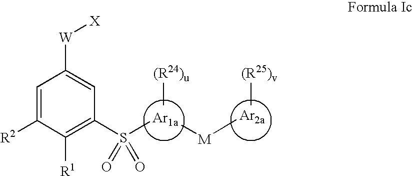

all salts, prodrugs, tautomers, and isomers thereof,
wherein:
X, W, M, R¹, and R² are as defined for Formula I; and
Ar₁ₐ, Ar₂ₐ, R²⁴, R²⁵, u and v are as defined for Formulae Ia and Ib.

In one embodiment, compounds of Formula I have the following sub-generic structure (Formula Id):

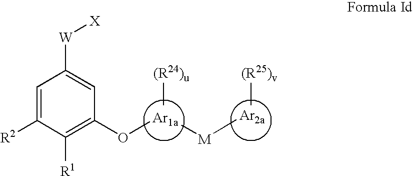

all salts, prodrugs, tautomers, and isomers thereof,
wherein:
X, W, M, R¹, and R² are as defined for Formula I; and
Ar₁ₐ, Ar₂ₐ, R²⁴, R²⁵, u and v are as defined for Formulae Ia and Ib, provided, however, that when Ar₂ₐ is phenyl,

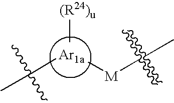

is not

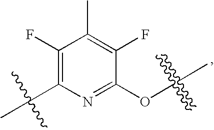

wherein

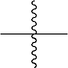

indicates the attachment point to O and

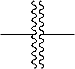

indicates the attachment point to Ar₂ₐ.

In one embodiment of the compounds of Formulae Ic or Id, at least one of R¹ and R² is other than hydrogen. In one embodiment, one of R¹ and R² is other than hydrogen and the other of R¹ and R² is hydrogen or halogen. In one embodiment, one of R¹ and R² is other than hydrogen and the other of R¹ and R² is hydrogen. In one embodiment, at least one of R¹ and R² is —SR⁹ or —OR⁹, preferably —OR⁹. In one embodiment, one of R¹ and R² is —SR⁹ or —OR⁹, preferably —OR⁹, and the other of R¹ and R² is hydrogen or halogen. In one embodiment, one of R¹ and R² is —SR⁹ or —OR⁹, preferably —OR⁹, and the other of R¹ and R² is hydrogen. In one embodiment, R¹ is —SR⁹ or —OR⁹, preferably —OR⁹, and R² is hydrogen. In one embodiment, R² is —SR⁹ or —OR⁹, preferably —OR⁹, and R¹ is hydrogen. In one embodiment, both R¹ and R² are hydrogen.

In one embodiment of the compounds of Formulae Ic or Id, at least one of R¹ and R² is halogen, lower alkyl, or C₃₋₆ cycloalkyl, wherein lower alkyl is optionally substituted with one or more substituents selected from the group consisting of fluoro, —OH, —NH₂, lower alkoxy, fluoro substituted lower alkoxy, lower alkylthio, fluoro substituted lower alkylthio, and C₃₋₆ cycloalkyl, wherein C₃₋₆ cycloalkyl, as R¹, R² or a substituent of lower alkyl, is optionally substituted with one or more substituents selected from the group consisting of halogen, —OH, —NH₂, lower alkyl, fluoro substituted lower alkyl, lower alkoxy, fluoro substituted lower alkoxy, lower alkylthio, and fluoro substituted lower alkylthio, preferably one of R¹ and R² is hydrogen, preferably R¹ is hydrogen and R² is fluoro, chloro, lower alkyl, fluoro substituted lower alkyl, C₃₋₆ cycloalkyl, or fluoro substituted C₃₋₆ cycloalkyl.

In one embodiment of the compounds of Formulae Ic or Id, one of R¹ and R², preferably R², is —SR⁹ or —OR⁹, preferably —OR⁹, the other of R¹ and R², preferably R¹, is hydrogen, and R⁹ is selected from the group consisting of lower alkyl, C₃₋₆ alkenyl, C₃₋₆ alkynyl, and cycloalkyl, wherein lower alkyl, C₃₋₆ alkenyl, C₃₋₆ alkynyl, and cycloalkyl are optionally substituted as described for R⁹ in Formula I. In one embodiment, one of R¹ and R², preferably R², is —SR⁹ or —OR⁹, preferably —OR⁹, the other of R¹ and R², preferably R¹, is hydrogen, and R⁹ is selected from the group consisting lower alkyl, C₃₋₆ alkenyl, C₃₋₆ alkynyl, and cycloalkyl, wherein cycloalkyl is optionally substituted with one or more substituents selected from the group consisting of fluoro, —OH, lower alkyl, fluoro substituted lower alkyl, lower alkoxy, fluoro substituted lower alkoxy, lower alkylthio, and fluoro substituted lower alkylthio, and wherein lower alkyl, C₃₋₆ alkenyl, and C₃₋₆ alkynyl are optionally substituted with one or more substituents selected from the group consisting of fluoro, —OH, lower alkoxy, fluoro substituted lower alkoxy, lower alkylthio, fluoro substituted lower alkylthio, and cycloalkyl, wherein the cycloalkyl substituent of alkyl, C₃₋₆ alkenyl, or C₃₋₆ alkynyl is optionally substituted with one or more substituents selected from the group consisting of fluoro, —OH, lower alkyl, fluoro substituted lower alkyl, lower alkoxy, fluoro substituted lower alkoxy, lower alkylthio, and fluoro substituted lower alkylthio. In one embodiment, one of R¹ and R², preferably R², is —SR⁹ or —OR⁹, preferably —OR⁹, the other of R¹ and R², preferably R¹, is hydrogen, and R⁹ is selected from the group consisting of lower alkyl, C₃₋₆ alkenyl, C₃₋₆ alkynyl, and cycloalkyl, wherein the lower alkyl, C₃₋₆ alkenyl, C₃₋₆ alkynyl, and cycloalkyl are optionally substituted with one or more substituents selected from the group consisting of fluoro, lower alkoxy, and lower alkylthio. In one embodiment, one of R¹ and R², preferably R², is —SR⁹ or —OR⁹, preferably —OR⁹, the other of R¹ and R², preferably R¹, is hydrogen, and R⁹ is lower alkyl optionally substituted with one or more substituents selected from the group consisting of fluoro, lower alkoxy, fluoro substituted lower alkoxy, lower alkylthio, fluoro substituted lower alkylthio, cycloalkyl, and fluoro substituted cycloalkyl. In one embodiment, one of R¹ and R², preferably R², is —SR⁹ or —OR⁹, preferably —OR⁹, the other of R¹ and R², preferably R¹, is hydrogen, and R⁹ is lower alkyl optionally substituted with one or more substituents selected from the group consisting of fluoro, lower alkoxy, and lower alkylthio.

In one embodiment of compounds of Formulae Ic or Id, W is selected from the group consisting of —NR⁵¹(CR⁴R⁵)₁₋₂—, —O—(CR⁴R⁵)₁₋₂—, —S—(CR⁴R⁵)₁₋₂—, —(CR⁴R⁵)₁₋₃—, and —CR⁶═CR⁷—, wherein R⁵¹ is hydrogen or lower alkyl optionally substituted with one or more substituents selected from the group consisting of fluoro, lower alkoxy, fluoro substituted lower alkoxy, lower alkylthio, and fluoro substituted lower alkylthio, and wherein R⁴, R⁵, R⁶ and R⁷ are independently hydrogen or lower alkyl optionally substituted with one or more substituents selected from the group consisting of fluoro, lower alkoxy, fluoro substituted lower alkoxy, lower alkylthio, and fluoro substituted lower alkylthio. In one embodiment W is selected from the group consisting of —(CR⁴R⁵)₁₋₃—, and —CR⁶═CR⁷—. In one embodiment, W is —(CR⁴R⁵)₁₋₂—. In one embodiment, W is —(CR⁴R⁵)—. In one embodiment W is selected from the group consisting of —(CR⁴R⁵)₁₋₃—, and —CR⁶═CR⁷—, wherein R⁴, R⁵, R⁶ and R⁷ are independently hydrogen or lower alkyl optionally substituted with one or more substituents selected from the group consisting of fluoro, lower alkoxy, fluoro substituted lower alkoxy, lower alkylthio, and fluoro substituted lower alkylthio. In one embodiment, W is —(CR⁴R⁵)₁₋₂—, preferably —(CR⁴R⁵)—, wherein R⁴ and R⁵ are independently hydrogen or lower alkyl optionally substituted with one or more substituents selected from the group consisting of fluoro, lower alkoxy, fluoro substituted lower alkoxy, lower alkylthio, and fluoro substituted lower alkylthio. In one embodiment, W is —CH₂CH₂— or —CH₂—, preferably —CH₂—.

In one embodiment of compounds of Formulae Ic or Id, X is —C(O)OR¹⁶ or a carboxylic acid isostere, preferably wherein X is —COOH. In one embodiment, W is —(CR⁴R⁵)₁₋₂— and X is —C(O)OR¹⁶ or a carboxylic acid isostere, preferably W is —CH₂CH₂— or —CH₂— and X is —COOH.

In one embodiment of compounds of Formulae Ic or Id, Ar₁ₐ is selected from the group consisting of phenyl, pyridinyl, pyrimidinyl, thiophenyl, oxazolyl, isoxazolyl, thiazolyl, isothiazolyl, imidazolyl, and pyrazolyl. In one embodiment of compounds of Formulae Ic or Id, Ar₁ₐ is selected from the group consisting of phenyl, pyridinyl, oxazolyl, thiazolyl, imidazolyl, and pyrazolyl, preferably phenyl, pyridinyl, oxazolyl, and thiazolyl.

In one embodiment of compounds of Formulae Ic or Id, R²⁴ is selected from the group consisting of halogen, lower alkyl, lower alkenyl, lower alkynyl, lower alkoxy and lower alkylthio, wherein lower alkyl, lower alkenyl, lower alkynyl, lower alkoxy or lower alkylthio are optionally substituted with one or more substituents selected from the group consisting of fluoro, —OR³⁶, —SR³⁶, and —NR³⁷R³⁸, where R³⁶, R³⁷ and R³⁸ are as defined in Formulae Ia and Ib. In one embodiment, R²⁴ is selected from the group consisting of halogen, lower alkyl, lower alkoxy, and lower alkylthio, wherein lower alkyl, lower alkoxy and lower alkylthio are optionally substituted with one or more substituents selected from the group consisting of fluoro, lower alkoxy, fluoro substituted lower alkoxy, lower alkylthio, and fluoro substituted lower alkylthio. In one embodiment, R²⁴ is selected from the group consisting of halogen, lower alkoxy, fluoro substituted lower alkoxy, lower alkylthio, fluoro substituted lower alkylthio and lower alkyl, wherein lower alkyl is optionally substituted with one or more substituents selected from the group consisting of fluoro, lower alkoxy, fluoro substituted lower alkoxy, lower alkylthio, and fluoro substituted lower alkylthio.

In one embodiment of compounds of Formulae Ic or Id, Ar₂ₐ is selected from the group consisting of phenyl, pyridinyl, pyrimidinyl, thiophenyl, oxazolyl, isoxazolyl, thiazolyl, isothiazolyl, imidazolyl, and pyrazolyl. In one embodiment, Ar₂ₐ is selected from the group consisting of phenyl, pyridinyl, and thiophenyl, preferably phenyl and thiophenyl.

In one embodiment of compounds of Formulae Ic or Id, R²⁵ is selected from the group consisting of halogen, —CN, lower alkyl, lower alkenyl, lower alkynyl, lower alkoxy, lower alkylthio, cycloalkyl, heterocycloalkyl, aryl and heteroaryl, wherein lower alkyl, lower alkenyl, lower alkynyl, cycloalkyl, heterocycloalkyl, aryl and heteroaryl are optionally substituted as described for R²⁵ in Formulae Ia or Ib, and wherein lower alkoxy and lower alkylthio are optionally substituted with one or more substituents selected from the group consisting of fluoro, —R³², OR³⁶, —SR³⁶ and —NR³⁷R³⁸, where R³², R³⁶, R³⁷ and R³⁸ are as defined in Formulae Ia and Ib. In one embodiment, R²⁵ is selected from the group consisting of halogen, —CN, lower alkyl, lower alkoxy, lower alkylthio, cycloalkyl, heterocycloalkyl, aryl and heteroaryl, wherein lower alkyl, lower alkoxy, and lower alkylthio are optionally substituted with one or more substituents selected from the group consisting of fluoro, lower alkoxy, fluoro substituted lower alkoxy, lower alkylthio, and fluoro substituted lower alkylthio, and wherein cycloalkyl, heterocycloalkyl, aryl and heteroaryl are optionally substituted with one or more substituents selected from the group consisting of fluoro, —CN, lower alkyl, fluoro substituted lower alkyl, lower alkoxy, fluoro substituted lower alkoxy, lower alkylthio, and fluoro substituted lower alkylthio. In one embodiment, R²⁵ is selected from the group consisting of halogen, lower alkyl, lower alkoxy, and lower alkylthio wherein lower alkyl, lower alkoxy, and lower alkylthio are optionally substituted with one or more substituents selected from the group consisting of fluoro, lower alkoxy, fluoro substituted lower alkoxy, lower alkylthio, and fluoro substituted lower alkylthio. In one embodiment, R²⁵ is perhaloalkyl, for example without limitation, CF₃ or CF₂CF₃.

In one embodiment of compounds of Formulae Ic or Id, M is selected from the group consisting of a covalent bond, —CR¹⁹R²⁰—, —O—, —S—, and —NR⁵³—, preferably M is a covalent bond or —O—.

In one embodiment of compounds of Formulae Ic or Id, one of R¹ and R², preferably R², is —OR⁹ and the other of R¹ and R², preferably R¹, is hydrogen, and W is selected from the group consisting of —(CR⁴R⁵)₁₋₃—, and —CR⁶═CR⁷—, preferably —CH₂CH₂— or —CH₂—.

In one embodiment of compounds of Formulae Ic or Id, one of R¹ and R², preferably R², is —OR⁹ and the other of R¹ and R², preferably R¹, is hydrogen, W is selected from the group consisting of —(CR⁴R⁵)₁₋₃—, and —CR⁶═CR⁷—, preferably —CH₂CH₂— or —CH₂—, Ar₁ₐ is selected from the group consisting of phenyl, pyridinyl, pyrimidinyl, thiophenyl, oxazolyl, isoxazolyl, thiazolyl, isothiazolyl, imidazolyl, and pyrazolyl, preferably phenyl, pyridinyl and thiophenyl, and Ar₂ₐ is selected from the group consisting of phenyl, pyridinyl, pyrimidinyl, thiophenyl, oxazolyl, isoxazolyl, thiazolyl, isothiazolyl, imidazolyl, and pyrazolyl.

In one embodiment of compounds of Formulae Ic or Id, one of R¹ and R², preferably R², is halogen, lower alkyl, or C₃₋₆ cycloalkyl, wherein lower alkyl is optionally substituted with one or more substituents selected from the group consisting of fluoro, —OH, —NH₂, lower alkoxy, fluoro substituted lower alkoxy, lower alkylthio, fluoro substituted lower alkylthio, and C₃₋₆ cycloalkyl, wherein C₃₋₆ cycloalkyl, as R¹, R² or a substituent of lower alkyl, is optionally substituted with one or more substituents selected from the group consisting of halogen, —OH, —NH₂, lower alkyl, fluoro substituted lower alkyl, lower alkoxy, fluoro substituted lower alkoxy, lower alkylthio, and fluoro substituted lower alkylthio, preferably fluoro, chloro, lower alkyl, fluoro substituted lower alkyl, C₃₋₆ cycloalkyl, or fluoro substituted C₃₋₆ cycloalkyl and the other of R¹ and R², preferably R¹, is hydrogen, W is selected from the group consisting of —(CR⁴R⁵)₁₋₃— and —CR⁶═CR⁷—, preferably —CH₂CH₂— or —CH₂—, Ar₁ₐ is selected from the group consisting of phenyl, pyridinyl, pyrimidinyl, thiophenyl, oxazolyl, isoxazolyl, thiazolyl, isothiazolyl, imidazolyl, and pyrazolyl, preferably phenyl, pyridinyl and thiophenyl, and Ar₂ₐ is selected from the group consisting of phenyl, pyridinyl, pyrimidinyl, thiophenyl, oxazolyl, isoxazolyl, thiazolyl, isothiazolyl, imidazolyl, and pyrazolyl.

In one embodiment of compounds of Formulae Ic or Id, one of R¹ and R², preferably R², is —OR⁹ and the other of R¹ and R², preferably R¹, is hydrogen, W is selected from the group consisting of —(CR⁴R⁵)₁₋₃—, and —CR⁶═CR⁷—, preferably —CH₂CH₂— or —CH₂—, Ar₁ₐ is selected from the group consisting of phenyl, pyridinyl, oxazolyl, thiazolyl, imidazolyl, and pyrazolyl, Ar₂ₐ is selected from the group consisting of phenyl, pyridinyl, pyrimidinyl, thiophenyl, oxazolyl, isoxazolyl, thiazolyl, isothiazolyl, imidazolyl, and pyrazolyl, and M is selected from the group consisting of a covalent bond, —CR¹⁹R²⁰—, —O—, —S—, and —NR⁵³—.

In one embodiment of compounds of Formulae Ic or Id, one of R¹ and R² preferably R², is halogen, lower alkyl, or C₃₋₆ cycloalkyl, wherein lower alkyl is optionally substituted with one or more substituents selected from the group consisting of fluoro, —OH, —NH₂, lower alkoxy, fluoro substituted lower alkoxy, lower alkylthio, fluoro substituted lower alkylthio, and C₃₋₆ cycloalkyl, wherein C₃₋₆ cycloalkyl, as R¹, R² or a substituent of lower alkyl, is optionally substituted with one or more substituents selected from the group consisting of halogen, —OH, —NH₂, lower alkyl, fluoro substituted lower alkyl, lower alkoxy, fluoro substituted lower alkoxy, lower alkylthio, and fluoro substituted lower alkylthio, preferably fluoro, chloro, lower alkyl, fluoro substituted lower alkyl, C₃₋₆ cycloalkyl, or fluoro substituted C₃₋₆ cycloalkyl and the other of R¹ and R², preferably R¹, is hydrogen, W is selected from the group consisting of —(CR⁴R⁵)₁₋₃— and —CR⁶═CR⁷—, preferably —CH₂CH₂— or —CH₂—, Ar₁ₐ is selected from the group consisting of phenyl, pyridinyl, oxazolyl, thiazolyl, imidazolyl, and pyrazolyl, Ar₂ₐ is selected from the group consisting of phenyl, pyridinyl, pyrimidinyl, thiophenyl, oxazolyl, isoxazolyl, thiazolyl, isothiazolyl, imidazolyl, and pyrazolyl, and M is selected from the group consisting of a covalent bond, —CR¹⁹R²⁰—, —O—, —S—, and —NR⁵³—.

In one embodiment of compounds of Formulae Ic or Id, R² is —OR⁹, R¹ is hydrogen, W is —CR⁴R⁵—, X is —C(O)OR¹⁶ or a carboxylic acid isostere, M is a covalent bond or —O—, Ar₁ₐ is phenyl, pyridinyl, oxazolyl, or thiazolyl, and Ar₂ₐ is phenyl or thiophenyl.

In one embodiment of compounds of Formulae Ic or Id, R² is fluoro, chloro, lower alkyl, fluoro substituted lower alkyl, C₃₋₆ cycloalkyl, or fluoro substituted C₃₋₆ cycloalkyl, R¹ is hydrogen, W is —CR⁴R⁵—, X is —C(O)OR¹⁶ or a carboxylic acid isostere, M is a covalent bond or —O—, Ar₁ₐ is phenyl, pyridinyl, oxazolyl, or thiazolyl, and Ar₂ₐ is phenyl or thiophenyl.

In one embodiment of compounds of Formulae Ic or Id, R² is —OR⁹, wherein R⁹ is lower alkyl optionally substituted as described for R⁹ in Formula I, R¹ is hydrogen, W is —CR⁴R⁵—, X is —C(O)OR¹⁶ or a carboxylic acid isostere, M is a covalent bond or —O—, Ar₁ₐ is phenyl, pyridinyl, oxazolyl, or thiazolyl, R²⁴ is selected from the group consisting of halogen, lower alkyl, lower alkenyl, lower alkynyl, lower alkoxy and lower alkylthio, wherein lower alkyl, lower alkenyl, lower alkynyl, lower alkoxy or lower alkylthio are optionally substituted with one or more substituents selected from the group consisting of fluoro, —OR³⁶, —SR³⁶, and —NR³⁷R³⁸, where R³⁶, R³⁷ and R³⁸ are as defined in Formulae Ia and Ib, Ar₂ₐ is phenyl or thiophenyl, preferably phenyl, and R²⁵ is selected from the group consisting of halogen, —CN, lower alkyl, lower alkenyl, lower alkynyl, lower alkoxy, lower alkylthio, cycloalkyl, heterocycloalkyl, aryl and heteroaryl, wherein lower alkyl, lower alkenyl, lower alkynyl, cycloalkyl, heterocycloalkyl, aryl and heteroaryl are optionally substituted as described for R²⁵ in Formulae Ia or Ib, and lower alkoxy and lower alkylthio are optionally substituted with one or more substituents selected from the group consisting of fluoro, —R³², —OR³⁶, —SR³⁶, and —NR³⁷R³⁸, where R³², R³⁶, R³⁷ and R³⁸ are as defined in Formulae Ia and Ib.

In one embodiment of compounds of Formulae Ic or Id, R² is —OR⁹, wherein R⁹ is lower alkyl optionally substituted with one or more substituents selected from the group consisting of fluoro, lower alkoxy, and lower alkylthio, R¹ is hydrogen, W is —CH₂—, X is —COOH, M is a covalent bond or —O—, Ar₁ₐ is phenyl, pyridinyl, oxazolyl, or thiazolyl, R²⁴ is selected from the group consisting of halogen, lower alkyl, lower alkoxy, and lower alkylthio, wherein lower alkyl, lower alkoxy and lower alkylthio are optionally substituted with one or more substituents selected from the group consisting of fluoro, lower alkoxy, fluoro substituted lower alkoxy, lower alkylthio, and fluoro substituted lower alkylthio, Ar₂ₐ is phenyl or thiophenyl, preferably phenyl, R²⁵ is selected from the group consisting of halogen, —CN, lower alkyl, lower alkoxy, lower alkylthio, cycloalkyl, heterocycloalkyl, aryl and heteroaryl, wherein lower alkyl, lower alkoxy, and lower alkylthio are optionally substituted with one or more substituents selected from the group consisting of fluoro, lower alkoxy, fluoro substituted lower alkoxy, lower alkylthio, and fluoro substituted lower alkylthio, and wherein cycloalkyl, heterocycloalkyl, aryl and heteroaryl are optionally substituted with one or more substituents selected from the group consisting of fluoro, —CN, lower alkyl, fluoro substituted lower alkyl, lower alkoxy, fluoro substituted lower alkoxy, lower alkylthio, and fluoro substituted lower alkylthio.

In one embodiment of compounds of Formulae Ic or Id, R² is fluoro, chloro, lower alkyl, fluoro substituted lower alkyl, C₃₋₆ cycloalkyl, or fluoro substituted C₃₋₆ cycloalkyl, R¹ is hydrogen, W is —CR⁴R⁵—, X is —C(O)OR¹⁶ or a carboxylic acid isostere, M is a covalent bond or —O—, Ar₁ₐ is phenyl, pyridinyl, oxazolyl, or thiazolyl, R²⁴ is selected from the group consisting of halogen, lower alkyl, lower alkenyl, lower alkynyl, lower alkoxy and lower alkylthio, wherein lower alkyl, lower alkenyl, lower alkynyl, lower alkoxy or lower alkylthio are optionally substituted with one or more substituents selected from the group consisting of fluoro, —OR³⁶, —SR³⁶, and —NR³⁷R³⁸, where R³⁶, R³⁷ and R³⁸ are as defined in Formulae Ia and Ib, Ar₂ₐ is phenyl or thiophenyl, preferably phenyl, and R²⁵ is selected from the group consisting of halogen, —CN, lower alkyl, lower alkenyl, lower alkynyl, lower alkoxy, lower alkylthio, cycloalkyl, heterocycloalkyl, aryl and heteroaryl, wherein lower alkyl, lower alkenyl, lower alkynyl, cycloalkyl, heterocycloalkyl, aryl and heteroaryl are optionally substituted as described for R²⁵ in Formulae Ia or Ib, and lower alkoxy and lower alkylthio are optionally substituted with one or more substituents selected from the group consisting of fluoro, —R³², —OR³⁶, —SR³⁶, and —NR³⁷R³⁸, where R³², R³⁶, R³⁷ and R³⁸ are as defined in Formulae Ia and Ib.

In one embodiment of compounds of Formulae Ic or Id, R² is fluoro, chloro, lower alkyl, fluoro substituted lower alkyl, C₃₋₆ cycloalkyl, or fluoro substituted C₃₋₆ cycloalkyl, R¹ is hydrogen, W is —CH₂—, X is —COOH, M is a covalent bond or —O—, Ar₁ₐ is phenyl, pyridinyl, oxazolyl, or thiazolyl, R²⁴ is selected from the group consisting of halogen, lower alkyl, lower alkoxy, and lower alkylthio, wherein lower alkyl, lower alkoxy and lower alkylthio are optionally substituted with one or more substituents selected from the group consisting of fluoro, lower alkoxy, fluoro substituted lower alkoxy, lower alkylthio, and fluoro substituted lower alkylthio, Ar₂ₐ is phenyl or thiophenyl, preferably phenyl, R²⁵ is selected from the group consisting of halogen, —CN, lower alkyl, lower alkoxy, lower alkylthio, cycloalkyl, heterocycloalkyl, aryl and heteroaryl, wherein lower alkyl, lower alkoxy, and lower alkylthio are optionally substituted with one or more substituents selected from the group consisting of fluoro, lower alkoxy, fluoro substituted lower alkoxy, lower alkylthio, and fluoro substituted lower alkylthio, and wherein cycloalkyl, heterocycloalkyl, aryl and heteroaryl are optionally substituted with one or more substituents selected from the group consisting of fluoro, —CN, lower alkyl, fluoro substituted lower alkyl, lower alkoxy, fluoro substituted lower alkoxy, lower alkylthio, and fluoro substituted lower alkylthio.

In one embodiment of compounds of Formulae Ic or Id, Ar₁ₐ is phenyl. In other embodiments, Ar₁ₐ is phenyl and M is bound to Ar₁ₐ para to the S(O)₂ of Formula Ic or the of Formula Id. In further embodiments, Ar₁ₐ is phenyl, M is bound to Ar₁ₐ para to the S(O)₂ of Formula Ic or the O of Formula Id, and Ar₂ₐ is phenyl.

In one embodiment of compounds of Formulae Ic or Id, Ar₁ₐ is phenyl and M is bound to Ar₁ₐ meta to the S(O)₂ of Formula Ic or the O of Formula Id. In further embodiments, Ar₁ₐ is phenyl, M is bound to Ar₁ₐ meta to the S(O)₂ of Formula Ic or the O of Formula Id, and Ar₂ₐ is phenyl.

In one embodiment of compounds of Formulae Ic or Id, Ar₁ₐ is phenyl, M is a covalent bond or —O— and is bound to Ar₁ₐ para to the S(O)₂ of Formula Ic or the O of Formula Id, u is 0, v is 1, Ar₂ₐ is phenyl, R² is —OR⁹, wherein R⁹ is lower alkyl optionally substituted with one or more substituents selected from the group consisting of fluoro, lower alkoxy, and lower alkylthio, R¹ is hydrogen, W is —CH₂—, X is —COOH, and R²⁵ is selected from the group consisting of halogen, lower alkyl, lower alkoxy, and lower alkylthio, wherein lower alkyl, lower alkoxy, and lower alkylthio are optionally substituted with one or more substituents selected from the group consisting of fluoro, lower alkoxy, fluoro substituted lower alkoxy, lower alkylthio, and fluoro substituted lower alkylthio.

In one embodiment of compounds of Formulae Ic or Id, Ar₁ₐ is phenyl, M is —O— and is bound to Ar₁ₐ para to the S(O)₂ of Formula Ic or the O of Formula Id, u is 0, v is 1, Ar₂ₐ is phenyl, R² is —OR⁹, wherein R⁹ is lower alkyl, R¹ is hydrogen, W is —CH₂—, X is —COOH, and R²⁵ is optionally fluoro substituted lower alkyl or optionally fluoro substituted lower alkoxy, wherein R²⁵ is bound to Ar₂ₐ para to M.

In one embodiment of compounds of Formulae Ic or Id, Ar₁ₐ is phenyl, M is —O— and is bound to Ar₁ₐ para to the S(O)₂ of Formula Ic or the O of Formula Id, u is 0, v is 1, Ar₂ₐ is phenyl, R² is —OR⁹, wherein R⁹ is lower alkyl, R¹ is hydrogen, W is —CH₂—, X is —COOH, and R²⁵ is optionally fluoro substituted lower alkyl or optionally fluoro substituted lower alkoxy, wherein R²⁵ is bound to Ar₂ₐ meta to M.

In one embodiment of compounds of Formulae Ic or Id, Ar₁ₐ is phenyl, M is a covalent bond or —O— and is bound to Ar₁ₐ meta to the S(O)₂ of Formula Ic or the O of Formula Id, u is 0, v is 1, Ar₂ₐ is phenyl, R² is —OR⁹, wherein R⁹ is lower alkyl optionally substituted with one or more substituents selected from the group consisting of fluoro, lower alkoxy, and lower alkylthio, R¹ is hydrogen, W is —CH₂—, X is —COOH, and R²⁵ is selected from the group consisting of halogen, lower alkyl, lower alkoxy, and lower alkylthio, wherein lower alkyl, lower alkoxy, and lower alkylthio are optionally substituted with one or more substituents selected from the group consisting of fluoro, lower alkoxy, fluoro substituted lower alkoxy, lower alkylthio, and fluoro substituted lower alkylthio.

In one embodiment of compounds of Formulae Ic or Id, Ar₁ₐ is phenyl, M is a covalent bond or —O— and is bound to Ar₁ₐ meta to the S(O)₂ of Formula Ic or the O of Formula Id, u is 0, v is 1, Ar₂ₐ is phenyl, R² is —OR⁹, wherein R⁹ is lower alkyl, R¹ is hydrogen, W is —CH₂—, X is —COOH, and R²⁵ is optionally fluoro substituted lower alkyl or optionally fluoro substituted lower alkoxy, wherein R²⁵ is bound to Ar₂ₐ para to M.

In one embodiment of compounds of Formulae Ic or Id, Ar₁ₐ is phenyl, M is a covalent bond or —O— and is bound to Ar₁ₐ meta to the S(O)₂ of Formula Ic or the O of Formula Id, u is 0, v is 1, Ar₂ₐ is phenyl, R² is —OR⁹, wherein R⁹ is lower alkyl, R¹ is hydrogen, W is —CH₂—, X is —COOH, and R²⁵ is optionally fluoro substituted lower alkyl or optionally fluoro substituted lower alkoxy, wherein R²⁵ is bound to Ar₂ₐ meta to M.

In embodiments of compounds of Formulae I, Ia, Ib, Ic or Id where Ar₁ or Ar₁ₐ is phenyl, pyridinyl, pyrimidinyl, thiophenyl, oxazolyl, isoxazolyl, thiazolyl, isothiazolyl, imidazolyl, or pyrazolyl, it is understood that the ring orientation and ring substitutions are such as to provide a stable compound. For example, when Ar₁ or Ar₁ₐ is phenyl, pyridinyl, pyrimidinyl, thiophenyl, oxazolyl, isoxazolyl, thiazolyl, isothiazolyl, imidazolyl, or pyrazolyl, Ar₁ or Ar₁ₐ is selected from the following structures, wherein A represents the point of attachment of Ar₁ or Ar₁ₐ to —[(CR⁴R⁵)ₘ—(Y)ₚ]ᵣ— (or L when r=0) of Formula I, —O—(CR⁴R⁵)ₛ(Y)ₚ— of Formula Ia, —S(O)₂—(CR⁴R⁵)ₜ(Y)ₚ— of Formula Ib, —S(O)₂— of Formula Ic or —O— of Formula Id, and B represents the point of attachment of Ar₁ or Ar₁ₐ to M (or to Ar₂ or Ar₂ₐ when M is a bond) in Formulae I, Ia, Ib, Ic, or Id:

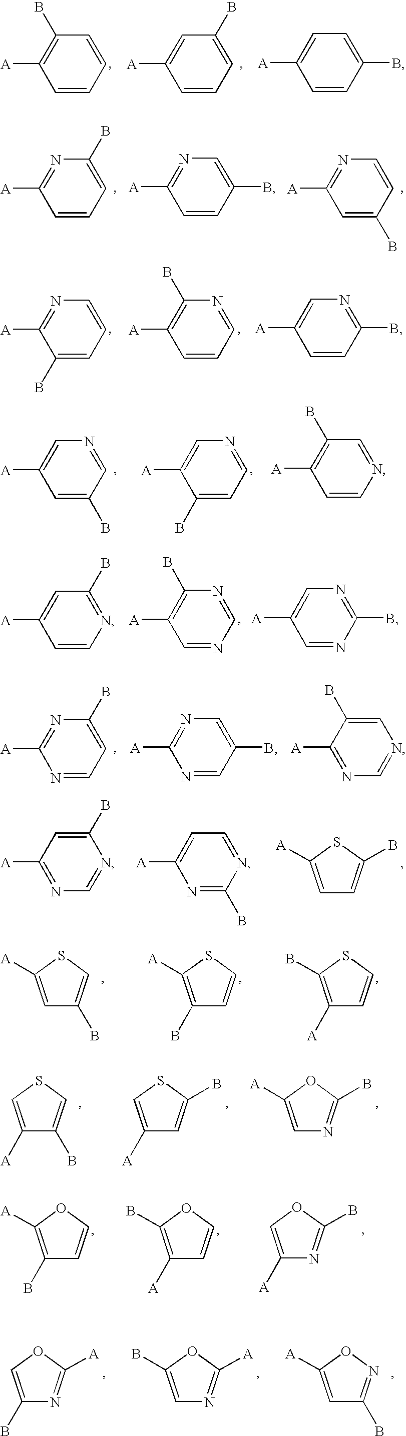

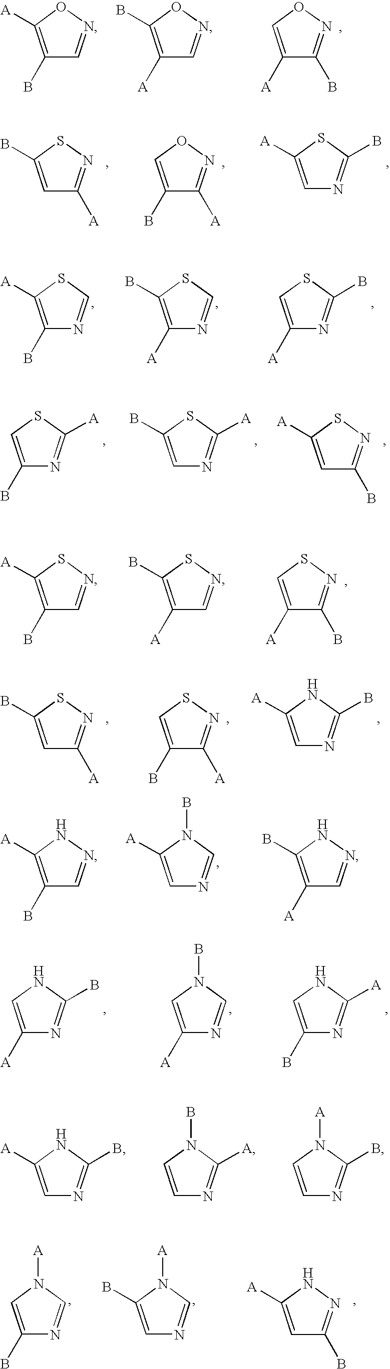

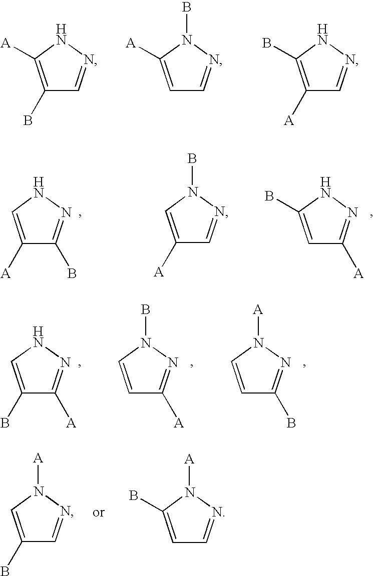

Further, these structures are optionally substituted at any one or more available ring atom(s), such as any available ring carbon atom or available ring nitrogen of imidazole or pyrazole (i.e. where the hydrogen of ═CH—, or —NH— of these structures is replaced by a substituent), as described for Formulae I, Ia, Ib, Ic or Id.

In one embodiment, compounds of Formula I have the following sub-generic structure (Formula Ie):

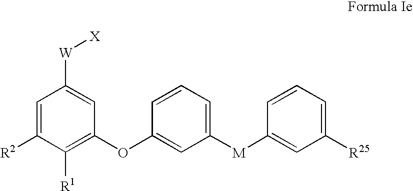

all salts, prodrugs, tautomers, and isomers thereof,
wherein:
X, W, M, R¹, and R² are as defined for Formula I; and
R²⁵ is as defined for Formulae Ia and Ib.

In one embodiment, compounds of Formula I have the following sub-generic structure (Formula If):

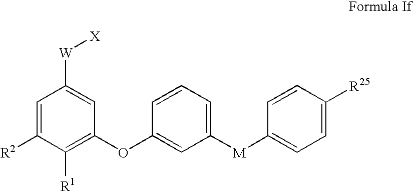

all salts, prodrugs, tautomers, and isomers thereof,
wherein:
X, W, M, R¹, and R² are as defined for Formula I; and
R²⁵ is as defined for Formulae Ia and Ib.

In one embodiment, compounds of Formula I have the following sub-generic structure (Formula Ig):

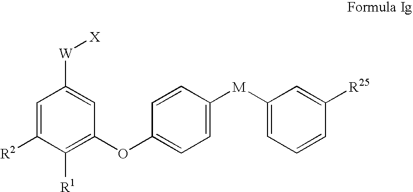

all salts, prodrugs, tautomers, and isomers thereof,
wherein:
X, W, M, R¹, and R² are as defined for Formula I; and
R²⁵ is as defined for Formulae Ia and Ib.

In one embodiment, compounds of Formula I have the following sub-generic structure (Formula Ih):

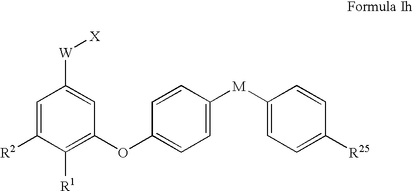

all salts, prodrugs, tautomers, and isomers thereof,
wherein:
X, W, M, R¹, and R² are as defined for Formula I; and
R²⁵ is as defined for Formulae Ia and Ib.

In one embodiment, compounds of Formula I have the following sub-generic structure (Formula Ii):

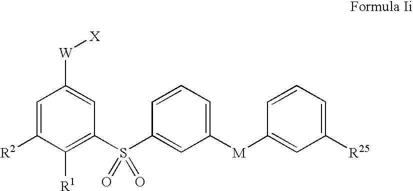

all salts, prodrugs, tautomers, and isomers thereof,
wherein:
X, W, M, R¹, and R² are as defined for Formula I; and
R²⁵ is as defined for Formulae Ia and Ib.

In one embodiment, compounds of Formula I have the following sub-generic structure (Formula Ij):

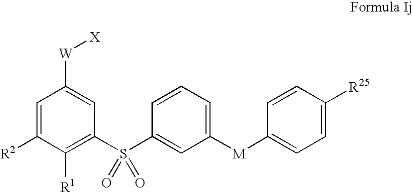

all salts, prodrugs, tautomers, and isomers thereof,
wherein:
X, W, M, R¹, and R² are as defined for Formula I; and
R²⁵ is as defined for Formulae Ia and Ib.

In one embodiment, compounds of Formula I have the following sub-generic structure (Formula Ik):

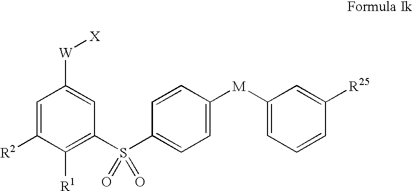

all salts, prodrugs, tautomers, and isomers thereof,
wherein:
X, W, M, R¹, and R² are as defined for Formula I; and
R²⁵ is as defined for Formulae Ia and Ib.

In one embodiment, compounds of Formula I have the following sub-generic structure (Formula Im):

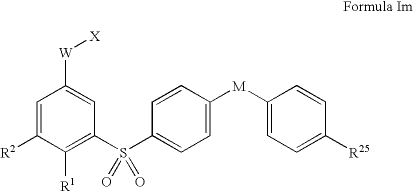

all salts, prodrugs, tautomers, and isomers thereof,
wherein:
X, W, M, R¹, and R² are as defined for Formula I; and
R²⁵ is as defined for Formulae Ia and Ib.

In one embodiment of the compounds of Formulae Ie, If, Ig, Ih, Ii, Ij, Ik, or Im, one of R¹ and R², preferably R², is —SR⁹ or —OR⁹, preferably —OR⁹, the other of R¹ and R², preferably R¹, is hydrogen, and R⁹ is selected from the group consisting of lower alkyl, C₃₋₆ alkenyl, C₃₋₆ alkynyl, and cycloalkyl, wherein lower alkyl, C₃₋₆ alkenyl, C₃₋₆ alkynyl, and cycloalkyl are optionally substituted as described for R⁹ in Formula I. In one embodiment, one of R¹ and R², preferably R², is —SR⁹ or —OR⁹, preferably —OR⁹, the other of R¹ and R², preferably R¹, is hydrogen, and R⁹ is selected from the group consisting lower alkyl, C₃₋₆ alkenyl, C₃₋₆ alkynyl, and cycloalkyl, wherein cycloalkyl is optionally substituted with one or more substituents selected from the group consisting of fluoro, —OH, lower alkyl, fluoro substituted lower alkyl, lower alkoxy, fluoro substituted lower alkoxy, lower alkylthio, and fluoro substituted lower alkylthio, and wherein lower alkyl, C₃₋₆ alkenyl, and C₃₋₆ alkynyl are optionally substituted with one or more substituents selected from the group consisting of fluoro, —OH, lower alkoxy, fluoro substituted lower alkoxy, lower alkylthio, fluoro substituted lower alkylthio, and cycloalkyl, wherein the cycloalkyl substituent of alkyl, C₃₋₆ alkenyl, or C₃₋₆ alkynyl is optionally substituted with one or more substituents selected from the group consisting of fluoro, —OH, lower alkyl, fluoro substituted lower alkyl, lower alkoxy, fluoro substituted lower alkoxy, lower alkylthio, and fluoro substituted lower alkylthio. In one embodiment, one of R¹ and R², preferably R², is —SR⁹ or —OR⁹, preferably —OR⁹, the other of R¹ and R², preferably R¹, is hydrogen, and R⁹ is selected from the group consisting of lower alkyl, C₃₋₆ alkenyl, C₃₋₆ alkynyl, and cycloalkyl, wherein the lower alkyl, C₃₋₆ alkenyl, C₃₋₆ alkynyl, and cycloalkyl are optionally substituted with one or more substituents selected from the group consisting of fluoro, lower alkoxy, and lower alkylthio. In one embodiment, one of R¹ and R², preferably R², is —SR⁹ or —OR⁹, preferably —OR⁹, the other of R¹ and R², preferably R¹, is hydrogen, and R⁹ is lower alkyl optionally substituted with one or more substituents selected from the group consisting of fluoro, lower alkoxy, fluoro substituted lower alkoxy, lower alkylthio, fluoro substituted lower alkylthio, cycloalkyl, and fluoro substituted cycloalkyl. In one embodiment, one of R¹ and R², preferably R², is —SR⁹ or —OR⁹, preferably —OR⁹, the other of R¹ and R², preferably R¹, is hydrogen, and R⁹ is lower alkyl optionally substituted with one or more substituents selected from the group consisting of fluoro, lower alkoxy, and lower alkylthio. In one embodiment, one of R¹ and R², preferably R², is —SR⁹ or —OR⁹, preferably —OR⁹, the other of R¹ and R², preferably R¹, is hydrogen, and R⁹ is perfluoroalkyl (e.g., CF₃ or CF₂CF₃) or perfluoroalkoxy (e.g., OCF₃ or OCF₂CF₃).

In one embodiment of compounds of Formulae Ie, If, Ig, Ih, Ii, Ij, Ik, or Im, W is selected from the group consisting of —NR⁵¹(CR⁴R⁵)₁₋₂—, —O—(CR⁴R⁵)₁₋₂—, —S—(CR⁴R⁵)₁₋₂—, —(CR⁴R⁵)₁₋₃—, and —CR⁶═CR⁷—, wherein R⁵¹ is hydrogen or lower alkyl optionally substituted with one or more substituents selected from the group consisting of fluoro, lower alkoxy, fluoro substituted lower alkoxy, lower alkylthio, and fluoro substituted lower alkylthio, and wherein R⁴, R⁵, R⁶ and R⁷ are independently hydrogen or lower alkyl optionally substituted with one or more substituents selected from the group consisting of fluoro, lower alkoxy, fluoro substituted lower alkoxy, lower alkylthio, and fluoro substituted lower alkylthio. In one embodiment W is selected from the group consisting of —(CR⁴R⁵)₁₋₃—, and —CR⁶═CR⁷—. In one embodiment, W is —(CR⁴R⁵)₁₋₂—. In one embodiment, W is —(CR⁴R⁵)—. In one embodiment W is selected from the group consisting of —(CR⁴R⁵)₁₋₃—, and —CR⁶═CR⁷—, wherein R⁴, R⁵, R⁶ and R⁷ are independently hydrogen or lower alkyl optionally substituted with one or more substituents selected from the group consisting of fluoro, lower alkoxy, fluoro substituted lower alkoxy, lower alkylthio, and fluoro substituted lower alkylthio. In one embodiment, W is —(CR⁴R⁵)₁₋₂—, preferably —(CR⁴R⁵)—, wherein R⁴ and R⁵ are independently hydrogen or lower alkyl optionally substituted with one or more substituents selected from the group consisting of fluoro, lower alkoxy, fluoro substituted lower alkoxy, lower alkylthio, and fluoro substituted lower alkylthio. In one embodiment, W is —CH₂CH₂— or —CH₂—, preferably —CH₂—.

In one embodiment of compounds of Formulae Ie, If, Ig, Ih, Ii, Ij, Ik, or Im, X is —C(O)OR¹⁶ or a carboxylic acid isostere, preferably X is —COOH. In one embodiment, W is —(CR⁴R⁵)₁₋₂— and X is —C(O)OR¹⁶ or a carboxylic acid isostere, preferably W is —CH₂CH₂— or —CH₂— and X is —COOH.

In one embodiment of compounds of Formulae Ie, If, Ig, Ih, Ii, Ij, Ik, or Im, R²⁵ is selected from the group consisting of halogen, —CN, lower alkyl, lower alkenyl, lower alkynyl, lower alkoxy, lower alkylthio, cycloalkyl, heterocycloalkyl, aryl and heteroaryl, wherein lower alkyl, lower alkenyl, lower alkynyl, cycloalkyl, heterocycloalkyl, aryl and heteroaryl are optionally substituted as described for R²⁵ in Formulae Ia or Ib, and wherein lower alkoxy and lower alkylthio are optionally substituted with one or more substituents selected from the group consisting of fluoro, —R³², —OR³⁶, —SR³⁶, and —NR³⁷R³⁸, where R³²R³⁶, R³⁷ and R³⁸ are as defined in Formulae Ia and Ib. In one embodiment, R²⁵ is selected from the group consisting of halogen, —CN, lower alkyl, lower alkoxy, lower alkylthio, cycloalkyl, heterocycloalkyl, aryl and heteroaryl, wherein lower alkyl, lower alkoxy, and lower alkylthio are optionally substituted with one or more substituents selected from the group consisting of fluoro, lower alkoxy, fluoro substituted lower alkoxy, lower alkylthio, and fluoro substituted lower alkylthio, and wherein cycloalkyl, heterocycloalkyl, aryl and heteroaryl are optionally substituted with one or more substituents selected from the group consisting of fluoro, —CN, lower alkyl, fluoro substituted lower alkyl, lower alkoxy, fluoro substituted lower alkoxy, lower alkylthio, and fluoro substituted lower alkylthio. In one embodiment, R²⁵ is selected from the group consisting of halogen, lower alkyl, lower alkoxy, and lower alkylthio, wherein lower alkyl, lower alkoxy, and lower alkylthio are optionally substituted with one or more substituents selected from the group consisting of fluoro, lower alkoxy, fluoro substituted lower alkoxy, lower alkylthio, and fluoro substituted lower alkylthio. In one embodiment, R²⁵ is optionally fluoro substituted lower alkyl or optionally fluoro substituted lower alkoxy. In one embodiment, R²⁵ is perfluoroalkyl (e.g., CF₃ or CF₂CF₃) or perfluoroalkoxy (e.g., OCF₃ or OCF₂CF₃).

In one embodiment of compounds of Formulae Ie, If, Ig, Ih, Ii, Ij, Ik, or Im, M is selected from the group consisting of a covalent bond, —CR¹⁹R²⁰—, —O—, —S—, and —NR⁵³—, preferably M is a covalent bond or —O—.

In one embodiment of compounds of Formulae Ie, If, Ig, Ih, Ii, Ij, Ik, or Im, one of R¹ and R², preferably R², is —OR⁹ and the other of R¹ and R², preferably R¹, is hydrogen, and W is selected from the group consisting of —(CR⁴R⁵)₁₋₃—, and —CR⁶═CR⁷—, preferably —CH₂CH₂— or —CH₂—.

In one embodiment of compounds of Formulae Ie, If, Ig, Ih, Ii, Ij, Ik, or Im, one of R¹ and R², preferably R², is —OR⁹ and the other of R¹ and R², preferably R¹, is hydrogen, W is selected from the group consisting of —(CR⁴R⁵)₁₋₃—, and —CR⁶═CR⁷—, preferably —CH₂CH₂— or —CH₂—, and M is selected from the group consisting of a covalent bond, —CR¹⁹R²⁰—, —O—, —S—, and —NR⁵³—, preferably M is a covalent bond or —O—.

In one embodiment of compounds of Formulae Ie, If, Ig, Ih, Ii, Ij, Ik, or Im, R² is —OR⁹, R¹ is hydrogen, W is —CR⁴R⁵—, X is —C(O)OR¹⁶ or a carboxylic acid isostere, and M is a covalent bond or —O—.

In one embodiment of compounds of Formulae Ie, If, Ig, Ih, Ii, Ij, Ik, or Im, R² is —OR⁹, wherein R⁹ is lower alkyl optionally substituted as described for R⁹ in Formula I, R¹ is hydrogen, W is —CR⁴R⁵—, X is —C(O)OR¹⁶ or a carboxylic acid isostere, M is a covalent bond or —O—, and R²⁵ is selected from the group consisting of halogen, —CN, lower alkyl, lower alkenyl, lower alkynyl, lower alkoxy, lower alkylthio, cycloalkyl, heterocycloalkyl, aryl and heteroaryl, wherein lower alkyl, lower alkenyl, lower alkynyl, cycloalkyl, heterocycloalkyl, aryl and heteroaryl are optionally substituted as described for R²⁵ in Formulae Ia or Ib, and lower alkoxy and lower alkylthio are optionally substituted with one or more substituents selected from the group consisting of fluoro, —R³², —OR³⁶, —SR³⁶, and —NR³⁷R³⁸, where R³², R³⁶, R³⁷ and R³⁸ are as defined in Formulae Ia and Ib.

In one embodiment of compounds of Formulae Ie, If, Ig, Ih, Ii, Ij, Ik, or Im, R² is —OR⁹, wherein R⁹ is lower alkyl optionally substituted with one or more substituents selected from the group consisting of fluoro, lower alkoxy, and lower alkylthio, R¹ is hydrogen, W is —CH₂—, X is —COOH, M is a covalent bond or —O—, and R²⁵ is selected from the group consisting of halogen, —CN, lower alkyl, lower alkoxy, lower alkylthio, cycloalkyl, heterocycloalkyl, aryl and heteroaryl, wherein lower alkyl, lower alkoxy, and lower alkylthio are optionally substituted with one or more substituents selected from the group consisting of fluoro, lower alkoxy, fluoro substituted lower alkoxy, lower alkylthio, and fluoro substituted lower alkylthio, and wherein cycloalkyl, heterocycloalkyl, aryl and heteroaryl are optionally substituted with one or more substituents selected from the group consisting of fluoro, —CN, lower alkyl, fluoro substituted lower alkyl, lower alkoxy, fluoro substituted lower alkoxy, lower alkylthio, and fluoro substituted lower alkylthio.

In one embodiment of compounds of Formulae Ie, If, Ig, Ih, Ii, Ij, Ik, or Im, R² is —OR⁹, wherein R⁹ is lower alkyl, R¹ is hydrogen, W is —CH₂—, X is —COOH, M is a covalent bond, and R²⁵ is optionally fluoro substituted lower alkyl, for example without limitation, perfluoroalkyl (e.g., CF₃ or CF₂CF₃).

In one embodiment of compounds of Formulae Ie, If, Ig, Ih, Ii, Ij, Ik, or Im, R² is —OR⁹, wherein R⁹ is lower alkyl, R¹ is hydrogen, W is —CH₂—, X is —COOH, M is a covalent bond, and R²⁵ is optionally fluoro substituted lower alkoxy, for example without limitation, perfluoroalkoxy (e.g., OCF₃ or OCF₂CF₃).

In one embodiment of compounds of Formulae Ie, If, Ig, Ih, Ii, Ij, Ik, or Im, R² is —OR⁹, wherein R⁹ is lower alkyl, R¹ is hydrogen, W is —CH₂—, X is —COOH, M is —O—, and R²⁵ is optionally fluoro substituted lower alkyl, for example without limitation, perfluoroalkyl (e.g., CF₃ or CF₂CF₃).

In one embodiment of compounds of Formulae Ie, If, Ig, Ih, Ii, Ij, Ik, or Im, R² is —OR⁹, wherein R⁹ is lower alkyl, R¹ is hydrogen, W is —CH₂—, X is —COOH, M is —O—, and R²⁵ is optionally fluoro substituted lower alkoxy, for example without limitation, perfluoroalkoxy (e.g., OCF₃ or OCF₂CF₃).

In some embodiments of the above compounds, compounds are excluded where N (except where N is a heteroaryl ring atom), O, or S is bound to a carbon that is also bound to N (except where N is a heteroaryl ring atom), O, or S; or where N (except where N is a heteroaryl ring atom), O, C(S), C(O), or S(O)ₙ (n is 0-2) is bound to an alkene carbon of an alkenyl group or bound to an alkyne carbon of an alkynyl group; accordingly, in some embodiments compounds that include linkages such as the following are excluded from the present invention: —NR—CH₂—NR—, —O—CH₂—NR—, —S—CH₂—NR—, —NR—CH₂—O—, —O—CH₂—O—, —S—CH₂—O—, —NR—CH₂—S—, —O—CH₂—S—, —S—CH₂—S—, —NR—CH═CH—, —CH═CH—NR—, —NR—C≡C—, —C≡C—NR—, —O—CH═CH—, —CH═CH—O—, —O—C≡C—, —C≡C—O—, —S(O)₀₋₂—CH═CH—, —CH═CH—S(O)₀₋₂—, —S(O)₀₋₂—C≡C—, —C≡C—S(O)₀₋₂—, —C(O)—CH═CH—, —CH═CH—C(O)—, —C≡C—C(O)—, —C(O)—C≡C—, —C(S)—CH═CH—, —CH═CH—C(S)—, —C≡C—C(S)—, or —C(S)—C≡C—.

Reference to compounds of Formula I herein includes specific reference to sub-groups and species of compounds of Formula I described herein (e.g., including Formulae Ia-Im, and all embodiments as described above) unless indicated to the contrary. In specifying a compound or compounds of Formula I, unless clearly indicated to the contrary, specification of such compound(s) includes pharmaceutically acceptable salts of the compound(s).

Another aspect of the invention relates to novel use of compounds of Formula I for the treatment of diseases associated with PPARs.

Another aspect of this invention provides compositions that include a therapeutically effective amount of a compound of Formula I and at least one pharmaceutically acceptable carrier, excipient, and/or diluent. The composition can include a plurality of different pharmacologically active compounds, including one or more compounds of Formula I.

In another aspect, compounds of Formula I can be used in the preparation of a medicament for the treatment of a PPAR-mediated disease or condition or a disease or condition in which modulation of a PPAR provides a therapeutic benefit. In a further aspect, the disease or condition is selected from the group consisting of weight disorders (e.g. obesity, overweight condition, bulimia, and anorexia nervosa), lipid disorders (e.g. hyperlipidemia, dyslipidemia including associated diabetic dyslipidemia and mixed dyslipidemia hypoalphalipoproteinemia, hypertriglyceridemia, hypercholesterolemia, and low HDL (high density lipoprotein)), metabolic disorders (e.g. Metabolic Syndrome, Type II diabetes mellitus, Type I diabetes, hyperinsulinemia, impaired glucose tolerance, insulin resistance, diabetic complication including neuropathy, nephropathy, retinopathy, diabetic foot ulcer and cataracts), cardiovascular disease (e.g. hypertension, coronary heart disease, heart failure, congestive heart failure, atherosclerosis, arteriosclerosis, stroke, cerebrovascular disease, myocardial infarction, peripheral vascular disease), inflammatory diseases (e.g. autoimmune diseases such as vitiligo, uveitis, pemphigus foliaceus, inclusion body myositis, polymyositis, dermatomyositis, scleroderma, Grave's disease, Hashimoto's disease, chronic graft versus host disease, rheumatoid arthritis, inflammatory bowel syndrome, Crohn's disease, systemic lupus erythematosis, Sjogren's Syndrome, and multiple sclerosis, diseases involving airway inflammation such as asthma and chronic obstructive pulmonary disease, and inflammation in other organs, such as polycystic kidney disease (PKD), polycystic ovary syndrome, pancreatitis, nephritis, and hepatitis), skin disorders (e.g. epithelial hyperproliferative diseases such as eczema and psoriasis, dermatitis, including atopic dermatitis, contact dermatitis, allergic dermatitis and chronic dermatitis, and impaired wound healing), neurodegenerative disorders (e.g. Alzheimer's disease, Parkinson's disease, amyotrophic lateral sclerosis, spinal cord injury, and demyelinating disease, including acute disseminated encephalomyelitis and Guillain-Barre syndrome), coagulation disorders (e.g. thrombosis), gastrointestinal disorders (e.g. infarction of the large or small intestine), genitourinary disorders (e.g. renal insufficiency, erectile dysfunction, urinary incontinence, and neurogenic bladder), ophthalmic disorders (e.g. ophthalmic inflammation, macular degeneration, and pathologic neovascularization), infections (e.g. HCV, HIV, and Helicobacter pylori), neuropathic or inflammatory pain, infertility, and cancer.

In another aspect, the invention provides kits that include a composition as described herein. In some embodiments, the composition is packaged, e.g., in a vial, bottle, flask, which may be further packaged, e.g., within a box, envelope, or bag; the composition is approved by the U.S. Food and Drug Administration or similar regulatory agency for administration to a mammal, e.g., a human; the composition is approved for administration to a mammal, e.g., a human for a PPAR-mediated disease or condition; the kit includes written instructions or other indication that the composition is suitable or approved for administration to a mammal, e.g., a human, for a PPAR-mediated disease or condition; the composition is packaged in unit dose or single dose form, e.g., single dose pills, capsules, or the like.

In another aspect, the invention provides a method of treating or prophylaxis of a disease or condition in an animal subject, e.g., a PPAR-mediated disease or condition or a disease or condition in which modulation of a PPAR provides a therapeutic benefit, by administering to the subject a therapeutically effective amount of a compound of Formula I, a prodrug of such compound, or a pharmaceutically acceptable salt of such compound or prodrug. The compound can be administered alone or can be administered as part of a pharmaceutical composition. In one aspect, the method involves administering to the subject an effective amount of a compound of Formula I in combination with one or more other therapies for the disease or condition.

In another aspect, the invention provides a method of treating or prophylaxis of a PPAR-mediated disease or condition or a disease or condition in which modulation of a PPAR provides a therapeutic benefit, wherein the method involves administering to the subject a therapeutically effective amount of a composition including a compound of Formula I.

In aspects and embodiments involving treatment or prophylaxis of a disease or condition, the disease or condition is selected from the group consisting of weight disorders (e.g. obesity, overweight condition, bulimia, and anorexia nervosa), lipid disorders (e.g. hyperlipidemia, dyslipidemia including associated diabetic dyslipidemia and mixed dyslipidemia hypoalphalipoproteinemia, hypertriglyceridemia, hypercholesterolemia, and low HDL (high density lipoprotein)), metabolic disorders (e.g. Metabolic Syndrome, Type II diabetes mellitus, Type I diabetes, hyperinsulinemia, impaired glucose tolerance, insulin resistance, diabetic complication including neuropathy, nephropathy, retinopathy, diabetic foot ulcer and cataracts), cardiovascular disease (e.g. hypertension, coronary heart disease, heart failure, congestive heart failure, atherosclerosis, arteriosclerosis, stroke, cerebrovascular disease, myocardial infarction, peripheral vascular disease), inflammatory diseases (e.g. autoimmune diseases such as vitiligo, uveitis, pemphigus foliaceus, inclusion body myositis, polymyositis, dermatomyositis, scleroderma, Grave's disease, Hashimoto's disease, chronic graft versus host disease, rheumatoid arthritis, inflammatory bowel syndrome, Crohn's disease, systemic lupus erythematosis, Sjogren's Syndrome, and multiple sclerosis, diseases involving airway inflammation such as asthma and chronic obstructive pulmonary disease, and inflammation in other organs, such as polycystic kidney disease (PKD), polycystic ovary syndrome, pancreatitis, nephritis, and hepatitis), skin disorders (e.g. epithelial hyperproliferative diseases such as eczema and psoriasis, dermatitis, including atopic dermatitis, contact dermatitis, allergic dermatitis and chronic dermatitis, and impaired wound healing), neurodegenerative disorders (e.g. Alzheimer's disease, Parkinson's disease, amyotrophic lateral sclerosis, spinal cord injury, and demyelinating disease, including acute disseminated encephalomyelitis and Guillain-Barre syndrome), coagulation disorders (e.g. thrombosis), gastrointestinal disorders (e.g. infarction of the large or small intestine), genitourinary disorders (e.g. renal insufficiency, erectile dysfunction, urinary incontinence, and neurogenic bladder), ophthalmic disorders (e.g. ophthalmic inflammation, macular degeneration, and pathologic neovascularization), infections (e.g. HCV, HIV, and Helicobacter pylori), neuropathic or inflammatory pain, infertility, and cancer.

In some embodiments of aspects involving compounds of Formula I, the compound is specific for any one or any two of PPARγ, PPARγ and PPARδ, e.g. specific for PPARα; specific for PPARδ; specific for PPARγ; specific for PPARα and PPARδ; specific for PPARα and PPARγ; or specific for PPARδ and PPARγ. Such specificity means that the compound has at least 5-fold greater activity (preferably at least 10-, 20-, 50-, or 100-fold or more greater activity) on the specific PPAR(s) than on the other PPAR(s), where the activity is determined using a biochemical assay suitable for determining PPAR activity, e.g., any assay known to one skilled in the art or as described herein. In another embodiment, compounds have significant activity on all three of PPARα, PPARδ, and PPARδ.

In some embodiments, a compound of Formula I will have an EC₅₀ of less than 100 nM, less than 50 nM, less than 20 nM, less than 10 nM, less than 5 nM, or less than 1 nM with respect to at least one of PPARα, PPARγ and PPARδ as determined in a generally accepted PPAR activity assay. In one embodiment, a compound of Formula I will have an EC₅₀ of less than 100 nM, less than 50 nM, less than 20 nM, less than 10 nM, less than 5 nM, or less than 1 nM with respect to at least any two of PPARα, PPARγ and PPARδ. In one embodiment, a compound of Formula I will have an EC₅₀ of less than 100 nM, less than 50 nM, less than 20 nM, less than 10 nM, less than 5 nM, or less than 1 nM with respect to all three of PPARα, PPARγ and PPARδ. Further to any of the above embodiments, a compound of the invention may be a specific agonist of any one of PPARα, PPARγ and PPARδ, or any two of PPARα, PPARγ and PPARδ. A specific agonist of one of PPARα, PPARγ and PPARδ is such that the EC₅₀ for one of PPARα, PPARγ and PPARδ will be at least about 5-fold, also 10-fold, also 20-fold, also 50-fold, or at least about 100-fold less than the EC₅₀ for the other two of PPARα, PPARγ and PPARδ. A specific agonist of two of PPARα, PPARγ and PPARδ is such that the EC₅₀ for each of two of PPARα, PPARγ and PPARδ will be at least about 5-fold, also 10-fold, also 20-fold, also 50-fold, or at least about 100-fold less than the EC₅₀ for the other of PPARα, PPARγ and PPARδ.

In some embodiments of the invention, the compounds of Formula I active on PPARs also have desirable pharmacologic properties. In some embodiments the desired pharmacologic property is PPAR pan-activity, PPAR selectivity for any individual PPAR (PPARα, PPARδ, or PPARγ), selectivity on any two PPARs (PPARα and PPARδ, PPARα and PPARγ, or PPARδ and PPARγ), or any one or more of serum half-life longer than 2 hr, also longer than 4 hr, also longer than 8 hr, aqueous solubility, and oral bioavailability more than 10%, also more than 20%.

Additional embodiments will be apparent from the Detailed Description and from the claims.

### DETAILED DESCRIPTION

As indicated in the Summary above, the present invention concerns the peroxisome proliferator-activated receptors (PPARs), which have been identified in humans and other mammals. A group of compounds have been identified, corresponding to Formula I, that are active on one or more of the PPARs, in particular compounds that are active on one or more human PPARs. Such compounds can be used as agonists on PPARs, including agonists of at least one of PPARα, PPARδ, and PPARγ, as well as dual PPAR agonists and pan-agonist, such as agonists of both PPARα and PPARγ, both PPARα and PPARδ, both PPARγ and PPARδ, or agonists of PPARα, PPARγ and PPARδ.

As used herein the following definitions apply unless otherwise indicated:

“Halogen”—alone or in combination refers to all halogens, that is, chloro (Cl), fluoro (F), bromo (Br), or iodo (I).

“Hydroxyl” or “hydroxy” refers to the group —OH.

“Thiol” refers to the group —SH.

“Lower alkyl” alone or in combination means an alkane-derived radical containing from 1 to 6 carbon atoms (unless specifically defined) that includes a straight chain alkyl or branched alkyl. The straight chain or branched alkyl group is attached at any available point to produce a stable compound. In many embodiments, a lower alkyl is a straight or branched alkyl group containing from 1-6, 1-4, or 1-2, carbon atoms, such as methyl, ethyl, propyl, isopropyl, butyl, t-butyl, and the like. “Substituted lower alkyl” denotes lower alkyl that is independently substituted with one or more substituents as indicated herein, for example, in the description of compounds of Formula I, including descriptions of substituted cycloalkyl, cycloheteroalkyl, aryl and heteroaryl, attached at any available atom to produce a stable compound. Preferably, substitution of lower alkyl is with 1, 2, 3, 4, or 5 substituents, also 1, 2, or 3 substituents. For example “fluoro substituted lower alkyl” denotes a lower alkyl group substituted with one or more fluoro atoms, such as perfluoroalkyl, where preferably the lower alkyl is substituted with 1, 2, 3, 4 or 5 fluoro atoms, also 1, 2, or 3 fluoro atoms.

“Lower alkenyl” alone or in combination means a straight or branched hydrocarbon containing 2-6 carbon atoms (unless specifically defined) and at least one, preferably 1-3, more preferably 1-2, most preferably one, carbon to carbon double bond. Carbon to carbon double bonds may be either contained within a straight chain or branched portion. Examples of lower alkenyl groups include ethenyl, propenyl, isopropenyl, butenyl, and the like. “Substituted lower alkenyl” denotes lower alkenyl that is independently substituted with one or more groups or substituents as indicated herein, for example, in the description of compounds of Formula I, including descriptions of substituted cycloalkyl, cycloheteroalkyl, aryl and heteroaryl, attached at any available atom to produce a stable compound. Preferably, substitution of lower alkenyl is with 1, 2, 3, 4, or 5 substituents, also 1, 2, or 3 substituents. For example “fluoro substituted lower alkenyl” denotes a lower alkenyl group substituted with one or more fluoro atoms, where preferably the lower alkenyl is substituted with 1, 2, 3, 4 or 5 fluoro atoms, also 1, 2, or 3 fluoro atoms. It is understood that substitutions are attached at any available atom to produce a stable compound, substitution of alkenyl groups are such that halogen, C(O), C(S), C(NH), S(O), S(O)₂, O, S, or N (except where N is a heteroaryl ring atom) are not bound to an alkene carbon thereof. Further, where alkenyl is a substituent of another moiety or an R group of a moiety such as —OR, —NHR, —C(O)R, and the like, substitution of the moiety is such that any C(O), C(S), S(O), S(O)₂, O, S, or N thereof (except where N is a heteroaryl ring atom) are not bound to an alkene carbon of the alkenyl substituent or R group. Further, where alkenyl is a substituent of another moiety or an R group of a moiety such as —OR, —NHR, —C(O)NHR, and the like, substitution of the alkenyl R group is such that substitution of the alkenyl carbon bound to any O, S, or N of the moiety (except where N is a heteroaryl ring atom) excludes substituents that would result in any O, S, or N of the substituent (except where N is a heteroaryl ring atom) being bound to the alkenyl carbon bound to any O, S, or N of the moiety. An “alkenyl carbon” refers to any carbon within an alkenyl group, whether saturated or part of the carbon to carbon double bond. An “alkene carbon” refers to a carbon within an alkenyl group that is part of a carbon to carbon double bond.

“Lower alkynyl” alone or in combination means a straight or branched hydrocarbon containing 2-6 carbon atoms (unless specifically defined) containing at least one, preferably one, carbon to carbon triple bond. Examples of alkynyl groups include ethynyl, propynyl, butynyl, and the like. “Substituted lower alkynyl” denotes lower alkynyl that is independently substituted with one or more groups or substituents as indicated herein, for example, in the description of compounds of Formula I, including descriptions of substituted cycloalkyl, cycloheteroalkyl, aryl and heteroaryl, attached at any available atom to produce a stable compound. Preferably, substitution of lower alkynyl is with 1, 2, 3, 4, or 5 substituents, also 1, 2, or 3 substituents. For example “fluoro substituted lower alkynyl” denotes a lower alkynyl group substituted with one or more fluoro atoms, where preferably the lower alkynyl is substituted with 1, 2, 3, 4 or 5 fluoro atoms, also 1, 2, or 3 fluoro atoms. It is understood that substitutions are attached at any available atom to produce a stable compound, substitution of alkynyl groups are such that halogen, C(O), C(S), C(NH), S(O), S(O)₂, O, S, or N (except where N is a heteroaryl ring atom) are not bound to an alkyne carbon thereof. Further, where alkynyl is a substituent of another moiety or an R group of a moiety such as —OR, —NHR, —C(O)R, and the like, substitution of the moiety is such that any C(O), C(S), S(O), S(O)₂, O, S, or N thereof (except where N is a heteroaryl ring atom) are not bound to an alkyne carbon of the alkynyl substituent or R group. Further, where alkynyl is a substituent of another moiety or an R group of a moiety such as —OR, —NHR, —C(O)NHR, and the like, substitution of the alkynyl R group is such that substitution of the alkynyl carbon bound to any O, S, or N of the moiety (except where N is a heteroaryl ring atom) excludes substituents that would result in any O, S, or N of the substituent (except where N is a heteroaryl ring atom) being bound to the alkynyl carbon bound to any O, S, or N of the moiety. An “alkynyl carbon” refers to any carbon within an alkynyl group, whether saturated or part of the carbon to carbon triple bond. An “alkyne carbon” refers to a carbon within an alkynyl group that is part of a carbon to carbon triple bond.

“Lower alkoxy” denotes the group —OR^{a}, where R^{a} is lower alkyl. “Substituted lower alkoxy” denotes lower alkoxy in which R^{a} is lower alkyl substituted with one or more substituents as indicated herein, for example, in the description of compounds of Formula I, including descriptions of substituted cycloalkyl, cycloheteroalkyl, aryl and heteroaryl, attached at any available atom to produce a stable compound. Preferably, substitution of lower alkoxy is with 1, 2, 3, 4, or 5 substituents, also 1, 2, or 3 substituents. For example “fluoro substituted lower alkoxy” denotes lower alkoxy in which the lower alkyl is substituted with one or more fluoro atoms, where preferably the lower alkoxy is substituted with 1, 2, 3, 4 or 5 fluoro atoms, also 1, 2, or 3 fluoro atoms. It is understood that substitutions on alkoxy are attached at any available atom to produce a stable compound, substitution of alkoxy is such that O, S, or N (except where N is a heteroaryl ring atom) are not bound to the alkyl carbon bound to the alkoxy O. Further, where alkoxy is described as a substituent of another moiety, the alkoxy oxygen is not bound to a carbon atom that is bound to an O, S, or N of the other moiety (except where N is a heteroaryl ring atom) or to an alkene or alkyne carbon of the other moiety.

“Lower alkylthio” denotes the group —SR^{b}, where R^{b} is lower alkyl. “Substituted lower alkylthio” denotes lower alkylthio in which R^{b} is lower alkyl substituted with one or more substituents as indicated herein, for example, in the description of compounds of Formula I, including descriptions of substituted cycloalkyl, cycloheteroalkyl, aryl and heteroaryl, attached at any available atom to produce a stable compound. Preferably, substitution of lower alkylthio is with 1, 2, 3, 4, or 5 substituents, also 1, 2, or 3 substituents. For example “fluoro substituted lower alkylthio” denotes lower alkylthio in which the lower alkyl is substituted with one or more fluoro atoms, where preferably the lower alkylthio is substituted with 1, 2, 3, 4 or 5 fluoro atoms, also 1, 2, or 3 fluoro atoms. It is understood that substitutions on alkylthio are attached at any available atom to produce a stable compound, substitution of alkylthio is such that O, S, or N (except where N is a heteroaryl ring atom) are not bound to the alkyl carbon bound to the alkylthio S. Further, where alkylthio is described as a substituent of another moiety, the alkylthio sulfur is not bound to a carbon atom that is bound to an O, S, or N of the other moiety (except where N is a heteroaryl ring atom) or to an alkene or alkyne carbon of the other moiety.

“Amino” or “amine” denotes the group —NH₂. “Mono-alkylamino” denotes the group —NHR^{c} where R^{c} is lower alkyl. “Di-alkylamino” denotes the group —NR^{c}R^{d}, where R^{c} and R^{d} are independently lower alkyl. “Cycloalkylamino” denotes the group —NR^{e}R^{f}, where R^{e} and R^{f} combine with the nitrogen to form a 5-7 membered heterocycloalkyl, where the heterocycloalkyl may contain an additional heteroatom within the ring, such as O, N, or S, and may also be further substituted with lower alkyl. Examples of 5-7 membered heterocycloalkyl include, but are not limited to, piperidine, piperazine, 4-methylpiperazine, morpholine, and thiomorpholine. It is understood that when mono-alkylamino, di-alkylamino, or cycloalkylamino are substituents on other moieties that are attached at any available atom to produce a stable compound, the nitrogen of mono-alkylamino, di-alkylamino, or cycloalkylamino as substituents is not bound to a carbon atom that is bound to an O, S, or N of the other moiety (except where N is a heteroaryl ring atom) or to an alkene or alkyne carbon of the other moiety.

“Carboxylic acid isostere” refers to a moiety selected from the group consisting of thiazolidine dione

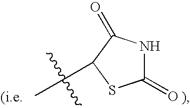

hydroxamic acid (i.e. —C(O)NHOH), acyl-cyanamide (i.e. —C(O)NHCN), tetrazole

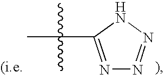

3- or 5-hydroxy isoxazole

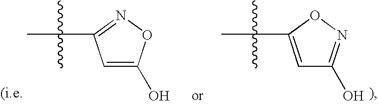

3- or 5-hydroxy isothiazole (i.e.

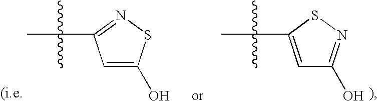

sulphonate (i.e. —S(O)₂OH), and sulfonamide (i.e. —S(O)₂NH₂). In functional terms, carboxylic acid isosteres mimic carboxylic acids by virtue of similar physical properties, including but not limited to molecular size, charge distribution or molecular shape. 3- or 5-hydroxy isoxazole or 3- or 5-hydroxy isothiazole may be optionally substituted with lower alkyl or lower alkyl substituted with 1, 2 or 3 substituents selected from the group consisting of fluoro, aryl and heteroaryl, wherein aryl or heteroaryl may further be optionally substituted with 1, 2, or 3 substituents selected from the group consisting of halogen, lower alkyl, fluoro substituted lower alkyl, lower alkoxy, fluoro substituted lower alkoxy, lower alkylthio, and fluoro substituted lower alkylthio. The nitrogen of the sulfonamide may be optionally substituted with a substituent selected from the group consisting of lower alkyl, fluoro substituted lower alkyl, acetyl (i.e. —C(O)CH₃), aryl and heteroaryl, wherein aryl or heteroaryl may further be optionally substituted with 1, 2, or 3 substituents selected from the group consisting of halogen, lower alkyl, fluoro substituted lower alkyl, lower alkoxy, fluoro substituted lower alkoxy, lower alkylthio, and fluoro substituted lower alkylthio.

“Aryl” alone or in combination refers to a monocyclic or bicyclic ring system containing aromatic hydrocarbons such as phenyl or naphthyl, which may be optionally fused with a cycloalkyl or heterocycloalkyl of preferably 5-7, more preferably 5-6, ring members. “Arylene” refers to a divalent aryl.

“Heteroaryl” alone or in combination refers to a monocyclic aromatic ring structure containing 5 or 6 ring atoms, or a bicyclic aromatic group having 8 to 10 atoms, containing one or more, preferably 1-4, more preferably 1-3, even more preferably 1-2, heteroatoms independently selected from the group consisting of O, S, and N. Heteroaryl is also intended to include oxidized S or N, such as sulfinyl, sulfonyl and N-oxide of a tertiary ring nitrogen. A carbon or nitrogen atom is the point of attachment of the heteroaryl ring structure such that a stable compound is produced. Examples of heteroaryl groups include, but are not limited to, pyridinyl, pyridazinyl, pyrazinyl, quinaoxalyl, indolizinyl, benzo[b]thienyl, quinazolinyl, purinyl, indolyl, quinolinyl, pyrimidinyl, pyrrolyl, pyrazolyl, oxazolyl, thiazolyl, thienyl, isoxazolyl, oxathiadiazolyl, isothiazolyl, tetrazolyl, imidazolyl, triazolyl, furanyl, benzofuryl, and indolyl. “Nitrogen containing heteroaryl” refers to heteroaryl wherein any heteroatoms are N. “Heteroarylene” refers to a divalent heteroaryl.

“Cycloalkyl” refers to saturated or unsaturated, non-aromatic monocyclic, bicyclic or tricyclic carbon ring systems of 3-10, also 3-8, more preferably 3-6, ring members per ring, such as cyclopropyl, cyclopentyl, cyclohexyl, adamantyl, and the like.

“Heterocycloalkyl” refers to a saturated or unsaturated non-aromatic cycloalkyl group having from 5 to 10 atoms in which from 1 to 3 carbon atoms in the ring are replaced by heteroatoms of O, S or N, and are optionally fused with benzo or heteroaryl of 5-6 ring members. Heterocycloalkyl is also intended to include oxidized S or N, such as sulfinyl, sulfonyl and N-oxide of a tertiary ring nitrogen. Heterocycloalkyl is also intended to include compounds in which one of the ring carbons is oxo substituted, i.e. the ring carbon is a carbonyl group, such as lactones and lactams. The point of attachment of the heterocycloalkyl ring is at a carbon or nitrogen atom such that a stable ring is retained. Examples of heterocycloalkyl groups include, but are not limited to, morpholino, tetrahydrofuranyl, dihydropyridinyl, piperidinyl, pyrrolidinyl, pyrrolidonyl, piperazinyl, dihydrobenzofuryl, and dihydroindolyl.

“Optionally substituted aryl”, “optionally substituted arylene”, “optionally substituted heteroaryl”, “optionally substituted heteroarylene”, “optionally substituted cycloalkyl”, and “optionally substituted heterocycloalkyl”, refers to aryl, arylene, heteroaryl, heteroarylene, cycloalkyl and heterocycloalkyl groups, respectively, which are optionally independently substituted, unless indicated otherwise, with one or more, preferably 1, 2, 3, 4 or 5, also 1, 2, or 3 substituents, attached at any available atom to produce a stable compound, wherein the substituents are selected from the group consisting of halogen, —OH, —NH₂, —NO₂, —CN, —C(O)OH, —C(S)OH, —C(O)NH₂, —C(S)NH₂, —S(O)₂NH₂, —NHC(O)NH₂, —NHC(S)NH₂, —NHS(O)₂NH₂, —C(NH)NH₂, —OR^{g}, —SR^{g}, —OC(O)R^{g}, —OC(S)R^{g}, —C(O)R^{g}, —C(S)R^{g}, —C(O)OR^{g}, —C(S)OR^{g}, —S(O)R^{g}, —S(O)₂R^{g}, —C(O)NHR^{g}, —C(S)NHR^{g}, —C(O)NR^{g}R^{g}, —C(S)NR^{g}R^{g}, —S(O)₂NHR^{g}, —S(O)₂NR^{g}R^{g}, —C(NH)NHR^{g}, —C(NH)NR^{g}R^{g}, —NHC(O)R^{g}, —NHC(S)R^{g}, —NR^{g}C(O)R^{g}, —NR^{g}C(S)R^{g}, —NHS(O)₂R^{g}, —NR^{g}S(O)₂R^{g}, —NHC(O)NHR^{g}, —NHC(S)NHR^{g}, —NR^{g}C(O)NH₂, —NR^{g}C(S)NH₂, —NR^{g}C(O)NHR^{g}, —NR^{g}C(S)NHR^{g}, —NHC(O)NR^{g}R^{g}, —NHC(S)NR^{g}R^{g}, —NR^{g}C(O)NR^{g}R^{g}, —NR^{g}C(S)NR^{g}R^{g}, —NHS(O)₂NHR^{g}, —NR^{g}S(O)₂NH₂, —NR^{g}S(O)₂NHR^{g}, —NHS(O)₂NR^{g}R^{g}, —NR^{g}S(O)₂NR^{g}R^{g}, —NHR^{g}, —NR^{g}R^{g}, —R^{j}, —R^{k}, and —R^{m};
wherein —R^{g}, —R^{h}, and —Rⁱ at each occurrence are independently selected from the group consisting of —Rⁿ, —R^{o}, and —R^{p}, or
—R^{h} and —Rⁱ combine with the nitrogen to which they are attached form a 5-7 membered heterocycloalkyl or a 5 or 7 membered nitrogen containing heteroaryl, wherein the 5-7 membered heterocycloalkyl or 5 or 7 membered nitrogen containing heteroaryl are optionally substituted with one or more, preferably 1, 2, 3, 4 or 5, also 1, 2, or 3 substituents selected from the group consisting of halogen, cycloalkylamino, —NO₂, —CN, —OH, —NH₂, —OR^{t}, —SR^{t}, —NHR^{t}, —NR^{t}R^{t}, —R^{q}, and —R^{u};
wherein each —R^{j} is independently lower alkyl optionally substituted with one or more, preferably 1, 2, 3, 4 or 5, also 1, 2 or 3 substituents selected from the group consisting of fluoro, cycloalkylamino, —OH, —NH₂, —OR^{t}, —OR^{u}, —SR^{t}, —SR^{u}, —NHR^{t}, —NHR^{u}, —NR^{t}R^{u}, —NR^{t}R^{t}, —NR^{u}R^{u}, and —R^{m};
wherein each —R^{k} is independently selected from the group consisting of lower alkenyl and lower alkynyl, wherein lower alkenyl or lower alkynyl are optionally substituted with one or more, preferably 1, 2, 3, 4 or 5, also 1, 2 or 3 substituents selected from the group consisting of fluoro, cycloalkylamino, —OH, —NH₂, —OR^{t}, —OR^{u}, —SR^{t}, —SR^{u}, —NHR^{t}, —NHR^{u}, —NR^{t}R^{u}, —NR^{t}R^{t}, —NR^{u}R^{u}, —R^{j}, and —R^{m};
wherein each —R^{m} is independently selected from the group consisting of cycloalkyl, heterocycloalkyl, aryl, and heteroaryl, wherein cycloalkyl, heterocycloalkyl, aryl, and heteroaryl are optionally substituted with one or more, preferably 1, 2, 3, 4 or 5, also 1, 2 or 3 substituents selected from the group consisting of halogen, cycloalkylamino, —NO₂, —CN, —OH, —NH₂, —OR^{t}, —OR^{u}, —SR^{t}, —SR^{u}, —NHR^{t}, —NHR^{u}, —NR^{t}R^{u}, —NR^{t}R^{t}, —NR^{u}R^{u}, —R^{q}, and —R^{u};
wherein each —Rⁿ is independently lower alkyl optionally substituted with one or more, preferably 1, 2, 3, 4 or 5, also 1, 2, or 3 substituents selected from the group consisting of fluoro, cycloalkylamino, —OH, —NH₂, —OR^{t}, —OR^{u}, —SR^{t}, —SR^{u}, —NHR^{t}, —NHR^{u}, —NR^{t}R^{u}, —NR^{t}R^{u}, —NR^{u}R^{u}, and —R^{m}, provided, however, that any substitution on the alkyl carbon bound to any O, S, or N of any OR^{g}, SR^{g}, or NR^{g} is selected from the group consisting of fluoro and —R^{m};
wherein each —R^{o} is independently selected from the group consisting of C₃₋₆ alkenyl and C₃₋₆ alkynyl, wherein C₃₋₆ alkenyl or C₃₋₆ alkynyl are optionally substituted with one or more, preferably 1, 2, 3, 4 or 5, also 1, 2, or 3 substituents selected from the group consisting of fluoro, cycloalkylamino, —OH, —NH₂, —OR^{t}, —OR^{u}, —SR^{t}, —SR^{u}, —NHR^{t}, —NHR^{u}, —NR^{t}R^{u}, —NR^{t}R^{t}, —NR^{u}R^{u}, —R^{j} and —R^{m}, provided, however, that any substitution on the alkenyl or alkynyl carbon bound to any O, S, or N of any —OR^{g}, —SR^{g}, or NR^{g} is selected from the group consisting of fluoro, —R^{j} and —R^{m};
wherein each R^{p} is independently selected from the group consisting of cycloalkyl, heterocycloalkyl, aryl, and heteroaryl, wherein cycloalkyl, heterocycloalkyl, aryl, and heteroaryl are optionally substituted with one or more, preferably 1, 2, 3, 4 or 5, also 1, 2, or 3 substituents selected from the group consisting of halogen, cycloalkylamino, —NO₂, —CN, —OH, —NH₂, —OR^{t}, —OR^{u}, —SR^{t}, —SR^{u}, —NHR^{t}, —NHR^{u}, —NR^{t}R^{u}, —NR^{t}R^{t}, —NR^{u}R^{u}, —R^{q}, and —R^{u};
wherein each —R^{q} is independently selected from the group consisting of lower alkyl, lower alkenyl and lower alkynyl, wherein lower alkyl is optionally substituted with one or more, preferably 1, 2, 3, 4 or 5, also 1, 2, or 3 substituents selected from the group consisting of —R^{u}, fluoro, lower alkoxy, fluoro substituted lower alkoxy, lower alkylthio, fluoro substituted lower alkylthio, mono-alkylamino, di-alkylamino, and cycloalkylamino, and wherein lower alkenyl or lower alkynyl are optionally substituted with one or more, preferably 1, 2, 3, 4 or 5, also 1, 2, or 3 substituents selected from the group consisting of —R^{u}, fluoro, lower alkyl, fluoro substituted lower alkyl, lower alkoxy, fluoro substituted lower alkoxy, lower alkylthio, fluoro substituted lower alkylthio, mono-alkylamino, di-alkylamino, and cycloalkylamino;
wherein each —R^{t} is independently selected from the group consisting of lower alkyl, C₃₋₆ alkenyl and C₃₋₆ alkynyl, wherein lower alkyl is optionally substituted with one or more, preferably 1, 2, 3, 4 or 5, also 1, 2, or 3 substituents selected from the group consisting of —R^{u}, fluoro, lower alkoxy, fluoro substituted lower alkoxy, lower alkylthio, fluoro substituted lower alkylthio, mono-alkylamino, di-alkylamino, and cycloalkylamino, provided, however, that any substitution of the lower alkyl carbon bound to the O of —OR^{t}, S of —SR^{t}, or N of —NHR^{t}, —NR^{t}R^{t}, or —NR^{t}R^{u} is fluoro or —R^{u}, and wherein C₃₋₆ alkenyl or C₃₋₆ alkynyl are optionally substituted with one or more, preferably 1, 2, 3, 4 or 5, also 1, 2, or 3 substituents selected from the group consisting of —R^{u}, fluoro, lower alkyl, fluoro substituted lower alkyl, lower alkoxy, fluoro substituted lower alkoxy, lower alkylthio, fluoro substituted lower alkylthio, mono-alkylamino, di-alkylamino, and cycloalkylamino, provided, however, that any substitution of the C₃₋₆ alkenyl or C₃₋₆ alkynyl carbon bound to the O of —OR^{t}, S of —SR^{t}, or N of —NHR^{t}, —NR^{t}R^{t}, or —NR^{t}R^{u} is fluoro, lower alkyl, fluoro substituted lower alkyl, or —R^{u};
wherein each —R^{u} is independently selected from the group consisting of cycloalkyl, heterocycloalkyl, aryl, and heteroaryl, wherein cycloalkyl, heterocycloalkyl, aryl, and heteroaryl are optionally substituted with one or more, preferably 1, 2, 3, 4 or 5, also 1, 2, or 3 substituents selected from the group consisting of halogen, —OH, —NH₂, —NO₂, —CN, lower alkyl, fluoro substituted lower alkyl, lower alkoxy, fluoro substituted lower alkoxy, lower alkylthio, fluoro substituted lower alkylthio, mono-alkylamino, di-alkylamino, and cycloalkylamino.

As used herein in connection with PPAR modulating compound, binding compounds or ligands, the term “specific for PPAR” and terms of like import mean that a particular compound binds to a PPAR to a statistically greater extent than to other biomolecules that may be present in or originally isolated from a particular organism, e.g., at least 2, 3, 4, 5, 10, 20, 50, 100, or 1000-fold greater binding. Also, where biological activity other than binding is indicated, the term “specific for PPAR” indicates that a particular compound has greater biological activity associated with binding to a PPAR than to other biomolecules (e.g., at a level as indicated for binding specificity). Similarly, the specificity can be for a specific PPAR with respect to other PPARs that may be present in or originally isolated from a particular organism.

Also in the context of compounds binding to a biomolecular target, the term “greater specificity” indicates that a compound binds to a specified target to a greater extent than to another biomolecule or biomolecules that may be present under relevant binding conditions, where binding to such other biomolecules produces a different biological activity than binding to the specified target. In some cases, the specificity is with reference to a limited set of other biomolecules, e.g., in the case of PPARs, in some cases the reference may be other receptors, or for a particular PPAR, it may be other PPARs. In some embodiments, the greater specificity is at least 2, 3, 4, 5, 8, 10, 50, 100, 200, 400, 500, or 1000-fold greater specificity. In the context of ligands interacting with PPARs, the terms “activity on”, “activity toward,” and like terms mean that such ligands have EC₅₀ less than 10 μM, less than 1 μM, less than 100 nM, less than 50 nM, less than 20 nM, less than 10 nM, less than 5 nM, or less than 1 nM with respect to at least one PPAR as determined in a generally accepted PPAR activity assay.

The term “composition” or “pharmaceutical composition” refers to a formulation suitable for administration to an intended animal subject for therapeutic purposes. The formulation includes a therapeutically significant quantity (i.e. a therapeutically effective amount) of at least one active compound and at least one pharmaceutically acceptable carrier or excipient, which is prepared in a form adapted for administration to a subject. Thus, the preparation is “pharmaceutically acceptable”, indicating that it does not have properties that would cause a reasonably prudent medical practitioner to avoid administration of the material to a patient, taking into consideration the disease or conditions to be treated and the respective route of administration. In many cases, such a pharmaceutical composition is a sterile preparation, e.g. for injectibles.

The term “PPAR-mediated” disease or condition and like terms refer to a disease or condition in which the biological function of a PPAR affects the development and/or course of the disease or condition, and/or in which modulation of PPAR alters the development, course, and/or symptoms of the disease or condition. Similarly, the phrase “PPAR modulation provides a therapeutic benefit” indicates that modulation of the level of activity of PPAR in a subject indicates that such modulation reduces the severity and/or duration of the disease, reduces the likelihood or delays the onset of the disease or condition, and/or causes an improvement in one or more symptoms of the disease or condition. In some cases the disease or condition may be mediated by any one or more of the PPAR isoforms, e.g., PPARγ, PPARα, PPARδ, PPARγ and PPARα, PPARγ and PPARδ, PPARα and PPARδ, or PPARγ, PPARα, and PPARδ.

The term “therapeutically effective” or “effective amount” indicates that the materials or amount of material is effective to prevent, alleviate, or ameliorate one or more symptoms of a disease or medical condition, and/or to prolong the survival of the subject being treated.

The term “PPAR” refers to a peroxisome proliferator-activated receptor as recognized in the art. As indicated above, the PPAR family includes PPARα (also referred to as PPARa or PPARalpha), PPARδ (also referred to as PPARd or PPARdelta), and PPARγ (also referred to as PPARg or PPARgamma). The individual PPARs can be identified by their sequences, where exemplary reference sequence accession numbers are as follows:

| Receptor | Sequence | Accession No. | SEQ ID NO: |
|---|---|---|---|
| hPPARa | cDNA | NM_005036 | |
| hPPARa | protein | NP_005027 | |
| hPPARg isoform 2 | cDNA | NM_015869 | |
| hPPARg isoform 2 | protein | NP_056953 | |
| hPPARd | cDNA | NM_006238 | |
| hPPARd | protein | NP_006229 | |

One of ordinary skill in the art will recognize that sequence differences will exist due to allelic variation, and will also recognize that other animals, particularly other mammals, have corresponding PPARs, which have been identified or can be readily identified using sequence alignment and confirmation of activity. Such homologous PPARs can also be used in the present invention, which homologous PPARs have sequence identity of, for example, at least 50%, 60%, 70%, 80%, 90%, 95%, 99%, or even 100%, over a region spanning 50, 100, 150, 200, 250, 300, 350, 400, 450, 500, or even more amino acids or nucleotides for proteins or nucleic acids, respectively. One of ordinary skill in the art will also recognize that modifications can be introduced in a PPAR sequence without destroying PPAR activity. Such modified PPARs can also be used in the present invention, e.g., if the modifications do not alter the binding site conformation to the extent that the modified PPAR lacks substantially normal ligand binding.

As used herein in connection with the design or development of ligands, the term “bind” and “binding” and like terms refer to a non-covalent energetically favorable association between the specified molecules (i.e., the bound state has a lower free energy than the separated state, which can be measured calorimetrically). For binding to a target, the binding is at least selective, that is, the compound binds preferentially to a particular target or to members of a target family at a binding site, as compared to non-specific binding to unrelated proteins not having a similar binding site. For example, BSA is often used for evaluating or controlling for non-specific binding. In addition, for an association to be regarded as binding, the decrease in free energy going from a separated state to the bound state must be sufficient so that the association is detectable in a biochemical assay suitable for the molecules involved.

By “assaying” is meant the creation of experimental conditions and the gathering of data regarding a particular result of the experimental conditions. For example, enzymes can be assayed based on their ability to act upon a detectable substrate. Likewise, for example, a compound or ligand can be assayed based on its ability to bind to a particular target molecule or molecules and/or to modulate an activity of a target molecule.

By “background signal” in reference to a binding assay is meant the signal that is recorded under standard conditions for the particular assay in the absence of a test compound, molecular scaffold, or ligand that binds to the target molecule. Persons of ordinary skill in the art will realize that accepted methods exist and are widely available for determining background signal.

By “clog P” is meant the calculated log P of a compound, “P” referring to the partition coefficient of the compound between a lipophilic and an aqueous phase, usually between octanol and water.

In the context of compounds binding to a target, the term “greater affinity” indicates that the compound binds more tightly than a reference compound, or than the same compound in a reference condition, i.e., with a lower dissociation constant. In some embodiments, the greater affinity is at least 2, 3, 4, 5, 8, 10, 50, 100, 200, 400, 500, 1000, or 10,000-fold greater affinity.

By binding with “moderate affinity” is meant binding with a K_{D} of from about 200 nM to about 1 μM under standard conditions. By “moderately high affinity” is meant binding at a K_{D} of from about 1 nM to about 200 nM. By binding at “high affinity” is meant binding at a K_{D} of below about 1 nM under standard conditions. The standard conditions for binding are at pH 7.2 at 37° C. for one hour. For example, typical binding conditions in a volume of 100 μl/well would comprise a PPAR, a test compound, HEPES 50 mM buffer at pH 7.2, NaCl 15 mM, ATP 2 μM, and bovine serum albumin (1 μg/well), at 37° C. for one hour.

Binding compounds can also be characterized by their effect on the activity of the target molecule. Thus, a “low activity” compound has an inhibitory concentration (IC₅₀) (for inhibitors or antagonists) or effective concentration (EC₅₀) (applicable to agonists) of greater than 1 μM under standard conditions. By “moderate activity” is meant an IC₅₀ or EC₅₀ of 200 nM to 1 μM under standard conditions. By “moderately high activity” is meant an IC₅₀ or EC₅₀ of 1 nM to 200 nM. By “high activity” is meant an IC₅₀ or EC₅₀ of below 1 nM under standard conditions. The IC₅₀ (or EC₅₀) is defined as the concentration of compound at which 50% of the activity of the target molecule (e.g., enzyme or other protein) activity being measured is lost (or gained) relative to activity when no compound is present. Activity can be measured using methods known to those of ordinary skill in the art, e.g., by measuring any detectable product or signal produced by occurrence of an enzymatic reaction, or other activity by a protein being measured. For PPAR agonists, activities can be determined as described in the Examples, or using other such assay methods known in the art.

By “protein” is meant a polymer of amino acids. The amino acids can be naturally or non-naturally occurring. Proteins can also contain modifications, such as being glycosylated, phosphorylated, or other common modifications.

By “protein family” is meant a classification of proteins based on structural and/or functional similarities. For example, kinases, phosphatases, proteases, and similar groupings of proteins are protein families. Proteins can be grouped into a protein family based on having one or more protein folds in common, a substantial similarity in shape among folds of the proteins, homology, or based on having a common function. In many cases, smaller families will be specified, e.g., the PPAR family.

By “specific biochemical effect” is meant a therapeutically significant biochemical change in a biological system causing a detectable result. This specific biochemical effect can be, for example, the inhibition or activation of an enzyme, the inhibition or activation of a protein that binds to a desired target, or similar types of changes in the body's biochemistry. The specific biochemical effect can cause alleviation of symptoms of a disease or condition or another desirable effect. The detectable result can also be detected through an intermediate step.

By “standard conditions” is meant conditions under which an assay is performed to obtain scientifically meaningful data. Standard conditions are dependent on the particular assay, and can be generally subjective. Normally the standard conditions of an assay will be those conditions that are optimal for obtaining useful data from the particular assay. The standard conditions will generally minimize background signal and maximize the signal sought to be detected.

By “standard deviation” is meant the square root of the variance. The variance is a measure of how spread out a distribution is. It is computed as the average squared deviation of each number from its mean. For example, for the numbers 1, 2, and 3, the mean is 2 and the variance is:

$${σ}^{2} = \frac{{( 1 - 2 )}^{2} + {( 2 - 2 )}^{2} + {( 3 - 2 )}^{2}}{3} = 0.667 .$$

In the context of this invention, by “target molecule” is meant a molecule that a compound, molecular scaffold, or ligand is being assayed for binding to. The target molecule has an activity that binding of the molecular scaffold or ligand to the target molecule will alter or change. The binding of the compound, scaffold, or ligand to the target molecule can preferably cause a specific biochemical effect when it occurs in a biological system. A “biological system” includes, but is not limited to, a living system such as a human, animal, plant, or insect. In most but not all cases, the target molecule will be a protein or nucleic acid molecule.

By “pharmacophore” is meant a representation of molecular features that are considered to be responsible for a desired activity, such as interacting or binding with a receptor. A pharmacophore can include 3-dimensional (hydrophobic groups, charged/ionizable groups, hydrogen bond donors/acceptors), 2D (substructures), and 1D (physical or biological) properties.

As used herein in connection with numerical values, the terms “approximately” and “about” mean ±10% of the indicated value.

### I. Applications of PPAR Agonists

The PPARs have been recognized as suitable targets for a number of different diseases and conditions. Some of those applications are described briefly below. Additional applications are known and the present compounds can also be used for those diseases and conditions.

(a) Insulin resistance and diabetes: In connection with insulin resistance and diabetes, PPARγ is necessary and sufficient for the differentiation of adipocytes in vitro and in vivo. In adipocytes, PPARγ increases the expression of numerous genes involved in lipid metabolism and lipid uptake. In contrast, PPARγ down-regulates leptin, a secreted, adipocyte-selective protein that has been shown to inhibit feeding and augment catabolic lipid metabolism. This receptor activity could explain the increased caloric uptake and storage noted in vivo upon treatment with PPARγ agonists. Clinically, TZDs, including troglitazone, rosiglitazone, and pioglitazone, and non-TZDs, including farglitazar, have insulin-sensitizing and anti-diabetic activity. (Berger, et al., Diabetes Tech. And Ther., 2002, 4:163-174).

PPARγ has been associated with several genes that affect insulin action. TNFα, a proinflammatory cytokine that is expressed by adipocytes, has been associated with insulin resistance. PPARγ agonists inhibit expression of TNFα in adipose tissue of obese rodents, and ablate the actions of TNFα in adipocytes in vitro. PPARγ agonists were shown to inhibit expression of 11β-hydroxysteroid dehydrogenase 1 (11β-HSD-1), the enzyme that converts cortisone to the glucocorticoid agonist cortisol, in adipocytes and adipose tissue of type 2 diabetes mouse models. This is noteworthy since hypercortico-steroidism exacerbates insulin resistance. Adipocyte Complement-Related Protein of 30 kDa (Acrp30 or adiponectin) is a secreted adipocyte-specific protein that decreases glucose, triglycerides, and free fatty acids. In comparison to normal human subjects, patients with type 2 diabetes have reduced plasma levels of Acrp30. Treatment of diabetic mice and non-diabetic human subjects with PPARγ agonists increases plasma levels of Acrp30. Induction of Acrp30 by PPARγ agonists might therefore also play a key role in the insulin-sensitizing mechanism of PPARγ agonists in diabetes. (Berger, et al., supra).

PPARγ is expressed predominantly in adipose tissue. Thus, it is believed that the net in vivo efficacy of PPARγ agonists involves direct actions on adipose cells with secondary effects in key insulin responsive tissues such as skeletal muscle and liver. This is supported by the lack of glucose-lowering efficacy of rosiglitazone in a mouse model of severe insulin resistance where white adipose tissue was essentially absent. Furthermore, in vivo treatment of insulin resistant rats produces acute (<24 h) normalization of adipose tissue insulin action whereas insulin-mediated glucose uptake in muscle was not improved until several days after the initiation of therapy. This is consistent with the fact that PPARγ agonists can produce an increase in adipose tissue insulin action after direct in vitro incubation, whereas no such effect could be demonstrated using isolated in vitro incubated skeletal muscles. The beneficial metabolic effects of PPARγ agonists on muscle and liver may be mediated by their ability to (a) enhance insulin-mediated adipose tissue uptake, storage (and potentially catabolism) of free fatty acids; (b) induce the production of adipose-derived factors with potential insulin sensitizing activity (e.g., Acrp30); and/or (c) suppress the circulating levels and/or actions of insulin resistance-causing adipose-derived factors such as TNFα or resistin. (Berger, et al., supra).

(b) Dyslipidemia and atherosclerosis: In connection with dyslipidemia and atherosclerosis, PPARα has been shown to play a critical role in the regulation of cellular uptake, activation, and β-oxidation of fatty acids. Activation of PPARα induces expression of fatty acid transport proteins and enzymes in the peroxisomal β-oxidation pathway. Several mitochondrial enzymes involved in the energy-harvesting catabolism of fatty acids are robustly upregulated by PPARα agonists. Peroxisome proliferators also activate expression of the CYP4As, a subclass of cytochrome P450 enzymes that catalyze the ω-hydroxylation of fatty acids, a pathway that is particularly active in the fasted and diabetic states. In sum, it is clear that PPARα is an important lipid sensor and regulator of cellular energy-harvesting metabolism. (Berger, et al., supra).

Atherosclerosis is a very prevalent disease in Westernized societies. In addition to a strong association with elevated LDL cholesterol, “dyslipidemia” characterized by elevated triglyceride-rich particles and low levels of HDL cholesterol is commonly associated with other aspects of a metabolic syndrome that includes obesity, insulin resistance, type 2 diabetes, and an increased risk of coronary artery disease. Thus, in 8,500 men with known coronary artery disease, 38% were found to have low HDL (<35 mg/dL) and 33% had elevated triglycerides (>200 mg/dL). In such patients, treatment with fibrates resulted in substantial triglyceride lowering and modest HDL-raising efficacy. More importantly, a recent large prospective trial showed that treatment with gemfibrozil produced a 22% reduction in cardiovascular events or death. Thus PPARα agonists can effectively improve cardiovascular risk factors and have a net benefit to improve cardiovascular outcomes. In fact, fenofibrate was recently approved in the United States for treatment of type IIA and IIB hyper-lipidemia. Mechanisms by which PPARα activation cause triglyceride lowering are likely to include the effects of agonists to suppress hepatic apo-CIII gene expression while also stimulating lipoprotein lipase gene expression. Dual PPARγ/α agonists, including KRP-297 and DRF 2725, possess potent lipid-altering efficacy in addition to anti-hyperglycemic activity in animal models of diabetes and lipid disorders.

The presence of PPARα and/or PPARγ expression in vascular cell types, including macrophages, endothelial cells, and vascular smooth muscle cells, suggests that direct vascular effects might contribute to potential antiatherosclerosis efficacy. PPARα and PPARα activation have been shown to inhibit cytokine-induced vascular cell adhesion and to suppress monocyte-macrophage migration. Several additional studies have also shown that PPARγ-selective compounds have the capacity to reduce arterial lesion size and attenuate monocyte-macrophage homing to arterial lesions in animal models of atherosclerosis. PPARγ is present in macrophages in human atherosclerotic lesions, and may play a role in regulation of expression of matrix metalloproteinase-9 (MMP-9), which is implicated in atherosclerotic plaque rupture (Marx et al., Am J Pathol. 1998, 153(1):17-23). Downregulation of LPS induced secretion of MMP-9 was also observed for both PPARα and PPARγ agonists, which may account for beneficial effects observed with PPAR agonists in animal models of atherosclerosis (Shu et al., Biochem Biophys Res Commun. 2000, 267(1):345-9). PPARγ is also shown to have a role in intercellular adhesion molecule-1 (ICAM-1) protein expression (Chen et al., Biochem Biophys Res Commun. 2001, 282(3):717-22) and vascular cell adhesion molecule-1 (VCAM-1) protein expression (Jackson et al., Arterioscler Thromb Vasc Biol. 1999, 19(9):2094-104) in endothelial cells, both of which play a role in the adhesion of monocytes to endothelial cells. In addition, two recent studies have suggested that either PPARα or PPARγ activation in macrophages can induce the expression of a cholesterol efflux “pump” protein.

It has been found that relatively selective PPARδ agonists produce minimal, if any, glucose- or triglyceride-lowering activity in murine models of type 2 diabetes in comparison with efficacious PPARγ or PPARα agonists. Subsequently, a modest increase in HDL-cholesterol levels was detected with PPARδ agonists in db/db mice. Recently, Oliver et al. (supra) reported that a potent, selective PPARδ agonist could induce a substantial increase in HDL-cholesterol levels while reducing triglyceride levels and insulin resistance in obese rhesus monkeys.

Thus, via multifactorial mechanisms that include improvements in circulating lipids, systemic and local anti-inflammatory effects, and, inhibition of vascular cell proliferation, PPARα, PPARγ, and PPARδ agonists can be used in the treatment or prevention of atherosclerosis. (Berger, et al., supra).

(c) Inflammation: Monocytes and macrophages are known to play an important part in the inflammatory process through the release of inflammatory cytokines and the production of nitric oxide by inducible nitric oxide synthase. Rosiglitazone has been shown to induce apoptosis of macrophages at concentrations that parallel its affinity for PPARγ. This ligand has also been shown to block inflammatory cytokine synthesis in colonic cell lines. This latter observation suggests a mechanistic explanation for the observed anti-inflammatory actions of TZDs in rodent models of colitis.

Additional studies have examined the relationship between macrophages, cytokines and PPARγ and agonists thereof (Jiang et al., Nature 1998, 391(6662):82-6, Ricote et al., Nature 1998, 391(6662):79-82, Hortelano et al., J Immunol. 2000, 165(11):6525-31, and Chawla et al., Nat Med. 2001, 7(1):48-52) suggesting a role for PPARγ agonists in treating inflammatory responses, for example in autoimmune diseases.

The migration of monocytes and macrophages plays a role in the development of inflammatory responses as well. PPAR ligands have been shown to have an effect on a variety of chemokines. Monocyte chemotactic protein-1 (MCP-1) directed migration of monocytes is attenuated by PPARγ and PPARα ligands in a monocytic leukemia cell line (Kintscher et al., Eur J Pharmacol. 2000, 401(3):259-70). MCP-1 gene expression was shown to be suppressed by PPARγ ligand 15-deoxy-Delta(12,14)PGJ2 (15d-PGJ2) in two monocytic cell lines, which also showed induction of IL-8 gene expression (Zhang et al., J Immunol. 2001, 166(12):7104-11).

Anti-inflammatory actions have been described for PPARα ligands that can be important in the maintenance of vascular health. Treatment of cytokine-activated human macrophages with PPARα agonists induced apoptosis of the cells. It was reported that PPARα agonists inhibit activation of aortic smooth muscle cells in response to inflammatory stimuli. (Staels et al., 1998, Nature 393:790-793.) In hyperlipidemic patients, fenofibrate treatment decreases the plasma concentrations of the inflammatory cytokine interleukin-6.

Anti-inflammatory pathways in airway smooth muscle cells were investigated with respect to PPARα and PPARγ Patel et al., 2003, The Journal of Immunology, 170:2663-2669). This study demonstrated an anti-inflammatory effect of a PPARγ ligand that may be useful in the treatment of COPD and steroid-insensitive asthma.

The anti-inflammatory effects of PPAR modulators have also been studied with respect to autoimmune diseases, such as chronic inflammatory bowel syndrome, arthritis, Crohn's disease and multiple sclerosis, and in neuronal diseases such as Alzheimer's disease and Parkinson's disease.

(d) Hypertension: Hypertension is a complex disorder of the cardiovascular system that has been shown to be associated with insulin resistance. Type 2 diabetes patients demonstrate a 1.5-2-fold increase in hypertension in comparison with the general population. Troglitazone, rosiglitazone, and pioglitazone therapy have been shown to decrease blood pressure in diabetic patients as well as troglitazone therapy in obese, insulin-resistant subjects. Since such reductions in blood pressure were shown to correlate with decreases in insulin levels, they can be mediated by an improvement in insulin sensitivity. However, since TZDs also lowered blood pressure in one-kidney one-clip Sprague Dawley rats, which are not insulin resistant, it was proposed that the hypotensive action of PPARα agonists is not exerted solely through their ability to improve insulin sensitivity. Other mechanisms that have been invoked to explain the anti-hypertensive effects of PPARγ agonists include their ability to (a) downregulate expression of peptides that control vascular tone such as PAI-I, endothelin, and type-c natriuretic peptide C or (b) alter calcium concentrations and the calcium sensitivity of vascular cells. (Berger et al., supra).

(e) Cancer: PPAR modulation has also been correlated with cancer treatment. (Burstein, et al.; Breast Cancer Res. Treat., 2003, 79(3):391-7; Alderd, et al.; Oncogene, 2003, 22(22):3412-6).

(f) Weight Control: Administration of PPARα agonists can induce satiety, and thus are useful in weight loss or maintenance. Such PPARα agonists can act preferentially on PPARα, or can also act on another PPAR, or can be PPAR pan-agonists. Thus, the satiety inducing effect of PPARα agonists can be used for weight control or loss.

(g) Autoimmune diseases: PPAR agonists may provide benefits in the treatment of autoimmune diseases. Agonists of PPAR isoforms may be involved in T cell and B cell trafficking or activity, the altering of oligodendrocyte function or differentiation, the inhibition of macrophage activity, the reduction of inflammatory responses, and neuroprotective effects, some or all of which may be important in a variety of autoimmune diseases.

Multiple sclerosis (MS) is a neurodegenerative autoimmune disease that involves the demyelination of axons and formation of plaques. PPARδ mRNA has been shown to be strongly expressed in immature oligodendrocytes (Granneman et al., J Neurosci Res. 1998, 51(5):563-73). PPARδ selective agonists or pan-agonists were shown to accelerate differentiation of oligodendrocytes, with no effect on differentiation observed with a PPARγ selective agonist. An alteration in the myelination of corpus callosum was observed in PPARδ null mice (Peters et al., Mol Cell Biol. 2000, 20(14):5119-28). It was also shown that PPARδ mRNA and protein is expressed throughout the brain in neurons and oligodendrocytes, but not in astrocytes (Woods et al., Brain Res. 2003, 975(1-2): 10-21). These observations suggest that PPARδ has a role in myelination, where modulation of such a role could be used to treat multiple sclerosis by altering the differentiation of oligodendrocytes, which may result in slowing of the demyelination, or even promoting the remyelination of axons. It has also been shown that oligodendrocyte-like B12 cells, as well as isolated spinal cord oligodendrocytes from rat, are affected by PPARγ agonists. Alkyl-dihydroxyacetone phosphate synthase, a key peroxisomal enzyme involved in the synthesis of plasmologens, which are a key component of myelin, is increased in PPARγ agonist treated B12 cells, while the number of mature cells in isolated spinal cord oligodendrocytes increases with PPARγ agonist treatment.

The role of PPAR in the regulation of B and T cells may also provide therapeutic benefits in diseases such as MS. For example, it has been shown that PPARγ agonists can inhibit the secretion of IL-2 by T cells (Clark et al., J Immunol. 2000, 164(3):1364-71) or may induce apoptosis in T cells (Harris et al., Eur J Immunol. 2001, 31(4):1098-105), suggesting an important role in cell-mediated immune responses. An antiproliferative and cytotoxic effect on B cells by PPARγ agonists has also been observed (Padilla et al., Clin Immunol. 2002, 103(1):22-33).

The anti-inflammatory effects of PPAR modulators, as discussed herein, may also be useful in treating MS, as well as a variety of other autoimmune diseases such as Type-1 diabetes mellitus, psoriasis, vitiligo, uveitis, Sjogren's disease, pemphigus foliaceus, inclusion body myositis, polymyositis, dermatomyositis, scleroderma, Grave's disease, Hashimoto's disease, chronic graft-versus host disease, rheumatoid arthritis, inflammatory bowel syndrome, and Crohn's disease. Using a mouse model, the PPARα agonists gemfibrozil and fenofibrate were shown to inhibit clinical signs of experimental autoimmune encephalomyelitis, suggesting that PPARα agonists may be useful in treating inflammatory conditions such as multiple sclerosis (Lovett-Racke et al., J Immunol. 2004, 172(9):5790-8).

Neuroprotective effects that appear to be associated with PPARs may also aid in the treatment of MS. The effects of PPAR agonists on LPS induced neuronal cell death were studied using cortical neuron-glial co-cultures. PPARγ agonists 15d-PGJ2, ciglitazone and troglitazone were shown to prevent the LPS-induced neuronal cell death, as well as abolish NO and PGE2 release and a reduction in iNOS and COX-2 expression (Kim et al., Brain Res. 2002, 941(1-2):1-10).

Rheumatoid arthritis (RA) is an autoimmune inflammatory disease that results in the destruction of joints. In addition to chronic inflammation and joint damage due in part to mediators such as IL-6 and TNF-alpha, osteoclast differentiation is also implicated in damage to the joints. PPAR agonists may regulate these pathways, providing therapeutic benefits in treatment of RA. In studies using PPARγ agonist troglitazone in fibroblast-like synovial cells (FLS) isolated from patients with rheumatoid arthritis, an inhibition of cytokine mediated inflammatory responses was observed (Yamasaki et al., Clin Exp Immunol., 2002, 129(2):379-84). PPARγ agonists have also demonstrated beneficial effects in a rat or mouse model of RA (Kawahito et al., J Clin Invest. 2000, 106(2): 189-97; Cuzzocrea et al., Arthritis Rheum. 2003, 48(12):3544-56). The effects of the PPARα ligand fenofibrate on rheumatoid synovial fibroblasts from RA patients also showed inhibition of cytokine production, as well as NF-KappaB activation and osteoclast differentiation. Fenofibrate was also shown to inhibit the development of arthritis in a rat model (Okamoto et al., Clin Exp Rheumatol. 2005, 23(3):323-30).

Psoriasis is a T cell mediated autoimmune disease, where T cell activation leads to release of cytokines and resulting proliferation of keratinocytes. In addition to anti-inflammatory effects, the differentiation of keratinocytes may also be a therapeutic target for PPAR agonists. Studies in a PPARδ null mouse model suggest using PPARδ ligand to selectively induce keratinocyte differentiation and inhibit cell proliferation (Kim et al., Cell Death Differ. 2005). Thiazolidinedione ligands of PPARγ have been shown to inhibit the proliferation of psoriatic keratinocytes in monolayer and organ culture, and when applied topically inhibit epidermal hyperplasia of human psoriatic skin transplanted to SCID mice (Bhagavathula et al., J Pharmacol Exp Ther. 2005, 315(3) 996-1004).

(h) Neurodegenerative diseases: The modulation of the PPARs may provide benefits in the treatment of neuronal diseases. For example, the anti-inflammatory effects of PPAR modulators discussed herein have also been studied with respect to neuronal diseases such as Alzheimer's disease and Parkinson's disease.

In addition to inflammatory processes, Alzheimer's disease is characterized by deposits of amyloid-beta (Abeta) peptides and neurofibrillary tangles. A decrease in the levels of Abeta peptide in neuronal and non-neuronal cells was observed with induced expression of PPARγ, or by activation of PPARγ using a thiazolidinedione (Camacho et al., J Neurosci. 2004, 24(48):10908-17). Treatment of APP7171 mice with PPARγ agonist pioglitazone showed several beneficial effects, including reduction in activated microglia and reactive astrocytes in the hippocampus and cortex, reduction in proinflammatory cyclooxygenase 2 and inducible nitric oxide synthase, decreased β-secretase-1 mRNA and protein levels, and a reduction in the levels of soluble Abeta1-42 peptide (Heneka et al., Brain. 2005, 128(Pt 6):1442-53).

Regions of degeneration of dopamine neurons in Parkinson's disease have been associated with increased levels of inflammatory cytokines (Nagatsu et al., J Neural Transm Suppl. 2000 (60):277-90). The effect of PPARγ agonist pioglitazone on dopaminergic nerve cell death and glial activation was studied in an MPTP mouse model of Parkinson's disease, wherein orally administered pioglitazone resulted in reduced glial activation as well as prevention of dopaminergic cell loss (Breidert et al. Journal of Neurochemistry, 2002, 82: 615).

(i) Other indications: PPARγ modulators have shown inhibition of VEGF-induced choroidal angiogenesis as well as repression of choroidal neovascularization effects, suggesting potential for treatment of retinal disorders. PPARδ has been shown to be expressed in implantation sites and in decidual cells in rats, suggesting a role in pregnancy, such as to enhance fertility. These studies were reviewed in Kota, et al., Pharmacological Research, 2005, 51: 85-94.

The management of pain, either neuropathic or inflammatory, is also suggested as a possible target for PPAR modulators. Burstein, S., Life Sci. 2005, 77(14):1674-84, suggests that PPARγ provides a receptor function for the activity of some cannabinoids. Lo Verme et al., Mol. Pharmacol. 2005, 67(1):15-9, identifies PPARα as a target responsible for pain and inflammation reducing effects of palmitoylethanolamide (PEA). PEA selectively activates PPARα in vitro, and induces expression of PPARα mRNA when applied topically to mice. In animal models of carrageenan-induced paw edema and phorbol ester-induced ear edema, inflammation in wild type mice is attenuated by PEA, which has no effect in PPARα deficient mice. PPARα agonists OEA, GW7647 and Wy-14643 demonstrate similar effects. Benani et al., Neurosci Lett. 2004, 369(1):59-63, uses a model of inflammation in rats to assess the PPAR response in the rat spinal cord following injection of complete Freund's adjuvant into the hind paw. It was shown that PPARα was activated, suggesting a role in pain pathways.

PPARα are also involved in some infections, and may be targeted in treating such infections. Dharancy et al. report that HCV infection is related to altered expression and function of the anti-inflammatory nuclear receptor PPARalpha, and identify hepatic PPARalpha as one mechanism underlying the pathogenesis of HCV infection, and as a new therapeutic target in traditional treatment of HCV-induced liver injury (Dharancy et al., Gastroenterology 2005, 128(2):334-42). J Raulin reports that among other effects, HIV infection induces alteration of cellular lipids, including deregulation of PPAR-gamma (J. Raulin, Prog Lipid Res 2002, 41(1):27-65). Slomiany and Slomiany report that PPARgamma activation leading to the impedance of Helicobacter pylori lipopolysaccharide (LPS) inhibitory effect on salivary mucin synthesis requires epidermal growth factor receptor (EGFR) participation. Further, they showed the impedance by ciglitazone was blunted in a concentration dependent fashion by a PPAR gamma agonist. (Slomiany and Slomiany, Inflammopharmacology 2004, 12(2):177-88).

Muto et al. (Human Molecular Genetics 2002, 11(15):1731-1742) showed that molecular defects observed in Pkd1^{-/-} embryos contribute to the pathogenesis of autosomal dominant polycystic kidney disease (ADPKD) and that thiazolidindiones have a compensatory effect on the pathway affected by the loss of polycystin-1. Thus pathways activated by thiazolidinediones may provide new therapeutic targets in ADPKD (Muto et al., supra). Glintborg et al. show an increase in growth hormone levels in subjects with polycystic ovary syndrome treated with pioglitazone (Glintborg et al., J Clin Endocrinol Metab 2005, 90(10):5605-12).

In accordance with the description above, isoforms of the PPAR family of nuclear receptors are clearly involved in the systemic regulation of lipid metabolism and serve as “sensors” for fatty acids, prostanoid metabolites, eicosanoids and related molecules. These receptors function to regulate a broad array of genes in a coordinate fashion. Important biochemical pathways that regulate insulin action, lipid oxidation, lipid synthesis, adipocyte differentiation, peroxisome function, cell apoptosis, and inflammation can be modulated through the individual PPAR isoforms. Strong therapeutic effects of PPARα and PPARγ agonists to favorably influence systemic lipid levels, glucose homeostasis, and atherosclerosis risk (in the case of PPARα activation in humans) have recently been discovered. PPARα and PPARγ agonists are presently used clinically to favorably alter systemic lipid levels and glucose homeostasis, respectively. Recent observations made using PPARS ligands suggest that this isoform is also an important therapeutic target for dyslipidemia and insulin resistance, as well.

Thus, PPAR agonists, such as those described herein by Formulae I, Ia, Ib, Ic and Id, can be used in the prophylaxis and/or therapeutic treatment of a variety of different diseases and conditions, such as weight disorders (e.g. obesity, overweight condition, bulimia, and anorexia nervosa), lipid disorders (e.g. hyperlipidemia, dyslipidemia including associated diabetic dyslipidemia and mixed dyslipidemia hypoalphalipoproteinemia, hypertriglyceridemia, hypercholesterolemia, and low HDL (high density lipoprotein)), metabolic disorders (e.g. Metabolic Syndrome, Type II diabetes mellitus, Type I diabetes, hyperinsulinemia, impaired glucose tolerance, insulin resistance, diabetic complication including neuropathy, nephropathy, retinopathy, diabetic foot ulcer and cataracts), cardiovascular disease (e.g. hypertension, coronary heart disease, heart failure, congestive heart failure, atherosclerosis, arteriosclerosis, stroke, cerebrovascular disease, myocardial infarction, peripheral vascular disease), inflammatory diseases (e.g. autoimmune diseases such as vitiligo, uveitis, pemphigus foliaceus, inclusion body myositis, polymyositis, dermatomyositis, scleroderma, Grave's disease, Hashimoto's disease, chronic graft versus host disease, rheumatoid arthritis, inflammatory bowel syndrome, Crohn's disease, systemic lupus erythematosis, Sjogren's Syndrome, and multiple sclerosis, diseases involving airway inflammation such as asthma and chronic obstructive pulmonary disease, and inflammation in other organs, such as polycystic kidney disease (PKD), polycystic ovary syndrome, pancreatitis, nephritis, and hepatitis), skin disorders (e.g. epithelial hyperproliferative diseases such as eczema and psoriasis, dermatitis, including atopic dermatitis, contact dermatitis, allergic dermatitis and chronic dermatitis, and impaired wound healing), neurodegenerative disorders (e.g. Alzheimer's disease, Parkinson's disease, amyotrophic lateral sclerosis, spinal cord injury, and demyelinating disease, including acute disseminated encephalomyelitis and Guillain-Barre syndrome), coagulation disorders (e.g. thrombosis), gastrointestinal disorders (e.g. infarction of the large or small intestine), genitourinary disorders (e.g. renal insufficiency, erectile dysfunction, urinary incontinence, and neurogenic bladder), ophthalmic disorders (e.g. ophthalmic inflammation, macular degeneration, and pathologic neovascularization), infections (e.g. HCV, HIV, and Helicobacter pylori), neuropathic or inflammatory pain, infertility, and cancer.

### II. PPAR Active Compounds

As indicated in the Summary and in connection with applicable diseases and conditions, a number of different PPAR agonists have been identified. In addition, the present invention provides PPAR agonist compounds described by Formulae I, Ia, Ib, Ic or Id as provided in the Summary above.

The activity of the compounds can be assessed using methods known to those of skill in the art, as well as methods described herein. Screening assays may include controls for purposes of calibration and confirmation of proper manipulation of the components of the assay. Blank wells that contain all of the other reactants but no compound active on PPARs are usually included. As another example, a known inhibitor (or activator) of an enzyme for which modulators are sought can be incubated with one sample of the assay, and the resulting decrease (or increase) in the enzyme activity used as a comparator or control. It will be appreciated that modulators can also be combined with the enzyme activators or inhibitors to find modulators which inhibit the enzyme activation or repression that is otherwise caused by the presence of the known enzyme modulator. Similarly, when ligands to a target are sought, known ligands of the target can be present in control/calibration assay wells.

(a) Enzymatic Activity Assays

A number of different assays can be utilized to assess activity of PPAR modulators and/or determine specificity of a modulator for a particular PPAR. In addition to the assays mentioned in the Examples below, one of ordinary skill in the art will know of other assays that can be utilized and can modify an assay for a particular application. For example, the assay can utilize AlphaScreen (amplified luminescent proximity homogeneous assay) format, e.g., AlphaScreening system (Packard BioScience). AlphaScreen is generally described in Seethala and Prabhavathi, Homogenous Assays: AlphaScreen, Handbook of Drug Screening, Marcel Dekkar Pub. 2001, pp. 106-110. Applications of the technique to PPAR receptor ligand binding assays are described, for example, in Xu, et al., Nature, 2002, 415:813-817.

(b) Assessment of Efficacy of Compounds in Disease Model Systems.

The utility of compounds of Formula I for the treatment of diseases such as autoimmune diseases and neurological diseases can be readily assessed using model systems known to those of skill in the art. For example, efficacy of PPAR modulators in models of Alzheimer's disease can be tested by mimicking inflammatory injury to neuronal tissues and measuring recovery using molecular and pharmacological markers (Heneka, et al., J. Neurosci., 2000, 20:6862-6867). Efficacy of PPAR modulators in multiple sclerosis has been monitored using the accepted model of experimental autoimmune encephalomyelitis (EAE) (Storer, et al., J Neuroimmunol., 2004, 161:113-122. See also: Niino, et al., J. Neuroimmunol., 2001, 116:40-48; Diab, et al. J. Immunol., 2002, 168:2508-2515; Natarajan, et al., Genes Immun., 2002, 3:59-70; Feinstein, et al., Ann. Neurol., 2002, 51:694-702.)

(c) Isomers, Prodrugs, and Active Metabolites

Compounds contemplated herein are described with reference to both generic formulae and specific compounds. In addition, the invention compounds may exist in a number of different forms or derivatives, all within the scope of the present invention. These include, for example, tautomers, stereoisomers, racemic mixtures, regioisomers, salts, prodrugs (e.g., carboxylic acid esters), solvated forms, different crystal forms or polymorphs, and active metabolites.

(d) Tautomers, Stereoisomers, Regioisomers, and Solvated Forms

It is understood that some compounds may exhibit tautomerism. In such cases, the formulae provided herein expressly depict only one of the possible tautomeric forms. It is therefore to be understood that the formulae provided herein are intended to represent any tautomeric form of the depicted compounds and are not to be limited merely to the specific tautomeric form depicted by the drawings of the formulae.

Likewise, some of the compounds according to the present invention may exist as stereoisomers, i.e. having the same atomic connectivity of covalently bonded atoms yet differing in the spatial orientation of the atoms. For example, compounds may be optical stereoisomers, which contain one or more chiral centers, and therefore, may exist in two or more stereoisomeric forms (e.g. enantiomers or diastereomers). Thus, such compounds may be present as single stereoisomers (i.e., essentially free of other stereoisomers), racemates, and/or mixtures of enantiomers and/or diastereomers. As another example, stereoisomers include geometric isomers, such as cis- or trans-orientation of substituents on adjacent carbons of a double bond. All such single stereoisomers, racemates and mixtures thereof are intended to be within the scope of the present invention. Unless specified to the contrary, all such steroisomeric forms are included within the formulae provided herein.

In some embodiments, a chiral compound of the present invention is in a form that contains at least 80% of a single isomer (60% enantiomeric excess (“e.e.”) or diastereomeric excess (“d.e.”)), or at least 85% (70% e.e. or d.e.), 90% (80% e.e. or d.e.), 95% (90% e.e. or d.e.), 97.5% (95% e.e. or d.e.), or 99% (98% e.e. or d.e.). As generally understood by those skilled in the art, an optically pure compound having one chiral center is one that consists essentially of one of the two possible enantiomers (i.e., is enantiomerically pure), and an optically pure compound having more than one chiral center is one that is both diastereomerically pure and enantiomerically pure. In some embodiments, the compound is present in optically pure form.

For compounds in which synthesis involves addition of a single group at a double bond, particularly a carbon-carbon double bond, the addition may occur at either of the double bond-linked atoms. For such compounds, the present invention includes both such regioisomers.

Additionally, the formulae are intended to cover solvated as well as unsolvated forms of the identified structures. For example, the indicated structures include both hydrated and non-hydrated forms. Other examples of solvates include the structures in combination with a suitable solvent, such as isopropanol, ethanol, methanol, DMSO, ethyl acetate, acetic acid, or ethanolamine.

(e) Prodrugs and Metabolites

In addition to the present formulae and compounds described herein, the invention also includes prodrugs (generally pharmaceutically acceptable prodrugs), active metabolic derivatives (active metabolites), and their pharmaceutically acceptable salts.

Prodrugs are compounds or pharmaceutically acceptable salts thereof which, when metabolized under physiological conditions or when converted by solvolysis, yield the desired active compound. Prodrugs include, without limitation, esters, amides, carbamates, carbonates, ureides, solvates, or hydrates of the active compound. Typically, the prodrug is inactive, or less active than the active compound, but may provide one or more advantageous handling, administration, and/or metabolic properties. For example, some prodrugs are esters of the active compound; during metabolysis, the ester group is cleaved to yield the active drug. Also, some prodrugs are activated enzymatically to yield the active compound, or a compound which, upon further chemical reaction, yields the active compound. In this context, a common example is an alkyl ester of a carboxylic acid.

As described in The Practice of Medicinal Chemistry, Ch. 31-32 (Ed. Wermuth, Academic Press, San Diego, Calif., 2001), prodrugs can be conceptually divided into two non-exclusive categories, bioprecursor prodrugs and carrier prodrugs. Generally, bioprecursor prodrugs are compounds that are inactive or have low activity compared to the corresponding active drug compound, that contain one or more protective groups and are converted to an active form by metabolism or solvolysis. Both the active drug form and any released metabolic products should have acceptably low toxicity. Typically, the formation of active drug compound involves a metabolic process or reaction that is one of the follow types:

Oxidative reactions: Oxidative reactions are exemplified without limitation to reactions such as oxidation of alcohol, carbonyl, and acid functionalities, hydroxylation of aliphatic carbons, hydroxylation of alicyclic carbon atoms, oxidation of aromatic carbon atoms, oxidation of carbon-carbon double bonds, oxidation of nitrogen-containing functional groups, oxidation of silicon, phosphorus, arsenic, and sulfur, oxidative N-dealkylation, oxidative O- and S-dealkylation, oxidative deamination, as well as other oxidative reactions.

Reductive reactions: Reductive reactions are exemplified without limitation to reactions such as reduction of carbonyl functionalities, reduction of alcohol functionalities and carbon-carbon double bonds, reduction of nitrogen-containing functional groups, and other reduction reactions.

Reactions without change in the oxidation state: Reactions without change in the state of oxidation are exemplified without limitation to reactions such as hydrolysis of esters and ethers, hydrolytic cleavage of carbon-nitrogen single bonds, hydrolytic cleavage of non-aromatic heterocycles, hydration and dehydration at multiple bonds, new atomic linkages resulting from dehydration reactions, hydrolytic dehalogenation, removal of hydrogen halide molecule, and other such reactions.

Carrier prodrugs are drug compounds that contain a transport moiety, e.g., that improves uptake and/or localized delivery to a site(s) of action. Desirably for such a carrier prodrug, the linkage between the drug moiety and the transport moiety is a covalent bond, the prodrug is inactive or less active than the drug compound, the prodrug and any release transport moiety are acceptably non-toxic. For prodrugs where the transport moiety is intended to enhance uptake, typically the release of the transport moiety should be rapid. In other cases, it is desirable to utilize a moiety that provides slow release, e.g., certain polymers or other moieties, such as cyclodextrins. (See, e.g., Cheng et al., U.S. Patent Publ. No. 20040077595, application Ser. No. 10/656,838, incorporated herein by reference.)

Such carrier prodrugs are often advantageous for orally administered drugs. Carrier prodrugs can, for example, be used to improve one or more of the following properties: increased lipophilicity, increased duration of pharmacological effects, increased site-specificity, decreased toxicity and adverse reactions, and/or improvement in drug formulation (e.g., stability, water solubility, suppression of an undesirable organoleptic or physiochemical property). For example, lipophilicity can be increased by esterification of hydroxyl groups with lipophilic carboxylic acids, or of carboxylic acid groups with alcohols, e.g., aliphatic alcohols. Wermuth, supra.

Prodrugs may proceed from prodrug form to active form in a single step or may have one or more intermediate forms which may themselves have activity or may be inactive.

Metabolites, e.g., active metabolites, overlap with prodrugs as described above, e.g., bioprecursor prodrugs. Thus, such metabolites are pharmacologically active compounds or compounds that further metabolize to pharmacologically active compounds that are derivatives resulting from metabolic processes in the body of a subject. Of these, active metabolites are such pharmacologically active derivative compounds. For prodrugs, the prodrug compound is generally inactive or of lower activity than the metabolic product. For active metabolites, the parent compound may be either an active compound or may be an inactive prodrug. Metabolites of a compound may be identified using routine techniques known in the art, and their activities determined using tests such as those described herein. For example, in some compounds, one or more alkoxy groups can be metabolized to hydroxyl groups while retaining pharmacologic activity and/or carboxyl groups can be esterified, e.g., glucuronidation. In some cases, there can be more than one metabolite, where an intermediate metabolite(s) is further metabolized to provide an active metabolite. For example, in some cases a derivative compound resulting from metabolic glucuronidation may be inactive or of low activity, and can be further metabolized to provide an active metabolite.

Prodrugs and active metabolites may be identified using routine techniques known in the art. See, e.g., Bertolini et al., 1997, J. Med. Chem., 40:2011-2016; Shan et al., 1997, J Pharm Sci 86(7):756-757; Bagshawe, 1995, Drug Dev. Res., 34:220-230; Wermuth, supra.

(f) Pharmaceutically Acceptable Salts

Compounds can be formulated as or be in the form of pharmaceutically acceptable salts. Contemplated pharmaceutically acceptable salt forms include, without limitation, mono, bis, tris, tetrakis, and so on. Pharmaceutically acceptable salts are non-toxic in the amounts and concentrations at which they are administered. The preparation of such salts can facilitate the pharmacological use by altering the physical characteristics of a compound without preventing it from exerting its physiological effect. Useful alterations in physical properties include lowering the melting point to facilitate transmucosal administration and increasing the solubility to facilitate administering higher concentrations of the drug. A compound of the invention may possess a sufficiently acidic, a sufficiently basic, or both functional groups, and accordingly react with any of a number of inorganic or organic bases, and inorganic and organic acids, to form a pharmaceutically acceptable salt.

Pharmaceutically acceptable salts include acid addition salts such as those containing sulfate, pyrosulfate, bisulfate, sulfite, bisulfite, chloride, bromide, iodide, hydrochloride, fumarate, maleate, phosphate, monohydrogenphosphate, dihydrogenphosphate, metaphosphate, pyrophosphate, sulfamate, acetate, citrate, lactate, tartrate, sulfonate, methanesulfonate, propanesulfonate, ethanesulfonate, benzenesulfonate, p-toluenesulfonate, naphthalene-1-sulfonate, naphthalene-2-sulfonate, xylenesulfonates, cyclohexylsulfamate, quinate, propionate, decanoate, caprylate, acrylate, formate, isobutyrate, caproate, heptanoate, propiolate, oxalate, malonate, succinate, suberate, sebacate, fumarate, maleate, butyne-1,4 dioate, hexyne-1,6-dioate, benzoate, chlorobenzoate, methylbenzoate, dinitrobenzoate, hydroxybenzoate, methoxybenzoate, phthalate, phenylacetate, phenylpropionate, phenylbutyrate, gamma-hydroxybutyrate, glycollate, and mandelate. Pharmaceutically acceptable salts can be obtained from acids such as hydrochloric acid, maleic acid, sulfuric acid, phosphoric acid, sulfamic acid, acetic acid, citric acid, lactic acid, tartaric acid, malonic acid, methanesulfonic acid, ethanesulfonic acid, benzenesulfonic acid, p-toluenesulfonic acid, cyclohexylsulfamic acid, fumaric acid, and quinic acid.

Pharmaceutically acceptable salts also include basic addition salts such as those containing benzathine, chloroprocaine, choline, diethanolamine, ethanolamine, t-butylamine, ethylenediamine, meglumine, procaine, aluminum, calcium, lithium, magnesium, potassium, sodium, ammonium, alkylamine, and zinc, when acidic functional groups, such as carboxylic acid or phenol are present. For example, see Remington's Pharmaceutical Sciences, 19^{th} ed., Mack Publishing Co., Easton, Pa., Vol. 2, p. 1457, 1995. Such salts can be prepared using the appropriate corresponding bases.

Pharmaceutically acceptable salts can be prepared by standard techniques. For example, the free-base form of a compound can be dissolved in a suitable solvent, such as an aqueous or aqueous-alcohol solution containing the appropriate acid and then isolated by evaporating the solution. In another example, a salt can be prepared by reacting the free base and acid in an organic solvent.

Thus, for example, if the particular compound is a base, the desired pharmaceutically acceptable salt may be prepared by any suitable method available in the art, for example, treatment of the free base with an inorganic acid, such as hydrochloric acid, hydrobromic acid, sulfuric acid, nitric acid, phosphoric acid, and the like, or with an organic acid, such as acetic acid, maleic acid, succinic acid, mandelic acid, fumaric acid, malonic acid, pyruvic acid, oxalic acid, glycolic acid, salicylic acid, a pyranosidyl acid, such as glucuronic acid or galacturonic acid, an alpha-hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like.

Similarly, if the particular compound is an acid, the desired pharmaceutically acceptable salt may be prepared by any suitable method, for example, treatment of the free acid with an inorganic or organic base, such as an amine (primary, secondary or tertiary), an alkali metal hydroxide or alkaline earth metal hydroxide, or the like. Illustrative examples of suitable salts include organic salts derived from amino acids, such as L-glycine, L-lysine, and L-arginine, ammonia, primary, secondary, and tertiary amines, and cyclic amines, such as hydroxyethylpyrrolidine, piperidine, morpholine or piperazine, and inorganic salts derived from sodium, calcium, potassium, magnesium, manganese, iron, copper, zinc, aluminum and lithium.

The pharmaceutically acceptable salt of the different compounds may be present as a complex. Examples of complexes include 8-chlorotheophylline complex (analogous to, e.g., dimenhydrinate: diphenhydramine 8-chlorotheophylline (1:1) complex; Dramamine) and various cyclodextrin inclusion complexes.

Unless specified to the contrary, specification of a compound herein includes pharmaceutically acceptable salts of such compound.

(g) Polymorphic Forms

In the case of agents that are solids, it is understood by those skilled in the art that the compounds and salts may exist in different crystal or polymorphic forms, all of which are intended to be within the scope of the present invention and specified formulae.

### III. Administration

The methods and compounds will typically be used in therapy for human subjects. However, they may also be used to treat similar or identical indications in other animal subjects. In this context, the terms “subject”, “animal subject”, and the like refer to human and non-human vertebrates, e.g., mammals such as non-human primates, sports and commercial animals, e.g., bovines, equines, porcines, ovines, rodents, and pets e.g., canines and felines.

Suitable dosage forms, in part, depend upon the use or the route of administration, for example, oral, transdermal, transmucosal, inhalant, or by injection (parenteral). Such dosage forms should allow the compound to reach target cells. Other factors are well known in the art, and include considerations such as toxicity and dosage forms that retard the compound or composition from exerting its effects. Techniques and formulations generally may be found in Remington: The Science and Practice of Pharmacy, 21^{st} edition, Lippincott, Williams and Wilkins, Philadelphia, Pa., 2005 (hereby incorporated by reference herein).

Compounds of the present invention (i.e. Formula I, including Formulae Ia-Im, and all sub-embodiments disclosed herein) can be formulated as pharmaceutically acceptable salts.

Carriers or excipients can be used to produce compositions. The carriers or excipients can be chosen to facilitate administration of the compound. Examples of carriers include calcium carbonate, calcium phosphate, various sugars such as lactose, glucose, or sucrose, or types of starch, cellulose derivatives, gelatin, vegetable oils, polyethylene glycols and physiologically compatible solvents. Examples of physiologically compatible solvents include sterile solutions of water for injection (WFI), saline solution, and dextrose.

The compounds can be administered by different routes including intravenous, intraperitoneal, subcutaneous, intramuscular, oral, transmucosal, rectal, transdermal, or inhalant. In some embodiments, oral administration is preferred. For oral administration, for example, the compounds can be formulated into conventional oral dosage forms such as capsules, tablets, and liquid preparations such as syrups, elixirs, and concentrated drops.

Pharmaceutical preparations for oral use can be obtained, for example, by combining the active compounds with solid excipients, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations, for example, maize starch, wheat starch, rice starch, potato starch, gelatin, gum tragacanth, methyl cellulose, hydroxypropylmethyl-cellulose, sodium carboxymethylcellulose (CMC), and/or polyvinylpyrrolidone (PVP: povidone). If desired, disintegrating agents may be added, such as the cross-linked polyvinylpyrrolidone, agar, or alginic acid, or a salt thereof such as sodium alginate.

Dragee cores are provided with suitable coatings. For this purpose, concentrated sugar solutions may be used, which may optionally contain, for example, gum arabic, talc, poly-vinylpyrrolidone, carbopol gel, polyethylene glycol (PEG), and/or titanium dioxide, lacquer solutions, and suitable organic solvents or solvent mixtures. Dye-stuffs or pigments may be added to the tablets or dragee coatings for identification or to characterize different combinations of active compound doses.

Pharmaceutical preparations that can be used orally include push-fit capsules made of gelatin (“gelcaps”), as well as soft, sealed capsules made of gelatin, and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols (PEGs). In addition, stabilizers may be added.

Alternatively, injection (parenteral administration) may be used, e.g., intramuscular, intravenous, intraperitoneal, and/or subcutaneous. For injection, the compounds of the invention are formulated in sterile liquid solutions, preferably in physiologically compatible buffers or solutions, such as saline solution, Hank's solution, or Ringer's solution. In addition, the compounds may be formulated in solid form and redissolved or suspended immediately prior to use. Lyophilized forms can also be produced.

Administration can also be by transmucosal, topical, transdermal, or inhalant means. For transmucosal, topical or transdermal administration, penetrants appropriate to the barrier to be permeated are used in the formulation. Such penetrants are generally known in the art, and include, for example, for transmucosal administration, bile salts and fusidic acid derivatives. In addition, detergents may be used to facilitate permeation. Transmucosal administration, for example, may be through nasal sprays or suppositories (rectal or vaginal).

The topical compositions of this invention are formulated preferably as oils, creams, lotions, ointments, and the like by choice of appropriate carriers known in the art. Suitable carriers include vegetable or mineral oils, white petrolatum (white soft paraffin), branched chain fats or oils, animal fats and high molecular weight alcohol (greater than C₁₂). The preferred carriers are those in which the active ingredient is soluble. Emulsifiers, stabilizers, humectants and antioxidants may also be included as well as agents imparting color or fragrance, if desired. Creams for topical application are preferably formulated from a mixture of mineral oil, self-emulsifying beeswax and water in which mixture the active ingredient, dissolved in a small amount solvent (e.g., an oil), is admixed. Additionally, administration by transdermal means may comprise a transdermal patch or dressing such as a bandage impregnated with an active ingredient and optionally one or more carriers or diluents known in the art. To be administered in the form of a transdermal delivery system, the dosage administration will, of course, be continuous rather than intermittent throughout the dosage regimen.

For inhalants, compounds of the invention may be formulated as dry powder or a suitable solution, suspension, or aerosol. Powders and solutions may be formulated with suitable additives known in the art. For example, powders may include a suitable powder base such as lactose or starch, and solutions may comprise propylene glycol, sterile water, ethanol, sodium chloride and other additives, such as acid, alkali and buffer salts. Such solutions or suspensions may be administered by inhaling via spray, pump, atomizer, or nebulizer, and the like. The compounds of the invention may also be used in combination with other inhaled therapies, for example corticosteroids such as fluticasone proprionate, beclomethasone dipropionate, triamcinolone acetonide, budesonide, and mometasone furoate; beta agonists such as albuterol, salmeterol, and formoterol; anticholinergic agents such as ipratroprium bromide or tiotropium; vasodilators such as treprostinal and iloprost; enzymes such as DNAase; therapeutic proteins; immunoglobulin antibodies; an oligonucleotide, such as single or double stranded DNA or RNA, siRNA; antibiotics such as tobramycin; muscarinic receptor antagonists; leukotriene antagonists; cytokine antagonists; protease inhibitors; cromolyn sodium; nedocril sodium; and sodium cromoglycate.

The amounts of various compounds to be administered can be determined by standard procedures taking into account factors such as the compound EC₅₀, the biological half-life of the compound, the age, size, and weight of the subject, and the disorder associated with the subject. The importance of these and other factors are well known to those of ordinary skill in the art. Generally, a dose will be between about 0.01 and 50 mg/kg, preferably 0.1 and 20 mg/kg of the subject being treated. Multiple doses may be used.

The compounds of the invention may also be used in combination with other therapies for treating the same disease. Such combination use includes administration of the compounds and one or more other therapeutics at different times, or co-administration of the compound and one or more other therapies. In some embodiments, dosage may be modified for one or more of the compounds of the invention or other therapeutics used in combination, e.g., reduction in the amount dosed relative to a compound or therapy used alone, by methods well known to those of ordinary skill in the art.

It is understood that use in combination includes use with other therapies, drugs, medical procedures etc., where the other therapy or procedure may be administered at different times (e.g. within a short time, such as within hours (e.g. 1, 2, 3, 4-24 hours), or within a longer time (e.g. 1-2 days, 2-4 days, 4-7 days, 1-4 weeks)) than a compound of the present invention, or at the same time as a compound of the invention. Use in combination also includes use with a therapy or medical procedure that is administered once or infrequently, such as surgery, along with a compound of the invention administered within a short time or longer time before or after the other therapy or procedure. In some embodiments, the present invention provides for delivery of compounds of the invention and one or more other drug therapeutics delivered by a different route of administration or by the same route of administration. The use in combination for any route of administration includes delivery of compounds of the invention and one or more other drug therapeutics delivered by the same route of administration together in any formulation, including formulations where the two compounds are chemically linked in such a way that they maintain their therapeutic activity when administered. In one aspect, the other drug therapy may be co-administered with one or more compounds of the invention. Use in combination by co-administration includes administration of co-formulations or formulations of chemically joined compounds, or administration of two or more compounds in separate formulations within a short time of each other (e.g. within an hour, 2 hours, 3 hours, up to 24 hours), administered by the same or different routes. Co-administration of separate formulations includes co-administration by delivery via one device, for example the same inhalant device, the same syringe, etc., or administration from separate devices within a short time of each other. Co-formulations of compounds of the invention and one or more additional drug therapies delivered by the same route includes preparation of the materials together such that they can be administered by one device, including the separate compounds combined in one formulation, or compounds that are modified such that they are chemically joined, yet still maintain their biological activity. Such chemically joined compounds may have a linkage that is substantially maintained in vivo, or the linkage may break down in vivo, separating the two active components.

### EXAMPLES

Examples related to the present invention are described below. In most cases, alternative techniques can be used. The examples are intended to be illustrative and are not limiting or restrictive to the scope of the invention.

### Example 1

### General Synthesis of Compounds of Formula I

Synthesis of compounds of Formula I where R³ is —Ar₁-M-Ar₂ can be achieved in three steps as described in Scheme 1.

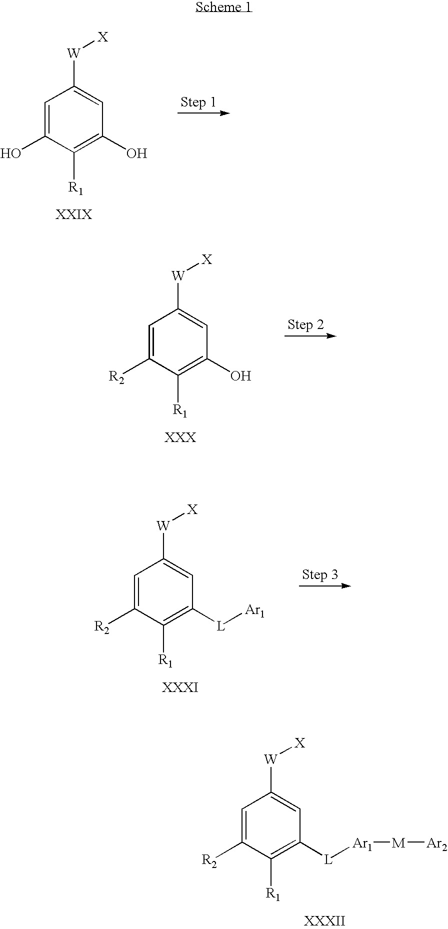

### Step 1: Preparation of Compound XXX

Intermediate XXX can be prepared from compound XXIX via an alkylation reaction with an alkyl halide with a base such as potassium carbonate in an inert solvent such as 2-butanone, or via a Mitsunobu reaction with a hydroxyl group with triphenyl phosphine with an activation reagent such as DEAD (diethylazodicarboxylate) in an inert solvent such as THF.

### Step 2: Preparation of Compound XXXI

Intermediate XXXI can be prepared via conversion of the hydroxyl group of intermediate XXX to a more labile group such as triflate through reaction with trifilic anhydride or tosyl sulfonyl chloride in an inert solvent such as pyridine, allowing a nucleophilic group of L-Ar₁ to displace the labile group. An alternative approach is to use the hydroxyl group of intermediate XXX in an alkylation reaction with an alkyl halide with a base such as potassium carbonate in an inert solvent such as 2-butanone, or via a Mitsonobu reaction with a hydroxyalkane with triphenyl phosphine with an activation reagent such as DEAD in an inert solvent such as THF. Similarly, intermediate XXXI can be prepared with the hydroxyl group of intermediate XXX undergoing an Ullman reaction with a ligand such as N,N-dimethylglycine with a catalyst such as cuprous iodide in an inert solvent such as 1,4-dioxane. L in this scheme is preferably —O— or —S(O)₂—.

### Step 3: Preparation of Compound XXXII

Compound XXXII can be prepared either through a Suzuki coupling of intermediate XXXI with a boronic acid with a palladium catalyst to generate a biaryl compound, or a SN₂Ar reaction to displace a labile functional group such as fluoride. Other means to introduce Ar₂ can be achieved through metal assisted displacement of a labile group by amino or alcohol.

Alternatively, the fragment/substituent can be assembled before coupling to the phenyl acetic acid methyl ester core, as outlined in Scheme 2.

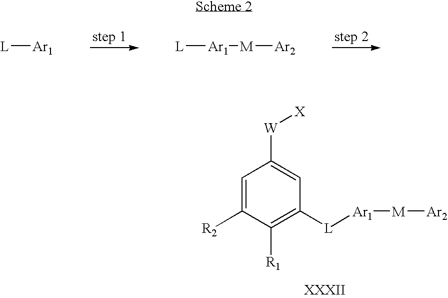

### Step 1: Preparation of Compound L-Ar₁-M-Ar₂

Compound L-Ar₁-M-Ar₂ can be prepared from compound L-Ar₁ either through a Suzuki coupling with a boronic acid with a palladium catalyst to generate a biaryl compound, or a SN₂Ar reaction to displace a labile functional group such as fluoride. Other means to introduce Ar₂ can be achieved through metal assisted displacement of a labile group by amino or alcohol.

### Step 2: Preparation of Compound XXXII

Compound XXXII can be prepared via conversion of the hydroxyl group of intermediate XXX prepared as in Scheme 1 to a more labile group such as triflate through reaction with trifilic anhydride or tosyl sulfonyl chloride in an inert solvent such as pyridine, allowing a nucleophilic group of L-Ar₁-M-Ar₂ to displace the labile group. An alternative approach is to use the hydroxyl group of intermediate XXX in an alkylation reaction with an alkyl halide with a base such as potassium carbonate in an inert solvent such as 2-butanone, or via a Mitsonobu reaction with a hydroxyalkane with triphenyl phosphine with an activation reagent such as DEAD in an inert solvent such as THF. Similarly, compound XXXII can be prepared with the hydroxyl group of intermediate XXX undergoing an Ullman reaction with a ligand such as N,N-dimethylglycine with a catalyst such as cuprous iodide in an inert solvent such as 1,4-dioxane.

A proposed alternate route to compound XXXII (Formula I where R³ is —Ar₁-M-Ar₂) is illustrated in Scheme 3. Compounds XXXII can be prepared from starting material XXXIII in three steps.

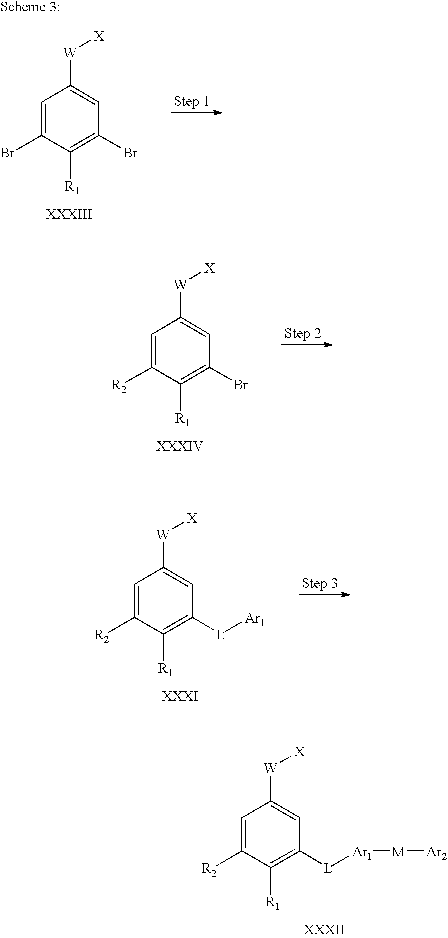

### Step 1: Preparation of Compound XXXIV

Intermediate XXXIV can be prepared via displacement of the bromide (or iodide) of intermediate XXXIII with a hydroxyl or thiol group with a catalyst such as palladium or copper in an inert solvent such as DMF or DMSO.

### Step 2: Preparation of Compound XXXI

Intermediate XXXI can be prepared through displacement of the bromide (or iodide) of intermediate XXXIV with a hydroxyl or thiol group with a catalyst such as palladium or copper in an inert solvent such as DMF or DMSO.

### Step 3: Preparation of Intermediate XXXII

Intermediate XXXII can be prepared either through a Suzuki coupling of intermediate XXXI with a boronic acid with a palladium catalyst to generate a biaryl compound, or a SN₂Ar reaction to displace a labile functional group such as fluoride. Other means to introduce Ar₂ can be achieved through metal assisted displacement of a labile group by amino or alcohol.

Alternatively, the fragment/substituent can be assembled before coupling to the phenyl acetic acid methyl ester core, as outlined in Scheme 2 above.

Synthesis of compounds of Formula I where W is CH₂—, X is —COOH, one of R¹ and R² is OR⁹ and the other is H, and L=—O— can be generated in three synthetic steps from the dihydroxyphenyl acetic acid ester II as illustrated in Scheme 4, where n, R are consistent with the definition of R³ for Formula I.

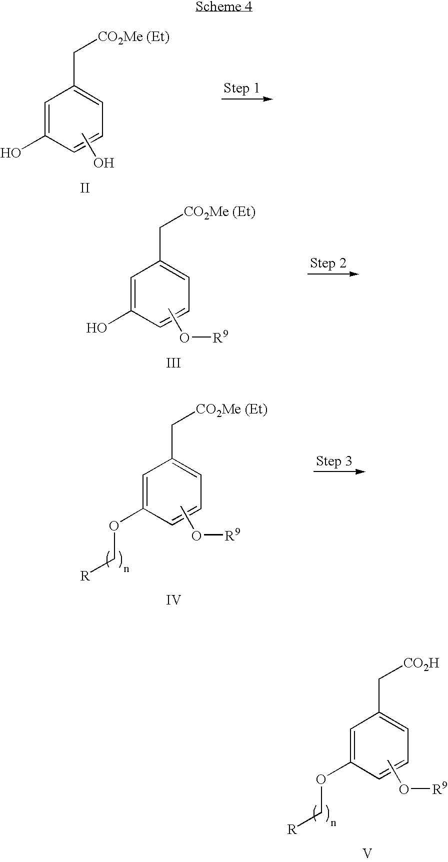

### Step 1: Preparation of Compound III

From II, Compound III can be prepared through reaction with an alkyl halide such as iodoethane with a non-nucleophilic base such as potassium carbonate in an inert solvent such as N,N-dimethylformamide (DMF) with heating.

### Step 2: Preparation of Compound IV

Compound IV can be prepared either through another round of alkylation similar to step 1, or through Mitsunobu reaction conditions with triphenylphosphine with a reagent such as diisopropyl azodicarboxylate in an inert solvent such as tetrahydrofuran at room temperature.

### Step 3: Preparation of Compound V

Compound V can be prepared through deprotection of the alkyl ester through standard saponification conditions with a 1:1 ratio of an inert organic solvent, such as THF and aqueous hydroxide solution (e.g., LiOH, NaOH, or KOH, 1M) at ambient condition.

Synthesis of compounds of Formula I where W is —CR⁴R⁵—, X is —COOH, one of R¹ and R² is OR⁹ and the other is H, and L=—O— is presented in Scheme 5. The synthetic pathway to generate compounds along this series involves a five-step process, where n and R are consistent with the definition of R³ for Formula I.

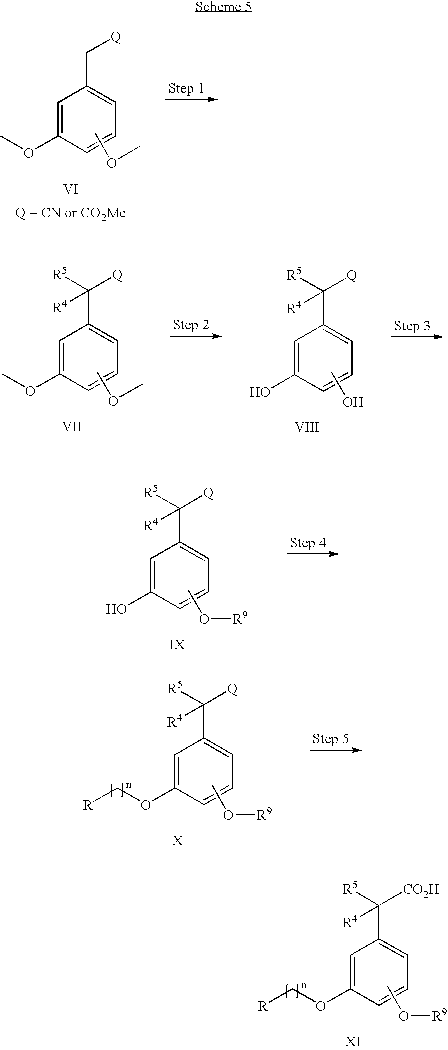

### Step 1: Preparation of Compound VII

Compound VII can be prepared through deprotonation through use of a base (such as sodium hydride or sodium hydroxide) and subsequent alkylation with alkyl halide (or 1,4-dibromobutane to from a cyclopentyl ring) in an inert solvent such as DMF or dimethyl sulfoxide (DMSO).

### Step 2: Preparation of Compound VIII

Compound VIII is prepared by de-methylation with an acid, such as boron tribromide at 0° C.

### Step 3: Preparation of Compound IX

Compound IX can be prepared through reaction with an alkyl halide such as iodoethane with a non-nucleophilic base such as potassium carbonate in an inert solvent such as DMF with heating.

### Step 4: Preparation of Compound X

Compound X can be prepared either through another round of alkylation similar to step 1, or through Mitsunobu reaction conditions with triphenylphosphine with a reagents such as diisopropyl azodicarboxylate in an inert solvent such as THF at room temperature.

### Step 5: Preparation of Compound XI

Compound XI can be prepared by deprotection of the alkyl esters through standard saponification conditions with a 1:1 ratio of an inert organic solvent, such as THF and aqueous hydroxide solution (e.g., LiOH, NaOH, or KOH, 1M) at ambient condition.

Synthesis of compounds of Formula I where W is —CH₂—, X is —COOH, one of R¹ and R² is OR⁹ and the other is H, L=—O—, and R³ is optionally substituted aryl or optionally substituted heteroaryl is presented in Scheme 6. The synthetic pathway to generate compounds along this series involves a two-step process, where R is optionally substituted aryl or optionally substituted heteroaryl.

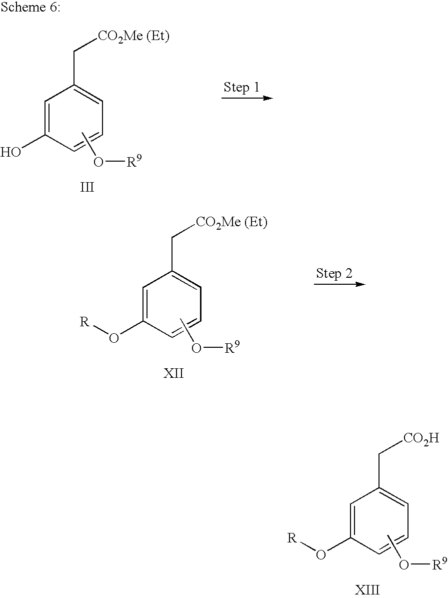

### Step 1: Preparation of Compound XII

Compound XII is prepared through Ullman coupling conditions of a phenol (III as prepared in Scheme 4, Step 1) with a halogenated aromatic ring such as iodobenzene with a catalyst such as cuprous iodide under basic conditions in an inert solvent such as dioxane.

### Step 2: Preparation of Compound XIII

Compound XIII can be prepared by deprotection of the alkyl esters XII through standard saponification conditions with a 1:1 ratio of an inert organic solvent, such as THF and aqueous hydroxide solution (e.g., LiOH, NaOH, or KOH, 1M) at ambient condition.

Synthesis of compounds of Formula I where W is —CH₂—, X is —COOH, one of R¹ and R² is OR⁹ and the other is H, and L=—S(O)₂— is presented in Scheme 7, where R is consistent with the definition of R³ in Formula I. Starting with Compound III, the products can be generated through a three-step process.

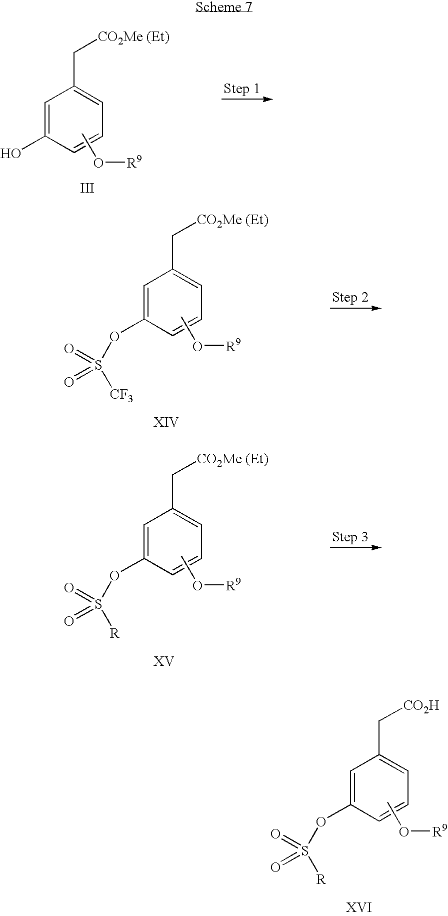

### Step 1: Preparation of Compound XIV

Compound XIV is prepared through a generation of a “triflate” from reacting the hydroxy moiety in III with trifluoromethylsulfonic anhydride in a buffered solvent such as pyridine.

### Step 2: Preparation of Compound XV

Compound XV is prepared by displacement of the triflate with a sulfinic salt, through a catalyst such as palladium acetate, in a basic environment with an inert solvent such as toluene.

### Step 3: Preparation of Compound XVI

Compound XVI can be prepared by deprotection of the alkyl esters through standard saponification conditions with a 1:1 ratio of an inert organic solvent, such as THF and aqueous hydroxide solution (e.g., LiOH, NaOH, or KOH, 1M) at ambient condition.

Synthesis of compounds of Formula I where W is —CH₂—, X is —COOH, one of R¹ and R² is OR⁹ and the other is H, L=—S(O)₂—, and R³ is optionally substituted aryl or optionally substituted heteroaryl is presented in Scheme 8, where R is optionally substituted aryl or optionally substituted heteroaryl. The synthetic pathway to generate compounds along this series involves a six-step process starting from Compound III, where R is optionally substituted aryl or optionally substituted heteroaryl.

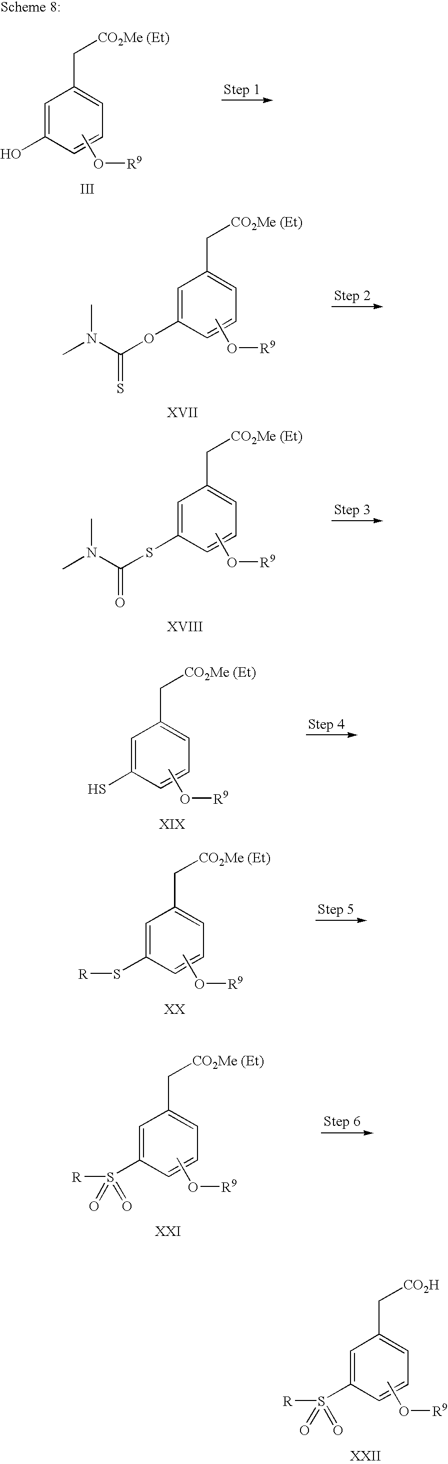

### Step 1: Preparation of Compound XVII

Compound III is treated with N,N,-dimethylthiocarbamoyl chloride under basic environment in an inert solvent such as DMF.

### Step 2: Preparation of Compound XVIII

The thiocarbamate XVII is thermally rearranged to afford compound XVIII, with the assistance of a microwave synthesizer, with an inert solvent such as DMSO or DMF.

### Step 3: Preparation of Compound XIX

Compound XIX can be prepared by hydrolysis of the thiocarbamate XVIII under basic conditions (e.g., aqueous KOH) in an inert solvent such as methanol.

### Step 4: Preparation of Compound XX

Compound XX is prepared through Ullman coupling conditions of the benzenethiol XIX with a halogenated aromatic ring such as iodobenzene with a catalyst such as cuprous iodide under basic environment in an inert solvent such as dioxane.

### Step 5: Preparation of Compound XXI

Biaryl thiol ether XX can be converted to the sulfone XXI through exposure to an oxidant such as m-chloroperbenzoic acid in an inert solvent such as dichloromethane.

### Step 6: Preparation of Compound XXII

Compound XXII can be prepared by deprotection of the alkyl esters XXI under standard saponification conditions with a 1:1 ratio of an inert organic solvent, such as THF and aqueous hydroxide solution (e.g., LiOH, NaOH, or KOH, 1M) at ambient condition.

Synthesis of compounds of Formula I where W is —OCH₂—, X is —COOH, one of R¹ and R² is OR⁹ and the other is H, and L=—S(O)₂— is presented in Scheme 9, where R is consistent with the definition of R³ in Formula I. The products can be generated through a five-step process.

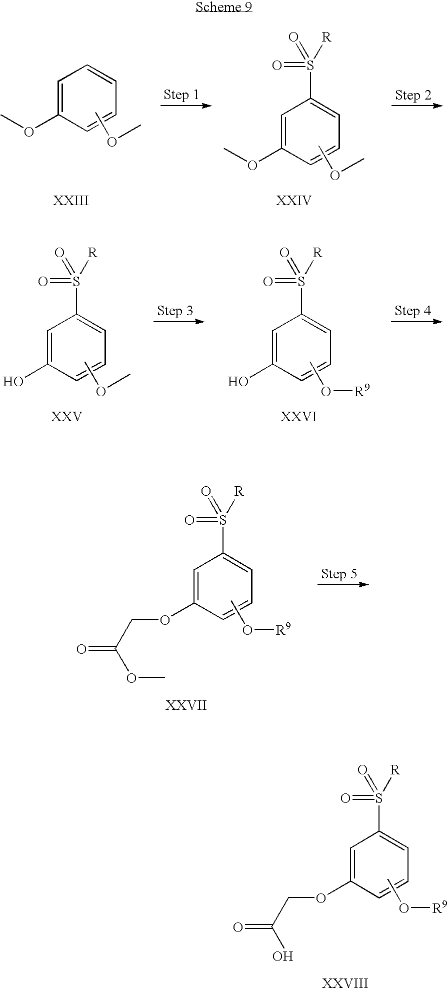

### Step 1: Preparation of Compound XXIV

Compound XXIV is prepared through Friedel-Craft Sulfonylation with a dimethoxybenzene XXIII under acidic conditions such as indium trichloride.

### Step 2: Preparation of Compound XXV

Compound XXV is prepared by de-methylation with an acid, such as boron tribromide at 0° C.

### Step 3: Preparation of Compound XXVI

From XXV, compound XXVI can be prepared by reacting with an alkyl halide such as iodoethane with a non-nucleophilic base such as potassium carbonate in an inert solvent such as DMF with heating.

### Step 4: Preparation of Compound XXVII

From XXVI, compound XXVII can be prepared by reaction with a bromo acetic acid esters and a non-nucleophilic base such as potassium carbonate in an inert solvent such as DMF with heating.

### Step 5: Preparation of Compound XXVIII

Compound XXVIII can be prepared by deprotection of the alkyl esters under standard saponification conditions with a 1:1 ratio of an inert organic solvent, such as THF and aqueous hydroxide solution (e.g., LiOH, NaOH, or KOH, 1M) at ambient condition.

Synthesis of compounds of Formula I where W is —CH₂—, X is —COOH, one of R¹ and R² is OR⁹ and the other is H, L=—S(O)₂— and R³ is optionally substituted imidazole, thiazole or oxazole (U is O, S, or NH, R¹⁰⁰ and R²⁰⁰ are independently hydrogen or a substituent as described for optionally substituted heteroaryl herein) is presented in Scheme 10. Compound XXXX can be generated through a three-step process.

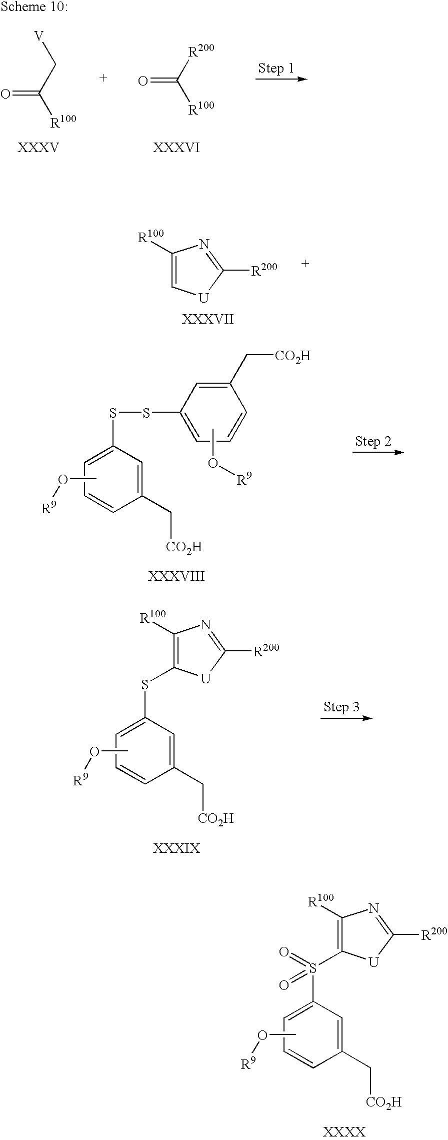

### Step 1: Preparation of Intermediate XXXVII

Compound XXXVII can be prepared from the coupling of an α-halogenated acetyl group (XXXV, where V=chloro or bromo) with an amide or thioamide (XXXVI, where U is O, S, or NH), with heating to afford the cyclized heterocycle XXXVII.

### Step 2: Preparation of Intermediate XXXIX

Compound XXXVIX can be prepared through deprotonation of the 5-proton on the heterocycle with a strong base such as sec-butyl lithium at −78° C. in an inert solvent such as THF, and then coupled with an electrophile XXXVIII to add the thiol ether at the 5-position of the heterocycle.

### Step 3: Preparation of Intermediate XXXX

Compound XXXX can be prepared through oxidation of the thiol ether with an oxidant such as mCPBA at ambient conditions in an inert solvent such as dichloromethane.

### Example 2

### Synthesis of 2-{3-[3-(4-acetyl-3-hydroxy-2-propyl-phenoxy)-propoxy]-5-butoxy-phenyl}-2-methyl-propionic acid (P-0002)

Compound P-0002 was synthesized in five steps from 3,5-dimethoxyphenylacetonitrile 1 as shown in Scheme 11.

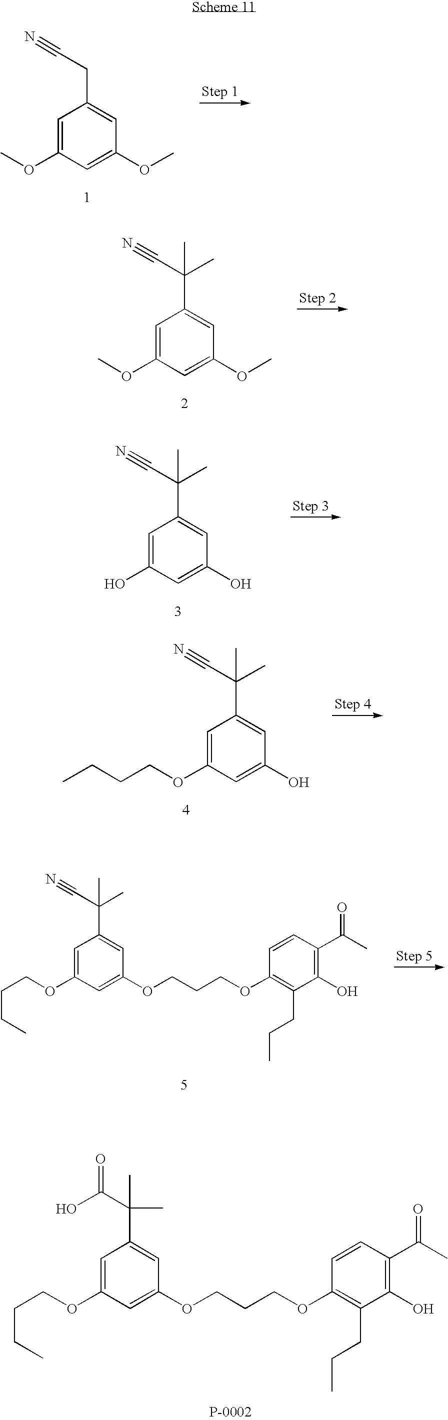

### Step 1: Preparation of 2-(3,5-dimethoxy-phenyl)-2-methyl-propionitrile (2)

To a solution of 3,5-dimethoxyphenylacetonitrile (1, 500 mg, 0.003 mol) in tetrahydrofuran (10 mL, 0.1 mol) at −78° C., 2.5M n-butyllithium in hexane (2.6 mL) was added within 5 minutes. The mixture was then stirred for 30 minutes. Methyl iodide (0.40 mL, 0.0065 mol) in 5 ml tetrahydrofuran was added over a 10 minute period. The mixture was allowed to stir overnight at 0° C. to room temperature. Water (5 ml) was added, followed by diethyl ether (10 ml). The aqueous phase was extracted with diethyl ether. The pooled organic phase was washed with brine and dried over sodium sulphate. Flash chromatography (0-5% ethyl acetate in hexanes) afforded a clear oil (2, 296 mg, 50%).

### Step 2: Preparation of 2-(3,5-dihydroxy-phenyl)-2-methyl-propionitrile (3)

To a solution of 2-(3,5-dimethoxy-phenyl)-2-methyl-propionitrile (2, 290 mg, 0.0014 mol) in dichloromethane (6 mL, 0.09 mol), 1M boron tribromide in heptane (3.5 mL) was added at room temperature and the mixture stirred for 6 hrs. The reaction was quenched with water and diluted with ethyl acetate. The phases were separated and the aqueous phase was extracted with ethyl acetate, which was washed with brine, dried with sodium sulfate and concentrated. The crude material was taken to the next step without further purification.

### Step 3: Preparation of (2-(3-butoxy-5-hydroxy-phenyl)-2-methyl-propionitrile (4)

To a solution of 2-(3,5-dihydroxy-phenyl)-2-methyl-propionitrile (3, 0.257 g, 0.00145 mol) in dimethyl formamide (10 mL, 0.2 mol), potassium carbonate (0.6 g, 0.004 mol) was added. The mixture was heated to 90° C. and 1-iodobutane (0.100 mL, 0.000878 mol) in dimethyl formamide (1 ml) was added drop wise. The reaction was stirred for 5 hours, after which the dimethyl formamide was removed in vacuo. Water and ethyl acetate were added, the water phase was acidified using 1M HCl and extracted with ethyl acetate. The organic phase was dried over sodium sulfate. Flash chromatography (0-5% ethyl acetate in hexanes) afforded the desired compound 4.

### Step 4: Preparation of 2-{3-[3-(4-acetyl-3-hydroxy-2-propyl-phenoxy)-propoxy]-5-butoxy-phenyl}-2-methyl-propionitrile (5)

To a solution of 2-(3-butoxy-5-hydroxy-phenyl)-2-methyl-propionitrile (4, 50 mg, 0.0002 mol) in acetonitrile (5 mL, 0.1 mol), potassium carbonate (89 mg, 0.00064 mol) was added followed by 1-[4-(3-Bromo-propoxy)-2-hydroxy-3-propyl-phenyl]-ethanone (100 mg, 0.00032 mol). The mixture was heated overnight at 80° C. The mixture was concentrated and water and ethyl acetate added. The aqueous phase was acidified with 1M HCl and extracted with ethyl acetate. The pooled organic phase was dried with sodium sulfate and concentrated. Purification was carried out by chromatography (25% ethyl acetate in hexanes). The desired compound was obtained as an oil (5, 15 mg, 10%).

### Step 5: Preparation of 2-{3-[3-(4-acetyl-3-hydroxy-2-propyl-phenoxy)-propoxy]-5-butoxy-phenyl}-2-methyl-propionic acid (P-0002)

To a solution of 2-{3-[3-(4-acetyl-3-hydroxy-2-propyl-phenoxy)-propoxy]-5-butoxy-phenyl}-2-methyl-propionitrile (5, 13 mg, 0.000028 mol) in methanol (1 mL, 0.02 mol), 2M lithium hydroxide in water (0.2 mL) was added and the mixture stirred for 2 days at 80° C. The mixture was transferred to a microwave reaction vessel and heated at 120° C. for 5 minutes in a microwave oven, followed by heating a total of 5 times at 160° C. for 15 minutes. The mixture was acidified with 1M HCl, extracted with ethyl acetate, dried over sodium sulfate, and the solvent removed under reduced pressure. The compound P-0002 was purified using normal phase chromatography (50% ethyl acetate in hexanes). Calculated molecular weight 486.60, MS (ESI) [M+H⁺]⁺=487.3, [M−H⁺]⁻=485.2.

Additional compounds were prepared using the same protocol as described in Scheme 11. P-0005 was prepared by replacing methyl iodide with 1,4-dibromobutane (1 equivalent) in Step 1. Compounds P-0002 and P-0005 were side products isolated after Step 5 hydrolysis of the nitrile, which also provided the corresponding amides P-0003 and P-0004, respectively. The compound names, structures and experimental mass spectrometry results for these additional compounds are provided in the following Table 1.

TABLE 1

| | | | Molecular weight | |
|---|---|---|---|---|
| Cmpd. | | | | Measured |
| number | Structure | Name | Calc. | MS(ESI) |
| P-0003 | 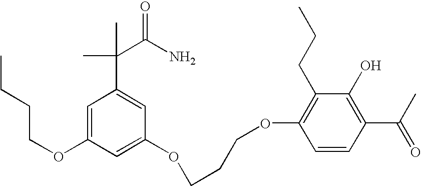 | 2-{3-[3-(4-acetyl-3-hydroxy-2-propyl-phenoxy)-propoxy]-5-butoxy-phenyl}-isobutyramide | 485.62 | [M + H⁺]⁺ =486.2[M − H⁺]⁻ =484.3 |
| P-0004 | 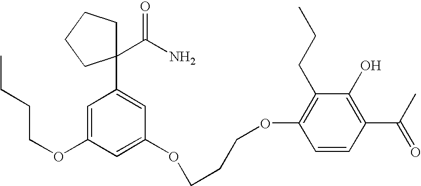 | 1-{3-[3-(4-acetyl-3-hydroxy-2-propyl-phenoxy)-propoxy]-5-butoxy-phenyl}-cyclopentanecarboxylic acid amide | 511.65 | [M + H⁺]⁺ =512.3[M − H⁺]⁻ =510.3 |
| P-0005 | 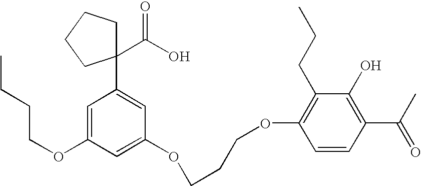 | 1-{3-[3-(4-acetyl-3-hydroxy-2-propyl-phenoxy)-propoxy]-5-butoxy-phenyl}-cyclopentanecarboxylic acid | 512.64 | [M + H⁺]⁺ =513.3[M − H⁺]⁻ =511.2 |

### Example 3

### Synthesis of {3-butoxy-5-[4-(4-trifluoromethoxy-phenoxy)-benzenesulfonyl]-phenyl}-acetic acid (P-0027)

Compound P-0027 was synthesized in five steps from (3,5-dihydroxy-phenyl)-acetic methyl ester 8 as shown in Scheme 12.

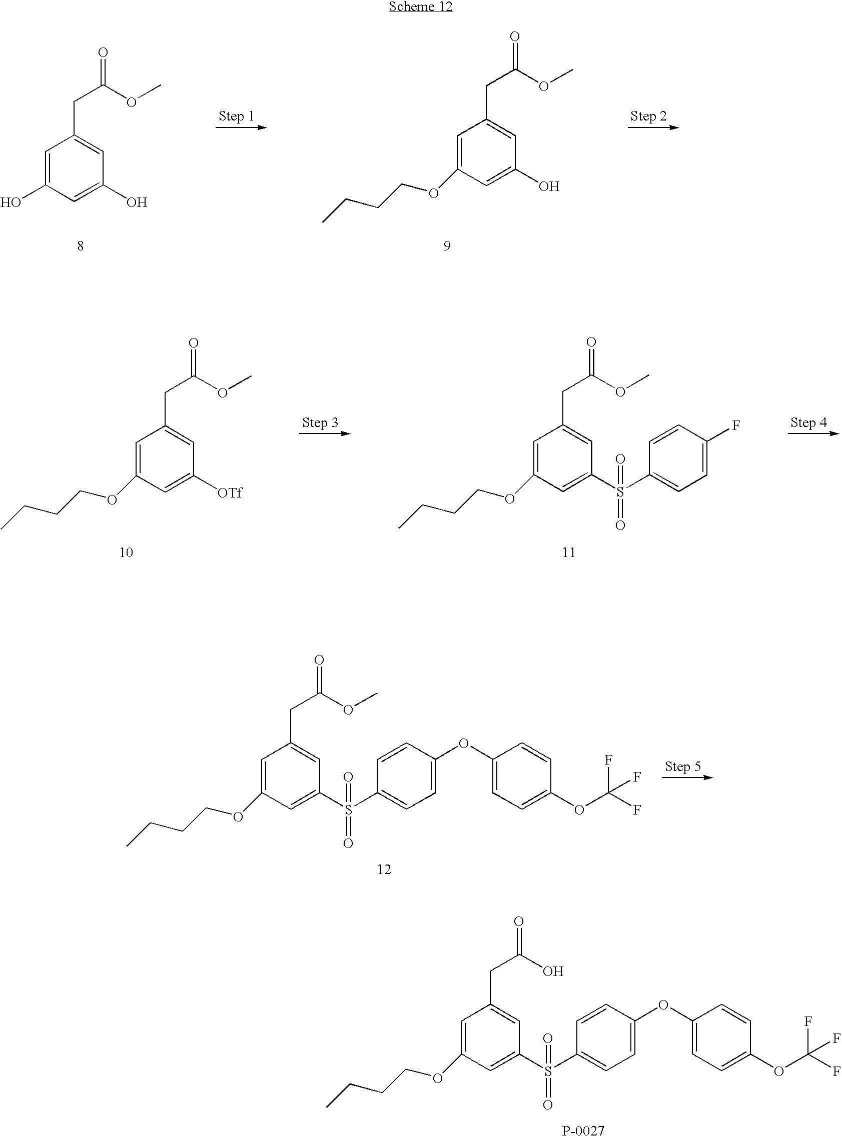

### Step 1: Preparation of (3-butoxy-5-hydroxy-phenyl)-acetic acid methyl ester (9)

To a solution of (3,5-dihydroxy-phenyl)-acetic acid methyl ester (8, 1.200 g, 0.006587 mol) in dimethyl formamide (50 mL, 0.7 mol), potassium carbonate (2.73 g, 0.0198 mol) was added in one portion. 1-Iodobutane (0.682 mL, 0.00599 mol) in dimethyl formamide was added drop wise and the reaction was heated to 90° C. and stirred overnight. The dimethyl formamide was removed in vacuo and water and ethyl acetate were added. The mixture was acidified with 1M HCl and the water phase was extracted with ethyl acetate. The pooled organic phase was dried over sodium sulfate and put on silica. Flash chromatography (25% ethyl acetate in hexanes) yielded the desired compound as an oil (9, 615 mg, 43%).

### Step 2: Preparation of (3-butoxy-5-trifluoromethanesulfonyloxy-phenyl)-acetic acid methyl ester (10)

To a solution of (3-butoxy-5-hydroxy-phenyl)-acetic acid methyl ester (9, 100 mg, 0.0004 mol) in pyridine (0.4 mL, 0.005 mol) over ice/water bath, trifluoromethanesulfonic anhydride (90.0 μL, 0.000535 mol) was added drop wise to the solution. The mixture was stirred for 15 minutes with cooling, then stirred for 2 hours at room temperature. Water (2 mL) and diethyl ether (5 mL) were added and the solution was acidified with 1 mL conc. HCl. The ether was separated, washed with 1M HCl, dried over sodium sulfate and concentrated. Purification by flash chromatography (hexane/ethyl acetate 3:1) yielded a clear oil (10, 95 mg of 60%).

### Step 3: Preparation of [3-butoxy-5-(4-fluoro-benzenesulfonyl)-phenyl]-acetic acid methyl ester (11)

To a solution of (3-butoxy-5-trifluoromethanesulfonyloxy-phenyl)-acetic acid methyl ester (10, 198 mg, 0.000535 mol) and sodium 4-fluoro-benzenesulfinate (120 mg, 0.00064 mol) dissolved in toluene (4 mL, 0.04 mol) in a sealable reaction vessel, tris(dibenzylideneacetone)dipalladium(0) (49 mg, 0.000053 mol), cesium carbonate (260 mg, 0.00080 mol), and xanthphos (60 mg, 0.0001 mol) were added under an argon atmosphere. The vessel was sealed and the mixture was heated overnight at 120° C. After cooling, the reaction mixture was diluted with ethyl acetate, washed with brine, dried over sodium sulfate, concentrated and put on silica. Flash chromatography (hexane/ethyl acetate 9:1) yielded the desired compound (11, 65 mg, 32%).

### Step 4: Preparation of {3-butoxy-5-[4-(4-trifluoromethoxy-phenoxy)-benzenesulfonyl]-phenyl}-acetic acid methyl ester (12)

To a solution of [3-butoxy-5-(4-fluoro-benzenesulfonyl)-phenyl]-acetic acid methyl ester (11, 25 mg, 0.000066 mol) in dimethyl sulfoxide (0.5 mL, 0.007 mol), potassium carbonate (10 mg, 0.000072 mol) and 4-trifluoromethoxy-phenol (9.4 μL, 0.000072 mol) were added. The mixture was heated in a microwave oven for 10 minutes at 120° C. The solvent was removed by freeze drying overnight. Ethyl acetate and water were added and the layers separated. The organic phase was washed with brine and dried over sodium sulfate. The desired product was purified by silica gel chromatography (hexane/ethyl acetate 3:1) to yield compound 12 (12 mg, 34%).

### Step 5: Preparation of {3-butoxy-5-[4-(4-trifluoromethoxy-phenoxy)-benzenesulfonyl]-phenyl}-acetic acid (P-0027)

To a solution of {3-butoxy-5-[4-(4-trifluoromethoxy-phenoxy)-benzenesulfonyl]-phenyl}-acetic acid methyl ester (12, 12 mg, 0.000022 mol) in tetrahydrofuran (2 mL, 0.02 mol), potassium hydroxide (1M, 1 mL) was added and stirred overnight at room temperature. Ethyl acetate (3 mL) was added and the mixture was acidified with 1M HCl. The aqueous phase was extracted with ethyl acetate. The organic phase was washed with brine, then dried with sodium sulfate and concentrated. The desired compound P-0027 was purified using silica gel chromatography (5% methanol in dichloromethane). Calculated molecular weight 524.51, MS (ESI) [M+H⁺]⁺=525.2 [M−H⁺]⁻=523.2.

Additional compounds were prepared using the same protocol as described in Scheme 12. P-0158 was prepared by replacing 1-iodobutane with 1-iodopropane and replacing (3,5-dihydroxy-phenyl)-acetic acid methyl ester 8 with (3,5-dihydroxy-phenyl)-propionic acid methyl ester in Step 1. P-0293 was prepared starting from Step 2 by replacing (3-butoxy-5-hydroxy-phenyl)-acetic acid methyl ester 9 with (3-hydroxy-phenyl)-acetic acid methyl ester in Step 2. Additional compounds were prepared by optionally replacing the 1-iodobutane with an appropriate iodoalkyl compound in Step 1, and/or optionally replacing the 4-trifluoromethoxy-phenol with an appropriate phenol or benzenethiol in Step 4. The following Table 2 indicates the reagent used in Step 1 and 4 for the indicated compound number.

TABLE 2

| Compound | | |
|---|---|---|
| number | Step 1 reagent | Step 4 reagent |
| P-0062 | iodoethane | 4-trifluoromethoxy-phenol |
| P-0057 | iodomethane | 4-trifluoromethoxy-phenol |
| P-0058 | 1-iodo-2-methoxyethane | 4-trifluoromethyl-phenol |
| P-0059 | 1-iodo-2-methoxyethane | 4-trifluoromethoxy-phenol |
| P-0141 | iodoethane | 3-ethoxy-phenol |
| P-0142 | iodoethane | 6-methyl-pyridin-2-ol |
| P-0143 | iodoethane | 3-methyl-pyridin-2-ol |
| P-0144 | 1-iodopropane | 3-ethoxy-phenol |
| P-0145 | 1-iodopropane | 6-methyl-pyridin-2-ol |
| P-0146 | 1-iodopropane | 3-methyl-pyridin-2-ol |
| P-0114 | iodoethane | 4-imidazol-1-yl-phenol |
| P-0115 | iodoethane | 3,4-dimethoxy-phenol |
| P-0116 | iodoethane | 3,4-dichloro-phenol |
| P-0117 | 1-iodopropane | 4-imidazol-1-yl-phenol |
| P-0118 | 1-iodopropane | 3,4-dimethoxy-phenol |
| P-0119 | 1-iodopropane | 3,4-dichloro-phenol |
| P-0235 | iodoethane | 3-methoxy-benzenethiol |
| P-0236 | iodoethane | 3-ethoxy-benzenethiol |
| P-0237 | 1-iodopropane | 3,4-dimethoxy-benzenethiol |
| P-0238 | 1-iodopropane | 3-methoxy-benzenethiol |
| P-0239 | 1-iodopropane | 4-trifluoromethyl-benzenethiol |
| P-0240 | 1-iodopropane | 3-ethoxy-benzenethiol |
| P-0241 | 1-iodopropane | 4-methoxy-benzenethiol |
| P-0242 | 1-iodopropane | 3-trifluoromethoxy-benzenethiol |
| P-0243 | iodomethyl-cyclopropane | 3-methoxy-benzenethiol |
| P-0244 | iodomethyl-cyclopropane | 4-trifluoromethyl-benzenethiol |
| P-0245 | iodomethyl-cyclopropane | 3-ethoxy-benzenethiol |
| P-0246 | iodomethyl-cyclopropane | 4-methoxy-benzenethiol |
| P-0247 | iodomethyl-cyclopropane | 3-trifluoromethoxy-benzenethiol |
| P-0248 | 1-iodo-2-methoxyethane | 3,4-dimethoxy-benzenethiol |
| P-0249 | 1-iodo-2-methoxyethane | 3-methoxy-benzenethiol |
| P-0250 | 1-iodo-2-methoxyethane | 3-ethoxy-benzenethiol |
| P-0251 | 1-iodo-2-methoxyethane | 4-methoxy-benzenethiol |
| P-0252 | 1-iodo-2-methoxyethane | 3-trifluoromethoxy-benzenethiol |
| P-0253 | iodoethane | pyridine-4-thiol |
| P-0254 | 1-iodopropane | pyridine-4-thiol |
| P-0255 | iodomethyl-cyclopropane | pyridine-4-thiol |
| P-0256 | 1-iodo-2-methoxyethane | pyridine-4-thiol |
| P-0281* | 1-iodopropane | 4-methanesulfonyl-phenol |
| P-0282 | 1-iodopropane | 4-methanesulfonyl-phenol |
| P-0261 | 1-iodo-2-methoxyethane | 4-methoxy-phenol |
| P-0262 | iodoethane | 4-methoxy-phenol |
| P-0263 | iodomethyl-cyclopropane | 4-methoxy-phenol |
| P-0264 | 1-iodopropane | 4-methoxy-phenol |
| P-0265 | 1-iodo-2-methoxyethane | 4-ethoxy-phenol |
| P-0266 | iodoethane | 4-ethoxy-phenol |
| P-0267 | iodomethyl-cyclopropane | 4-ethoxy-phenol |
| P-0268 | 1-iodopropane | 4-ethoxy-phenol |
| P-0269 | 1-iodo-2-methoxyethane | 4-propoxy-phenol |
| P-0270 | iodoethane | 4-propoxy-phenol |
| P-0271 | iodomethyl-cyclopropane | 4-propoxy-phenol |
| P-0272 | 1-iodopropane | 4-propoxy-phenol |
| P-0273 | 1-iodo-2-methoxyethane | 4-tert-butoxy-phenol |
| P-0274 | iodoethane | 4-tert-butoxy-phenol |
| P-0275 | iodomethyl-cyclopropane | 4-tert-butoxy-phenol |
| P-0276 | 1-iodopropane | 4-tert-butoxy-phenol |
| P-0277 | iodomethyl-cyclopropane | 4-trifluoromethoxy-phenol |
| P-0280 | 1-iodopropane | 4-methysulfanyl-phenol |
| P-0088* | iodoethane | 4-trifluoromethoxy-phenol |
| P-0207 | 1-iodo-2-methoxyethane | 3-ethoxy-phenol |
| P-0208 | iodomethyl-cyclopropane | 4-imidazol-1-yl-phenol |
| P-0212 | 1-iodo-2-methoxyethane | 3,4-dichloro-phenol |
| P-0213 | iodomethyl-cyclopropane | 3,4-dichloro-phenol |
| P-0214 | 1-iodo-2-methoxyethane | 3,4-dimethoxy-phenol |
| P-0215 | iodomethyl-cyclopropane | 3,4-dimethoxy-phenol |
| P-0216 | iodomethyl-cyclopropane | 3-ethoxy-phenol |
| P-0217 | 1-iodo-2-methoxyethane | 4-imidazol-1-yl-phenol |
| P-0229 | iodomethyl-cyclopropane | 6-methyl-pyridin-2-ol |
| P-0230 | 1-iodo-2-methoxyethane | 6-methyl-pyridin-2-ol |
| P-0233 | 1-iodo-2-methoxyethane | 3-methyl-pyridin-2-ol |

*Methyl ester isolated after Step 4.

The compound structures, names and mass spectrometry results for these compounds are provided in the following Table 3.

TABLE 3

| | | | Molecular weight | |
|---|---|---|---|---|
| Cmpd. | | | | Measured |
| number | Structure | Name | Calc. | MS(ESI) |
| P-0158 | 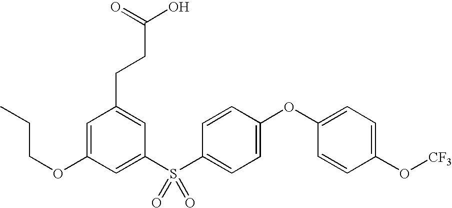 | 3-{3-Propoxy-5-[4-(4-trifluoromethoxy-phenoxy)-benzenesulfonyl]-phenyl}-propionic acid | 524.51 | [M + H⁺]⁻ =523.13 |
| P-0293 | 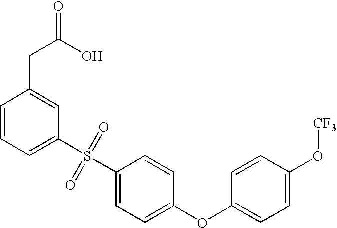 | {3-[4-(4-Trifluoromethoxy-phenoxy)-benzenesulfonyl]-phenyl}-acetic acid | 452.40 | [M + H⁺]⁺ =453.19[M − H⁺]⁻ =451.07 |
| P-0062 | 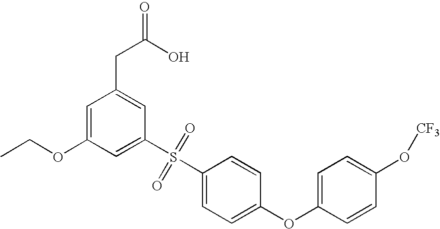 | {3-Ethoxy-5-[4-(4-trifluoromethoxy-phenoxy)-benzenesulfonyl]-phenyl}-acetic acid | 496.46 | [M + H⁺]⁺ =497.2[M − H⁺]⁻ =495.1 |
| P-0057 | 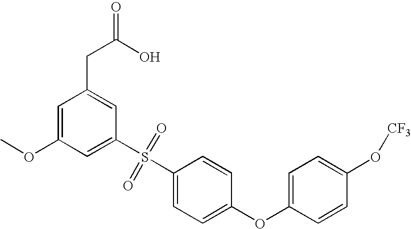 | {3-Methoxy-5-[4-(4-trifluoromethoxy-phenoxy)-benzenesulfonyl]-phenyl}-acetic acid | 482.43 | [M + H⁺]⁺ =483.2[M − H⁺]⁻ =481.1 |
| P-0058 | 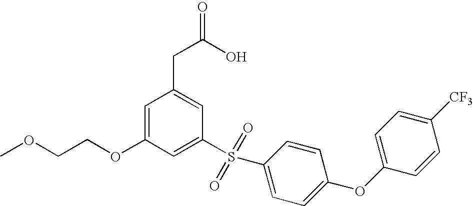 | {3-(2-Methoxy-ethoxy)-5-[4-(4-trifluoromethyl-phenoxy)-benzenesulfonyl]-phenyl}-acetic acid | 510.48 | [M + H⁺]⁺ =511.23[M − H⁺]⁻ =509.5 |
| P-0059 | 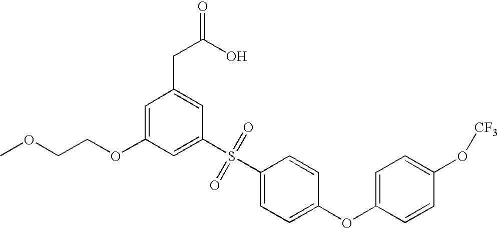 | {3-(2-Methoxy-ethoxy)-5-[4-(4-trifluoromethoxy-phenoxy)-benzenesulfonyl]-phenyl}-acetic acid | 526.48 | [M + H⁺]⁺ =527.23[M − H⁺]⁻ =525.13 |
| P-0141 | 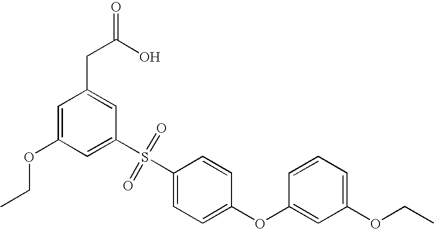 | {3-Ethoxy-5-[4-(3-ethoxy-phenoxy)-benzenesulfonyl]-phenyl}-acetic acid | 456.51 | [M + H⁺]⁺ =457.1 |
| P-0142 | 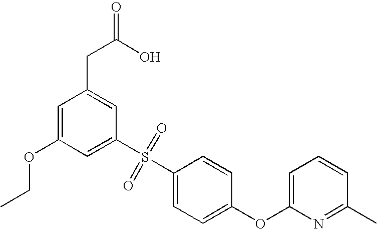 | {3-Ethoxy-5-[4-(6-methyl-pyridin-2-yloxy)-benzenesulfonyl]-phenyl}-acetic acid | 427.47 | [M + H⁺]⁺ =427.9 |
| P-0143 | 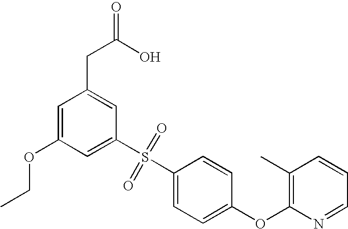 | {3-Ethoxy-5-[4-(3-methyl-pyridin-2-yloxy)-benzenesulfonyl]-phenyl}-acetic acid | 427.47 | [M + H⁺]⁺ =427.9 |
| P-0144 | 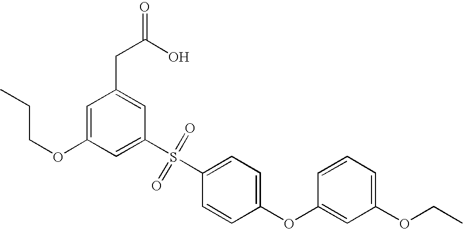 | {3-[4-(3-Ethoxy-phenoxy)-benzenesulfonyl]-5-propoxy-phenyl}-acetic acid | 470.54 | [M + H⁺]⁺ =471.1 |
| P-0145 | 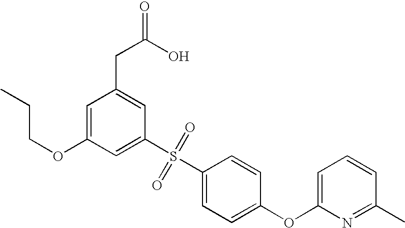 | {3-[4-(6-Methyl-pyridin-2-yloxy)-benzenesulfonyl]-5-propoxy-phenyl}-acetic acid | 441.50 | [M + H⁺]⁺ =442.3 |
| P-0146 | 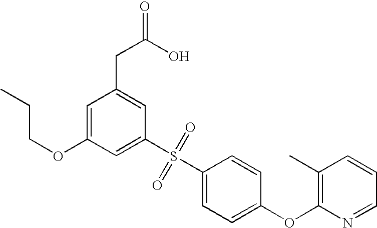 | {3-[4-(3-Methyl-pyridin-2-yloxy)-benzenesulfonyl]-5-propoxy-phenyl}-acetic acid | 441.50 | [M + H⁺]⁺ =442.3 |
| P-0114 | 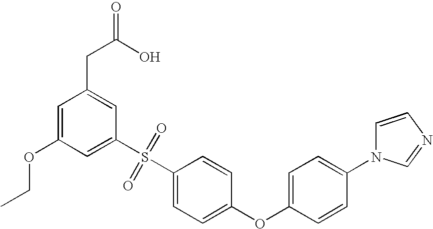 | {3-Ethoxy-5-[4-(4-imidazol-1-yl-phenoxy)-benzenesulfonyl]-phenyl}-acetic acid | 478.52 | [M + H⁺]⁺ =479.1 |
| P-0115 | 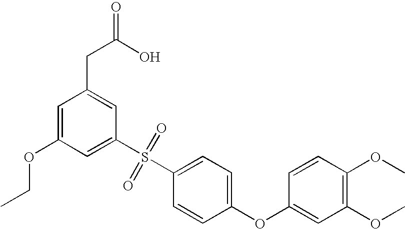 | {3-[4-(3,4-Dimethoxy-phenoxy)-benzenesulfonyl]-5-ethoxy-phenyl}-acetic acid | 472.51 | [M + H⁺]⁺ =473.1 |
| P-0116 | 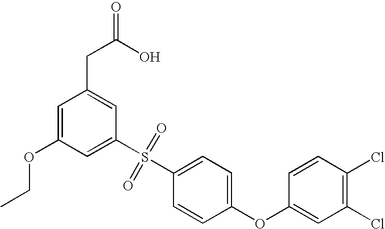 | {3-[4-(3,4-Dichloro-phenoxy)-benzenesulfonyl]-5-ethoxy-phenyl}-acetic acid | 481.35 | [M + H⁺]⁺ =481.1 |
| P-0117 | 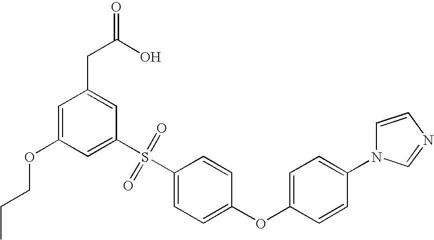 | {3-[4-(4-Imidazol-1-yl-phenoxy)-benzenesulfonyl]-5-propoxy-phenyl}-acetic acid | 492.55 | [M + H⁺]⁺ =493.1 |
| P-0118 | 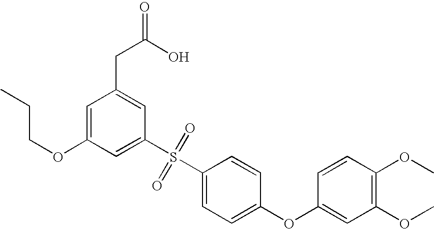 | {3-[4-(3,4-Dimethoxy-phenoxy)-benzenesulfonyl]-5-propoxy-phenyl}-acetic acid | 486.54 | [M + H⁺]⁺ =487.1 |
| P-0119 | 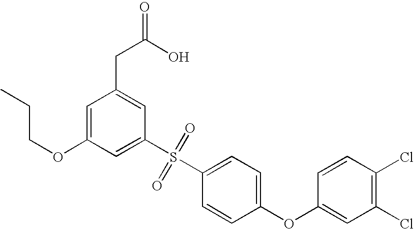 | {3-[4-(3,4-Dichloro-phenoxy)-benzenesulfonyl]-5 -propoxy-phenyl}-acetic acid | 495.38 | [M + H⁺]⁺ =495.1 |
| P-0235 | 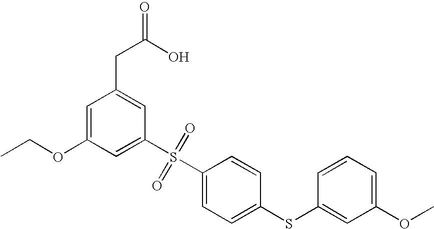 | {3-Ethoxy-5-[4-(3-methoxy-phenylsulfanyl)-benzenesulfonyl]-phenyl}-acetic acid | 458.55 | [M + H⁺]⁺ =459.1 |
| P-0236 | 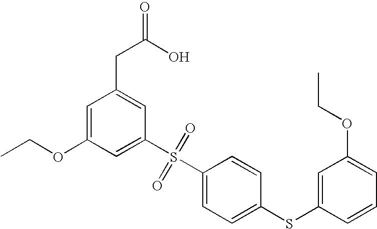 | {3-Ethoxy-5-[4-(3 -ethoxy-phenylsulfanyl)-benzenesulfonyl]-phenyl}-acetic acid | 472.58 | [M + H⁺]⁺ =473.1 |
| P-0237 | 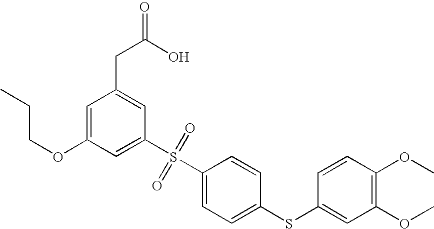 | {3-[4-(3,4-Dimethoxy-phenylsulfanyl)-benzenesulfonyl]-5-propoxy-phenyl}-acetic acid | 502.61 | [M + H⁺]⁺ =503.1 |
| P-0238 | 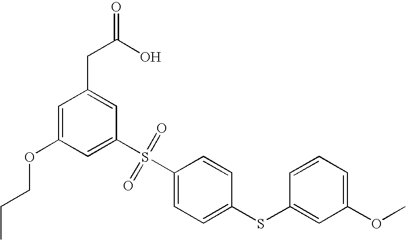 | {3-[4-(3-Methoxy-phenylsulfanyl)-benzenesulfonyl]-5-propoxy-phenyl}-acetic acid | 472.58 | [M + H⁺]⁺ =473.1 |
| P-0239 | 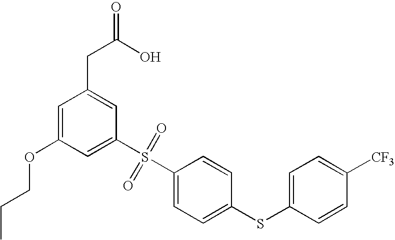 | {3-Propoxy-5-[4-(4-trifluoromethyl-phenylsulfanyl)-benzenesulfonyl]-phenyl}-acetic acid | 510.55 | [M + H⁺]⁺ =511.5 |
| P-0240 | 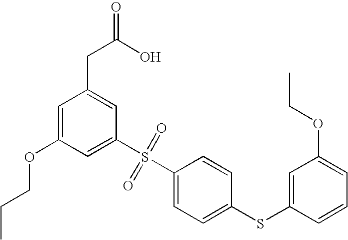 | {3-[4-(3-Ethoxy-phenylsulfanyl)-benzenesulfonyl]-5-propoxy-phenyl}-acetic acid | 486.61 | [M + H⁺]⁺ =487.1 |
| P-0241 | 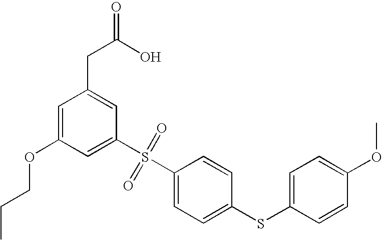 | {3-[4-(4-Methoxy-phenylsulfanyl)-benzenesulfonyl]-5-propoxy-phenyl}-acetic acid | 472.58 | [M + H⁺]⁺ =473.1 |
| P-0242 | 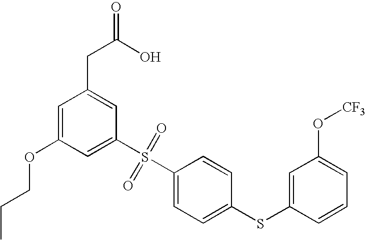 | {3-Propoxy-5-[4-(3-trifluoromethoxy-phenylsulfanyl)-benzenesulfonyl]-phenyl}-acetic acid | 526.55 | [M + H⁺]⁺ =527.1 |
| P-0243 | 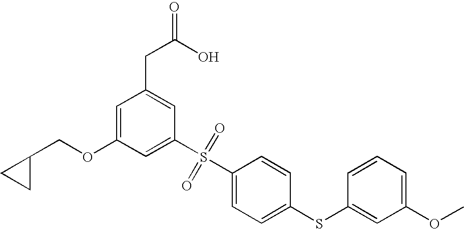 | {3-Cyclopropylmethoxy-5-[4-(3-methoxy-phenylsulfanyl)-benzenesulfonyl]-phenyl}-acetic acid | 484.59 | [M + H⁺]⁺ =485.1 |
| P-0244 | 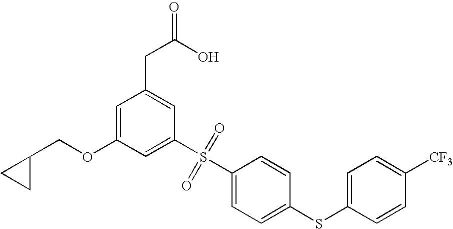 | {3-Cyclopropylmethoxy-5-[4-(4-trifluoromethyl-phenylsulfanyl)-benzenesulfonyl]-phenyl}-acetic acid | 522.56 | [M + H⁺]⁺ =523.1 |
| P-0245 | 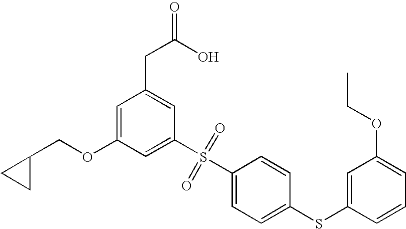 | {3-Cyclopropylmethoxy-5-[4-(3-ethoxy-phenylsulfanyl)-benzenesulfonyl]-phenyl}-acetic acid | 498.62 | [M + H⁺]⁺ =499.1 |
| P-0246 | 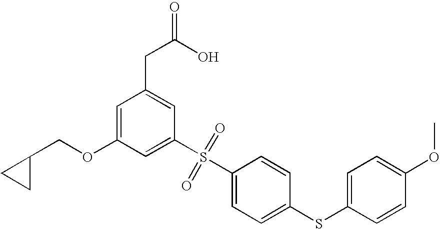 | {3-Cyclopropylmethoxy-5-[4-(4-methoxy-phenylsulfanyl)-benzenesulfonyl]-phenyl}-acetic acid | 484.59 | [M + H⁺]⁺ =485.5 |
| P-0247 | 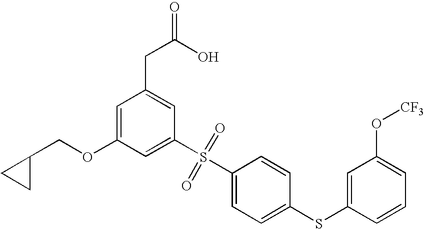 | {3-Cyclopropylmethoxy-5-[4-(3-trifluoromethoxy-phenylsulfanyl)-benzenesulfonyl]-phenyl}-acetic acid | 538.56 | [M + H⁺]⁺ =539.1 |
| P-0248 | 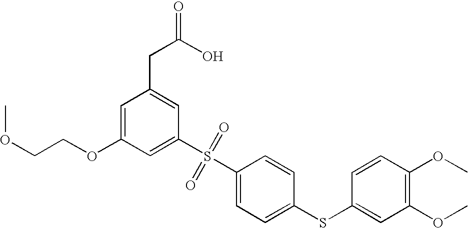 | [3-[4-(3,4-Dimethoxy-phenylsulfanyl)-benzenesulfonyl]-5-(2-methoxy-ethoxy)-phenyl]-acetic acid | 518.60 | [M + H⁺]⁺ =519.1 |
| P-0249 | 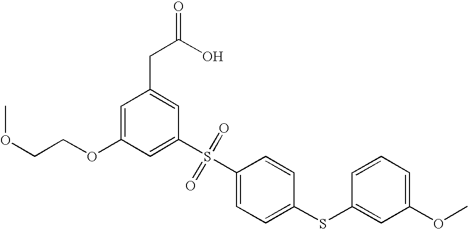 | {3-(2-Methoxy-ethoxy)-5-[4-(3-methoxy-phenylsulfanyl)-benzenesulfonyl]-phenyl}-acetic acid | 488.58 | [M + H⁺]⁺ =489.01 |
| P-0250 | 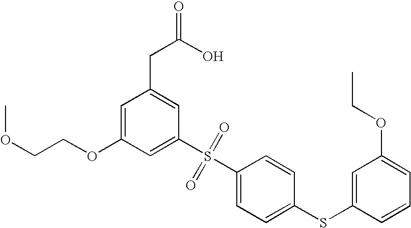 | [3-[4-(3-Ethoxy-phenylsulfanyl)-benzenesulfonyl]-5-(2-methoxy-ethoxy)-phenyl]-acetic acid | 502.61 | [M + H⁺]⁺ =503.1 |
| P-0251 | 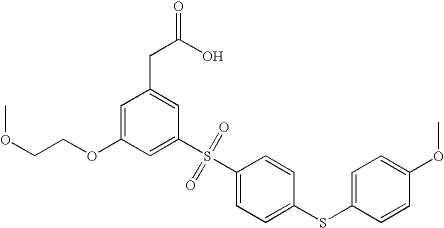 | {3-(2-Methoxy-ethoxy)-5-[4-(4-methoxy-phenylsulfanyl)-benzenesulfonyl]-phenyl}-acetic acid | 488.58 | [M + H⁺]⁺ =489.01 |
| P-0252 | 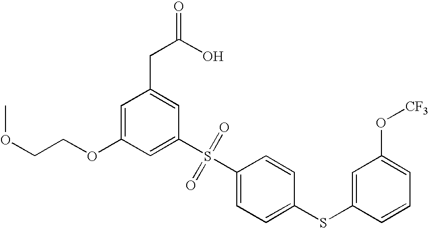 | {3-(2-Methoxy-ethoxy)-5-[4-(3-trifluoromethoxy-phenylsulfanyl)-benzenesulfonyl]-phenyl}-acetic acid | 542.55 | [M + H⁺]⁺ =543.1 |
| P-0253 | 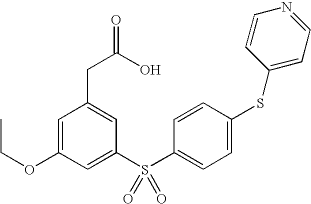 | {3-Ethoxy-5-[4-(pyridin-4-ylsulfanyl)-benzenesulfonyl]-phenyl}-acetic acid | 429.52 | [M + H⁺]⁺ =429.9 |
| P-0254 | 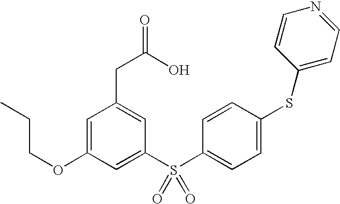 | {3-Propoxy-5-[4-(pyridin-4-ylsulfanyl)-benzenesulfonyl]-phenyl}-acetic acid | 443.54 | [M + H⁺]⁺ =444.3 |
| P-0255 | 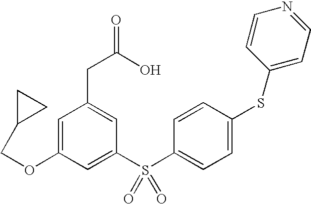 | {3-Cyclopropylmethoxy-5-[4-(pyridin-4-ylsulfanyl)-benzenesulfonyl]-phenyl}-acetic acid | 455.55 | [M + H⁺]⁺ =455.9 |
| P-0256 | 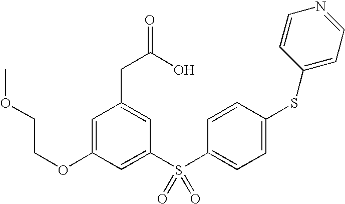 | {3-(2-Methoxy-ethoxy)-5-[4-(pyridin-4-ylsulfanyl)-benzenesulfonyl]-phenyl}-acetic acid | 459.54 | [M + H⁺]⁺ =459.9 |
| P-0281 | 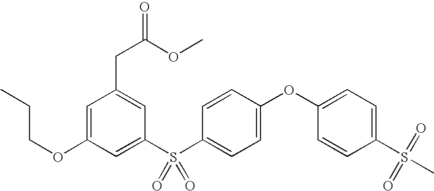 | {3-[4-(4-Methanesulfonyl-phenoxy)-benzenesulfonyl]-5-propoxy-phenyl}-acetic acidmethyl ester | 518.60 | [M + H⁺]⁺ =519.2 |
| P-0282 | 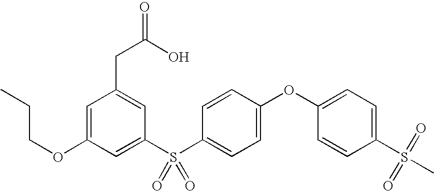 | {3-[4-(4-Methanesulfonyl-phenoxy)-benzenesulfonyl]-5-propoxy-phenyl}-acetic acid | 504.58 | [M + H⁺]⁺ =503.2 |
| P-0261 | 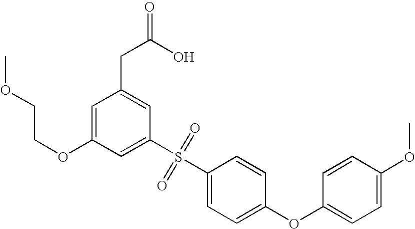 | {3-(2-Methoxy-ethoxy)-5-[4-(4-methoxy-phenoxy)-benzenesulfonyl]-phenyl}-acetic acid | 472.51 | [M + H⁺]⁺ =473.1 |
| P-0262 | 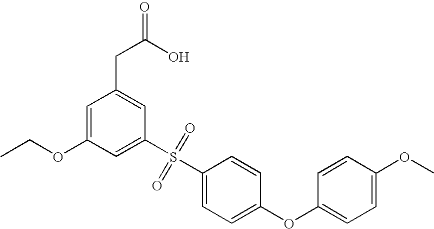 | {3-Ethoxy-5-[4-(4-methoxy-phenoxy)-benzenesulfonyl]-phenyl}-acetic acid | 442.49 | [M + H⁺]⁺ =443.1 |
| P-0263 | 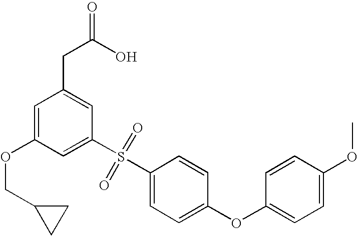 | {3-Cyclopropylmethoxy-5-[4-(4-methoxy-phenoxy)-benzenesulfonyl]-phenyl}-acetic acid | 468.52 | [M + H⁺]⁺ =469.1 |
| P-0264 | 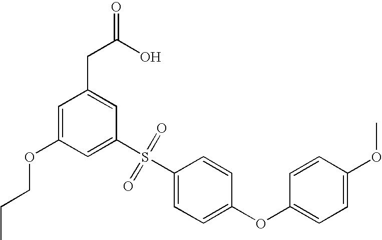 | {3-[4-(4-Methoxy-phenoxy)-benzenesulfonyl]-5-propoxy-phenyl}-acetic acid | 456.51 | [M + H⁺]⁺ =457.1 |
| P-0265 | 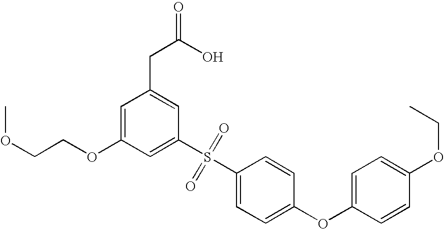 | [3-[4-(4-Ethoxy-phenoxy)-benzenesulfonyl]-5-(2-methoxy-ethoxy)-phenyl]-acetic acid | 486.54 | [M + H⁺]⁺ =487.1 |
| P-0266 | 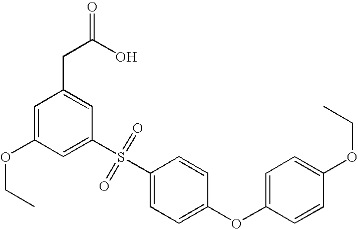 | {3-Ethoxy-5-[4-(4-ethoxy-phenoxy)-benzenesulfonyl]-phenyl}-acetic acid | 456.51 | [M + H⁺]⁺ =457.1 |
| P-0267 | 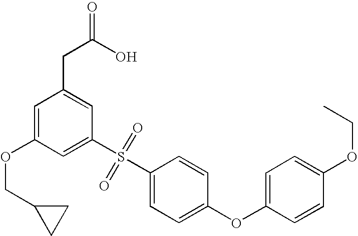 | {3-Cyclopropylmethoxy-5-[4-(4-ethoxy-phenoxy)-benzenesulfonyl]-phenyl}-acetic acid | 482.55 | [M + H⁺]⁺ =483.1 |
| P-0268 | 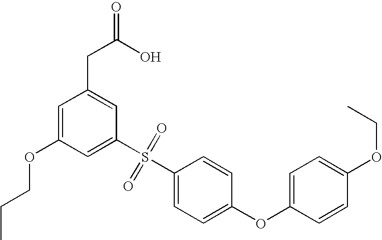 | {3-[4-(4-Ethoxy-phenoxy)-benzenesulfonyl]-5-propoxy-phenyl}-acetic acid | 470.54 | [M + H⁺]⁺ =471.1 |
| P-0269 | 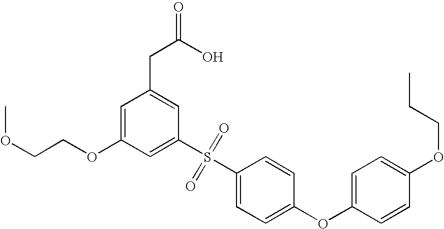 | {3-(2-Methoxy-ethoxy)-5-[4-(4-propoxy-phenoxy)-benzenesulfonyl]-phenyl}-acetic acid | 500.57 | [M + H⁺]⁺ =501.1 |
| P-0270 | 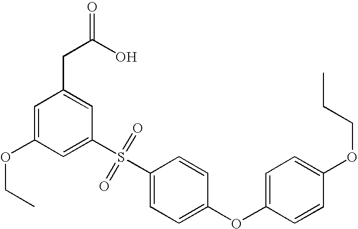 | {3-Ethoxy-5-[4-(4-propoxy-phenoxy)-benzenesulfonyl]-phenyl}-acetic acid | 470.54 | [M + H⁺]⁺ =471.1 |
| P-0271 | 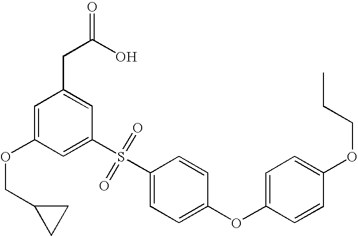 | {3-Cyclopropylmethoxy-5-[4-(4-propoxy-phenoxy)-benzenesulfonyl]-phenyl}-acetic acid | 496.58 | [M + H⁺]⁺ =457.1 |
| P-0272 | 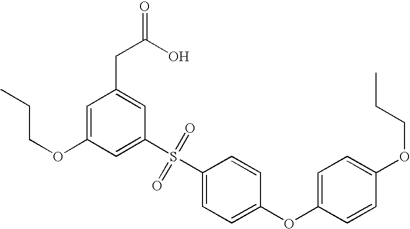 | {3-Propoxy-5-[4-(4-propoxy-phenoxy)-benzenesulfonyl]-phenyl}-acetic acid | 484.57 | [M + H⁺]⁺ =497.1 |
| P-0273 | 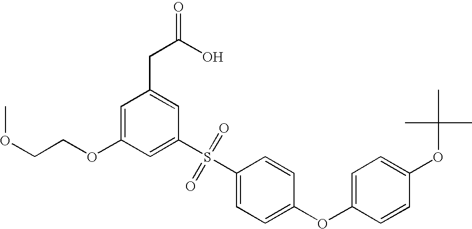 | [3-[4-(4-tert-Butoxy-phenoxy)-benzenesulfonyl]-5-(2-methoxy-ethoxy)-phenyl]-acetic acid | 514.59 | [M + H⁺]⁺ =485.1 |
| P-0274 | 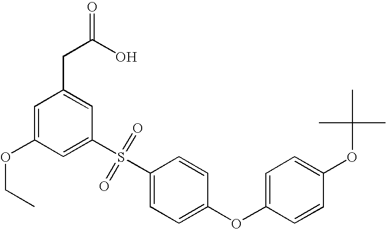 | {3-[4-(4-tert-Butoxy-phenoxy)-benzenesulfonyl]-5-ethoxy-phenyl}-acetic acid | 484.57 | [M + H⁺]⁺ +DMSO =593.2 |
| P-0275 | 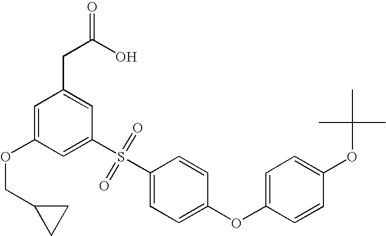 | {3-[4-(4-tert-Butoxy-phenoxy)-benzenesulfonyl]-5-cyclopropylmethoxy-phenyl}-acetic acid | 510.60 | [M + H⁺]⁺ +DMSO =563.2 |
| P-0276 | 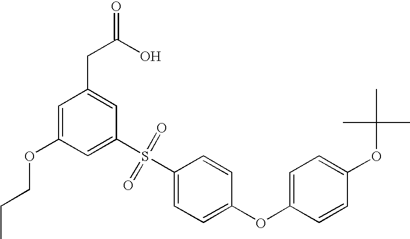 | {3-[4-(4-tert-Butoxy-phenoxy)-benzenesulfonyl]-5-propoxy-phenyl}-acetic acid | 498.59 | [M + H⁺]⁺ =511.1 |
| P-0277 | 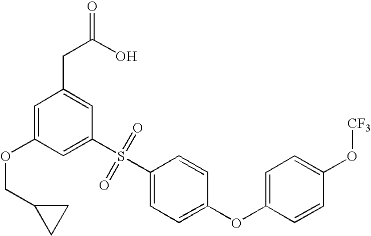 | {3-Cyclopropylmethoxy-5-[4-(4-trifluoromethoxy-phenoxy)-benzenesulfonyl]-phenyl}-acetic acid | 522.49 | [M + H⁺]⁺ =523.1 |
| P-0280 | 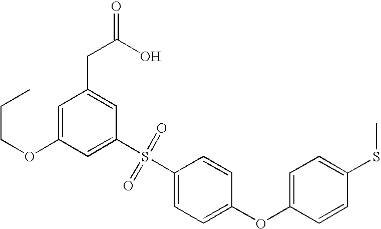 | {3-[4-(4-Methylsulfanyl-phenoxy)-benzenesulfonyl]-5-propoxy-phenyl}-acetic acid | 472.58 | [M + H⁺]⁺ =471.2 |
| P-0088 | 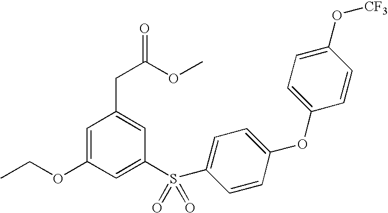 | {3-Ethoxy-5-[4-(4-trifluoromethoxy-phenoxy)-benzenesulfonyl]-phenyl}-acetic acid methylester | 510.48 | [M + H⁺]⁺ =511.3 |
| P-0207 | 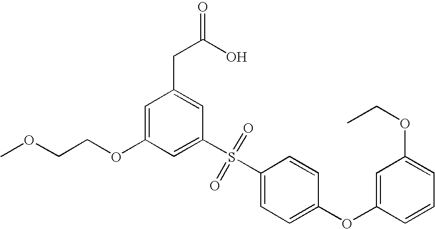 | [3-[4-(3-Ethoxy-phenoxy)-benzenesulfonyl]-5-(2-methoxy-ethoxy)-phenyl]-acetic acid | 486.54 | [M + H⁺]⁺ =487.1 |
| P-0208 | 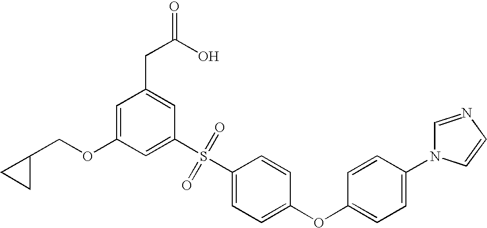 | {3-Cyclopropylmethoxy-5-[4-(4-imidazol-1-yl-phenoxy)-benzenesulfonyl]-phenyl}-acetic acid | 504.56 | [M + H⁺]⁺ =505.1 |
| P-0212 | 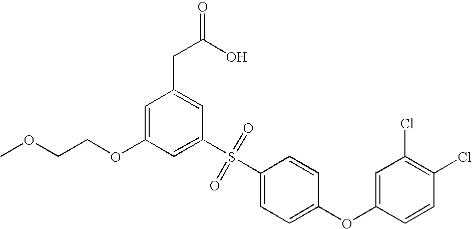 | [3-[4-(3,4-Dichloro-phenoxy)-benzenesulfonyl]-5-(2-methoxy-ethoxy)-phenyl]-acetic acid | 511.38 | [M + H⁺]⁺ =511.1 |
| P-0213 | 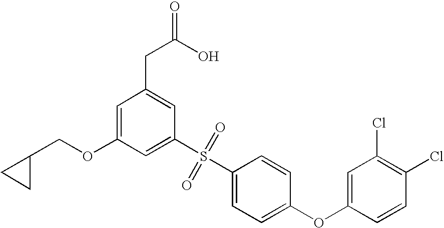 | {3-Cyclopropylmethoxy-5-[4-(3,4-dichloro-phenoxy)-benzenesulfonyl]-phenyl}-acetic acid | 507.39 | [M + H⁺]⁺ =507.1 |
| P-0214 | 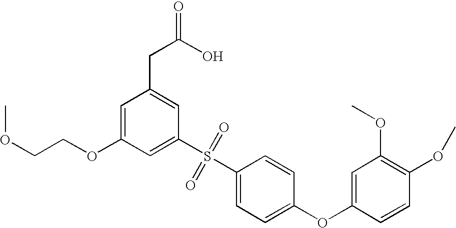 | [3-[4-(3,4-Dimethoxy-phenoxy)-benzenesulfonyl]-5-(2-methoxy-ethoxy)-phenyl]-acetic acid | 502.54 | [M + H⁺]⁺ =503.1 |
| P-0215 | 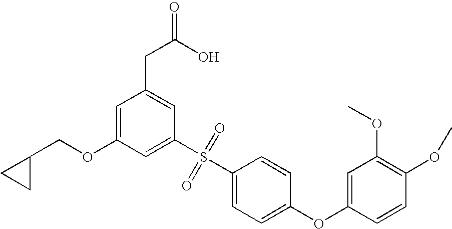 | {3-Cyclopropylmethoxy-5-[4-(3,4-dimethoxy-phenoxy)-benzenesulfonyl]-phenyl}-acetic acid | 498.55 | [M + H⁺]⁺ =499.1 |
| P-0216 | 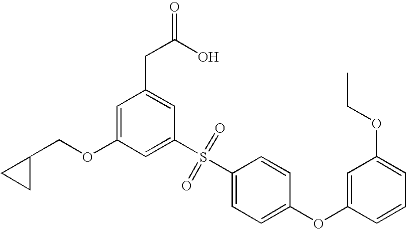 | {3-Cyclopropylmethoxy-5-[4-(3-ethoxy-phenoxy)-benzenesulfonyl]-phenyl}-acetic acid | 482.55 | [M + H⁺]⁺ =483.1 |
| P-0217 | 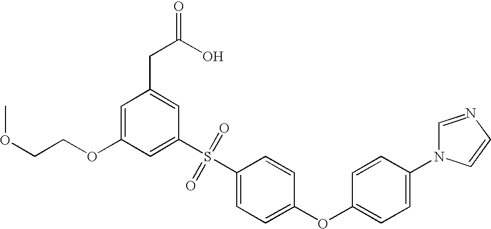 | [3-[4-(4-Imidazol-1-yl-phenoxy)-benzenesulfonyl]-5-(2-methoxy-ethoxy)-phenyl]-acetic acid | 508.55 | [M + H⁺]⁺ =509.1 |
| P-0229 | 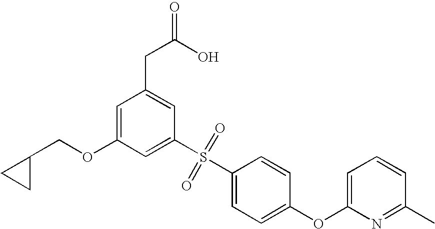 | {3-Cyclopropylmethoxy-5-[4-(6-methyl-pyridin-2-yloxy)-benzenesulfonyl]-phenyl}-acetic acid | 453.51 | [M + H⁺]⁺ =453.9 |
| P-0230 | 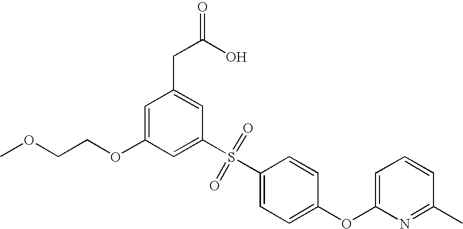 | {3-(2-Methoxy-ethoxy)-5-[4-(6-methyl-pyridin-2-yloxy)-benzenesulfonyl]-phenyl}-acetic acid | 457.50 | [M + H⁺]⁺ =458.3 |
| P-0233 | 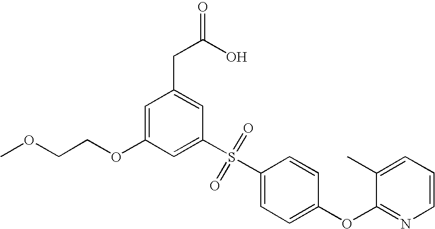 | {3-(2-Methoxy-ethoxy)-5-[4-(3-methyl-pyridin-2-yloxy)-benzenesulfonyl]-phenyl}-acetic acid | 457.50 | [M + H⁺]⁺ =458.3 |

### Example 4

### Synthesis of (3-butoxy-5-phenoxy-phenyl)-acetic acid (P-0006)

Compound P-0006 was synthesized in two steps from (3-butoxy-5-hydroxy-phenyl)-acetic acid methyl ester 9 as shown in Scheme 13.

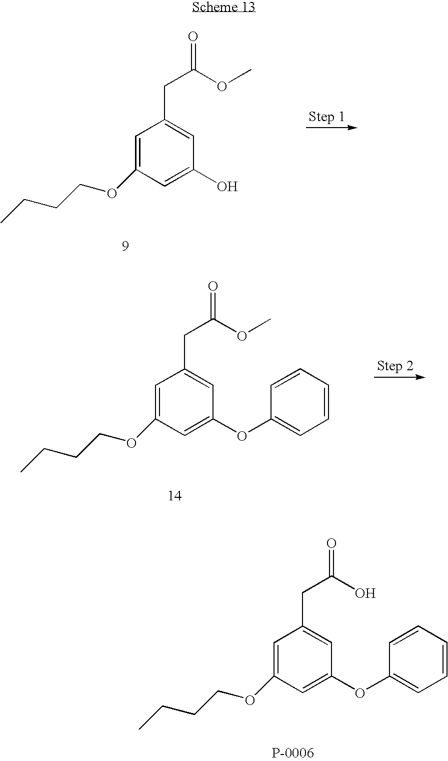

### Step 1: Preparation of (3-butoxy-5-phenoxy-phenyl)-acetic acid methyl ester (14)

To a solution of (3-butoxy-5-hydroxy-phenyl)-acetic acid methyl ester (9, 200 mg, 0.0008 mol, prepared as described in Step 1 of Scheme 12, Example 3) dissolved in 1,4-dioxane (10 mL, 0.1 mol), cesium carbonate (550 mg, 0.0017 mol), iodobenzene (140 μL, 0.0012 mol), L-proline (30 mg, 0.0002 mol) and copper(I) iodide (20 mg, 0.00008 mol) were added. The mixture was heated overnight at 90° C. Ethyl acetate was added and the mixture was acidified using 1M HCl. The aqueous layer was extracted 3 times with ethyl acetate, dried with sodium sulfate and concentrated. Purification using flash chromatography (10-20% ethyl acetate in hexanes) yielded the desired compound (14, 19 mg, 7%).

### Step 2: Preparation of (3-butoxy-5-phenoxy-phenyl)-acetic acid (P-0006)

To a solution of (3-butoxy-5-phenoxy-phenyl)-acetic acid methyl ester (14, 18 mg, 0.000057 mol) in tetrahydrofuran (2 mL, 0.02 mol), potassium hydroxide in water (1M, 0.6 mL) was added and the mixture stirred overnight at room temperature. Ethyl acetate was added and the mixture was acidified with 1M HCl. The aqueous phase was extracted with ethyl acetate. The organic phase was washed with brine, dried over sodium sulfate and concentrated to yield the desired compound (P-0006, 15 mg, 84%). Calculated molecular weight 300.35, MS (ESI) [M+H⁺]⁺=301.2 [M−H⁺]⁻=299.1.

### Example 5

### Synthesis of [3-butoxy-5-(3-methoxy-benzenesulfonyl)-phenyl]-acetic acid (P-0025)

Compound P-0025 was synthesized in four steps from (3,5-dihydroxy-phenyl)-acetic methyl ester 8 as shown in Scheme 14.

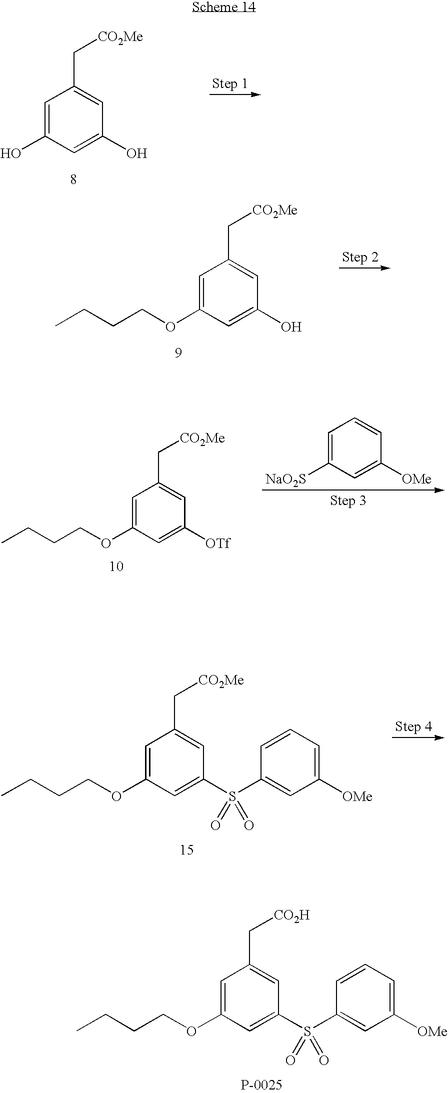

### Step-1: Preparation of (3-butoxy-5-hydroxy-phenyl)-acetic acid methyl ester (9)

Into an oven dried, then flame dried round bottom flask, (3,5-dihydroxy-phenyl)-acetic acid methyl ester (8, 5.0 g, 0.027 mol) and potassium carbonate (3.81 g, 0.0276 mol) were dissolved in 2-butanone (500 mL, 5.55 mol). The reaction vessel was purged with argon and heated at 97° C. Into an addition funnel, 2-butanone (50 mL, 0.55 mol) and 1-iodobutane (4.59 g, 0.0249 mol) were combined. The addition funnel was attached onto the reaction vessel and the contents added to the reaction over 2 hours. After the final addition, the funnel was replaced with a condenser and the reaction was heated overnight. The following morning, TLC (20% ethyl acetate/hexane) showed three spots (R_{f}=0.8, 0.3, and 0.02). The solid was filtered off and the solvent was removed. Water and ethyl acetate were added. The solution was neutralized using 1M HCl, and the water phase extracted with ethyl acetate. The pooled organic phase was dried (Na₂SO₄) and absorbed onto silica. Flash chromatography with silica column was utilized with step gradient solvents (4, 7, 10, 20% ethyl acetate in hexanes) to isolate the desired methyl ester (R_{f}=0.3), which was taken on to the next step. ¹H NMR (CDCl₃) consistent with compound structure.

### Step-2: Preparation of (3-butoxy-5-trifluoromethanesulfonyloxy-phenyl)-acetic acid methyl ester (10)

Into a round bottom flask (3-butoxy-5-hydroxy-phenyl)-acetic acid methyl ester (9, 2.3 g, 0.0096 mol) was dissolved in pyridine (8 mL, 0.1 mol). The flask was placed on an ice bath and cooled to 0° C. Trifluoromethanesulfonic anhydride (3.3 g, 0.012 mol) was added drop wise to the solution over 15 minutes. The reaction was stirred for 4 hours and allowed to warm to ambient conditions. The flask was placed on a new ice bath and 40 mL water was added to the vessel, followed by diethyl ether (90 mL) and concentrated HCl (6 mL). The reaction was stirred vigorously throughout this process. After 40 minutes, the organic layer was separated, washed with 1N HCl solution and dried under MgSO₄. Solvent was removed under reduced pressure to give a dark yellow oil. A silica plug was used to isolate the desired compound as a yellow oil. ¹H NMR consistent with compound structure.

### Step 3: Preparation of [3-butoxy-5-(3-methoxybenzenesulfonyl)-phenyl]-acetic acid methyl ester (15)

Into a dry round bottom flask, (3-butoxy-5-trifluoromethanesulfonyloxy-phenyl)-acetic acid methyl ester (10, 150 mg, 0.00040 mol) was added under argon flow. 3-Methoxyphenyl sulfinic acid sodium salt (97 mg, 0.00050 mol) and toluene (8 mL, 0.08 mol) were added and the vessel purged with argon. Cesium carbonate (205 mg, 0.000629 mol), tris(dibenzylideneacetone)dipalladium (0) (4 mg, 0.000004 mol), and xanthphos (4 mg, 0.000007 mol) were quickly added and the reaction was heated at 110° C. overnight, after which TLC analysis of the reaction (20% ethyl acetate/hexane) showed that the desired compound was formed (R_{f}=0.3). The reaction was allowed to cool to room temperature and diluted with water. The reaction was extracted with ethyl acetate 3× and the combined organic layers were washed with brine 2×, dried over sodium sulfate, and evaporated under reduced pressure to afford the crude compound as a brown oil. The oil was absorbed onto silica, and purified via flash chromatography with a step gradient (5, 7, 10% ethyl acetate in hexanes) to isolate the desired compound. ¹H NMR consistent with compound structure.

### Step 4: Preparation of [3-butoxy-5-(3-methoxy-benzenesulfonyl)-phenyl]-acetic acid (P-0025)

Into a flask, [3-Butoxy-5-(3-methoxy-benzenesulfonyl)-phenyl]-acetic acid methyl ester 15 was treated with a 5 mL mixture of tetrahydrofuran/1N KOH (4:1) and stirred vigorously overnight. The reaction was acidified by adding 1N HCl (acidic via pH paper) and extracted with ethyl acetate (3 times the reaction volume) and dried over MgSO₄. Trituration: Hexane/dichloromethane were added (3 mL each) and the flask stirred for about an hour. At this point, the solvent was removed via filtration. Off white/brown solids were placed under a high vacuum over the weekend. ¹H NMR (CD₃OD) consistent with compound structure. Calculated molecular weight 378.44, MS (ESI) [M−H⁺]⁻=377.13.

Additional compounds were prepared by optionally replacing the 1-iodobutane with an appropriate iodoalkyl compound in Step 1, and/or optionally replacing the 3-methoxyphenyl sulfinic acid sodium salt with an appropriate sulfinic acid sodium salt in Step 3. In addition to these optional changes in Steps 1 or 3, compounds P-0149 through P-0157 were prepared by replacing (3,5-dihydroxy-phenyl)-acetic acid methyl ester 8 with (3,5-dihydroxy-phenyl)-propionic acid methyl ester in Step 1, compounds P-0147, P-0148, and P-0159 were prepared by replacing (3,5-dihydroxy-phenyl)-acetic acid methyl ester 8 with (3-hydroxy-phenyl)-propionic acid methyl ester, used in Step 2 (no Step 1), and compounds P-0258, P-0294, and P-0295 were prepared by replacing (3,5-dihydroxy-phenyl)-acetic acid methyl ester 8 with (3-hydroxy-phenyl)-acetic acid methyl ester, used in Step 2 (no Step 1). The following Table 4 indicates the appropriate iodoalkyl and sulfinic acid reagents used in Step 1 and 3, respectively, for the indicated compound.

TABLE 4

| Cmpd. | Step 1 iodoalkyl | |
|---|---|---|
| number | compound | Step 3 sulfinic acid sodium salt |
| P-0011 | 1-iodobutane | phenyl |
| P-0022 | 1-iodobutane | 4-trifluoromethylphenyl |
| P-0023 | 1-iodobutane | 4-methoxyphenyl |
| P-0024 | 1-iodobutane | 4-trifluoromethoxyphenyl |
| P-0026 | 1-iodobutane | 5-(1-methyl-5-trifluoromethyl-1H- |
| | | pyrazol-3-yl)-thiophen-2-yl |
| P-0028 | iodoethane | 5-(1-methyl-5-trifluoromethyl-1H- |
| | | pyrazol-3-yl)-thiophen-2-yl |
| P-0029 | iodoethane | 4-(4-trifluoromethyl-phenoxy)-phenyl |
| P-0030 | iodoethane | 4-methoxyphenyl |
| P-0050 | 1-iodopropane | 4-fluorophenyl |
| P-0051 | 1-iodo-2- | 4-methoxyphenyl |
| | methoxyethane | |
| P-0052 | 1-iodo-2- | 5-(1-methyl-5-trifluoromethyl-1H- |
| | methoxyethane | pyrazol-3-yl)-thiophen-2-yl |
| P-0053 | iodomethane | 4-methoxyphenyl |
| P-0054* | 1-iodopropane | 5-(1-methyl-5-trifluoromethyl-1H- |
| | | pyrazol-3-yl)-thiophen-2-yl |
| P-0055 | 1-iodopropane | 5-(1-methyl-5-trifluoromethyl-1H- |
| | | pyrazol-3-yl)-thiophen-2-yl |
| P-0056 | 1-iodopropane | 4-methoxyphenyl |
| P-0060 | iodomethane | 4-(4-trifluoromethyl-phenoxy)-phenyl |
| P-0061 | iodomethyl-benzene | 4-(4-trifluoromethyl-phenoxy)-phenyl |
| P-0063 | 1-iodobutane | 4-(4-trifluoromethyl-phenoxy)-phenyl |
| P-0065 | iodomethane | 5-(1-methyl-5-trifluoromethyl-1H- |
| | | pyrazol-3-yl)-thiophen-2-yl |
| P-0066 | iodomethyl-benzene | 5-(1-methyl-5-trifluoromethyl-1H- |
| | | pyrazol-3-yl)-thiophen-2-yl |
| P-0067 | 1-iodopropane | 4-(4-trifluoromethyl-phenoxy)-phenyl |
| P-0068 | iodomethyl- | 4-(4-trifluoromethyl-phenoxy)-phenyl |
| | cyclopropane | |
| P-0069 | iodomethyl-benzene | 4-methoxyphenyl |
| P-0070 | iodoethane | 4′-methyl-biphen-2-yl |
| P-0071 | 1-iodopropane | 4′-methyl-biphen-2-yl |
| P-0072 | 1-iodopropane | 4-butoxyphenyl |
| P-0073 | 1-iodopropane | 4-butylphenyl |
| P-0074 | 1-iodopropane | 3-(4-trifluoromethyl-phenoxy)-phenyl |
| P-0075 | 1-iodopropane | 3-(4-methoxy-phenoxy)-phenyl |
| P-0076 | 1-iodopropane | 3-(2-methoxy-phenoxy)-phenyl |
| P-0077 | iodoethane | 4-(3-butyl-ureido)-phenyl |
| P-0078 | iodoethane | 3,4-dichlorophenyl |
| P-0084 | iodoethane | 2-(4-methyl-phenoxy)-phenyl |
| P-0085* | iodoethane | 4-fluorophenyl |
| P-0086 | iodoethane | 4-fluorophenyl |
| P-0147 | no Step 1 | 5-(1-methyl-5-trifluoromethyl-1H- |
| | | pyrazol-3-yl)-thiophen-2-yl |
| P-0148 | no Step 1 | 4-methoxyphenyl |
| P-0149 | 1-iodopropane | 5-(1-methyl-5-trifluoromethyl-1H- |
| | | pyrazol-3-yl)-thiophen-2-yl |
| P-0150 | 1-iodopropane | 4-(4-trifluoromethyl-phenoxy)-phenyl |
| P-0151 | 1-iodobutane | 5-(1-methyl-5-trifluoromethyl-1H- |
| | | pyrazol-3-yl)-thiophen-2-yl |
| P-0152 | 1-iodobutane | 4-methoxyphenyl |
| P-0153 | 1-iodobutane | 4-(4-trifluoromethyl-phenoxy)-phenyl |
| P-0154 | iodoethane | 5-(1-methyl-5-trifluoromethyl-1H- |
| | | pyrazol-3-yl)-thiophen-2-yl |
| P-0155 | iodoethane | 4-(4-trifluoromethyl-phenoxy)-phenyl |
| P-0156 | iodoethane | 4-methoxyphenyl |
| P-0157 | 1-iodopropane | 4-methoxyphenyl |
| P-0159 | no Step 1 | 4-(4-trifluoromethyl-phenoxy)-phenyl |
| P-0258 | no Step 1 | 4′-trifluoromethyl-biphen-3-yl |
| P-0175 | iodemethane | 4′-trifluoromethyl-biphen-3-yl |
| P-0206 | iodoethane | 2,5-dimethyl-thiophen-3-yl |
| P-0286 | iodoethane | thiophen-2-yl |
| P-0294 | no Step 1 | 5-(1-methyl-5-trifluoromethyl-1H- |
| | | pyrazol-3-yl)-thiophen-2-yl |
| P-0295 | no Step 1 | 4-(4-trifluoromethyl-phenoxy)-phenyl |

*Methyl ester isolated after Step 3.

The compound structures, names and mass spectrometry results for these compounds are provided in the following Table 5.

TABLE 5

| | | | Molecular weight | |
|---|---|---|---|---|
| Cmpd. | | | | Measured |
| number | Structure | Name | Calc. | MS(ESI) |
| P-0011 | 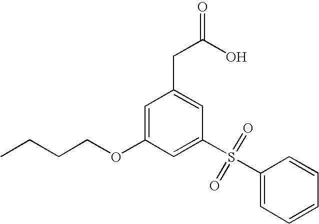 | (3-Benzenesulfonyl-5-butoxy-phenyl)-acetic acid | 348.42 | [M + H⁺]⁺ =349.2[M − H⁺]⁻ =347.2 |
| P-0022 | 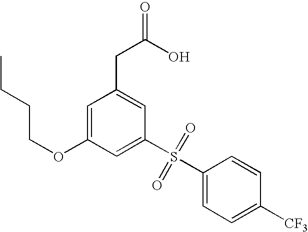 | [3-Butoxy-5-(4-trifluoromethyl-benzenesulfonyl)-phenyl]-acetic acid | 416.41 | [M + H⁺]⁺ =417.3[M − H⁺]⁻ =415.2 |
| P-0023 | 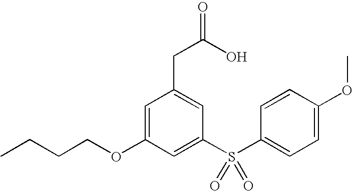 | [3-Butoxy-5-(4-methoxy-benzenesulfonyl)-phenyl]-acetic acid | 378.44 | [M + H⁺]⁺ =379.2[M − H⁺]⁻ =377.2 |
| P-0024 | 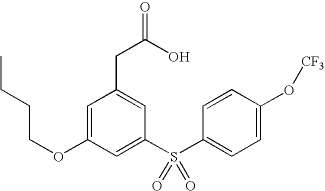 | [3-Butoxy-5-(4-trifluoromethoxy-benzenesulfonyl)-phenyl]-acetic acid | 432.41 | [M + H⁺]⁺ =433.2[M − H⁺]⁻ =431.1 |
| P-0026 | 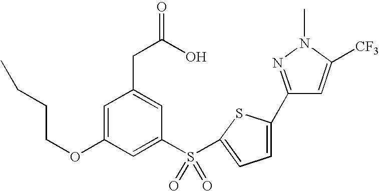 | {3-Butoxy-5-[5-(1-methyl-5-trifluoromethyl-1H-pyrazol-3-yl)-thiophene-2-sulfonyl]-phenyl}-acetic acid | 502.53 | [M + H⁺]⁺ =502.2 |
| P-0028 | 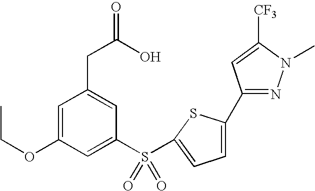 | {3-Ethoxy-5-[5-(1-methyl-5-trifluoromethyl-1H-pyrazol-3-yl)-thiophene-2-sulfonyl]-phenyl}-acetic acid | 474.48 | [M − H⁺]⁻ =472.41 |
| P-0029 | 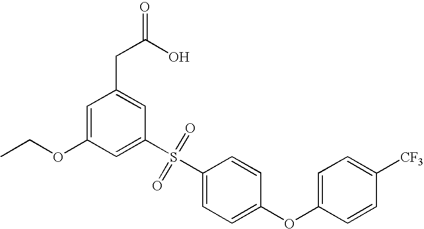 | {3-Ethoxy-5-[4-(4-trifluoromethyl-phenoxy)-benzenesulfonyl]-phenyl}-acetic acid | 480.46 | [M + H⁺]⁺ =481.2[M − H⁺]⁻ =479.0 |
| P-0030 | 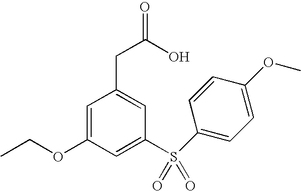 | [3-Ethoxy-5-(4-methoxy-benzenesulfonyl)-phenyl]-acetic acid | 350.39 | [M + H⁺]⁺ =351.1[M − H⁺]⁻ =349.0 |
| P-0050 | 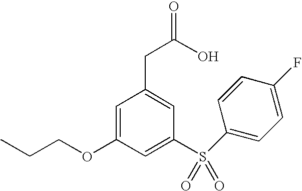 | [3-(4-Fluoro-benzenesulfonyl)-5-propoxy-phenyl]-acetic acid | 352.38 | [M + H⁺]⁺ =353.0[M − H⁺]⁻ =351.0 |
| P-0051 | 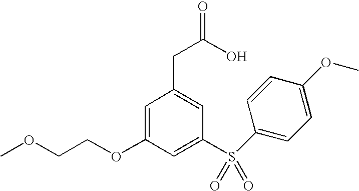 | [3-(4-Methoxy-benzenesulfonyl)-5-(2-methoxy-ethoxy)-phenyl]-acetic acid | 380.42 | [M + H⁺]⁺ =381.11[M − H⁺]⁻ =379.16 |
| P-0052 | 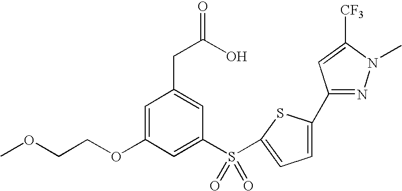 | {3-(2-Methoxy-ethoxy)-5-[5-(1-methyl-5-trifluoromethyl-1H-pyrazol-3-yl)-thiophene-2-sulfonyl]-phenyl}-aceticacid | 504.50 | [M + H⁺]⁺ =505.42 |
| P-0053 | 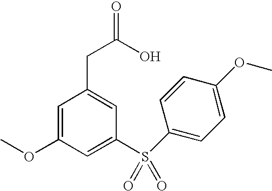 | [3-Methoxy-5-(4-methoxy-benzenesulfonyl)-phenyl]-acetic acid | 336.36 | [M + H⁺]⁺ =337.1[M − H⁺]⁻ =335.0 |
| P-0054 | 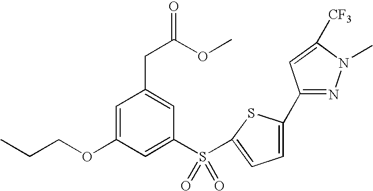 | {3-[5-(1-Methyl-5-trifluoromethyl-1H-pyrazol-3-yl)-thiophene-2-sulfonyl]-5-propoxy-phenyl}-aceticacid methyl ester | 502.53 | [M + H⁺]⁺ =503.2 |
| P-0055 | 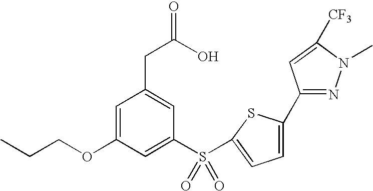 | {3-[5-(1-Methyl-5-trifluoromethyl-1H-pyrazol-3-yl)-thiophene-2-sulfonyl]-5-propoxy-phenyl}-aceticacid | 488.51 | [M + H⁺]⁺ =489.2 |
| P-0056 | 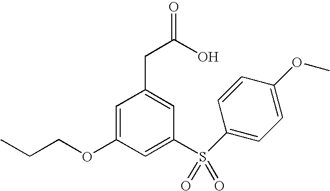 | [3-(4-Methoxy-benzenesulfonyl)-5-propoxy-phenyl]-acetic acid | 364.42 | [M − H⁺]⁻ =363.1 |
| P-0060 | 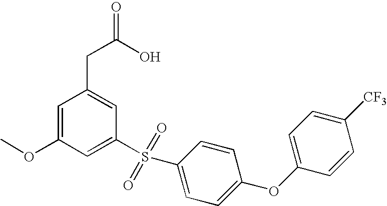 | {3-Methoxy-5-[4-(4-trifluoromethyl-phenoxy)-benzenesulfonyl]-phenyl}-acetic acid | 466.43 | [M + H⁺]⁺ =467.2[M − H⁺]⁻ =465.1 |
| P-0061 | 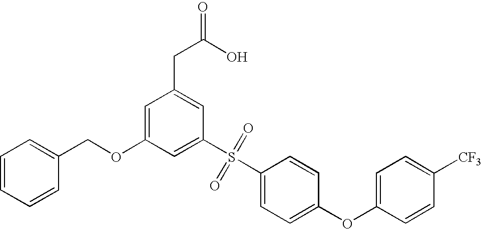 | {3-Benzyloxy-5-[4-(4-trifluoromethyl-phenoxy)-benzenesulfonyl]-phenyl}-acetic acid | 542.53 | [M + H⁺]⁺ =543.2[M − H⁺]⁻ =541.1 |
| P-0063 | 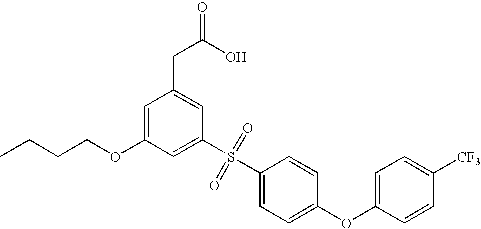 | {3-Butoxy-5-[4-(4-trifluoromethyl-phenoxy)-benzenesulfonyl]-phenyl}-acetic acid | 508.51 | [M + H⁺]⁺ =509.2[M − H⁺]⁻ =507.1 |
| P-0065 | 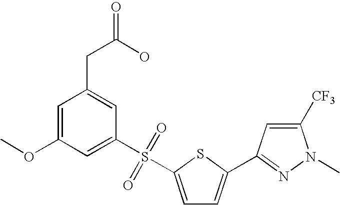 | {3-Methoxy-5-[5-(1-methyl-5-trifluoromethyl-1H-pyrazol-3-yl)-thiophene-2-sulfonyl]-phenyl}-acetic acid | 460.45 | [M − H⁺]⁻ =459.2 |
| P-0066 | 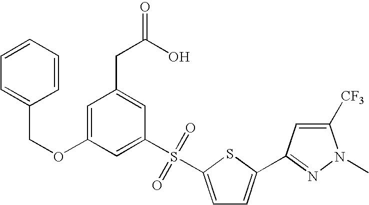 | {3-Benzyloxy-5-[5-(1-methyl-5-trifluoromethyl-1H-pyrazol-3-yl)-thiophene-2-sulfonyl]-phenyl}-aceticacid | 536.55 | [M + H⁺]⁺ =537.5 |
| P-0067 | 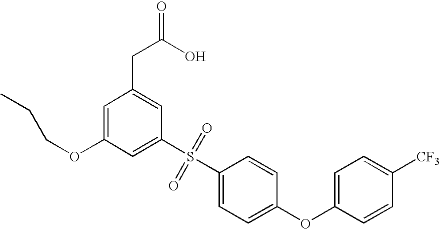 | {3-Propoxy-5-[4-(4-trifluoromethyl-phenoxy)-benzenesulfonyl]-phenyl}-acetic acid | 494.48 | [M − H⁺]⁻ =493.2 |
| P-0068 | 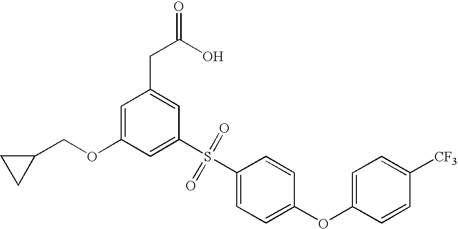 | {3-Cyclopropylmethoxy-5-[4-(4-trifluoromethyl-phenoxy)-benzenesulfonyl]-phenyl}-acetic acid | 506.49 | [M − H⁺]⁻ =505.1 |
| P-0069 | 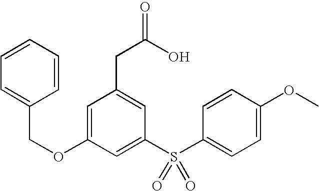 | [3-Benzyloxy-5-(4-methoxy-benzenesulfonyl)-phenyl]-acetic acid | 412.46 | [M + H⁺]⁺ =413.1[M − H⁺]⁻ =411.1 |
| P-0070 | 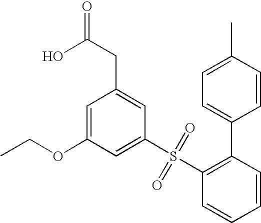 | [3-Ethoxy-5-(4′-methyl-biphenyl-2-sulfonyl)-phenyl]-acetic acid | 410.49 | NA |
| P-0071 | 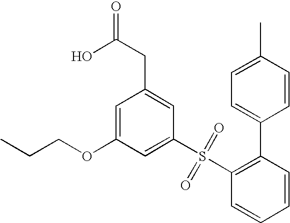 | [3-(4′-Methyl-biphenyl-2-sulfonyl)-5-propoxy-phenyl]-acetic acid | 424.51 | NA |
| P-0072 | 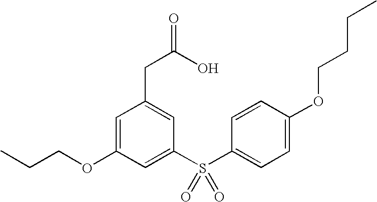 | [3-(4-Butoxy-benzenesulfonyl)-5-propoxy-phenyl]-acetic acid | 406.50 | [M + H⁺]⁺ =407.1[M − H⁺]⁻ =405.1 |
| P-0073 | 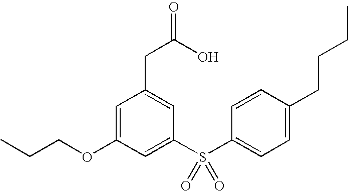 | [3-(4-Butyl-benzenesulfonyl)-5-propoxy-phenyl]-acetic acid | 390.50 | [M + H⁺]⁺ =391.2[M − H⁺]⁻ =389.1 |
| P-0074 | 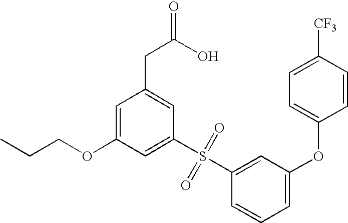 | {3-Propoxy-5-[3-(4-trifluoromethyl-phenoxy)-benzenesulfonyl]-phenyl}-acetic acid | 494.48 | [M + H⁺]⁺ =493.1 |
| P-0075 | 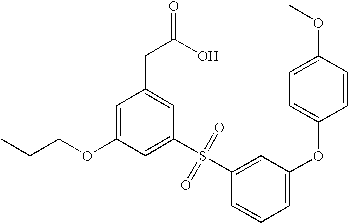 | {3-[3-(4-Methoxy-phenoxy)-benzenesulfonyl]-5-propoxy-phenyl}-acetic acid | 456.51 | [M + H⁺]⁺ =457.2[M − H⁺]⁻ =455.1 |
| P-0076 | 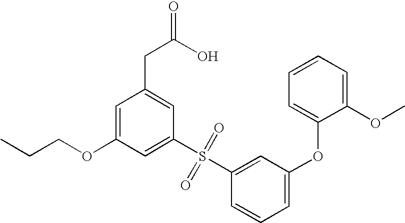 | {3-[3-(2-Methoxy-phenoxy)-benzenesulfonyl]-5-propoxy-phenyl}-acetic acid | 456.51 | [M + H⁺]⁺ =457.2[M − H⁺]⁻ =455.1 |
| P-0077 | 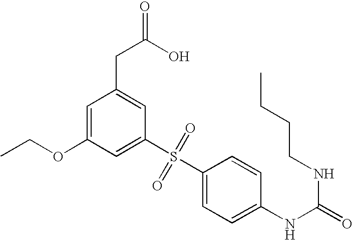 | {3-[4-(3-Butyl-ureido)-benzenesulfonyl]-5-ethoxy-phenyl}-acetic acid | 434.51 | [M + H⁺]⁺ =435.2[M − H⁺]⁻ =433.1 |
| P-0078 | 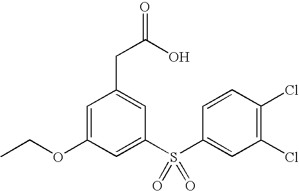 | [3-(3,4-Dichloro-benzenesulfonyl)-5-ethoxy-phenyl]-acetic acid | 389.25 | [M − H⁺]⁻ =386.9, 388.9,390.9 |
| P-0084 | 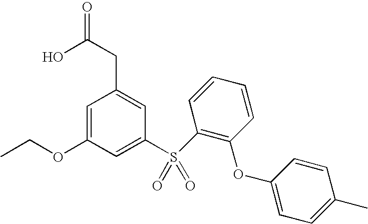 | [3-Ethoxy-5-(2-p-tolyloxy-benzenesulfonyl)-phenyl]-acetic acid | 426.49 | NA |
| P-0085 | 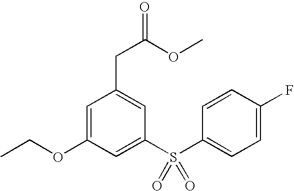 | [3-Ethoxy-5-(4-fluoro-benzenesulfonyl)-phenyl]-acetic acid methyl ester | 352.38 | [M + H⁺]⁺ =353.2 |
| P-0086 | 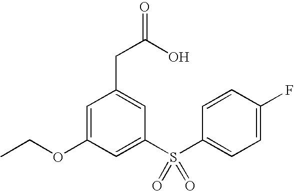 | [3-Ethoxy-5-(4-fluoro-benzenesulfonyl)-phenyl]-acetic acid | 338.35 | [M − H⁺]⁻ =337.0 |
| P-0147 | 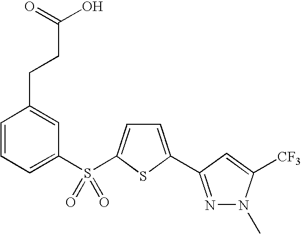 | 3-{3-[5-(1-Methyl-5-trifluoromethyl-1H-pyrazol-3-yl)-thiophene-2-sulfonyl]-phenyl}-propionic acid | 444.45 | [M − H⁺]⁻ =444.45 |
| P-0148 | 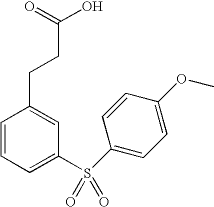 | 3-[3-(4-Methoxy-benzenesulfonyl)-phenyl]-propionic acid | 320.36 | [M − H⁺]⁻ =319.09 |
| P-0149 | 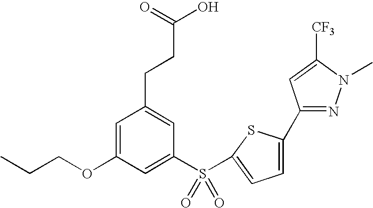 | 3-{3-[5-(1-Methyl-5-trifluoromethyl-1H-pyrazol-3-yl)-thiophene-2-sulfonyl]-5-propoxy-phenyl}-propionic acid | 502.53 | [M − H⁺]⁻ =500.95 |
| P-0150 | 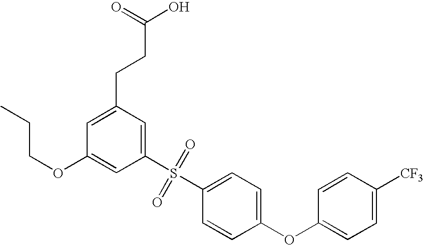 | 3-{3-Propoxy-5-[4-(4-trifluoromethyl-phenoxy)-benzenesulfonyl]-phenyl}-propionic acid | 508.51 | [M − H⁺]⁻ =507.03 |
| P-0151 | 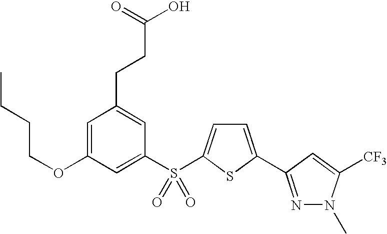 | 3-{3-Butoxy-5-[5-(1-methyl-5-trifluoromethyl-1H-pyrazol-3-yl)-thiophene-2-sulfonyl]-phenyl}-propionicacid | 516.56 | [M − H⁺]⁻ =515.53 |
| P-0152 | 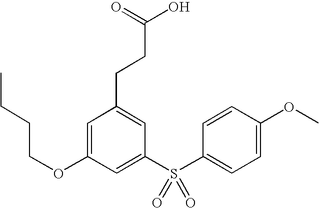 | 3-[3-Butoxy-5-(4-methoxy-benzenesulfonyl)-phenyl]-propionic acid | 392.47 | [M − H⁺]⁻ =391.11 |
| P-0153 | 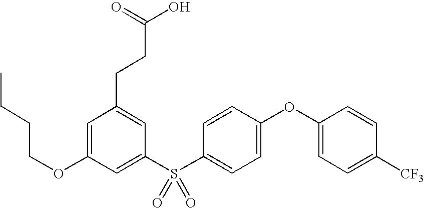 | 3-{3-Butoxy-5-[4-(4-trifluoromethyl-phenoxy)-benzenesulfonyl]-phenyl}-propionic acid | 522.54 | [M + H⁺]⁺ =521.13 |
| P-0154 | 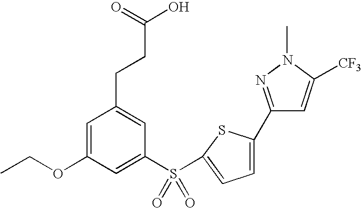 | 3-{3-Ethoxy-5-[5-(1-methyl-5-trifluoromethyl-1H-pyrazol-3-yl)-thiophene-2-sulfonyl]-phenyl}-propionicacid | 488.51 | [M + H⁺]⁺ =488.2 |
| P-0155 | 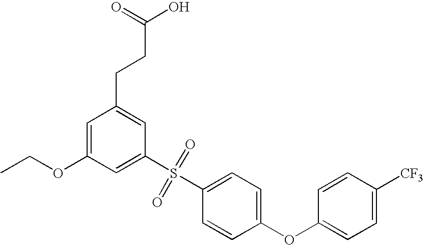 | 3-{3-Ethoxy-5-[4-(4-trifluoromethyl-phenoxy)-benzenesulfonyl]-phenyl}-propionic acid | 494.48 | [M − H⁺]⁻ =493.1 |
| P-0156 | 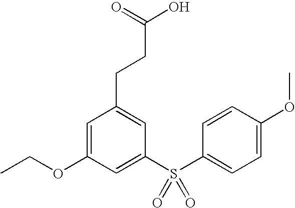 | 3-[3-Ethoxy-5-(4-methoxy-benzenesulfonyl)-phenyl]-propionic acid | 364.42 | [M − H⁺]⁻ =363.1 |
| P-0157 | 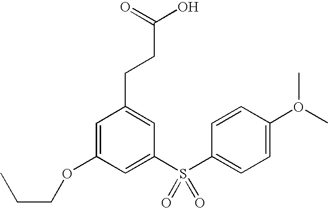 | 3-[3-(4-Methoxy-benzenesulfonyl)-5-propoxy-phenyl]-propionicacid | 378.44 | [M + H⁺]⁺ =379.2 |
| P-0159 | 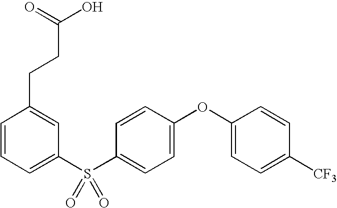 | 3-{3-[4-(4-Trifluoromethyl-phenoxy)-benzenesulfonyl]-phenyl}-propionicacid | 450.43 | [M − H⁺]⁻ =449.07 |
| P-0258 | 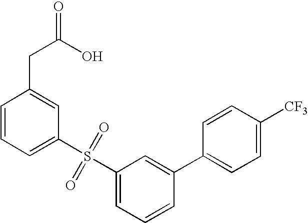 | [3-(4′-Trifluoromethyl-biphenyl-3-sulfonyl)-phenyl]-acetic acid | 420.41 | [M − H⁺]⁻ =419.1 |
| P-0175 | 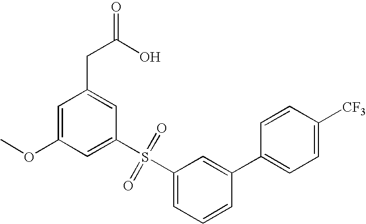 | [3-Methoxy-5-(4′-trifluoromethyl-biphenyl-3-sulfonyl)-phenyl]-acetic acid | 450.43 | [M − H⁺]⁻ =449.1 |
| P-0206 | 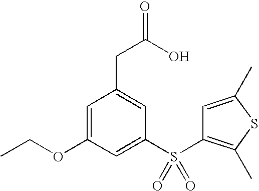 | [3-(2,5-Dimethyl-thiophene-sulfonyl)-5-ethoxy-phenyl]-acetic acid | 354.45 | [M + H⁺]⁺ =355.39 |
| P-0286 | 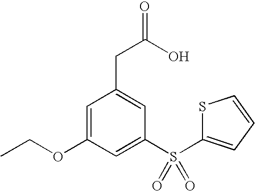 | [3-Ethoxy-5-(thiophene-2-sulfonyl)-phenyl]-acetic acid | 326.39 | [M − H⁺]⁻ =261.10 |
| P-0294 | 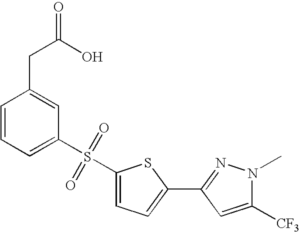 | {3-[5-(1-Methyl-5-trifluoromethyl-1H-pyrazol-3-yl)-thiophene-2-sulfonyl]-phenyl}-acetic acid | 430.43 | |
| P-0295 | 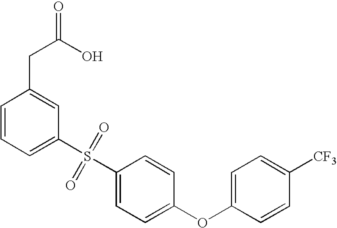 | {3-[4-(4-Trifluoromethyl-phenoxy)-benzenesulfonyl]-phenyl}-acetic acid | 436.40 | |

### Example 6

### Synthesis of [3-ethoxy-5-(4′-trifluoromethyl-biphenyl-3-sulfonyl)phenyl]-acetic acid (P-0080)

Compound P-0080 was synthesized in four steps from (3,5-dihydroxy-phenyl)-acetic acid methyl ester 8 as shown in Scheme 15.

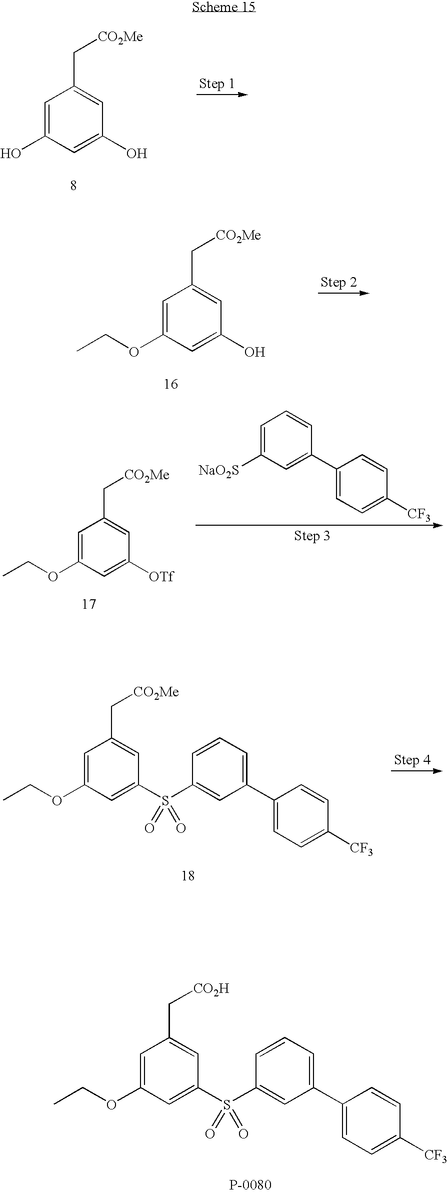

### Step-1. Preparation of (3-ethoxy-5-hydroxy-phenyl)-acetic acid methyl ester (16)

Into a flask, (3,5-dihydroxy-phenyl)-acetic acid methyl ester (8, 4 g, 0.02 mol) was dissolved in 2-butanone (80 mL, 0.8 mol). Potassium carbonate (9.10 g, 0.0659 mol) was added in one portion and iodoethane (1.60 mL, 0.0200 mol) was added drop wise. The reaction was heated to 80° C. and left stirring for 5 hours. The solid was filtered off and the solvent was removed. Water and ethyl acetate were added. The solution was neutralized with 1M HCl and the water phase was extracted with ethyl acetate. The pooled organic phase was dried (Na₂SO₄) and absorbed onto silica. Flash chromatography eluting with 20-40% ethyl acetate in hexanes afforded the desired compound as a clear yellow oil. ¹H NMR consistent with compound structure.

### Step-2: Preparation of (3-ethoxy-5-trifluoromethanesulfonyloxy-phenyl)-acetic acid methyl ester (17)

Into a round bottom flask (3-ethoxy-5-hydroxy-phenyl)-acetic acid methyl ester (16, 4 g, 0.02 mol) was dissolved in pyridine (60 mL, 0.7 mol) at 0° C. Trifluoromethanesulfonic anhydride (7 mL, 0.04 mol) was added in portions, and the reaction was left stirring for 16 hours and allowed to come to ambient conditions. The reaction was acidifed with concentrated HCl and extracted with diethyl ether 3×. The combined organic layers were then washed with brine 2×, dried over sodium sulfate, and evaporated to yield a red-orange oil. The oil was then purified via flash chromatography with 20-35% ethyl acetate in hexane on silica to yield the desired compound as a yellow oil. ¹H NMR was consistent with the desired compound.

### Step-3: Preparation of [3-ethoxy-5-(4′-trifluoromethyl-biphenyl-3-sulfonyl)phenyl]-acetic acid methyl ester (18)

Into a round bottom flask 4′-trifluoromethyl-biphenyl-3-sulfinic acid sodium salt (71 mg, 0.00023 mol), (3-ethoxy-5-trifluoromethanesulfonyloxy-phenyl)-acetic acid methyl ester (17, 109 mg, 0.000318 mol), xanthphos (12 mg, 0.000021 mol) and cesium carbonate (174 mg, 0.000534 mol) were stirred in toluene (7 mL, 0.06 mol) under an argon flow. Bis(dibenzylideneacetone)palladium(0) (10 mg, 0.000017 mol) was quickly added and the reaction placed on an oil bath pre heated at 110° C. for 16 hours, after which TLC (20% ethyl acetate/hexane) showed multiple spots and absence of starting material. Solvent was removed and the crude compound plated onto a silica plate. The desired compound was isolated. ¹H NMR consistent with compound structure.

### Step-4: Preparation of [3-ethoxy-5-(4′-trifluoromethyl-biphenyl-3-sulfonyl)phenyl]-acetic acid (P-0080)

Saponification: The crude reaction product was dissolved in a 2 mL mixture of tetrahydrofuran/1N KOH (4:1) and stirred vigorously overnight, after which TLC (20% ethyl acetate/hexane) indicated absence of starting material and a new spot around the baseline. The reaction was acidified by adding 1N HCl (acidic via pH paper), extracted with ethyl acetate (3 times the reaction volume), and dried over MgSO₄. ¹H NMR (CDCl₃) consistent with compound structure. Calculated molecular weight 426.48, MS (ESI) [M+H⁺]⁺=427.12, [M−H⁺]⁻=425.06.

Additional compounds were prepared by through an alternative route for Steps 3-5, carrying out metal assisted biaryl coupling such as Suzuki coupling as described in the following Scheme 15a.

Compound P-0094 was synthesized in four steps from (3,5-dihydroxy-phenyl)-acetic methyl ester 8 as shown in Scheme 15a.

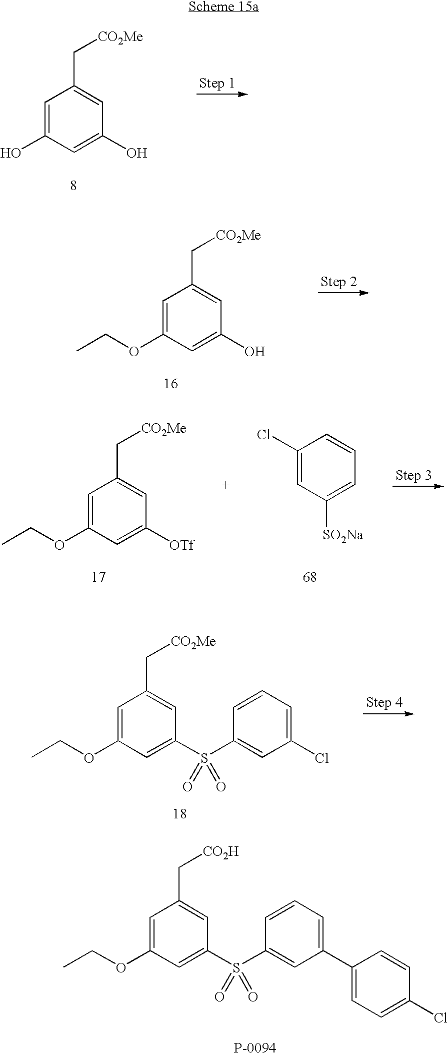

### Step-1 and Step-2

See Scheme 15 above.

### Step 3: Preparation of [3-(3-chloro-benzenesulfonyl)-5-ethoxy-phenyl]-acetic acid methyl ester (69)

Into a round bottom flask, (3-ethoxy-5-trifluoromethanesulfonyloxy-phenyl)-acetic acid methyl ester (17, 1.26 g, 0.00368 mol), 3-chlorophenyl sulfinic acid sodium salt (68, 1.26 g, 0.00634 mol), toluene (30 mL, 0.3 mol), xanthphos (0.30 g, 0.00052 mol), tris(dibenzylideneacetone)dipalladium(0) (0.50 g, 0.00055 mol), and cesium carbonate (1.3 g, 0.0040 mol) were combined and heated at 108° C. for 16 hours. The reaction was allowed to cool to room temperature and diluted with water. The reaction was extracted with ethyl acetate 4×. The combined organic layers were washed with water 2×, brine 1×, and dried over sodium sulfate. Evaporation of solvent led to a yellow-orange oil. The oil was then purified via flash chromatography (20-40% ethyl acetate in hexane) to yield the desired compound as a yellow oil. The oil was dissolved and treated for 16 hours before workup. The reaction was acidified with 10% HCl to pH 1-2 and extracted 4× with ethyl acetate. The combined organic layers were washed 1× with brine, and dried over sodium sulfate. Evaporation of solvent led to a yellow oil. The oil was then purified via flash chromatography at 9% methanol in dichloromethane to afford the desired compound as a lightly yellowish oil, which upon drying on high vac afforded a white solid. ¹H NMR consistent with compound structure.

### Step 4: Preparation of [3-(4′-chloro-biphenyl-3-sulfonyl)-5-ethoxy-phenyl]-acetic acid (P-0094)

10 mg of [3-(3-chloro-benzenesulfonyl)-5-ethoxy-phenyl]-acetic acid methyl ester 69 was dissolved in 400 μL of acetonitrile and 2 equivalents of 4-chlorophenyl boronic acid was added. 200 μL of 1M K₂CO₃ was added and 10 μL of a 0.2M solution in toluene of Pd(AOc)₂/di-t-butylbiphenylphosphine was added. The reaction mixture was heated for 10 minutes at 160° C. in the microwave. The solution was neutralized with acetic acid and the solvents removed under vacuum. The crude material was dissolved in 500 μL of dimethylsulfoxide and purified by HPLC eluting with a water/0.1% trifluoro acetic acid and acetonitrile/0.1% trifluoro acetic acid gradient, 20-100% acetonitrile over 16 minutes. Calculated molecular weight 430.91, MS (ESI) [M−H⁺]⁻=429.03.

Compound P-0290 was prepared following the protocol of Steps 2-5 of Scheme 15a, replacing (3-ethoxy-5-hydroxy-phenyl)-acetic acid methyl ester 16 with (3-hydroxy-phenyl)-acetic acid methyl ester in Step 2 and replacing 4-chlorophenyl boronic acid with 2-methoxy-pyrimidine-5-boronic acid in Step 4. Additional compounds were prepared following the protocol of Scheme 15a, optionally replacing the iodoethane with an appropriate iodoalkyl compound in Step 1, and/or optionally replacing the 4-chlorophenyl boronic acid with an appropriate boronic acid in Step 4. The following Table 6 indicates the appropriate iodoalkyl and boronic acid reagents used in Steps 1 and 4 of Scheme 15a, respectively, to provide the indicated compound.

TABLE 6

| Cmpd. | Step 1 iodoalkyl | |
|---|---|---|
| number | compound | Step 4 boronic acid |
| P-0290 | No Step 1 | 2-methoxy-prymidin-5-yl |
| P-0095 | 1-iodopropane | 4-fluoro-phenyl |
| P-0096 | iodoethane | 4-fluoro-phenyl |
| P-0105 | 1-iodopropane | 4-chloro-phenyl |
| P-0106 | 1-iodopropane | 2-methoxy-phenyl |
| P-0107 | 1-iodopropane | 4-methoxy-phenyl |
| P-0108 | 1-iodopropane | 3-chloro-4-fluoro-phenyl |
| P-0109 | 1-iodopropane | 2-trifluoromethyl-phenyl |
| P-0110 | 1-iodopropane | 4-trifluoromethoxy-phenyl |
| P-0111 | 1-iodopropane | 3-trifluoromethyl-phenyl |
| P-0112 | 1-iodopropane | 6-methoxy-pyridin-3-yl |
| P-0113 | 1-iodopropane | 3-fluoro-4-methoxy-phenyl |
| P-0134 | iodoethane | 2-methoxy-phenyl |
| P-0135 | iodoethane | 3-chloro-4-fluoro-phenyl |
| P-0136 | iodoethane | 4-ethoxy-phenyl |
| P-0137 | iodoethane | 3-trifluoromethoxy-phenyl |
| P-0138 | iodoethane | 4-trifluoromethoxy-phenyl |
| P-0139 | iodoethane | 6-methoxy-pyridin-3-yl |
| P-0140 | iodoethane | 3-fluoro-4-methoxy-phenyl |
| P-0187 | 1-iodopropane | 4-trifluoromethyl-phenyl |
| P-0188 | 1-iodopropane | 1H-pyrazol-4-yl |
| P-0189 | 1-iodopropane | 1-methyl-1H-pyrazol-4-yl |
| P-0190 | 1-iodopropane | 1-isobutyl-1H-pyrazol-4-yl |
| P-0191 | 1-iodopropane | 1-(3-methyl-butyl)-1H-pyrazol-4-yl |
| P-0192 | iodoethane | 1H-pyrazol-4-yl |
| P-0193 | iodoethane | 1-isobutyl-1H-pyrazol-4-yl |
| P-0194 | 1-iodo-2-methoxyethane | 4-chloro-phenyl |
| P-0195 | 1-iodo-2-methoxyethane | 2-methoxy-phenyl |
| P-0196 | 1-iodo-2-methoxyethane | 4-methoxy-phenyl |
| P-0197 | 1-iodo-2-methoxyethane | 3-chloro-4-fluoro-phenyl |
| P-0198 | 1-iodo-2-methoxyethane | 4-ethoxy-phenyl |
| P-0199 | 1-iodo-2-methoxyethane | 3-trifluoromethoxy-phenyl |
| P-0200 | 1-iodo-2-methoxyethane | 4-trifluoromethoxy-phenyl |
| P-0201 | 1-iodo-2-methoxyethane | 3-trifluoromethyl-phenyl |
| P-0202 | 1-iodo-2-methoxyethane | 4-trifluoromethyl-phenyl |
| P-0203 | 1-iodo-2-methoxyethane | 6-methoxy-pyridin-3-yl |
| P-0204 | 1-iodo-2-methoxyethane | 1H-pyrazol-4-yl |
| P-0205 | 1-iodo-2-methoxyethane | 1-isobutyl-1H-pyrazol-4-yl |
| P-0259 | 1-iodopropane | 4-ethoxy-phenyl |
| P-0260 | 1-iodopropane | 3-trifluoromethoxy-phenyl |
| P-0081 | iodoethane | 4-methoxy-phenyl |

The compound structures, names and mass spectrometry results for these compounds are provided in the following Table 7.

TABLE 7

| | | | Molecular weight | |
|---|---|---|---|---|
| Cmpd. | | | | Measured |
| number | Structure | Name | Calc. | MS(ESI) |
| P-0290 | 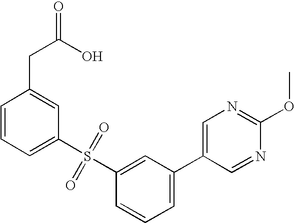 | {3-[3-(2-Methoxy-pyrimidin-5-yl)-benzenesulfonyl]-phenyl}-acetic acid | 381.41 | |
| P-0095 | 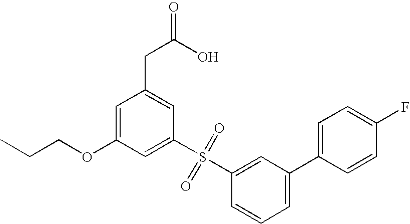 | [3-(4′-Fluoro-biphenyl-3-sulfonyl)-5-propoxy-phenyl]-acetic acid | 428.48 | [M − H⁺]⁻ =427.07 |
| P-0096 | 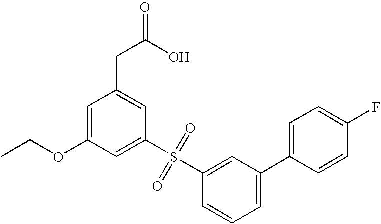 | [3-Ethoxy-5-(4′-fluoro-biphenyl-3-sulfonyl)-phenyl]-acetic acid | 414.45 | [M − H⁺]⁻ =413.03 |
| P-0105 | 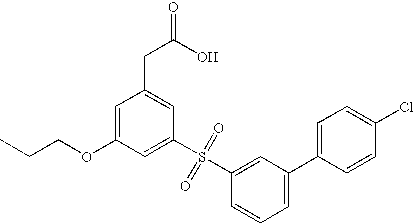 | [3-(4′-Chloro-biphenyl-3-sulfonyl)-5-propoxy-phenyl]-acetic acid | 444.93 | [M + H⁺]⁺ =445.1 |
| P-0106 | 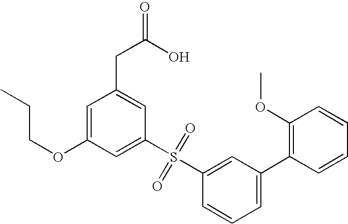 | [3-(2′-Methoxy-biphenyl-3-sulfonyl)-5-propoxy-phenyl]-acetic acid | 440.51 | [M + H⁺]⁺ =441.1 |
| P-0107 | 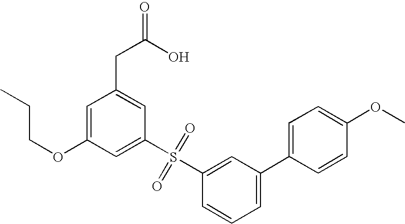 | [3-(4′-Methoxy-biphenyl-3-sulfonyl)-5-propoxy-phenyl]-acetic acid | 440.51 | [M + H⁺]⁺ =441.1 |
| P-0108 | 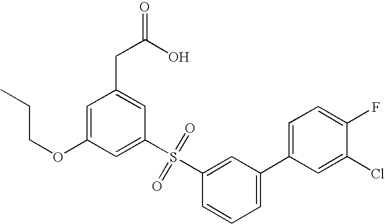 | [3-(3′-Chloro-4′-fluoro-biphenyl-3-sulfonyl)-5-propoxy-phenyl]-acetic acid | 462.92 | [M + H⁺]⁺ =463.1 |
| P-0109 | 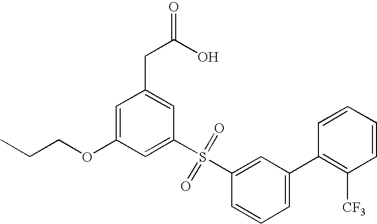 | [3-Propoxy-5-(2′-trifluoromethyl-biphenyl-3-sulfonyl)-phenyl]-acetic acid | 478.48 | [M + H⁺]⁺ =479.1 |
| P-0110 | 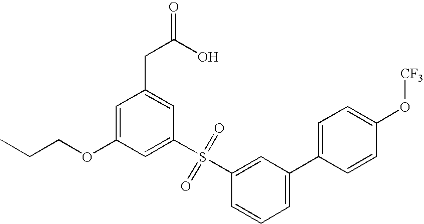 | [3-Propoxy-5-(4′-trifluoromethoxy-biphenyl-3-sulfonyl)-phenyl]-acetic acid | 494.48 | [M + H⁺]⁺ =495.1 |
| P-0111 | 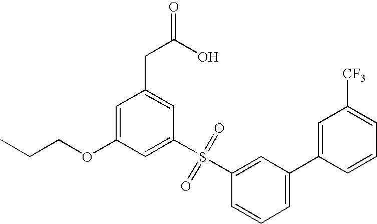 | [3-Propoxy-5-(3′-trifluoromethyl-biphenyl-3-sulfonyl)-phenyl]-acetic acid | 478.48 | [M + H⁺]⁺ =478.7 |
| P-0112 | 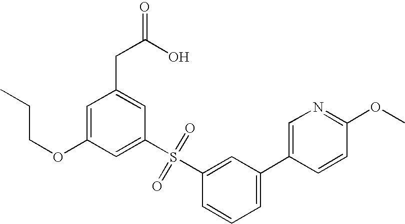 | {3-[3-(6-Methoxy-pyridin-3-yl)-benzenesulfonyl]-5-propoxy-phenyl}-acetic acid | 441.50 | [M + H⁺]⁺ =442.3 |
| P-0113 | 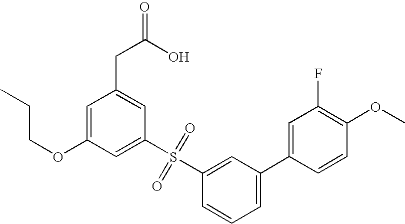 | [3-(3′-Fluoro-4′-methoxy-biphenyl-3-sulfonyl)-5-propoxy-phenyl]-acetic acid | 458.50 | [M + H⁺]⁺ =459.1 |
| P-0134 | 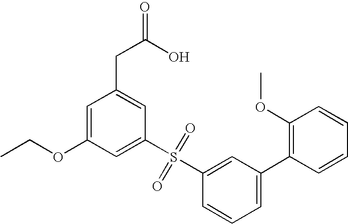 | [3-Ethoxy-5-(2′-methoxy-biphenyl-3-sulfonyl)-phenyl]-acetic acid | 426.49 | [M + H⁺]⁺ =427.1 |
| P-0135 | 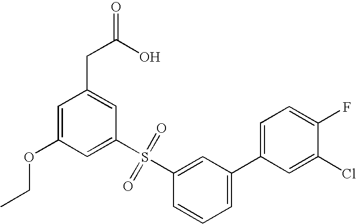 | [3-(3′-Chloro-4′-fluoro-biphenyl-3-sulfonyl)-5-ethoxy-phenyl]-acetic acid | 448.90 | [M + H⁺]⁺ =449.1 |
| P-0136 | 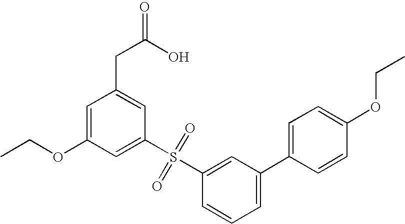 | [3-Ethoxy-5-(4′-ethoxy-biphenyl-3-sulfonyl)-phenyl]-acetic acid | 440.51 | [M + H⁺]⁺ =441.1 |
| P-0137 | 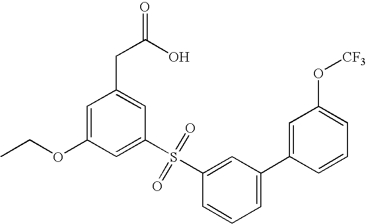 | [3-Ethoxy-5-(3′-trifluoromethoxy-biphenyl-3-sulfonyl)-phenyl]-acetic acid | 480.46 | [M + H⁺]⁺ =481.1 |
| P-0138 | 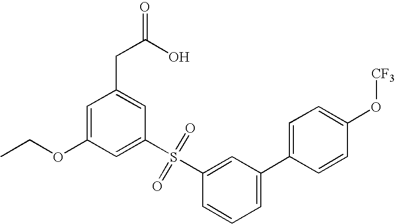 | [3-Ethoxy-5-(4′-trifluoromethoxy-biphenyl-3-sulfonyl)-phenyl]-acetic acid | 480.46 | [M + H⁺]⁺ =480.7 |
| P-0139 | 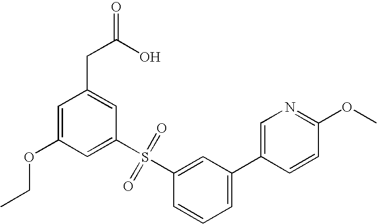 | {3-Ethoxy-5-[3-(6-methoxy-pyridin-3-yl)-benzenesulfonyl]-phenyl}-acetic acid | 427.47 | [M + H⁺]⁺ =427.9 |
| P-0140 | 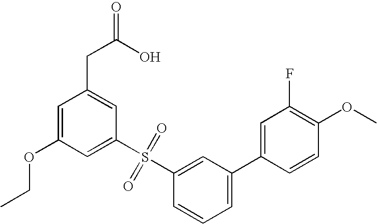 | [3-Ethoxy-5-(3′-fluoro-4′-methoxy-biphenyl-3-sulfonyl)-phenyl]-acetic acid | 444.48 | [M + H⁺]⁺ =445.1 |
| P-0187 | 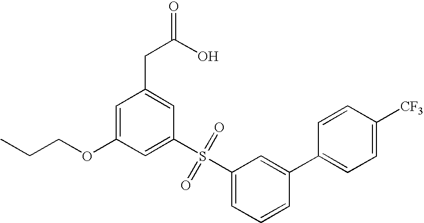 | [3-Propoxy-5-(4′-trifluoromethyl-biphenyl-3-sulfonyl)-phenyl]-acetic acid | 478.48 | [M + H⁺]⁺ =478.7 |
| P-0188 | 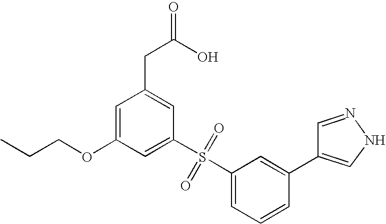 | {3-Propoxy-5-[3-(1H-pyrazol-4-yl)-benzenesulfonyl]-phenyl}-acetic acid | 400.45 | [M + H⁺]⁺ =401.1 |
| P-0189 | 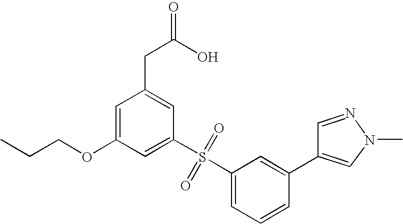 | {3-[3-(1-Methyl-1H-pyrazol-4-yl)-benzenesulfonyl]-5-propoxy-phenyl}-acetic acid | 414.48 | [M + H⁺]⁺ =415.1 |
| P-0190 | 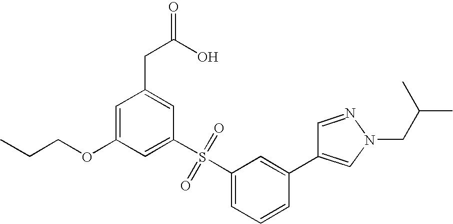 | {3-[3-(1-Isobutyl-1H-pyrazol-4-yl)-benzenesulfonyl]-5-propoxy-phenyl}-acetic acid | 456.56 | [M + H⁺]⁺ =457.1 |
| P-0191 | 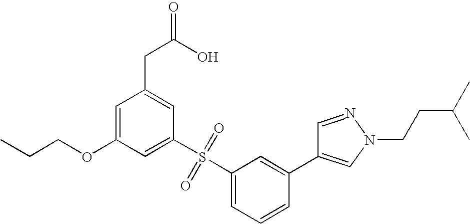 | (3-{3-[1-(3-Methyl-butyl)-1H-pyrazol-4-yl]-benzenesulfonyl}-5-propoxy-phenyl)-acetic acid | 470.59 | [M + H⁺]⁺ =471.5 |
| P-0192 | 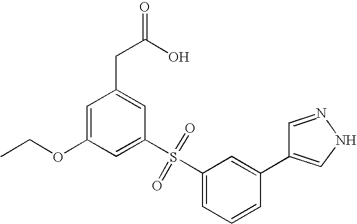 | {3-Ethoxy-5-[3-(1H-pyrazol-4-yl)-benzenesulfonyl]-phenyl}-acetic acid | 386.43 | [M + H⁺]⁺ =387.1 |
| P-0193 | 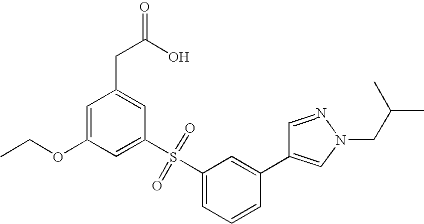 | {3-Ethoxy-5-[3-(1-isobutyl-1H-pyrazol-4-yl)-benzenesulfonyl]-phenyl}-acetic acid | 442.53 | [M + H⁺]⁺ =443.1 |
| P-0194 | 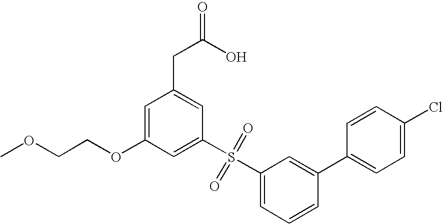 | [3-(4′-Chloro-biphenyl-3-sulfonyl)-5-(2-methoxy-ethoxy)-phenyl]-acetic acid | 460.93 | MS(ESI)[M + H+]+ =461.1 |
| P-0195 | 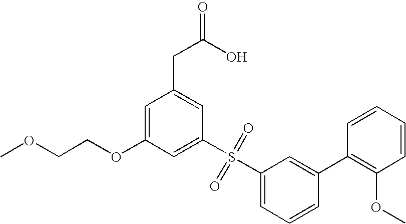 | [3-(2′-Methoxy-biphenyl-3-sulfonyl)-5-(2-methoxy-ethoxy)-phenyl]-acetic acid | 456.51 | [M + H⁺]⁺ =457.1 |
| P-0196 | 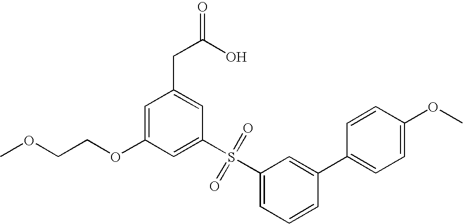 | [3-(4′-Methoxy-biphenyl-3-sulfonyl)-5-(2-methoxy-ethoxy)-phenyl]-acetic acid | 456.51 | [M + H⁺]⁺ =457.1 |
| P-0197 | 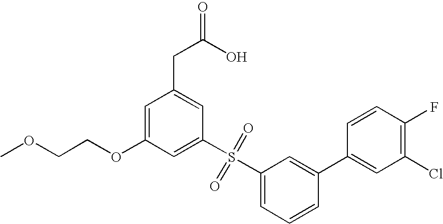 | [3-(3′-Chloro-4′-fluoro-biphenyl-3-sulfonyl)-5-(2-methoxy-ethoxy)-phenyl]-acetic acid | 478.92 | [M + H⁺]⁺ =479.1 |
| P-0198 | 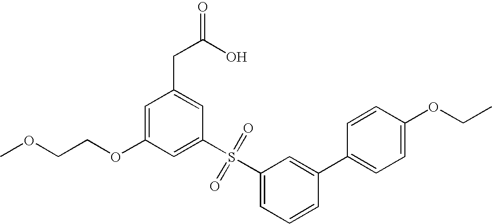 | [3-(4′-Ethoxy-biphenyl-3-sulfonyl)-5-(2-methoxy-ethoxy)-phenyl]-acetic acid | 470.54 | [M + H⁺]⁺ =471.5 |
| P-0199 | 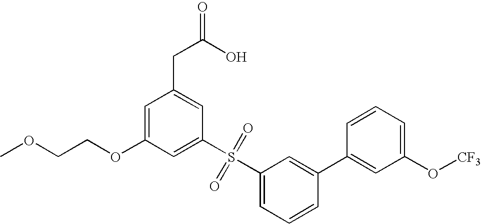 | [3-(2-Methoxy-ethoxy)-5-(3′-trifluoromethoxy-biphenyl-3-sulfonyl)-phenyl]-acetic acid | 510.48 | [M + H⁺]⁺ =511.9 |
| P-0200 | 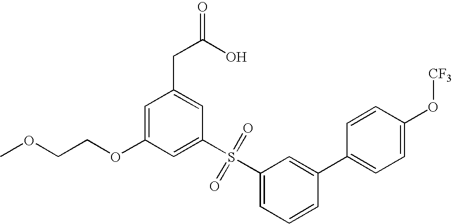 | [3-(2-Methoxy-ethoxy)-5-(4′-trifluoromethoxy-biphenyl-3-sulfonyl)-phenyl]-acetic acid | 510.48 | [M + H⁺]⁺ =511.5 |
| P-0201 | 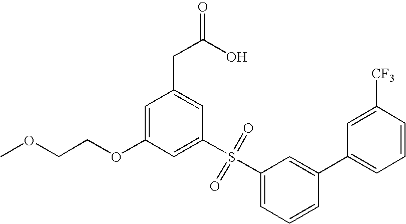 | [3-(2-Methoxy-ethoxy)-5-(3′-trifluoromethyl-biphenyl-3-sulfonyl)-phenyl]-acetic acid | 494.48 | [M + H⁺]⁺ =495.1 |
| P-0202 | 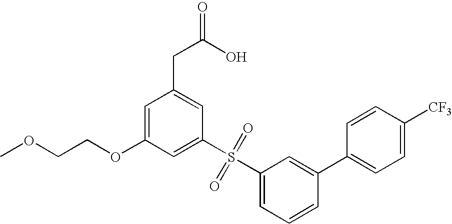 | [3-(2-Methoxy-ethoxy)-5-(4′-trifluoromethyl-biphenyl-3-sulfonyl)-phenyl]-acetic acid | 494.48 | [M + H⁺]⁺ =495.1 |
| P-0203 | 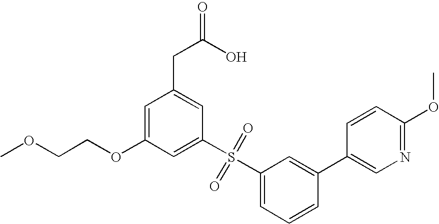 | {3-(2-Methoxy-ethoxy)-5-[3-(6-methoxy-pyridin-3-yl)-benzenesulfonyl]-phenyl}-acetic acid | 457.50 | [M + H⁺]⁺ =458.3 |
| P-0204 | 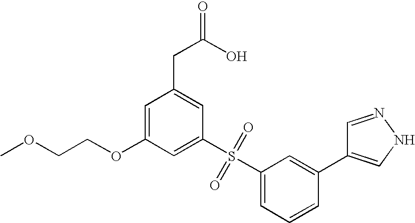 | {3-(2-Methoxy-ethoxy)-5-[3-(1H-pyrazol-4-yl)-benzenesulfonyl]-phenyl}-acetic acid | 416.45 | [M + H⁺]⁺ =417.5 |
| P-0205 | 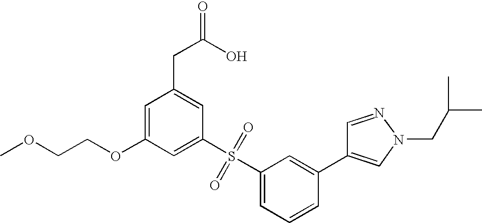 | [3-[3-(1-Isobutyl-1H-pyrazol-4-yl)-benzenesulfonyl]-5-(2-methoxy-ethoxy)-phenyl]-acetic acid | 472.56 | [M + H⁺]⁺ =473.1 |
| P-0259 | 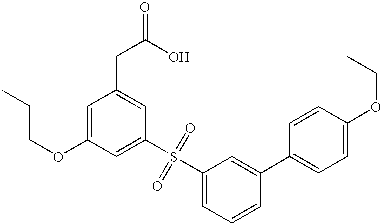 | [3-(4′-Ethoxy-biphenyl-3-sulfonyl)-5-propoxy-phenyl]-acetic acid | 454.54 | MS(ESI)[M + H+]+ =455.1 |
| P-0260 | 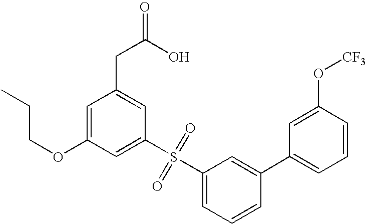 | [3-Propoxy-5-(3′-trifluoromethoxy-biphenyl-3-sulfonyl)-phenyl]-acetic acid | 494.48 | [M + H⁺]⁺ =495.1 |
| P-0081 | 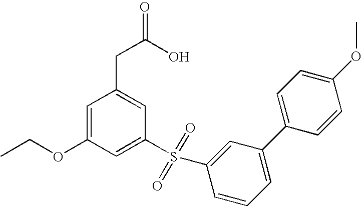 | [3-Ethoxy-5-(4′-methoxy-biphenyl-3-sulfonyl)-phenyl]-acetic acid | 426.49 | [M + H⁺]⁺ =427.12[M − H⁺]⁻ =425.06 |

### Example 7

### Synthesis of [3-ethoxy-5-(4′-trifluoromethyl-biphenyl-3-yloxy)-phenyl]-acetic acid (P-0082)

Compound P-0082 was synthesized in two steps from (3-ethoxy-5-hydroxy-phenyl)-acetic acid methyl ester (16) as shown in Scheme 16.

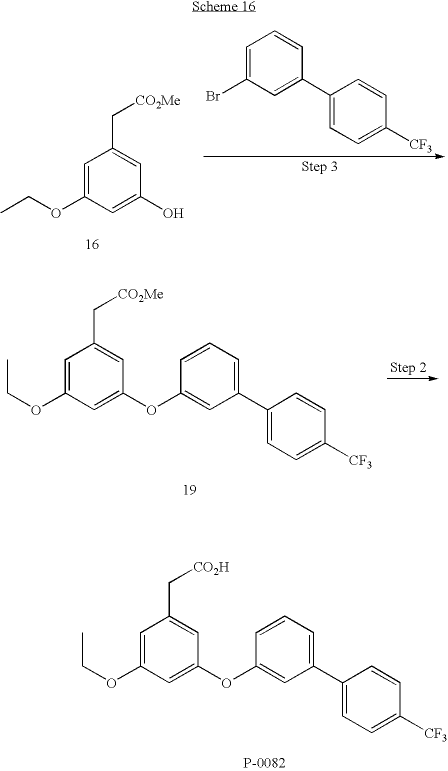

### Step 1: Preparation of [3-ethoxy-5-(4′-trifluoromethyl-biphenyl-3-yloxy)-phenyl]-acetic acid methyl ester (19)

Into a flask, (3-ethoxy-5-hydroxy-phenyl)-acetic acid methyl ester (16, 120 mg, 0.00057 mol, prepared as per Step 1 of Scheme 15, Example 6) was dissolved in 1,4-dioxane (2 mL, 0.02 mol). Cesium carbonate (370 mg, 0.0011 mol), 3-bromo-4′-trifluoromethyl-biphenyl (260 mg, 0.00086 mol), dimethylamino-acetic acid (20 mg, 0.0002 mol) and copper(I) iodide (10 mg, 0.00006 mol) were added. The mixture was heated at 90° C. overnight under an atmosphere of argon, after which TLC showed full conversion of starting material. Ethyl acetate was added followed by a mixture of ammonium chloride/ammonium hydroxide (4:1). The layers were separated, and the organic layer was dried over sodium sulfate. Absorbing the crude material onto silica, flash chromatography with 10-20% ethyl acetate in hexanes was used to isolate the desired compound, which was taken to the next step. ¹H NMR consistent with compound structure.

### Step 2: Preparation of [3-ethoxy-5-(4′-trifluoromethyl-biphenyl-3-yloxy)-phenyl]-acetic acid (P-0082)

[3-Ethoxy-5-(4′-trifluoromethyl-biphenyl-3-yloxy)-phenyl]-acetic acid methyl ester (19, 20 mg, 0.00005 mol) was dissolved in tetrahydrofuran (4 mL, 0.05 mol). 1M lithium hydroxide in water (1 mL) was added and the mixture was stirred overnight at room temperature. The mixture was acidified using 1M HCl (pH 1-2) and extracted with ethyl acetate. The organic layer was separated from the aqueous and dried over sodium sulfate. Evaporation of solvent under reduced pressure afforded an oil. The final compound was isolated after purification with prep. TLC (5% methanol in dichloromethane). ¹H NMR consistent with compound structure. Calculated molecular weight 416.39, MS (ESI) [M+H⁺]⁺=417.2, [M−H⁺]⁻=415.0.

Compound P-0079, [3-Ethoxy-5-(4′-trifluoromethyl-biphenyl-4-yloxy)-phenyl]-acetic acid,

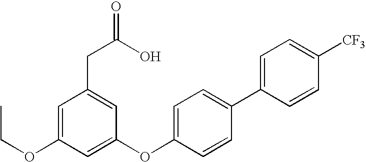

was prepared following the protocol of Scheme 16, replacing 3-bromo-4′-trifluoromethyl-biphenyl with 4-bromo-4′-trifluoromethyl-biphenyl in Step 1. Calculated molecular weight 416.39, MS (ESI) [M+H⁺]⁺=417.2, [M−H⁺]⁻=415.0.

Compound P-0291, [3-Methoxy-5-(4′-trifluoromethyl-biphenyl-3-yloxy)-phenyl]-acetic acid,

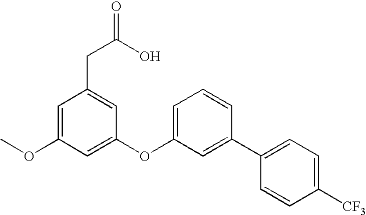

was prepared following the protocol of Scheme 16, replacing (3-ethoxy-5-hydroxy-phenyl)-acetic acid methyl ester 16 with (3-hydroxy-5-methoxy-phenyl)-acetic acid methyl ester in Step 1. Calculated molecular weight 402.37, MS (ESI) [M+H⁺]⁺=403.1, [M−H⁺]⁻=401.1.

Compound P-0292, [3-(4′-Trifluoromethyl-biphenyl-3-yloxy)-phenyl]-acetic acid,

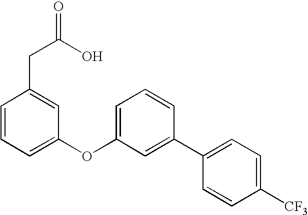

was prepared following the protocol of Scheme 16, replacing (3-ethoxy-5-hydroxy-phenyl)-acetic acid methyl ester 16 with (3-hydroxy-phenyl)-acetic acid methyl ester in Step 1. Calculated molecular weight 372.34, MS (ESI) [M−H⁺]⁻=371.1.

### Example 8

### Synthesis of {3-propoxy-5-[4-(4-trifluoromethoxy-phenoxy)-benzenesulfonyl]-phenyl}-acetic acid (P-0064)

Compound P-0064 was synthesized in five steps from (3,5-dihydroxy-phenyl)-acetic methyl ester 8 as shown in Scheme 17.

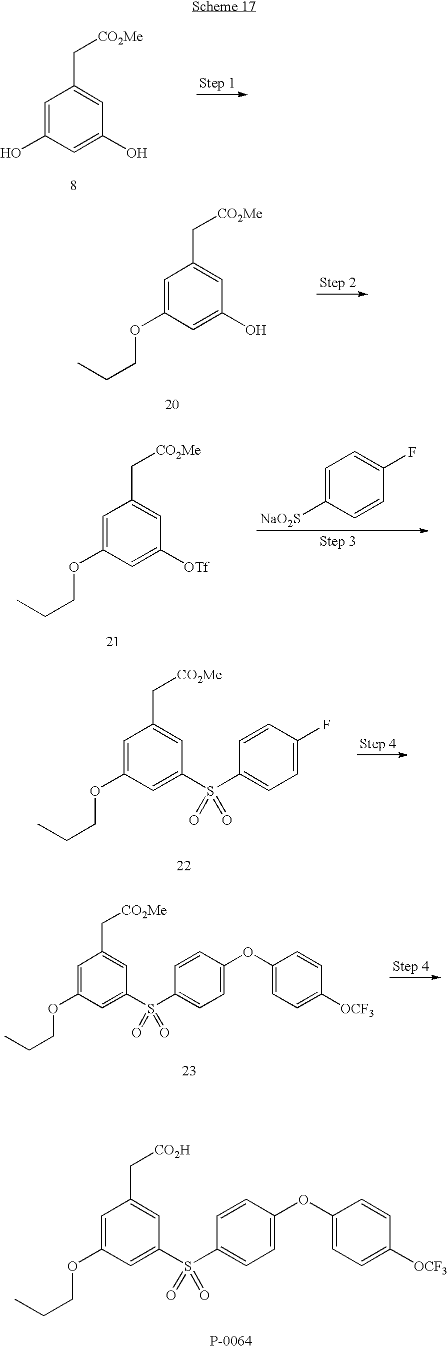

### Step 1: Preparation of (3-hydroxy-5-propoxy-phenyl)-acetic acid methyl ester (20)

Into a flask, (3,5-dihydroxy-phenyl)-acetic acid methyl ester (8, 10.376 g, 0.056957 mol) was dissolved in 2-butanone (200 mL, 2 mol). Potassium carbonate (21.5 g, 0.155 mol) was added in one portion and 1-iodopropane (5.06 mL, 0.0518 mol) was added drop wise. The reaction was heated to 80° C. and left stirring overnight. The solid was filtered off and the solvent was removed. Water and ethyl acetate were added and the solution was neutralized using 1M HCl. The water phase was extracted with ethyl acetate. The pooled organic phase was dried (Na₂SO₄) and absorbed onto silica. Flash Chromatography eluting with 20-40% ethyl acetate in hexanes afforded the desired compound as a clear yellow oil. ¹H NMR consistent with compound structure.

### Step 2: Preparation of (3-propoxy-5-trifluoromethanesulfonyloxy-phenyl)-acetic acid methyl ester (21)

Into a round bottom flask cooled to 0° C., (3-hydroxy-5-propoxy-phenyl)-acetic acid methyl ester (20, 2.36 g, 0.0105 mol) was dissolved in pyridine (35 mL, 0.43 mol). Trifluoromethanesulfonic anhydride (4 mL, 0.02 mol) was added in portions via a syringe. The reaction was allowed to proceed for 16 hours before workup. The reaction was acidified with 2-3 mL of concentrated HCl and extracted 4× with ethyl ether. The combined ether layers were washed with 1N HCl 1×, water 1×, brine 1×, and dried over sodium sulfate. Evaporation of solvent led to a brown oil, which was used in the next step. TLC showed the desired compound as the major product. ¹H NMR analysis showed that the triflate 21 is the major product (>90%).

### Step 3: preparation of [3-(4-fluoro-benzenesulfonyl)-5-propoxy-phenyl]-acetic acid methyl ester (22)

(3-Propoxy-5-trifluoromethanesulfonyloxy-phenyl)-acetic acid methyl ester (21, 129.4 mg, 0.0003633 mol), (4-fluorophenyl)sulfinic acid sodium salt (116 mg, 0.36 mmol), toluene (2.7090 mL, 0.025432 mol), tris(dibenzylideneacetone)-dipalladium(0) (20 mg, 0.00002 mol), cesium carbonate (177.56 mg, 5.4497E-4 mol), and xanthphos (21.02 mg, 3.633E-5 mol) were added to a high pressure tube and purged with nitrogen before sealing with a teflon stopcock. The mixture was heated at 120° C. overnight. The reaction was allowed to cool and diluted with ethyl acetate. The layers were separated and the organic layer was washed with saturated sodium bicarbonate and dried over MgSO₄. Solvent was removed under reduced pressure to afford crude material, which was purified using prep plate chromatography (7:3 hexane:ethyl acetate). The desired compound was isolated and ¹H NMR was consistent with compound structure. MS (ESI) [M+H⁺]⁺=367.2.

### Step 4: preparation of 3-propoxy-5-[4-(4-trifluoromethoxy-phenoxy)-benzenesulfonyl]-phenyl-acetic acid methyl ester (23)

[3-(4-Fluoro-benzenesulfonyl)-5-propoxy-phenyl]-acetic acid methyl ester (22, 24 mg, 0.000066 mol) was dissolved in dimethyl sulfoxide (0.5 mL, 0.007 mol) and potassium carbonate (10 mg, 0.000072 mol) and 4-trifluoromethoxy-phenol (9.4 μL, 0.000072 mol) were added in a microwave reaction vessel. This mixture was heated at 120° C. for 10 minutes. The solvent was removed by freeze drying overnight. Ethyl acetate and water were added to the crude material, and the layers separated. The organic phase was washed with brine and dried with sodium sulfate. The crude material was purified via prep TLC (hexane:ethyl acetate 7:3). ¹H NMR consistent with compound structure. MS (ESI) [M+H⁺]⁺=525.2.

### Step 5: Preparation of {3-propoxy-5-[4-(4-trifluoromethoxy-phenoxy)-benzenesulfonyl]-phenyl}-acetic acid (P-0064)

3-Propoxy-5-[4-(4-trifluoromethoxy-phenoxy)-benzenesulfonyl]-phenyl-acetic acid methyl ester (23, 20.000 mg, 3.8131E-5 mol), lithium hydroxide (1M, 0.30 mL) and tetrahydrofuran (1.0 mL, 0.012 mol) were added to a small vial and the reaction mixture was stirred for 3 days at ambient conditions. The reaction was acidified with 1M HCl and diluted with water and ethyl acetate. The organic layer was separated and dried over MgSO₄, and concentrated at reduced pressure to obtain an off-white solid (11 mg). ¹H NMR consistent with compound structure. Calculated molecular weight 510.48, MS (ESI) [M+H⁺]⁺=511.2.

### Example 9

### Synthesis of {3-butoxy-5-[4-methyl-2-(4-trifluoromethyl-phenyl)-thiazol-5-ylmethoxy]-phenyl}-acetic acid (P-0009)

Compound P-0009 was synthesized in two steps from (3-butoxy-5-hydroxy-phenyl)-acetic acid methyl ester 9 as shown in Scheme 18.

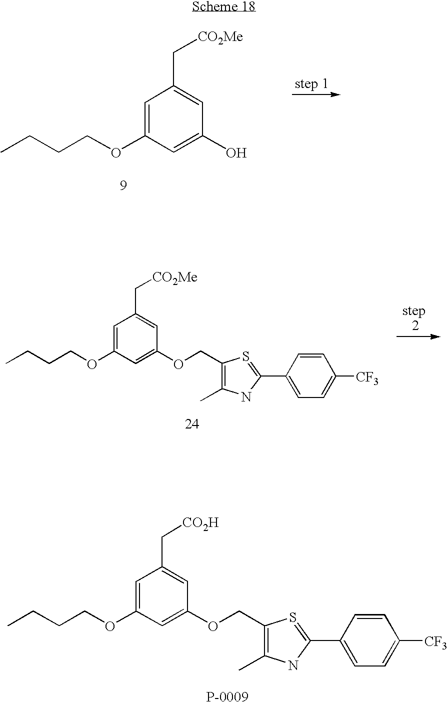

### Step 1: Preparation of {3-butoxy-5-[4-methyl-2-(4-trifluoromethyl-phenyl)-thiazol-5-ylmethoxy]-phenyl}-acetic acid methyl ester (24)

Into a flask, (3-butoxy-5-hydroxy-phenyl)-acetic acid methyl ester (9, 103 mg, 0.000432 mol, prepared as per Step 1 of Scheme 14, Example 5) was dissolved in N,N-dimethylformamide (4 mL, 0.05 mol). Potassium carbonate (180 mg, 0.0013 mol) and 5-(chloromethyl)-4-methyl-2-[4-(trifluoromethyl)phenyl]-1,3-thiazole (0.21 g, 0.00073 mol) were added. The reaction mixture was stirred at 90° C. for 5 hours. The mixture was concentrated under reduced pressure and diluted with water and ethyl acetate. The mixture was acidified with 1M HCl. The aqueous phase was extracted with ethyl acetate and the organic layers were dried with sodium sulfate and evaporated under reduced pressure. The crude material was absorbed onto silica and purified by flash chromatography with solvent of 100% hexane, then 10% ethyl acetate in hexane. ¹H NMR consistent with compound structure.

### Step 2: Preparation of {3-butoxy-5-[4-methyl-2-(4-trifluoromethyl-phenyl)-thiazol-5-ylmethoxy]-phenyl}-acetic acid (P-0009)

Into a flask, {3-butoxy-5-[4-methyl-2-(4-trifluoromethyl-phenyl)-thiazol-5-ylmethoxy]-phenyl}-acetic acid methyl ester (24, 101 mg, 0.000205 mol) was dissolved in tetrahydrofuran (5 mL, 0.06 mol). 1M potassium hydroxide in water (2 mL) was added, and the mixture was stirred overnight at room temperature. The mixture was acidified with 1M HCl and the aqueous phase was extracted with ethyl acetate 3×. The organic phase was washed with brine, dried with sodium sulfate and concentrated. A small impurity was seen by ¹H NMR. The product was further purified on prep TLC plate, eluting with 5% methanol in dichloromethane. ¹H NMR consistent with compound structure. Calculated molecular weight 479.52, MS (ESI) [M+H⁺]⁺=480.2; [M−H⁺]⁻=478.2.

Additional compounds were prepared by optionally replacing the 5-(chloromethyl)-4-methyl-2-[4-(trifluoromethyl)phenyl]-1,3-thiazole with an appropriate chloroalkyl compound in Step 1, and/or optionally replacing the (3-butoxy-5-hydroxy-phenyl)-acetic acid methyl ester 9 with an appropriate acetic acid methyl ester in Step 1, where the acetic acid methyl ester is prepared according to Step 1 of Scheme 14, Example by replacing 1-iodobutane with an appropriate iodoalkyl compound. The following Table 8 indicates the appropriate acetic acid methyl ester and chloroalkyl compounds used in Step 1 for the indicated compound.

TABLE 8

| Cmpd. | | |
|---|---|---|
| Number | Acetic acid methyl ester | Chloroalkyl compound |
| P-0001 | 3-butoxy-5-hydroxy-phenyl | 1-[4-(3-chloro- |
| | | propoxy)-2-hydroxy-3- |
| | | propyl-phenyl]- |
| | | ethanone |
| P-0007 | 3-butoxy-5-hydroxy-phenyl | chloromethyl-benzene |
| P-0008 | 3-butoxy-5-hydroxy-phenyl | 2-chloro-ethyl-benzene |
| P-0010 | 3-butoxy-5-hydroxy-phenyl | 4-(2-chloro-ethyl)-5- |
| | | methyl-2-phenyl-oxazole |
| P-0012 | 3-butoxy-5-hydroxy-phenyl | 5-chloromethyl-2-phenoxy- |
| | | pyridine |
| P-0013 | 3-butoxy-5-hydroxy-phenyl | 4-chloromethyl-3-(2,6- |
| | | dichloro-phenyl)-5- |
| | | isopropyl-isoxazole |
| P-0014 | 3-butoxy-5-hydroxy-phenyl | 1-(2-chloro-ethyl)-4- |
| | | trifluoromethyl-benzene |
| P-0015 | 3-butoxy-5-hydroxy-phenyl | 1-(2-chloro-ethyl)-3- |
| | | trifluoromethyl-benzene |
| P-0016 | 3,5-dihydroxy-phenyl | 5-Chloromethyl-4-methyl- |
| | | 2-(4-trifluoromethyl- |
| | | phenyl)-thiazole |
| P-0017 | 3-butoxy-5-hydroxy-phenyl | 1-chloromethyl-4-(4- |
| | | trifluoromethyl-phenoxy)- |
| | | benzene |
| P-0018 | 3-cyclopropylmethoxy-5- | 5-Chloromethyl-4-methyl- |
| | hydroxy-phenyl | 2-(4-trifluoromethyl- |
| | | phenyl)-thiazole |
| P-0019 | 3-ethoxy-5-hydroxy-phenyl | 5-Chloromethyl-4-methyl- |
| | | 2-(4-trifluoromethyl- |
| | | phenyl)-thiazole |
| P-0020 | 3-hydroxy-5-isopropoxy- | 5-Chloromethyl-4-methyl- |
| | phenyl | 2-(4-trifluoromethyl- |
| | | phenyl)-thiazole |
| P-0021 | 3-hydroxy-5-(2-methoxy- | 5-Chloromethyl-4-methyl- |
| | ethoxy)-phenyl | 2-(4-trifluoromethyl- |
| | | phenyl)-thiazole |
| P-0046* | 3-hydroxy-4-methoxy-phenyl | 1-[4-(3-chloro- |
| | | propoxy)-2-hydroxy-3- |
| | | propyl-phenyl]- |
| | | ethanone |
| P-0047 | 3-hydroxy-4-methoxy-phenyl | 1-[4-(3-chloro- |
| | | propoxy)-2-hydroxy-3- |
| | | propyl-phenyl]- |
| | | ethanone |

*Methyl ester isolated after Step 1.

The compound structures, names and mass spectrometry results for these compounds are provided in the following Table 9.

TABLE 9

| | | | Molecular weight | |
|---|---|---|---|---|
| Cmpd. | | | | Measured |
| number | Structure | Name | Calc. | MS(ESI) |
| P-0001 | 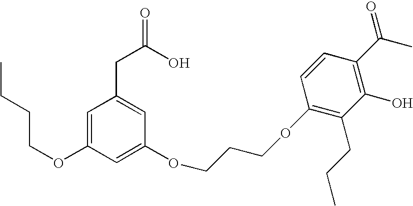 | {3-[3-(4-Acetyl-3-hydroxy-2-propyl-phenoxy)-propoxy]-5-butoxy-phenyl}-acetic acid | 458.55 | [M + H⁺]⁺ =459.2[M − H⁺]⁻ =457.2 |
| P-0007 | 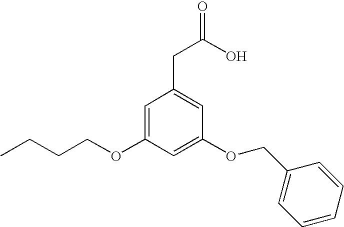 | (3-Benzyloxy-5-butoxy-phenyl)-acetic acid | 314.38 | [M + H⁺]⁺ =315.2[M − H⁺]⁻ =313.2 |
| P-0008 | 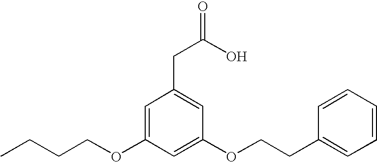 | (3-Butoxy-5-phenethyloxy-phenyl)-acetic acid | 328.41 | [M + H⁺]⁺ =329.2[M − H⁺]⁻ =327.2 |
| P-0010 | 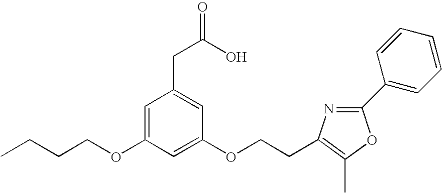 | {3-Butoxy-5-[2-(5-methyl-2-phenyl-oxazol-4-yl)-ethoxy]-phenyl}-acetic acid | 409.48 | [M + H⁺]⁺ =410.2[M − H⁺]⁻ =408.2 |
| P-0012 | 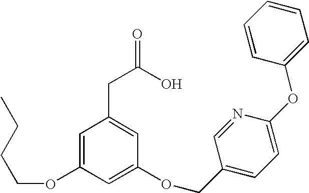 | [3-Butoxy-5-(6-phenoxy-pyridin-3-ylmethoxy)-phenyl]-acetic acid | 407.46 | [M + H⁺]⁺ =408.3[M − H⁺]⁻ =406.2 |
| P-0013 | 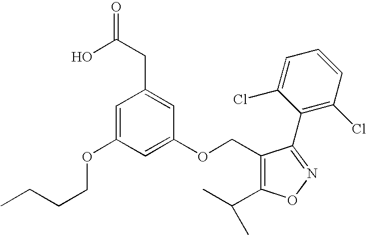 | {3-Butoxy-5-[3-(2,6-dichloro-phenyl)-5-isopropyl-isoxazol-4-ylmethoxy]-phenyl}-acetic acid | 492.40 | [M + H⁺]⁺ =492.2[M − H⁺]⁻ =490.2 |
| P-0014 | 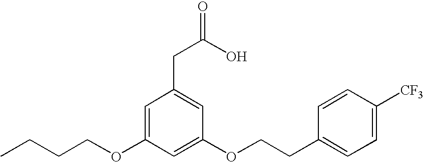 | 3-Butoxy-5-[2-(4-trifluoromethyl-phenyl)-ethoxy]-phenyl}-acetic acid | 396.40 | [M − H⁺]⁻ =395.2 |
| P-0015 | 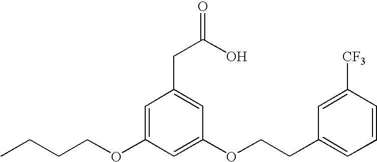 | {3-Butoxy-5-[2-(3-trifluoromethyl-phenyl)-ethoxy]-phenyl}-acetic acid | 396.40 | [M − H⁺]⁻ =395.2 |
| P-0016 | 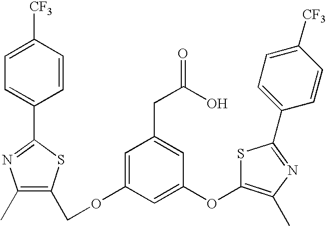 | {3,5-Bis-[4-methyl-2-(4-trifluoromethyl-phenyl)-thiazol-5-ylmethoxy]-phenyl}-aceticacid | 678.67 | [M + H⁺]⁺ =679.4[M − H⁺]⁻ =677.4 |
| P-0017 | 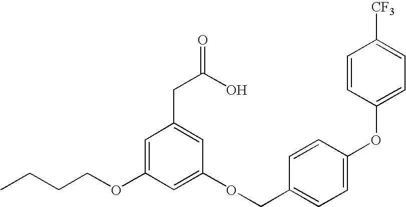 | {3-Butoxy-5-[4-(4-trifluoromethyl-phenoxy)-benzyloxy]-phenyl}-acetic acid | 474.47 | [M − H⁺]⁻ =473.3 |
| P-0018 | 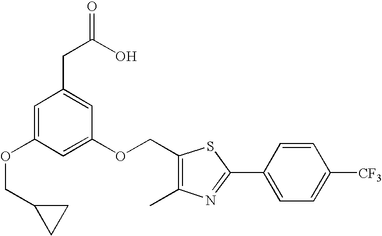 | {3-Cyclopropyl methoxy-5-[4-methyl-2-(4-trifluoromethyl-phenyl)-thiazol-5-ylmethoxy]-phenyl}-acetic acid | 477.50 | [M + H⁺]⁺ =478.3[M − H⁺]⁻ =476.3 |
| P-0019 | 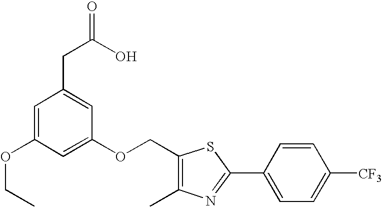 | {3-Ethoxy-5-[4-methyl-2-(4-trifluoromethyl-phenyl)-thiazol-5-ylmethoxy]-phenyl}-aceticacid | 451.46 | [M + H⁺]⁺ =452.3[M − H⁺]⁻ =450.2 |
| P-0020 | 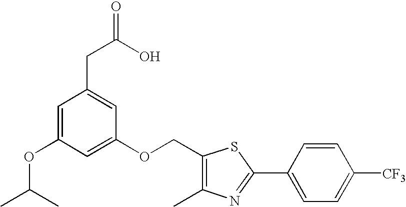 | {3-Isopropoxy-5-[4-methyl-2-(4-trifluoromethyl-phenyl)-thiazol-5-ylmethoxy]-phenyl}-aceticacid | 465.49 | [M + H⁺]⁺ =466.4[M − H⁺]⁻ =464.3 |
| P-0021 | 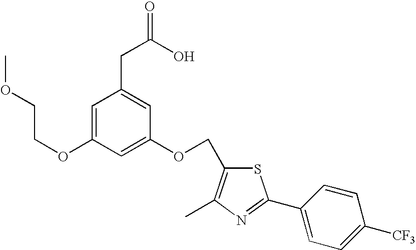 | {3-(2-Methoxy-ethoxy)-5-[4-methyl-2-(4-trifluoromethyl-phenyl)-thiazol-5-ylmethoxy]-phenyl}-acetic acid | 481.49 | [M + H⁺]⁺ =482.2[M − H⁺]⁻ =480.2 |
| P-0046 | 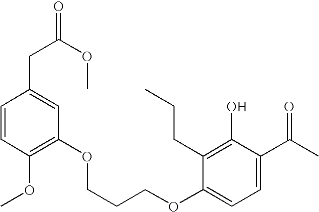 | {3-[3-(4-Acetyl-3-hydroxy-2-propyl-phenoxy)-propoxy]-4-methoxy-phenyl}-acetic acidmethyl ester | 430.49 | [M + H⁺]⁺ =431.29 |
| P-0047 | 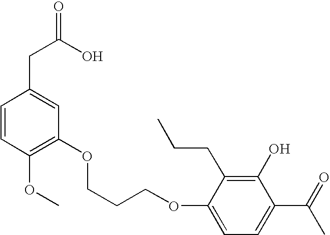 | {3-[3-(4-Acetyl-3-hydroxy-2-propyl-phenoxy)-propoxy]-4-methoxy-phenyl}-acetic acid | 416.47 | [M − H⁺]⁻ =415;[M + H⁺]⁺ =417 |

### Example 10

### Synthesis of {3-ethoxy-5-[3-(6-methoxy-pyridin-3-yl)-phenoxy]-phenyl}-acetic acid (P-0089)

Compound P-0089 was synthesized in three steps from (3-ethoxy-5-hydroxy-phenyl)-acetic acid methyl ester 16 as shown in Scheme 19.

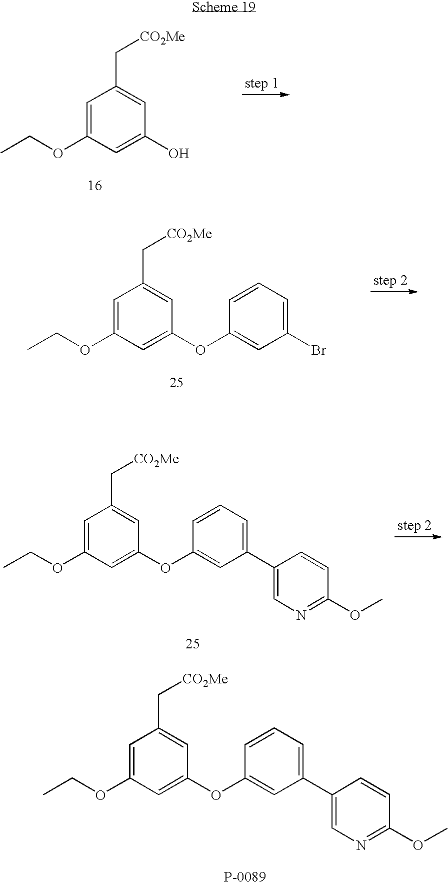

### Step 1: Preparation of [3-(3-bromo-phenoxy)-5-ethoxy-phenyl]-acetic acid methyl ester (25)

To a solution of (3-ethoxy-5-hydroxy-phenyl)-acetic acid methyl ester (16, 200 mg, 0.001 mol, prepared as per Step 1 of Scheme 15, Example 6) dissolved in 1,4-dioxane (3 mL, 0.04 mol), cesium carbonate (620 mg, 0.0019 mol), 1-bromo-3-iodo-benzene (180 μL, 0.0014 mol), dimethylamino-acetic acid (30 mg, 0.0003 mol) and copper(I) iodide (20 mg, 0.0001 mol) were added. The mixture was heated at 90° C. overnight under an atmosphere of argon. The reaction was diluted with a mixture of ammonium chloride:ammonium hydroxide 4:1 and extracted with ethyl acetate 3×. The combined organic layers were dried over sodium sulfate, concentrated under reduced pressure, and absorbed onto silica for flash chromatography. Using a gradient of 10-20% ethyl acetate in hexanes, the pure compound 25 was isolated. ¹H NMR was consistent with the desired compound. MS (ESI) [M+H⁺]⁺=417.2 [M−H⁺]⁻=365.1, 367.1.

### Step 2. Preparation of {3-ethoxy-5-[3-(6-methoxy-pyridin-3-yl)-phenoxy]-phenyl}-acetic acid methyl ester (26)

To a solution of [3-(3-bromo-phenoxy)-5-ethoxy-phenyl]-acetic acid methyl ester (25, 71 mg, 0.00019 mol) in tetrahydrofuran (5 mL, 0.07 mol) was added 2-methoxypyridyl boronic acid (44 mg, 0.00029 mol) and [1,1′-bis(diphenylphosphino)-ferrocene]dichloropalladium(II), complex with dichloromethane (1:1) (16 mg, 0.000019 mol) and 1M potassium carbonate in water (0.6 mL). The reaction was stirred at 90° C. overnight. After cooling, water was added to dilute the reaction. The reaction was extracted with ethyl acetate 3×. The combined organic layers were washed with brine 1×, and dried over sodium sulfate. After concentration under reduced pressure, the crude product was absorbed onto silica and purified via flash chromatography with a gradient of 10-20% ethyl acetate in hexanes to isolate the desired compound as a clear oil. ¹H NMR consistent with compound structure.

### Step 3: Preparation of {3-ethoxy-5-[3-(6-methoxy-pyridin-3-yl)-phenoxy]-phenyl}-acetic acid (P-0089)

Into a flask, 3-ethoxy-5-[3-(6-methoxy-pyridin-3-yl)-phenoxy]-phenyl-acetic acid methyl ester 26 was dissolved in THF (3 ml), 1 mL LiOH (1M) was also added, and the reaction stirred overnight at ambient conditions. The reaction was acidified to pH 1-2 with 1M HCl. The reaction was extracted with ethyl acetate 2× and the combined organic layers were dried over Na₂SO₄, concentrated under reduced pressure, and purified on prep. TLC plates with 5% methanol in dichloromethane. ¹H NMR consistent with compound structure. Calculated molecular weight 379.41, MS (ESI) [M−H⁺]⁺=380.2, [M−H⁺]⁻=379.1.

Additional compounds were prepared by optionally replacing the 2-methoxypyridyl boronic acid with an appropriate boronic acid compound in Step 2, and/or optionally replacing the (3-ethoxy-5-hydroxy-phenyl)-acetic acid methyl ester 16 with an appropriate acetic acid methyl ester in Step 1, where the acetic acid methyl ester is prepared according to Step 1 of Scheme 15, Example 6 by replacing iodoethane with an appropriate iodoalkyl compound. The following Table 10 indicates the appropriate acetic acid methyl ester and boronic acid compounds used in Step 1 and 2, respectively, for the indicated compound.

TABLE 10

| Cmpd. | | |
|---|---|---|
| Number | Acetic acid methyl ester | Boronic acid |
| P-0087 | 3-ethoxy-5-hydroxy-phenyl | 4-ethoxy-phenyl |
| P-0090 | ″ | 3-fluoro-4-methoxy-phenyl |
| P-0091 | ″ | 2,6-dimethoxy-pyridin-3-yl |
| P-0097 | ″ | 4-chloro-phenyl |
| P-0098 | ″ | 2-methoxy-phenyl |
| P-0099 | ″ | 4-methoxy-phenyl |
| P-0100 | ″ | 3-chloro-4-fluoro-phenyl |
| P-0101 | ″ | 2-trifluoromethyl-phenyl |
| P-0102 | ″ | 3-trifluoromethoxy-phenyl |
| P-0103 | ″ | 4-trifluoromethoxy-phenyl |
| P-0104 | ″ | 3-trifluoromethyl-phenyl |
| P-0122 | 3-hydroxy-5-propoxy-phenyl | 4-chloro-phenyl |
| P-0123 | ″ | 2-methoxy-phenyl |
| P-0124 | ″ | 4-methoxy-phenyl |
| P-0125 | ″ | 3-chloro-4-fluoro-phenyl |
| P-0126 | ″ | 2-trifluoromethyl-phenyl |
| P-0127 | ″ | 4-ethoxy-phenyl |
| P-0128 | ″ | 3-trifluoromethoxy-phenyl |
| P-0129 | ″ | 4-trifluoromethoxy-phenyl |
| P-0130 | ″ | 3-trifluoromethyl-phenyl |
| P-0131 | ″ | 6-methoxy-pyridin-3-yl |
| P-0132 | ″ | 3-fluoro-4-methoxy-phenyl |
| P-0133 | ″ | 2,4-dimethoxy-pyrimidin-5-yl |
| P-0234 | 3-hydroxy-5-(2-methoxy- | 1,3,5-trimethyl-1H-pyrazol-4-yl |
| | ethoxy)-phenyl | |
| P-0257 | 3-hydroxy-5-(2-methoxy- | 2,4-dimethoxy-pyrimidin-5-yl |
| | ethoxy)-phenyl | |
| P-0160 | 3-cyclopropylmethoxy-5- | 2-methoxy-phenyl |
| | hydroxy-phenyl | |
| P-0161 | 3-cyclopropylmethoxy-5- | 4-methoxy-phenyl |
| | hydroxy-phenyl | |
| P-0162 | 3-cyclopropylmethoxy-5- | 4-chloro-phenyl |
| | hydroxy-phenyl | |
| P-0163 | 3-cyclopropylmethoxy-5- | 3-chloro-4-fluoro-phenyl |
| | hydroxy-phenyl | |
| P-0164 | 3-cyclopropylmethoxy-5- | 2-trifluoromethyl-phenyl |
| | hydroxy-phenyl | |
| P-0165 | 3-cyclopropylmethoxy-5- | 4-ethoxy-phenyl |
| | hydroxy-phenyl | |
| P-0166 | 3-cyclopropylmethoxy-5- | 3-trifluoromethoxy-phenyl |
| | hydroxy-phenyl | |
| P-0167 | 3-cyclopropylmethoxy-5- | 4-trifluoromethoxy-phenyl |
| | hydroxy-phenyl | |
| P-0168 | 3-cyclopropylmethoxy-5- | 3-trifluoromethyl-phenyl |
| | hydroxy-phenyl | |
| P-0169 | 3-cyclopropylmethoxy-5- | 4-trifluoromethyl-phenyl |
| | hydroxy-phenyl | |
| P-0170 | 3-cyclopropylmethoxy-5- | 6-methoxy-pyridin-3-yl |
| | hydroxy-phenyl | |
| P-0171 | 3-cyclopropylmethoxy-5- | 3-fluoro-4-methoxy-phenyl |
| | hydroxy-phenyl | |
| P-0172 | 3-cyclopropylmethoxy-5- | 1-methyl-1H-pyrazol-4-yl |
| | hydroxy-phenyl | |
| P-0173 | 3-cyclopropylmethoxy-5- | 1,3,5-trimethyl-1H-pyrazol-4-yl |
| | hydroxy-phenyl | |
| P-0174 | 3-cyclopropylmethoxy-5- | 1-(3-methyl-butyl)-1H-pyrazol- |
| | hydroxy-phenyl | 4-yl |
| P-0176 | 3-ethoxy-5-hydroxy-phenyl | 1H-pyrazol-4-yl |
| P-0177 | ″ | 1-methyl-1H-pyrazol-4-yl |
| P-0178 | ″ | 1,3,5-trimethyl-1H-pyrazol-4-yl |
| P-0179 | ″ | 1-isobutyl-1H-pyrazol-4-yl |
| P-0180 | ″ | 1-(3-methyl-butyl)-1H-pyrazol- |
| | | 4-yl |
| P-0181 | 3-hydroxy-5-propoxy-phenyl | 4-trifluoromethyl-phenyl |
| P-0182 | ″ | 1H-pyrazol-4-yl |
| P-0183 | ″ | 1-methyl-1H-pyrazol-4-yl |
| P-0184 | ″ | 1,3,5-trimethyl-1H-pyrazol-4-yl |
| P-0185 | ″ | 1-isobutyl-1H-pyrazol-4-yl |
| P-0186 | ″ | 1-(3-methyl-butyl)-1H-pyrazol- |
| | | 4-yl |
| P-0209 | 3-hydroxy-5-(2-methoxy- | 1-(3-methyl-butyl)-1H-pyrazol- |
| | ethoxy)-phenyl | 4-yl |
| P-0210 | 3-hydroxy-5-(2-methoxy- | 1-isobutyl-1H-pyrazol-4-yl |
| | ethoxy)-phenyl | |
| P-0211 | 3-hydroxy-5-(2-methoxy- | 6-methoxy-pyridin-3-yl |
| | ethoxy)-phenyl | |
| P-0218 | 3-hydroxy-5-(2-methoxy- | 4-chloro-phenyl |
| | ethoxy)-phenyl | |
| P-0219 | 3-hydroxy-5-(2-methoxy- | 2-methoxy-phenyl |
| | ethoxy)-phenyl | |
| P-0220 | 3-hydroxy-5-(2-methoxy- | 4-methoxy-phenyl |
| | ethoxy)-phenyl | |
| P-0221 | 3-hydroxy-5-(2-methoxy- | 3-chloro-4-fluoro-phenyl |
| | ethoxy)-phenyl | |
| P-0222 | 3-hydroxy-5-(2-methoxy- | 2-trifluoromethyl-phenyl |
| | ethoxy)-phenyl | |
| P-0223 | 3-hydroxy-5-(2-methoxy- | 4-ethoxy-phenyl |
| | ethoxy)-phenyl | |
| P-0224 | 3-hydroxy-5-(2-methoxy- | 3-trifluoromethoxy-phenyl |
| | ethoxy)-phenyl | |
| P-0225 | 3-hydroxy-5-(2-methoxy- | 4-trifluoromethoxy-phenyl |
| | ethoxy)-phenyl | |
| P-0226 | 3-hydroxy-5-(2-methoxy- | 3-trifluoromethyl-phenyl |
| | ethoxy)-phenyl | |
| P-0227 | 3-hydroxy-5-(2-methoxy- | 4-trifluoromethyl-phenyl |
| | ethoxy)-phenyl | |
| P-0228 | 3-hydroxy-5-(2-methoxy- | 3-fluoro-4-methoxy-phenyl |
| | ethoxy)-phenyl | |
| P-0231 | 3-hydroxy-5-(2-methoxy- | 1H-pyrazol-4-yl |
| | ethoxy)-phenyl | |
| P-0232 | 3-hydroxy-5-(2-methoxy- | 1-methyl-1H-pyrazol-4-yl |
| | ethoxy)-phenyl | |

The compound structures, names and mass spectrometry results for these compounds are provided in the following Table 11.

TABLE 11

| | | | Molecular weight | |
|---|---|---|---|---|
| Cmpd. | | | | Measured |
| number | Structure | Name | Calc. | MS(ESI) |
| P-0087 | 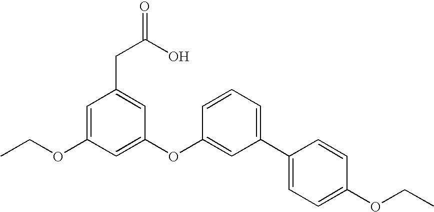 | [3-Ethoxy-5-(4′-ethoxy-biphenyl-3-yloxy)-phenyl]-acetic acid | 392.45 | [M − H⁺]⁻ =393.1, 391.1 |
| P-0090 | 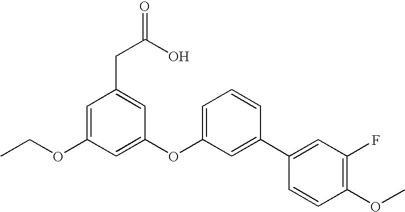 | [3-Ethoxy-5-(3′-fluoro-4′-methoxy-biphenyl-3-yloxy)-phenyl]-acetic acid | 396.41 | [M + H⁺]⁺ =397.2[M − H⁺]⁻ =395.1 |
| P-0091 | 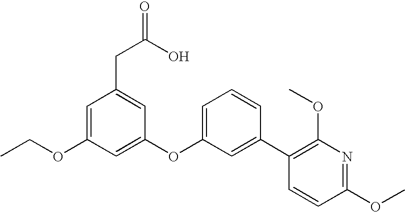 | {3-[3-(2,6-Dimethoxy-pyridin-3-yl)-phenoxy]-5-ethoxy-phenyl}-acetic acid | 409.44 | [M + H⁺]⁺ =410.2[M − H⁺]⁻ =408.1 |
| P-0097 | 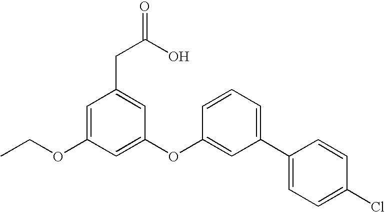 | [3-(4′-Chloro-biphenyl-3-yloxy)-5-ethoxy-phenyl]-aceticacid | 382.84 | [M + H⁺]⁺ =383.1 |
| P-0098 | 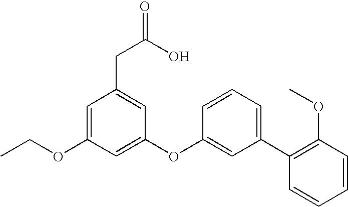 | [3-Ethoxy-5-(2′-methoxy-biphenyl-3-yloxy)-phenyl]-acetic acid | 378.42 | [M + H⁺]⁺ =379.1 |
| P-0099 | 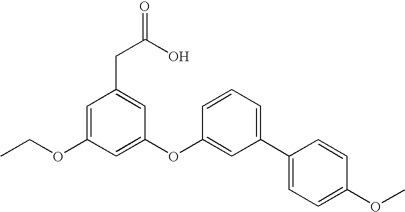 | [3-Ethoxy-5-(4′-methoxy-biphenyl-3-yloxy)-phenyl]-acetic acid | 378.42 | [M + H⁺]⁺ =379.1 |
| P-0100 | 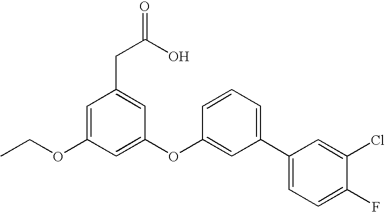 | [3-(3′-Chloro-4′-fluoro-biphenyl-3-yloxy)-5-ethoxy-phenyl]-acetic acid | 400.83 | [M + H⁺]⁺ =401.1 |
| P-0101 | 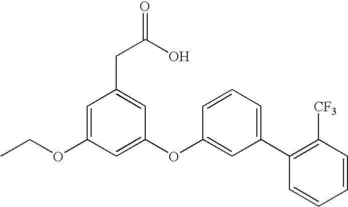 | [3-Ethoxy-5-(2′-trifluoromethyl-biphenyl-3-yloxy)-phenyl]-acetic acid | 416.39 | [M + H⁺]⁺ =417.5 |
| P-0102 | 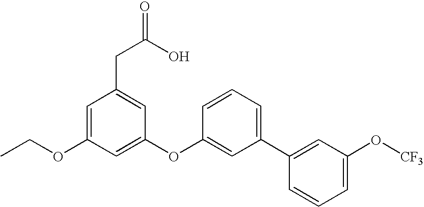 | [3-Ethoxy-5-(3′-trifluoromethoxy-biphenyl-3-yloxy)-phenyl]-acetic acid | 432.39 | [M + H⁺]⁺ =433.1 |
| P-0103 | 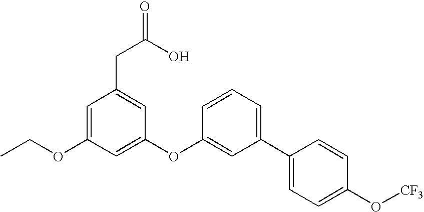 | [3-Ethoxy-5-(4′-trifluoromethoxy-biphenyl-3-yloxy)-phenyl]-acetic acid | 432.39 | [M + H⁺]⁺ =433.1 |
| P-0104 | 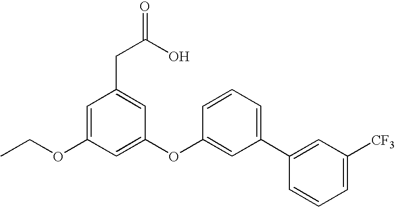 | [3-Ethoxy-5-(3′-trifluoromethyl-biphenyl-3-yloxy)-phenyl]-acetic acid | 416.39 | [M + H⁺]⁺ =417.1 |
| P-0122 | 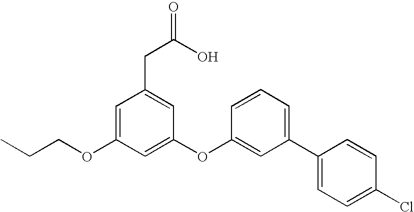 | [3-(4′-Chloro-biphenyl-3-yloxy)-5-propoxy-phenyl]-acetic acid | 396.87 | [M + H⁺]⁺ =397.1 |
| P-0123 | 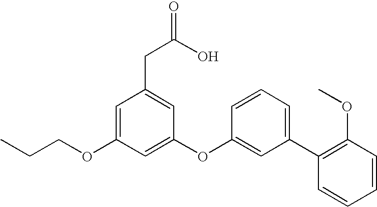 | [3-(2′-Methoxy-biphenyl-3-yloxy)-5-propoxy-phenyl]-acetic acid | 392.45 | [M + H⁺]⁺ =393.1 |
| P-0124 | 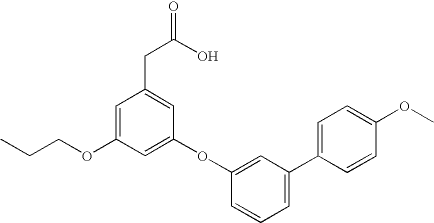 | [3-(4′-Methoxy-biphenyl-3-yloxy)-5-propoxy-phenyl]-acetic acid | 392.45 | [M + H⁺]⁺ =393.1 |
| P-0125 | 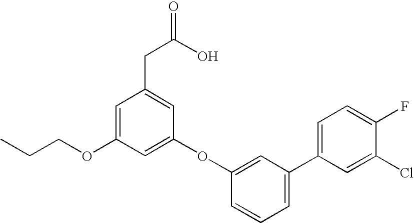 | [3-(3′-Chloro-4′-fluoro-biphenyl-3-yloxy)-5-propoxy-phenyl]-acetic acid | 414.86 | [M + H⁺]⁺ =415.1 |
| P-0126 | 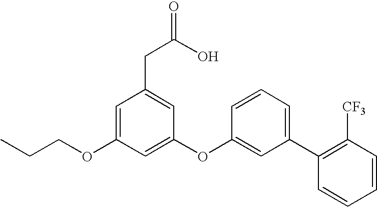 | [3-Propoxy-5-(2′-trifluoromethyl-biphenyl-3-yloxy)-phenyl]-acetic acid | 430.42 | [M + H⁺]⁺ =431.1 |
| P-0127 | 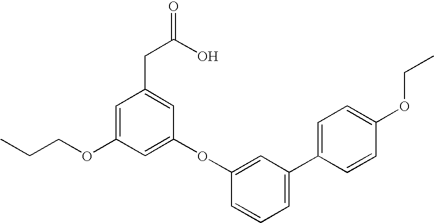 | [3-(4′-Ethoxy-biphenyl-3-yloxy)-5-propoxy-phenyl]-acetic acid | 406.48 | [M + H⁺]⁺ =407.1 |
| P-0128 | 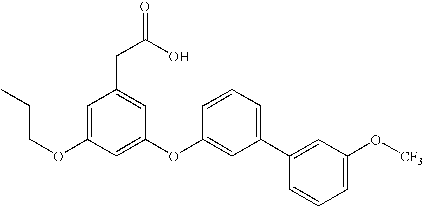 | [3-Propoxy-5-(3′-trifluoromethoxy-biphenyl-3-yloxy)-phenyl]-acetic acid | 446.42 | [M + H⁺]⁺ =447.1 |
| P-0129 | 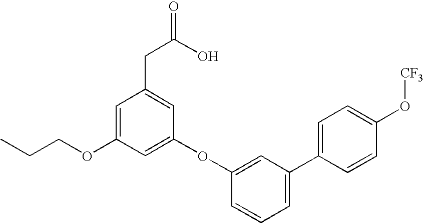 | [3-Propoxy-5-(4′-trifluoromethoxy-biphenyl-3-yloxy)-phenyl]-acetic acid | 446.42 | [M + H⁺]⁺ =447.1 |
| P-0130 | 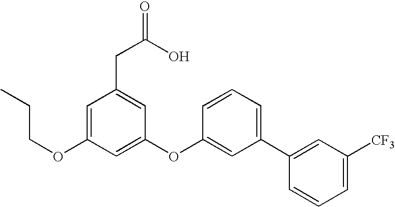 | [3-Propoxy-5-(3′-trifluoromethyl-biphenyl-3-yloxy)-phenyl]-acetic acid | 430.42 | [M + H⁺]⁺ =431.1 |
| P-0131 | 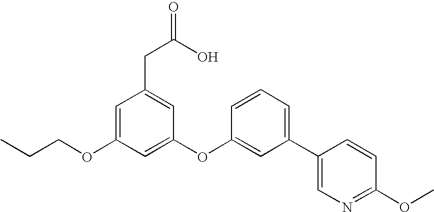 | {3-[3-(6-Methoxy-pyridin-3-yl)-phenoxy]-5-propoxy-phenyl}-acetic acid | 393.44 | [M + H⁺]⁺ =394.3 |
| P-0132 | 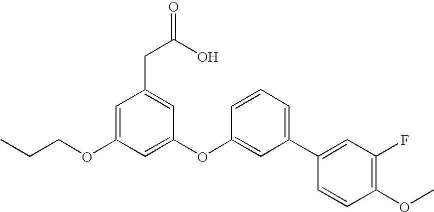 | [3-(3′-Fluoro-4′-methoxy-biphenyl-3-yloxy)-5-propoxy-phenyl]-acetic acid | 410.44 | [M + H⁺]⁺ =411.1 |
| P-0133 | 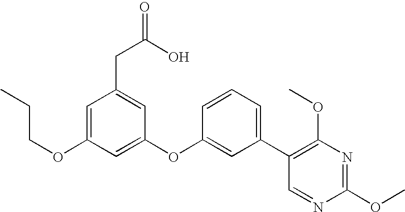 | {3-[3-(2,4-Dimethoxy-pyrimidin-5-yl)-phenoxy]-5-propoxy-phenyl}-acetic acid | 424.45 | [M + H⁺]⁺ =425.1 |
| P-0234 | 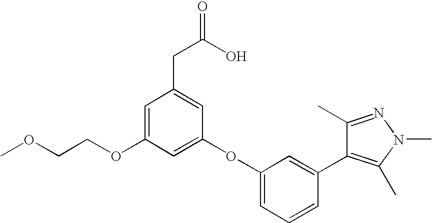 | {3-(2-Methoxy-ethoxy)-5-[3-(1,3,5-trimethyl-1H-pyrazol-4-yl)-phenoxy]-phenyl}-aceticacid | 410.47 | [M + H⁺]⁺ =411.1 |
| P-0257 | 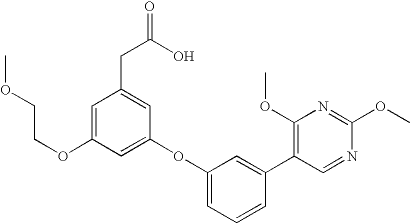 | [3-[3-(2,4-Dimethoxy-pyrimidin-5-yl)-phenoxy]-5-(2-methoxy-ethoxy)-phenyl]-acetic acid | 440.45 | [M + H⁺]⁺ =441.1 |
| P-0160 | 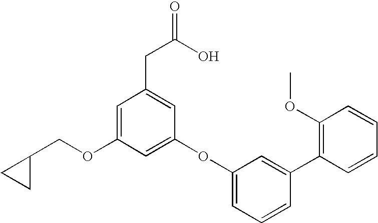 | [3-Cyclopropylmethoxy-5-(2′-methoxy-biphenyl-3-yloxy)-phenyl]-acetic acid | 404.46 | [M + H⁺]⁺ =405.5 |
| P-0161 | 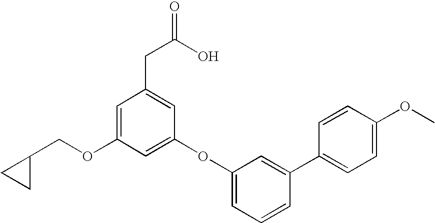 | [3-Cyclopropylmethoxy-5-(4′-methoxy-biphenyl-3-yloxy)-phenyl]-acetic acid | 404.46 | [M + H⁺]⁺ =405.5 |
| P-0162 | 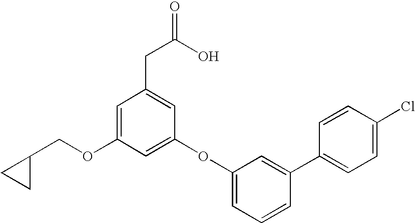 | [3-(4′-Chloro-biphenyl-3-yloxy)-5-cyclopropylmethoxy-phenyl]-acetic acid | 408.88 | [M + H⁺]⁺ =409.1 |
| P-0163 | 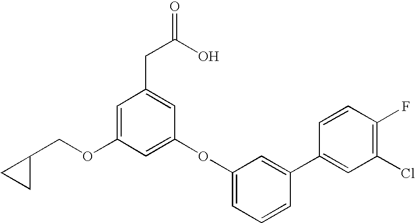 | [3-(3′-Chloro-4′-fluoro-biphenyl-3-yloxy)-5-cyclopropylmethoxy-phenyl]-acetic acid | 426.87 | [M + H⁺]⁺ =427.1 |
| P-0164 | 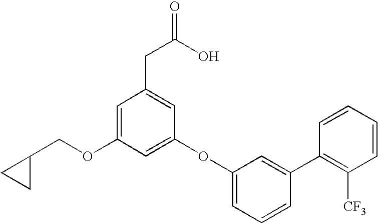 | [3-Cyclopropylmethoxy-5-(2′-trifluoromethyl-biphenyl-3-yloxy)-phenyl]-acetic acid | 442.43 | [M + H⁺]⁺ =443.1 |
| P-0165 | 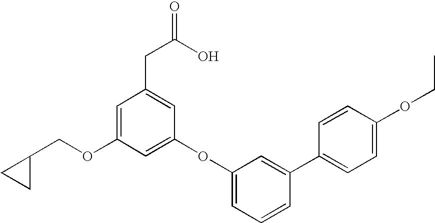 | [3-Cyclopropylmethoxy-5-(4′-ethoxy-biphenyl-3-yloxy)-phenyl]-acetic acid | 418.49 | [M + H⁺]⁺ =419.1 |
| P-0166 | 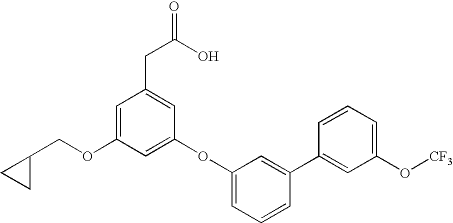 | [3-Cyclopropylmethoxy-5-(3′-trifluoromethoxy-biphenyl-3-yloxy)-phenyl]-acetic acid | 458.43 | [M + H⁺]⁺ =459.1 |
| P-0167 | 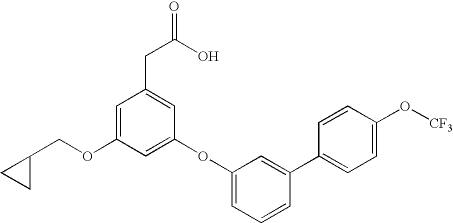 | [3-Cyclopropylmethoxy-5-(4′-trifluoromethoxy-biphenyl-3-yloxy)-phenyl]-acetic acid | 458.43 | [M + H⁺]⁺ =459.1 |
| P-0168 | 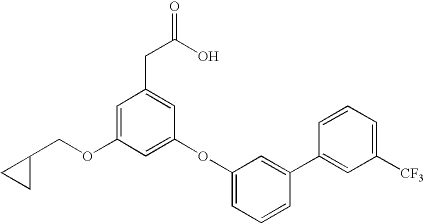 | [3-Cyclopropylmethoxy-5-(3′-trifluoromethyl-biphenyl-3-yloxy)-phenyl]-acetic acid | 442.43 | [M + H⁺]⁺ =443.1 |
| P-0169 | 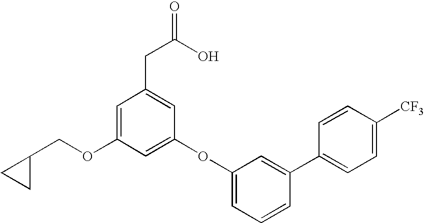 | [3-Cyclopropylmethoxy-5-(4′-trifluoromethyl-biphenyl-3-yloxy)-phenyl]-acetic acid | 442.43 | [M + H⁺]⁺ =443.1 |
| P-0170 | 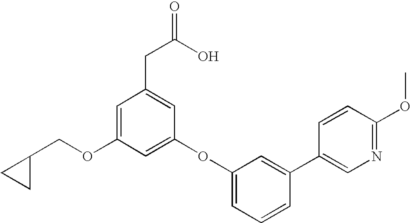 | {3-Cyclopropylmethoxy-5-[3-(6-methoxy-pyridin-3-yl)-phenoxy]-phenyl}-acetic acid | 405.45 | [M + H⁺]⁺ =405.3 |
| P-0171 | 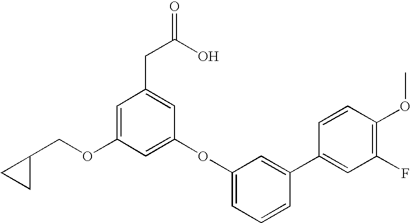 | [3-Cyclopropylmethoxy-5-(3′-fluoro-4′-methoxy-biphenyl-3-yloxy)-phenyl]-acetic acid | 422.45 | [M + H⁺]⁺ =423.1 |
| P-0172 | 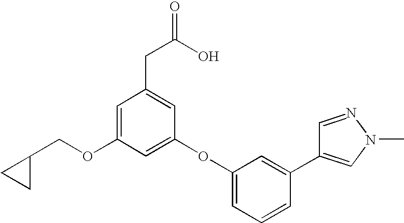 | {3-Cyclopropylmethoxy-5-[3-(1-methyl-1H-pyrazol-4-yl)-phenoxy]-phenyl}-acetic acid | 378.43 | [M + H⁺]⁺ =379.1 |
| P-0173 | 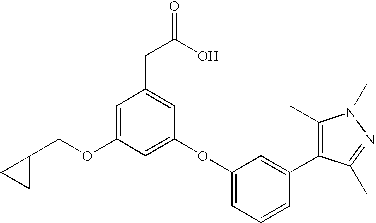 | {3-Cyclopropylmethoxy-5-[3-(1,3,5-trimethyl-1H-pyrazol-4-yl)-phenoxy]-phenyl}-aceticacid | 406.48 | [M + H⁺]⁺ =407.1 |
| P-0174 | 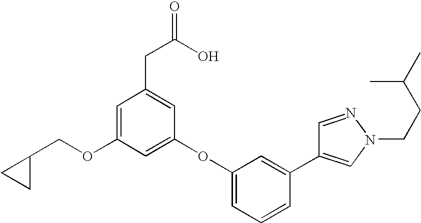 | (3-Cyclopropylmethoxy-5-{3-[1-(3-methyl-butyl)-1H-pyrazol-4-yl]-phenoxy}-phenyl)-acetic acid | 434.53 | [M + H⁺]⁺ =435.1 |
| P-0176 | 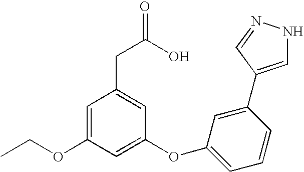 | {3-Ethoxy-5-[3-(1H-pyrazol-4-yl)-phenoxy]-phenyl}-aceticacid | 338.36 | [M + H⁺]⁺ =339.1 |
| P-0177 | 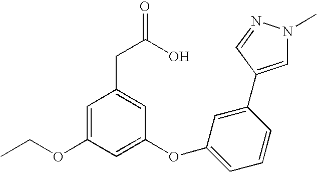 | {3-Ethoxy-5-[3-(1-methyl-1H-pyrazol-4-yl)-phenoxy]-phenyl}-acetic acid | 352.39 | [M + H⁺]⁺ =353.1 |
| P-0178 | 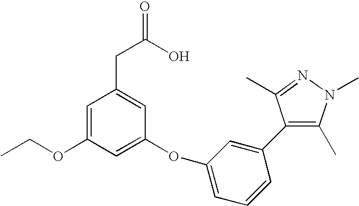 | {3-Ethoxy-5-[3-(1,3,5-trimethyl-1H-pyrazol-4-yl)-phenoxy]-phenyl}-acetic acid | 380.44 | [M + H⁺]⁺ =381.1 |
| P-0179 | 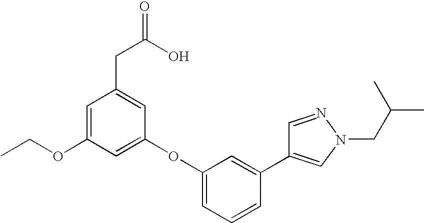 | {3-Ethoxy-5-[3-(1-isobutyl-1H-pyrazol-4-yl)-phenoxy]-phenyl}-acetic acid | 394.47 | [M + H⁺]⁺ =395.1 |
| P-0180 | 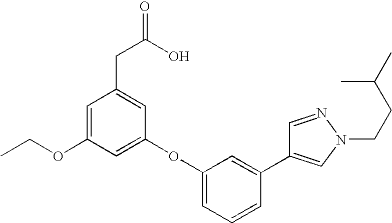 | (3-Ethoxy-5-{3-[1-(3-methyl-butyl)-1H-pyrazol-4-yl]-phenoxy}-phenyl)-acetic acid | 408.50 | [M + H⁺]⁺ =409.1 |
| P-0181 | 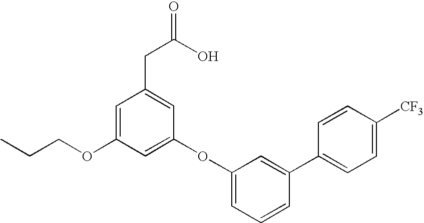 | [3-Propoxy-5-(4′-trifluoromethyl-biphenyl-3-yloxy)-phenyl]-acetic acid | 430.42 | [M + H⁺]⁺ =431.1 |
| P-0182 | 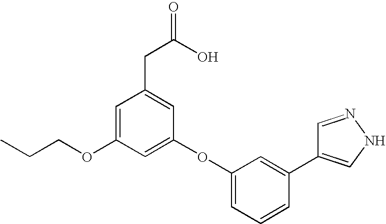 | {3-Propoxy-5-[3-(1H-pyrazol-4-yl)-phenoxy]-phenyl}-aceticacid | 352.39 | [M + H⁺]⁺ =353.1 |
| P-0183 | 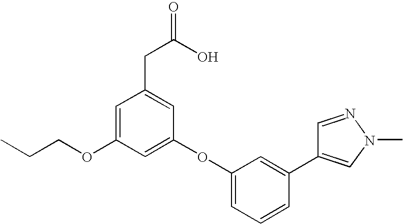 | {3-[3-(1-Methyl-1H-pyrazol-4-yl)-phenoxy]-5-propoxy-phenyl}-acetic acid | 366.41 | [M + H⁺]⁺ =367.1 |
| P-0184 | 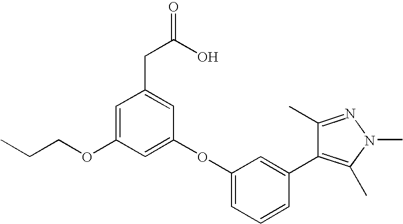 | {3-Propoxy-5-[3-(1,3,5-trimethyl-1H-pyrazol-4-yl)-phenoxy]-phenyl}-acetic acid | 394.47 | [M + H⁺]⁺ =395.1 |
| P-0185 | 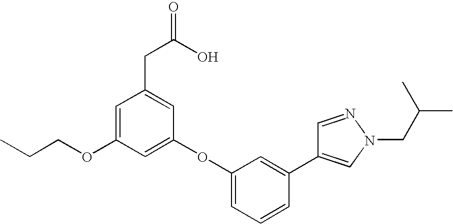 | {3-[3-(1-Isobutyl-1H-pyrazol-4-yl)-phenoxy]-5-propoxy-phenyl}-acetic acid | 408.50 | [M + H⁺]⁺ =409.1 |
| P-0186 | 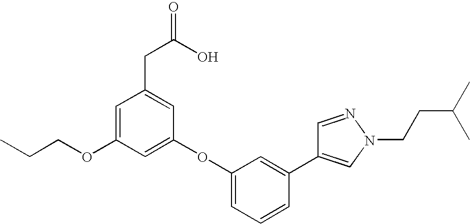 | (3-{3-[1-(3-Methyl-butyl)-1H-pyrazol-4-yl]-phenoxy}-5-propoxy-phenyl)-acetic acid | 422.52 | [M + H⁺]⁺ =423.1 |
| P-0209 | 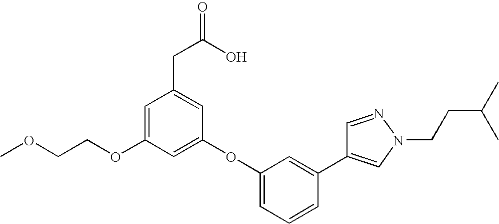 | (3-(2-Methoxy-ethoxy)-5-{3-[1-(3-methyl-butyl)-1H-pyrazol-4-yl]-phenoxy}-phenyl)-acetic acid | 438.52 | [M + H⁺]⁺ =439.1 |
| P-0210 | 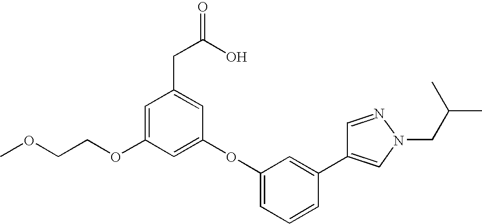 | [3-[3-(1-Isobutyl-1H-pyrazol-4-yl)-phenoxy]-5-(2-methoxy-ethoxy)-phenyl]-acetic acid | 424.49 | [M + H⁺]⁺ =425.1 |
| P-0211 | 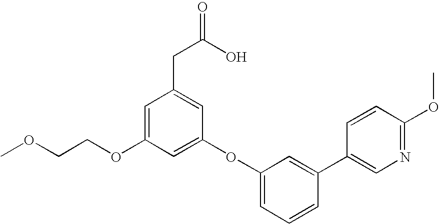 | {3-(2-Methoxy-ethoxy)-5-[3-(6-methoxy-pyridin-3-yl)-phenoxy]-phenyl}-acetic acid | 409.44 | [M + H⁺]⁺ =410.3 |
| P-0218 | 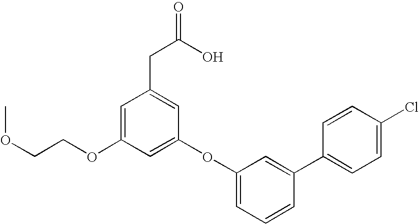 | [3-(4′-Chloro-biphenyl-3-yloxy)-5-(2-methoxy-ethoxy)-phenyl]-acetic acid | 412.87 | [M + H⁺]⁺ =413.1 |
| P-0219 | 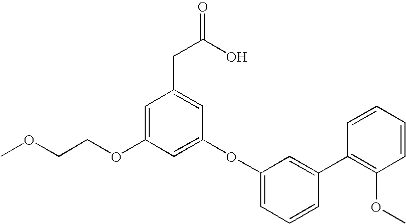 | [3-(2′-Methoxy-biphenyl-3-yloxy)-5-(2-methoxy-ethoxy)-phenyl]-acetic acid | 408.45 | [M + H⁺]⁺ =409.1 |
| P-0220 | 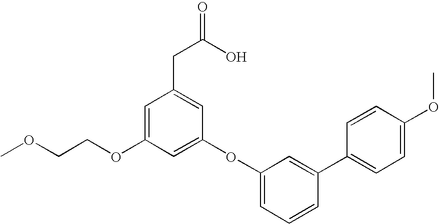 | [3-(4′-Methoxy-biphenyl-3-yloxy)-5-(2-methoxy-ethoxy)-phenyl]-acetic acid | 408.45 | [M + H⁺]⁺ =409.1 |
| P-0221 | 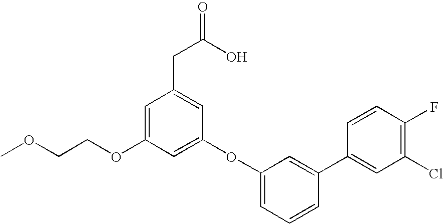 | [3-(3′-Chloro-4′-fluoro-biphenyl-3-yloxy)-5-(2-methoxy-ethoxy)-phenyl]-acetic acid | 430.86 | [M + H⁺]⁺ =431.1 |
| P-0222 | 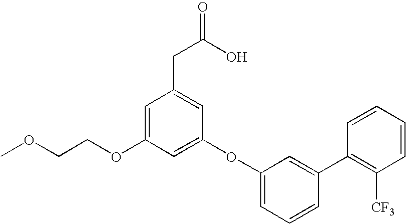 | [3-(2-Methoxy-ethoxy)-5-(2′-trifluoromethyl-biphenyl-3-yloxy)-phenyl]-acetic acid | 446.42 | [M + H⁺]⁺ =447.1 |
| P-0223 | 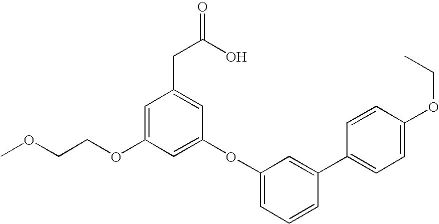 | [3-(4′-Ethoxy-biphenyl-3-yloxy)-5-(2-methoxy-ethoxy)-phenyl]-acetic acid | 422.47 | [M + H⁺]⁺ =423.1 |
| P-0224 | 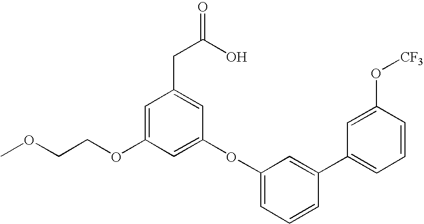 | [3-(2-Methoxy-ethoxy)-5-(3′-trifluoromethoxy-biphenyl-3-yloxy)-phenyl]-acetic acid | 462.42 | [M + H⁺]⁺ =463.1 |
| P-0225 | 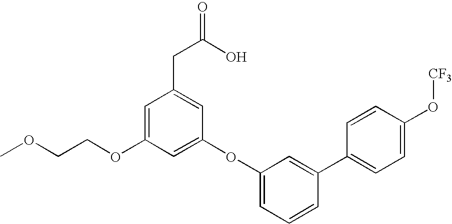 | [3-(2-Methoxy-ethoxy)-5-(4′-trifluoromethoxy-biphenyl-3-yloxy)-phenyl]-acetic acid | 462.42 | [M + H⁺]⁺ =463.1 |
| P-0226 | 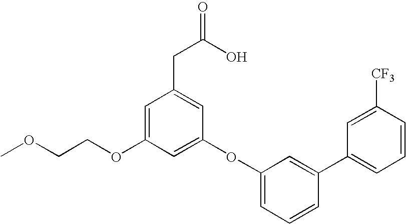 | [3-(2-Methoxy-ethoxy)-5-(3′-trifluoromethoxy-biphenyl-3-yloxy)-phenyl]-acetic acid | 446.42 | [M + H⁺]⁺ =447.1 |
| P-0227 | 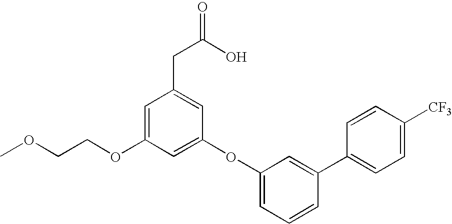 | [3-(2-Methoxy-ethoxy)-5-(4′-trifluoromethyl-biphenyl-3-yloxy)-phenyl]-acetic acid | 446.42 | [M + H⁺]⁺ =447.1 |
| P-0228 | 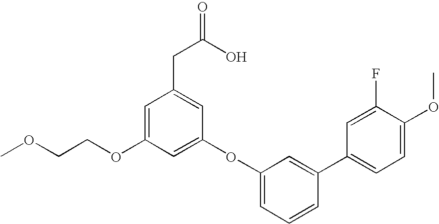 | [3-(3′-Fluoro-4′-methoxy-biphenyl-3-yloxy)-5-(2-methoxy-ethoxy)-phenyl]-acetic acid | 426.44 | [M + H⁺]⁺ =427.1 |
| P-0231 | 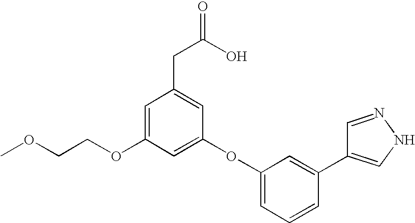 | {3-(2-Methoxy-ethoxy)-5-[3-(1H-pyrazol-4-yl)-phenoxy]-phenyl}-acetic acid | 368.39 | [M + H⁺]⁺ =369.1 |
| P-0232 | 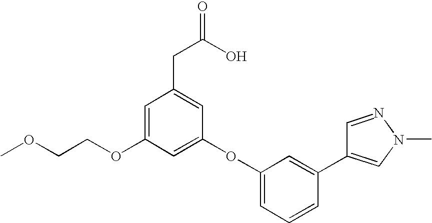 | {3-(2-Methoxy-ethoxy)-5-[3-(1-methyl-1H-pyrazol-4-yl)-phenoxy]-phenyl}-acetic acid | 382.41 | [M + H⁺]⁺ =383.1 |

### Example 11

### Synthesis of {3-ethoxy-5-[5-methyl-4-(4-trifluoromethyl-phenyl)-thiophene-2-sulfonyl]-phenyl}-acetic acid (P-0093)

Compound P-0093 was synthesized in five steps as shown in Scheme 20.

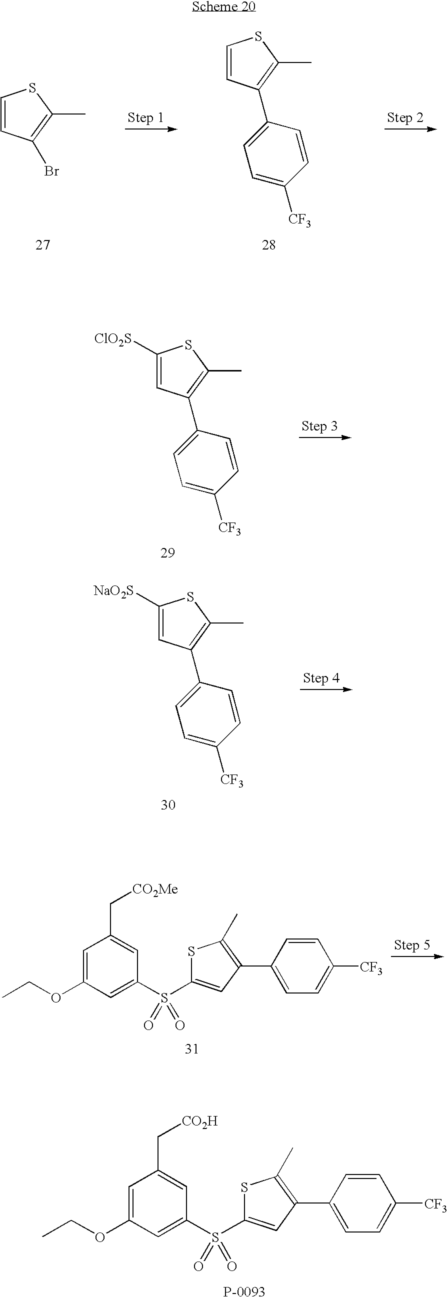

### Step 1: Preparation of 2-methyl-3-(4-trifluoromethyl-phenyl)-thiophene (28)

Into a microwave test tube, 2-methyl-3-bromothiophene (27, 130.0 mg, 0.0007342 mol), 4-(trifluoromethyl)phenylboronic acid (189 mg, 0.000995 mol), tetrakis(triphenylphosphine)palladium(0) (10 mg, 0.000009 mol), and 1N K₂CO₃ (0.3 mL) were stirred in 1,2-ethanediol (3 mL, 0.05 mol). The reaction vessel was heated at 98° C. for 30 minutes, then an additional 20 minutes at 300 watts power. The reaction was transferred to a round bottom flask and solvent was removed via azeotroping with ethyl acetate to an oil. The crude material was then absorbed onto silica, and purified via flash chromatography with a gradient of 10-20% ethyl acetate in hexane to isolate the desired compound. ¹H NMR consistent with compound structure.

### Step 2: Preparation of 5-methyl-4-(4-trifluoromethyl-phenyl)-thiophene-2-sulfonyl chloride (29)

Into a dry round bottom flask, chlorosulfonic acid (620 mg, 0.0053 mol) was dissolved in dichloromethane (10 mL, 0.2 mol). The flask was placed on an ice-bath and cooled for 10-15 minutes under a gentle argon flow. Phosphorus pentachloride (410 mg, 0.0020 mol) was added and the reaction stirred vigorously. The solution was stirred until the phosphorus pentachloride fully dissolved, after which 2-methyl-3-(4-trifluoromethyl phenyl) thiophene (28, 4.00E2 mg, 0.00165 mol) dissolved in 3 mL dichloromethane was added in one portion to the reaction. The color of the reaction turned from yellow to dark green. After 3 hours, the reaction was poured into an ice/water mixture, and stirred until all of the ice melted. The reaction was poured into a separatory funnel, and the reaction was extracted with dichloromethane (2×30 mL). The combined organic layers were washed with water (2×10 mL), 1× with brine (15 mL), and dried over MgSO₄. Solvent was evaporated under reduced pressure, and absorbed onto silica. Flash chromatography with a gradient of 0-30% ethyl acetate in hexanes led to isolation of the desired compound. ¹H NMR consistent with compound structure.

### Step 3: Preparation of 5-methyl-4-(4-trifluoromethylphenyl)-thiophene-2-sulfinic acid sodium salt (30)

Into a round bottom flask, sodium sulfite (308 mg, 0.00244 mol) was dissolved in water (13 mL, 0.72 mol). The flask was placed on an oil bath, pre-heated at 90° C. The reaction was stirred for 20 minutes until all of the sodium sulfite dissolved. Sodium bicarbonate (105 mg, 0.00125 mol) and 5-methyl-4-(4-trifluoromethylphenyl)thiophene-2-sulfonyl chloride (29, 356.0 mg, 0.001045 mol) were added simultaneously to the flask, and the reaction heated at 103° C. for 4 hours. The flask was cooled to room temperature and lyophilized for 2 days. Ethanol (40 mL) was added to the salt and the vessel heated at 100° C. for 40 minutes and subjected to hot gravity filtration. The salt was rinsed generously with hot ethanol. The filtrate was evaporated under reduced pressure to afford the 5-methyl-4-(4-trifluoromethylphenyl)-thiophene-2-sulfinic acid sodium salt 30 as a white solid.

### Step 4: Preparation of {3-ethoxy-5-[5-methyl-4-(4-trifluoromethyl-phenyl)-thiophene-2-sulfonyl]-phenyl}-acetic acid methyl ester (31)

Into a screw capped reaction vessel, 3-ethoxy-5-trifluoromethanesulfonyloxy-phenyl-acetic acid methyl ester (17, 110 mg, 0.00032 mol, prepared as in Step 2 of Scheme 15, Example 6), 5-methyl-4-(4-trifluoromethylphenyl)-thiophene-2-sulfinic acid sodium salt (30, 130 mg, 0.00041 mol), xanthphos (10 mg, 0.00002 mol), and cesium carbonate (245 mg, 0.000752 mol) were dissolved in toluene (6 mL, 0.06 mol). The reaction vessel was purged with argon for 3-5 minutes and tris(dibenzylideneacetone)-dipalladium(0) (10 mg, 0.00001 mol) was added and the reaction was capped and placed on an oil bath pre-heated at 120° C. The reaction was heated overnight. The solvent was removed under reduced pressure. The crude product was purified via prep TLC plate, using 20% ethyl acetate in hexanes to isolate the desired compound as an oil. ¹H NMR consistent with compound structure.

### Step 5: Preparation of {3-ethoxy-5-[5-methyl-4-(4-trifluoromethyl-phenyl)-thiophene-2-sulfonyl]-phenyl}-acetic acid (P-0093)

Into a flask, {3-ethoxy-5-[5-methyl-4-(4-trifluoromethyl-phenyl)-thiophene-2-sulfonyl]-phenyl}-acetic acid methyl ester 31 was dissolved in a 3 mL mixture of tetrahydrofuran/1N LiOH (4:1), and the reaction stirred vigorously for 4 hours. The reaction was acidified by adding 1N HCl (pH 1-2), and the reaction was extracted with ethyl acetate (3×). The organic layers were dried over MgSO₄, and solvent was evaporated under reduced pressure. The crude product was subjected to prep TLC plate purification, eluting with 3% methanol in chloroform to isolate the desired compound. ¹H NMR consistent with compound structure. Calculated molecular weight 484.51, MS (ESI) [M−H⁺]⁻=484.21.

### Example 12

### Synthesis of [3-ethoxy-5-(5-phenyl-thiophen-2-yloxy)-phenyl]-acetic acid (P-0083)

Compound P-0083 was synthesized in two steps as shown in Scheme 21.

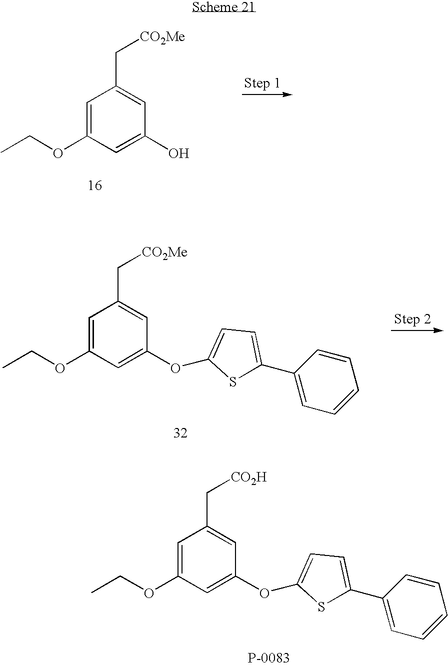

### Step 1: Preparation of [3-ethoxy-5-(5-phenyl-thiophen-2-yloxy)-phenyl]-acetic acid methyl ester (31)

Into a flask, (3-ethoxy-5-hydroxy-phenyl)-acetic acid methyl ester (16, 120 mg, 0.00057 mol, prepared as in Step 1 of Scheme 15, Example 6) was dissolved in 1,4-dioxane (2 mL, 0.02 mol). Cesium carbonate (370 mg, 0.0011 mol), 2-bromo-5-phenyl-thiophene (2.0E2 mg, 0.00086 mol), dimethylamino-acetic acid (20 mg, 0.0002 mol) and copper(I) iodide (10 mg, 0.00006 mol) were combined and stirred at 90° C. overnight under an atmosphere of argon. After cooling, the reaction was diluted with ethyl acetate, followed by a mixture of ammonium chloride:ammonium hydroxide 4:1. The layers were separated, the organic layer was dried with sodium sulfate, and the solvent was removed under reduced pressure. The crude material was absorbed onto silica and purified via flash chromatography with a gradient of 10-20% ethyl acetate in hexane to yield the desired compound. ¹H NMR consistent with compound structure.

### Step 2: Preparation of [3-ethoxy-5-(5-phenyl-thiophen-2-yloxy)-phenyl]-acetic acid (P-0083)

Into a flask, [3-ethoxy-5-(5-phenyl-thiophen-2-yloxy)-phenyl]-acetic acid methyl ester (31, 20 mg, 0.00005 mol) was dissolved in tetrahydrofuran (4 mL, 0.05 mol). 1M lithium hydroxide in water (1 mL) was added and the mixture was stirred overnight at room temperature. The mixture was first diluted with ethyl acetate and acidified using 1M HCl to pH 1-2. The layers were separated. The organic phase was dried over sodium sulfate and the solvent was removed under reduced pressure. The crude compound P-0083 was purified via prep TLC eluting with 5% methanol in dichloromethane to afford the desired compound. ¹H NMR consistent with compound structure. Calculated molecular weight 354.42, MS (ESI) [M+H⁺]⁺=355.1 [M−H⁺]⁻=353.0.

### Example 13

### Synthesis of 3-[4-Methyl-2-(4-trifluoromethyl-phenyl)-oxazole-5-sulfonyl]-phenyl-acetic acid (P-0284)

Compound P-0284 was synthesized in three steps as shown in Scheme 22.

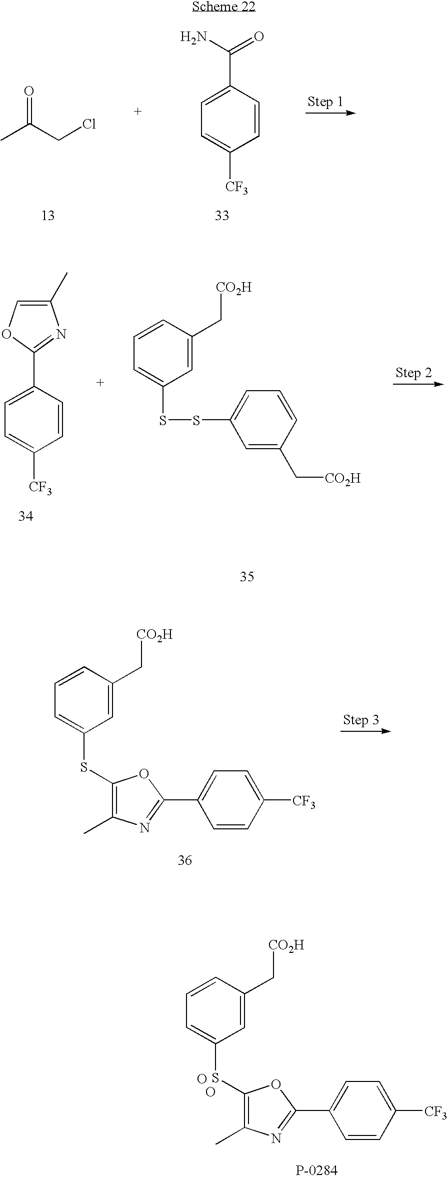

### Step 1: Preparation of 4-methyl-2-(4-trifluoromethyl-phenyl)-oxazole (34)

4-Trifluoromethyl-benzamide (33, 1.00 g, 0.00529 mol) was put in a microwave reaction vessel together with chloroacetone (13, 20 mL, 0.2 mol). The mixture was heated to at 120° C. for 40 minutes in the microwave. Starting material still remained. The solvent was evaporated and the crude reaction products put on silica and purified via flash chromatography (ethyl acetate in hexanes) to provide compound 34. ¹H NMR consistent with compound structure.

### Step 2: Preparation of 3-[4-methyl-2-(4-trifluoromethyl-phenyl)-oxazol-5-ylsulfanyl]-phenyl-acetic acid (36)

4-Methyl-2-(4-trifluoromethyl-phenyl)-oxazole (34, 200 mg, 0.0009 mol) was dissolved in tetrahydrofuran (5 mL, 0.06 mol). The mixture was cooled to at −76° C. sec-Butyllithium in hexane (1.4M, 2200 μL) was added dropwise and the mixture was stirred for 20 minutes. [3-(3-Carboxymethyl-phenyldisulfanyl)-phenyl]-acetic acid (35, 210 mg, 0.00063 mol) in tetrahydrofuran was added slowly to the solution. The mixture was allowed to reach room temperature and was stirred overnight. The reaction mixture was diluted with ethyl acetate and acidified using 1M HCl. The phases were separated and the aqueous phase was extracted with ethyl acetate. The pooled organic extract was dried with sodium sulfate and concentrated in vacuo. The reaction products were purified using flash chromatography (ethyl acetate in hexanes) to provide compound 36. ¹H NMR consistent with compound structure. MS (ESI) [M+H⁺]⁺=394.1, [M−H⁺]⁻=392.

### Step 3: Preparation of 3-[4-methyl-2-(4-trifluoromethyl-phenyl)-oxazole-5-sulfonyl]-phenyl-acetic acid (P-0284)

3-[4-Methyl-2-(4-trifluoromethyl-phenyl)-oxazol-5-ylsulfanyl]-phenyl-acetic acid (36, 20 mg, 0.00005 mol) was dissolved in dichloromethane (1 mL, 0.02 mol). m-Chloroperbenzoic acid (29 mg, 0.00017 mol) was added and the mixture stirred overnight at room temperature. TLC showed a spot with identical R_{f} to starting material, mass spectrometry showed mass of the desired compound. The mixture was concentrated and dissolved in methanol. The desired compound was purified utilizing reverse phase prep HPLC. ¹H NMR consistent with compound structure. Calculated molecular weight 425.38, MS (ESI) [M+H⁺]⁺=426.0, [M−H⁺]⁻=424.0.

### Example 14

### Synthesis of {3-[1-(4-trifluoromethyl-phenyl)-1H-pyrazole-4-sulfonyl]-phenyl}-acetic acid (P-0287) and related compounds

Compound P-0287 was synthesized in three steps as shown in Scheme 23.

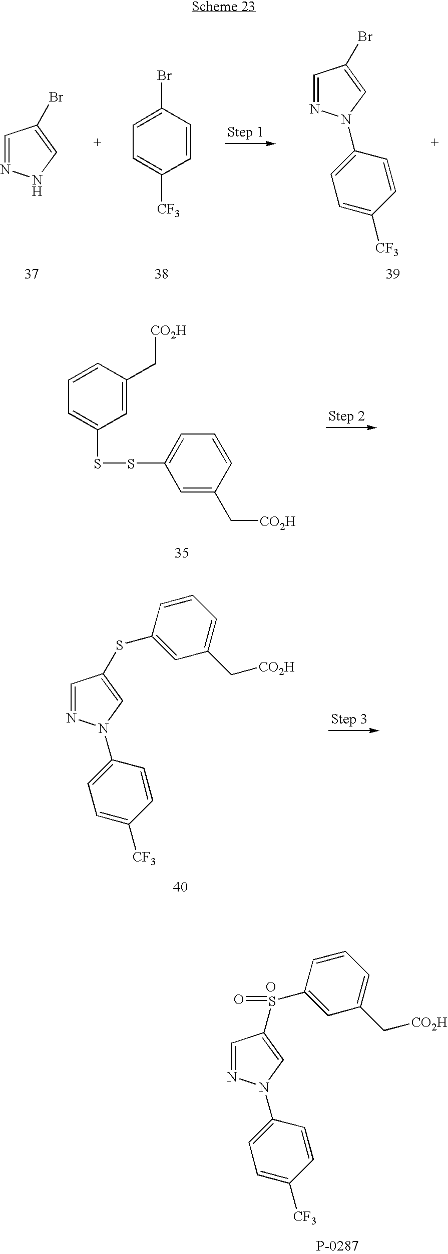

### Step 1: Preparation of 4-bromo-1-(4-trifluoromethylphenyl)-1H-pyrazole (39)

Into a round bottom flask (flame dried and under an inert condition) 1-bromo-4-trifluoromethyl benzene (38, 2.0 g, 0.0089 mol), salicylaldoxime (40 mg, 0.0003 mol), cesium carbonate (6 g, 0.02 mol), copper(I) oxide (44 mg, 0.00031 mol) and 4-bromopyrazole (37, 2.0 g, 0.013 mol) were combined in acetonitrile (15 mL, 0.21 mol). The combined mixture was heated at 110° C. for 3 days. The crude reaction was filtered using a Buchner funnel. The filtrate was reduced to half of the original volume and silica was added, then the solvent was completely removed by roto evaporation. Flash chromatography was executed with a gradient solvent (0 to 35% ethyl acetate/hexane) to obtain the desire compound. The intermediate was used without further characterization. ¹H NMR was consistent with compound structure.

### Step 2: Preparation of 3-[1-(4-trifluoromethylphenyl)1H-pyrazol-4-yl sulfonyl]phenyl acetic acid (40)

Into a round bottom flask (flame dried and under an inert condition) 4-bromo-1-(4-trifluoromethylphenyl)-1H-pyrazole (39, 180.00 mg, 6.1841E-4 mol) was dissolved in tetrahydrofuran (8 mL, 0.1 mol). The flask was placed in an acetone-dry ice bath and stirred for 10 minutes, providing the lithiated pyrazole solution. sec-Butyllithium (0.063 mL, 0.00074 mol) was added and the reaction stirred for 10 minutes. In another dry flask, under an argon purge, [3-(3-carboxymethyl-phenyldisulfanyl)-phenyl]-acetic acid (35, 206.8 mg, 0.0006184 mol) was dissolved in tetrahydrofuran (10 mL). sec-Butyllithium (0.126 mL, 0.00148 mol) was added and the reaction stirred for 10 minutes, providing the disulfide solution. The lithiated pyrazole solution was added to the disulfide solution using a cannula and the reaction stirred overnight under an inert atmosphere, after which TLC (20% ethyl acetate/hexane) indicated absence of phenyl pyrazole and mass spectrometry of the crude reaction was consistent with the desired compound. Methanol was added (3 mL) to quench the butyllithium and solvent was roto evaporated to dryness. The crude compound was absorbed onto silica and purified via flash chromatography using gradient solvent conditions (0 to 8% methanol/dichloromethane). ¹H NMR structural characterization indicated methylene peak. The compound was carried on to the next step without further purification.

### Step 3: Preparation of {3-[1-(4-trifluoromethyl-phenyl)-1H-pyrazole-4-sulfonyl]-phenyl}-acetic acid (P-0287)

Crude compound 40 from Step 2 was dissolved in 4 mL dichloromethane and m-CPBA (4 eq) was added. The reaction was stirred at room temperature for 6 hours and an aliquot taken at this time indicated desired product by mass spectrometry. Silica was added to the crude mixture and the solvent evaporated. Flash chromatography was executed using gradient conditions of 0 to 8% methanol/dichloromethane. The appropriate fractions indicated by mass spectrometry were combined and evaporated. This was re-dissolved in acetonitrile and subjected to reverse phase HPLC to isolate the desired compound. ¹H NMR (CD₃OD) consistent with compound structure, purity >90%. Calculated molecular weight 410.37, MS (ESI) [M−H⁺]⁻=409.01.

Additional compounds were prepared following the protocol of Scheme 23. P-0284 was prepared by replacing 4-bromopyrazole with 5-bromo-4-methyl-oxazole in Step 1. P-0285 was prepared by replacing 4-bromopyrazole with 5-bromo-thiazole and replacing 1-bromo-4-trifluoromethyl benzene with 1-bromo-4-chloro-benzene in Step 1. P-0288 was prepared by replacing 4-bromopyrazole with 5-bromo-4-methyl-oxazole and replacing 1-bromo-4-trifluoromethyl benzene with 1-bromo-4-trifluoromethoxy-benzene in Step 1. The compound names, structures and experimental mass spectrometry results are provided in the following Table 12.

TABLE 12

| Cmpd. | | | Molecular weight | |
|---|---|---|---|---|
| number | Structure | Name | Calc. | Measured |
| P-0284 | 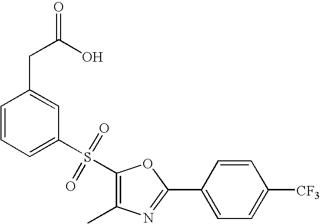 | {3-[4-Methyl-2-(4-trifluoromethyl-phenyl)-oxazole-5-sulfonyl]-phenyl}-acetic acid | 425.38 | MS(ESI)[M + H⁺]⁺ =426.0[M − H⁺]⁻ =424.0 |
| P-0285 | 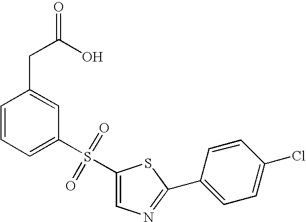 | {3-[2-(4-Chloro-phenyl)-thiazole-5-sulfonyl]-phenyl}-acetic acid | 393.87 | MS(ESI)[M − H⁺]^{-392.0; 394.0} |
| P-0288 | 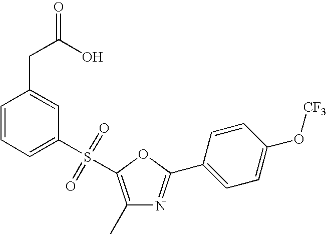 | {3-[4-Methyl-2-(4-trifluoromethoxy-phenyl)-oxazole-5-sulfonyl]-phenyl}-acetic acid | 441.38 | MS(ESI)[M − H⁺]⁻ =440.0 |

### Example 15

### Synthesis of (3-{4-[2-(5-Methyl-2-phenyl-oxazol-4-yl)-ethoxy]-benzenesulfonyl}-phenyl)-acetic acid (P-0289)

Compound P-0289 was synthesized in six steps as shown in Scheme 24.

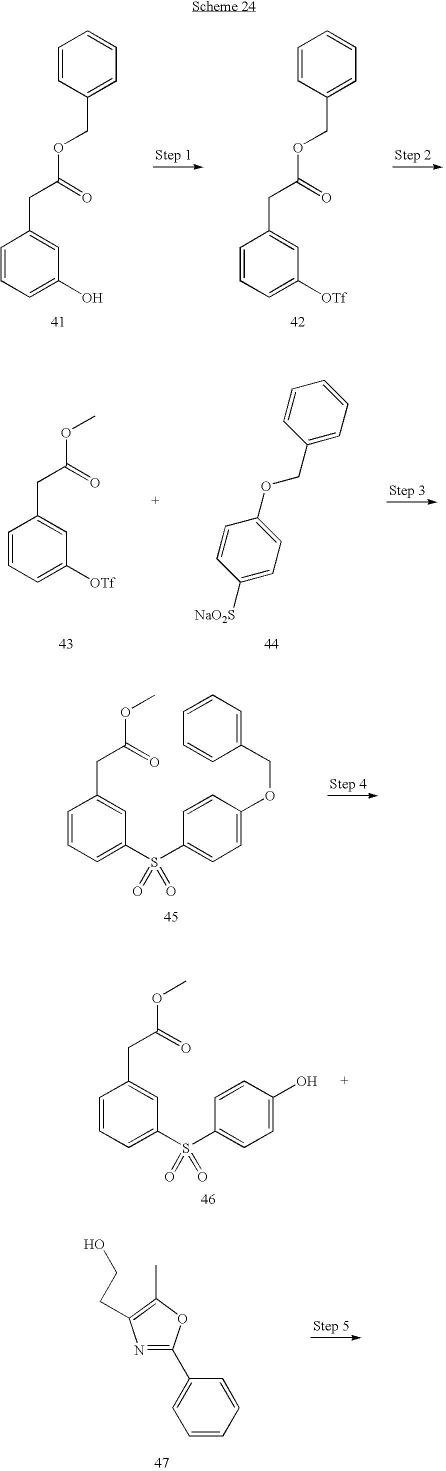

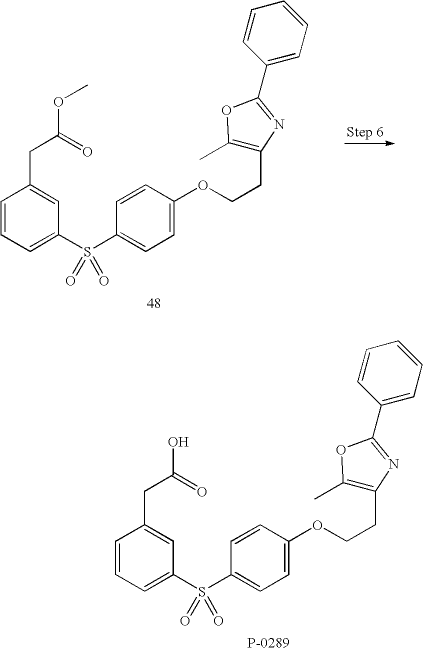

### Step 1: Preparation of (3-trifluoromethanesulfonyloxy-phenyl)-acetic acid benzyl ester (42)

(3-Hydroxy-phenyl)-acetic acid benzyl ester (41, 2000 mg, 0.008 mol) was dissolved in pyridine (9 mL, 0.1 mol). With cooling, trifluoromethanesulfonic anhydride (1.77 mL, 0.0105 mol) was added dropwise to the solution. The mixture was stirred for 30 minutes with cooling, then left stirring overnight at room temperature. The mixture was cooled and water was added followed by diethyl ether. The mixture was acidified to pH1 using 6M HCl. The ether was separated and washed twice with 1M HCl, then brine, dried with sodium sulfate and concentrated to provide an oil. This was used without further purification. ¹H NMR consistent with compound structure.

### Step 2: Preparation of (3-trifluoromethanesulfonyloxy-phenyl)-acetic acid methyl ester (43)

To a solution of (3-trifluoromethanesulfonyloxy-phenyl)-acetic acid benzyl ester (42, 1.13 g, 0.00302 mol) in methanol (4 mL, 0.1 mol) was added sulfuric acid (0.2 mL, 0.004 mol). The mixture was stirred overnight at room temperature. The mixture was concentrated in vacuo. Ethyl acetate and water were added and the layers separated. The organic phase was washed twice with saturated NaHCO₃ and concentrated. ¹H NMR consistent with compound structure.

### Step 3: Preparation of [3-(4-Benzyloxy-benzenesulfonyl)-phenyl]-acetic acid methyl ester (45)

(3-Trifluoromethanesulfonyloxy-phenyl)-acetic acid methyl ester (43, 630 mg, 0.0021 mol) and sodium 4-benzyloxy-benzenesulfinate (44, 856 mg, 0.00317 mol) were placed in a reaction flask in toluene (10 mL, 0.1 mol). Tris(dibenzylideneacetone)dipalladium(0) (190 mg, 0.00021 mol), cesium carbonate (1.0E3 mg, 0.0032 mol), and xanthphos (200 mg, 0.0004 mol) were added. Under an atmosphere of argon, the mixture was heated at 120° C. overnight. After cooling, the reaction mixture was diluted with ethyl acetate, washed with brine, dried over sodium sulfate, concentrated and put on silica. The products were separated on Isco 40 g column (10-30% ethyl acetate in hexanes) and the desired compound was isolated. ¹H NMR consistent with compound structure.

### Step 4: Preparation of [3-(4-hydroxy-benzenesulfonyl)-phenyl]-acetic acid methyl ester (46)

[3-(4-Benzyloxy-benzenesulfonyl)-phenyl]-acetic acid methyl ester (45, 220 mg, 0.00055 mol) was dissolved in tetrahydrofuran (10 mL, 0.1 mol) and 5% Pd/C (5:95, palladium:carbon, 100 mg) was added. This mixture was stirred under an atmosphere of hydrogen at room temp overnight. The catalyst was filtered off and the solvent evaporated, providing the desired compound, used without further purification. ¹H NMR consistent with compound structure.

### Step 5: Preparation of (3-{4-[2-(5-methyl-2-phenyl-oxazol-4-yl)-ethoxy]-benzenesulfonyl}-phenyl)-acetic acid methyl ester (48)

A stirring solution of [3-(4-hydroxy-benzenesulfonyl)-phenyl]-acetic acid methyl ester (46, 100 mg, 0.0003 mol), 2-(5-methyl-2-phenyl-oxazol-4-yl)-ethanol (47, 73.0 mg, 0.000359 mol) and triphenylphosphine (128 mg, 0.000490 mol) in tetrahydrofuran (3 mL, 0.04 mol) was treated with diisopropyl azodicarboxylate (96.4 μL, 0.000490 mol) in 1 mL tetrahydrofuran via dropwise addition. The mixture was stirred overnight at room temperature. Ethyl acetate and water were added and the phases separated, the aqueous phase further extracted with ethyl acetate. The pooled organic phases were washed with brine and dried with sodium sulfate. The reaction material was loaded on silica and purified on Isco Companion 12 g column (10-30% ethyl acetate in hexane) to provide the desired compound. ¹H NMR consistent with compound structure.

### Step 6. Preparation of (3-{4-[2-(5-Methyl-2-phenyl-oxazol-4-yl)-ethoxy]-benzenesulfonyl}-phenyl)-acetic acid (P-0289)

The (3-4-[2-(5-methyl-2-phenyl-oxazol-4-yl)-ethoxy]-benzenesulfonyl-phenyl)-acetic acid methyl ester 48 was hydrolysed using 1 ml KOH (1M) and 3 ml tetrahydrofuran stirring at room temperature overnight. Ethyl acetate was added to the mixture, then acidified using 1M HCl. The organic phase was washed with brine and dried. The desired compound was isolated on Prep. TLC (5% methanol in dichloromethane). ¹H NMR consistent with compound structure. Calculated molecular weight 477.53, MS (ESI) [M+H⁺]⁺=478.1, [M−H⁺]⁻=476.1.

### Example 16

### Preparation of {3-Ethoxy-5-[-5-methyl-4-(4-trifluoromethoxyphenyl) thiophene-2-sulfonyl]-phenyl}-acetic acid (P-0120)

Compound P-0120 was synthesized in five steps as shown in Scheme 25.

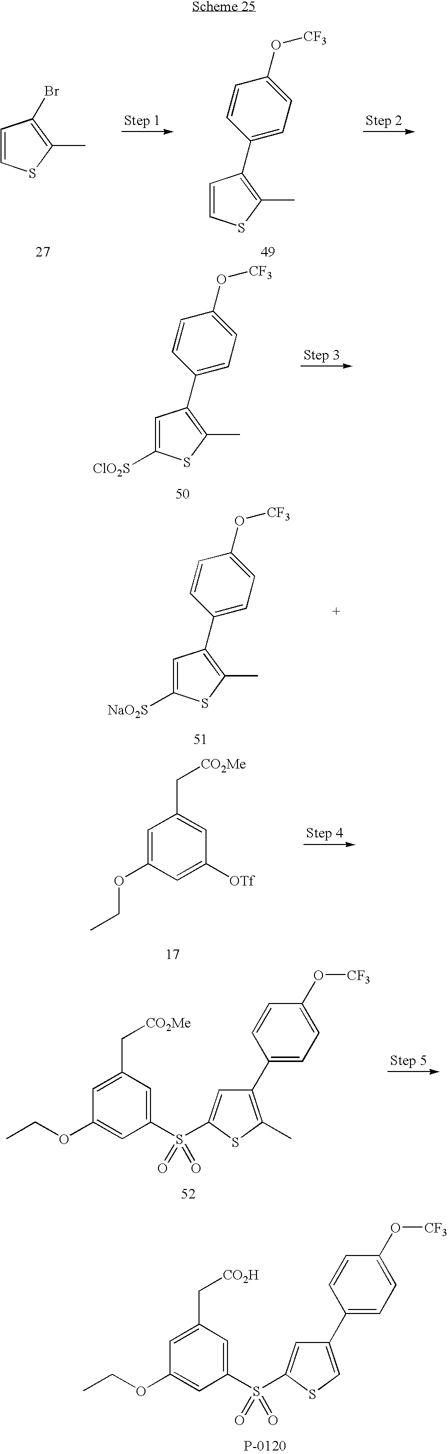

### Step 1: Preparation of 2-methyl-3-(4-trifluoromethoxyphenyl)thiophene (49)

Into a round microwave test tube reaction vessel was added 2-methyl-3-bromothiophene (27, 2.40E2 mg, 0.00136 mol), 4-trifluoromethoxyphenyl boronic acid (590 mg, 0.0028 mol), and 1N K₂CO₃ (0.2 mL) in 1,2-ethanediol (3 mL, 0.05 mol). The vessel was purged with argon for 2-3 minutes, then tetrakis(triphenylphosphine)palladium(0) (4 mg, 0.000003 mol) was added. The reaction was microwaved for 30 minutes at 120° C. TLC analysis (hexane solvent) showed formation of the desired compound. The reaction was adhered onto silica and the desired compound isolated by flash chromatography using straight hexane and used without further purification in the following step. ¹H NMR consistent with compound structure.

### Step 2: Preparation of 5-methyl-4-(4-trifluoromethoxyphenyl)-thiophene-2-sulfonyl chloride (50)

Into an oven dried round bottom flask, chlorosulfonic acid (330 mg, 0.0028 mol) was dissolved in dichloromethane (6 mL, 0.09 mol). The flask was placed on an ice-bath and cooled for 10-15 minutes under a gentle argon flow. Phosphorus pentachloride (340 mg, 0.0016 mol) was added and the reaction stirred vigorously, resulting in violent evolution of gas. The solution was stirred for 20-35 minutes until the solid chunks of pentachloride dissolved. 2-Methyl-3-(4-trifluoromethoxyphenyl)thiophene (49, 350 mg, 0.0014 mol) dissolved in 3 mL dichloromethane was taken up in a syringe and added in one portion to the mixture, resulting in the color of the reaction turning from yellow to dark green over time. The progress of the reaction was monitored by TLC for 3 hours. The reaction was poured into ice and stirred until the ice melted. The reaction was poured into a separatory funnel and extracted with dichloromethane (2×30 mL). The organic layers were washed with water (2×10 mL) and brine (15 mL) and dried over MgSO₄. The solvent was concentrated under reduced pressure and the reaction products absorbed onto silica. The desired compound was isolated by flash chromatography using gradient solvent conditions of 0 to 30% ethyl acetate/hexane over 25 minutes. ¹H NMR (CDCl₃) consistent with compound structure, purity >90%.

### Step 3: Preparation of 5-methyl-4-(4-trifluoromethoxyphenyl)-thiophene-2-sulfinic acid sodium salt (51)

Into a round bottom flask, sodium sulfite (4.0E2 mg, 0.0031 mol) was dissolved in water (15 mL, 0.83 mol). The reaction was placed in an oil bath pre-heated at 98° C. After about 20 minutes, the salt was fully dissolved and a combined mixture of sodium bicarbonate (99 mg, 0.0012 mol) and 5-methyl-4-(4-trifluoromethoxyphenyl)-thiophene-2-sulfonyl chloride (50, 3.50E2 mg, 0.000981 mol) were added in one portion. The progress of the reaction was monitored every hour by TLC (20% ethyl acetate/hexane). The reaction was heated overnight, after which TLC indicated absence of starting material. The reaction vessel was cooled to room temperature and the contents frozen in an acetone-dry ice bath. Water was removed overnight by lyophilization. The sulfinic acid salt was dissolved in ethanol (40 mL) and heated at 98° C. for 30 minutes, then hot filtered. The white salt residue was rinsed generously with hot ethanol (40 mL). The collected filtrate was roto evaporated to give the desired compound as a white gummy solid, which was used without further purification. ¹H NMR (CD₃OD) consistent with compound structure.

### Step 4: Preparation of {3-ethoxy-5-[-5-methyl-4-(4-trifluoromethoxyphenyl) thiophene-2-sulfonyl]-phenyl}-acetic acid methyl ester (52)

Into a flame dried 40 mL vial, (3-ethoxy-5-trifluoromethanesulfonyloxy-phenyl)-acetic acid methyl ester (17, 116 mg, 0.000339 mol, prepared as in Step 2 of Scheme 15, Example 6), 5-methyl-4-(4-trifluoromethoxyphenyl)-thiophene-2-sulfinic acid sodium salt (51, 195 mg, 0.000566 mol), cesium carbonate (289 mg, 0.000887 mol) and xanthphos (8 mg, 0.00001 mol) were added with toluene (5 mg, 0.00005 mol). The vessel was then flushed with argon and tris(dibenzylideneacetone)dipalladium(0) (8 mg, 0.000009 mol) quickly added. The reaction was stirred under an atmosphere of argon for 3-4 minutes more. After this time the reaction vessel was transferred to a heating block pre-set at 117° C. and heated overnight. The vial was cooled to room temperature and TLC (20% ethyl acetate/hexane) indicated absence of starting material. The crude reaction mixture was transferred to a flask and the solvent removed under reduced pressure. This was diluted with ethyl acetate (60 mL) and water (30 mL). The organic layer was separated and the aqueous layer washed with ethyl acetate (2×40 mL). The organic fractions were combined, washed with brine, dried over MgSO₄ and filtered. The filtrate was concentrated and adhered onto silica for chromatography. The desired compound was isolated by flash chromatography using gradient solvent conditions of 0 to 35% ethyl acetate/hexane in 40 minutes and taken on to the following step. ¹H NMR consistent with compound structure.

### Step 5: Preparation of {3-ethoxy-5-[-5-methyl-4-(4-trifluoromethoxyphenyl) thiophene-2-sulfonyl]-phenyl}-acetic acid (P-0120)

The methyl ester 52 was dissolved in a 5 mL mixture of tetrahydrofuran/1N LiOH (4:1) and stirred vigorously overnight. The reaction was acidified by adding 1N HCl (pH 0-1 by pH paper), extracted with ethyl acetate (3 times the reaction volume) and dried over MgSO₄. The desired compound was isolated by flash chromatography using 2% methanol/chloroform. ¹H NMR (CDCl₃) consistent with structure, purity >96%.

Additional compounds were prepared following the protocol of Scheme 25. P-0121 was prepared by replacing (3-ethoxy-5-trifluoromethanesulfonyloxy-phenyl)-acetic acid methyl ester 17 with (3-propoxy-5-trifluoromethanesulfonyloxy-phenyl)-acetic acid methyl ester (prepared as in Step 2 of Scheme 15, Example 6 by replacing iodoethane with 1-iodopropane in Step 1) in Step 4. P-0092 was also prepared using (3-propoxy-5-trifluoromethanesulfonyloxy-phenyl)-acetic acid methyl ester in Step 4, further replacing the 4-trifluoromethoxyphenyl boronic acid with 4-trifluoromethylphenyl boronic acid in Step 1. The compound structures, names and mass spectrometry results for these compounds are provided in the following Table 13.

TABLE 13

| Cmpd. | | | Molecular weight | |
|---|---|---|---|---|
| number | Structure | Name | Calc. | Measured |
| P-0092 | 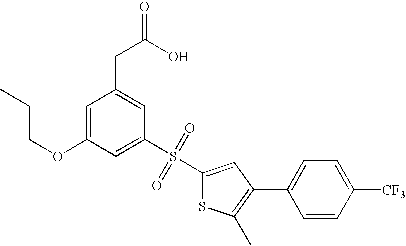 | {3-[5-Methyl-4-(4-trifluoromethyl-phenyl)-thiophene-2-sulfonyl]-5-propoxy-phenyl}-acetic acid | 498.54 | n/a |
| P-0121 | 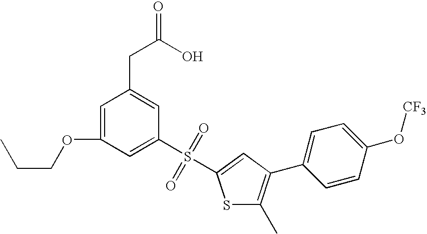 | {3-[5-Methyl-4-(4-trifluoromethoxy-phenyl)-thiophene-2-sulfonyl]-5-propoxy-phenyl}-acetic acid | 514.54 | n/a |

### Example 17

### Preparation of {3-Ethoxy-5-[2-methyl-5-(4-trifluoromethylphenyl) thiophene-3-sulfonyl]-phenyl}-acetic acid (P-0283)

Compound P-0283 was synthesized in five steps as shown in Scheme 26.

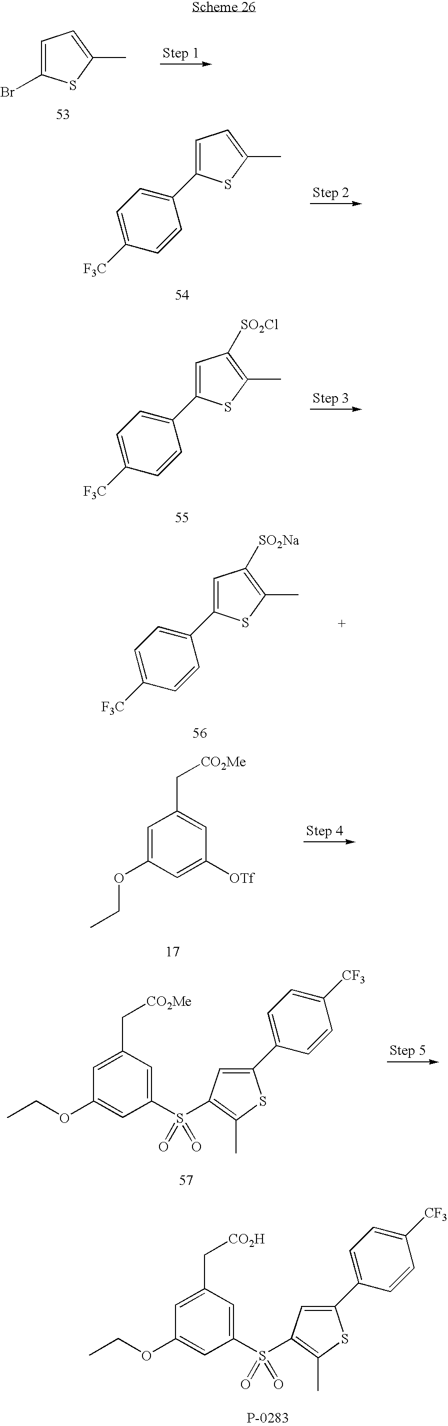

### Step 1: Preparation of 2-methyl-5-(4-trifluoromethylphenyl)-thiophene (54)

Into a microwave tube, 2-bromo-5-methyl-thiophene (53, 400 mg, 0.002 mol), 4-(trifluoromethyl)phenylboronic acid (640 mg, 0.0034 mol), and 1N K₂CO₃ were combined in tetrahydrofuran (3 mL, 0.04 mol). The vessel was purged with argon, then tetrakis(triphenylphosphine)palladium(0) (10 mg, 0.000009 mol) was quickly added. The reaction vessel was placed in a microwave chamber and heated at 110° C. for 30 minutes, after which TLC analysis (hexane) still showed starting material and a fluorescent spot near the starting material. The solvent was partially removed, and the crude reaction mixture was absorbed onto silica. The desired compound was isolated by flash chromatography using 100% hexane and used in the next step. ¹H NMR consistent with compound structure.

### Step 2: Preparation of 2-methyl-5-(4-trifluoromethylphenyl)thiophene-2-sulfonyl chloride (55)

A round bottom flask, flame dried and under an inert conditions, was placed on an ice bath and chlorosulfonic acid (250 mg, 0.0021 mol) and dry dichloromethane (6 mL, 0.09 mol) were combined. The reaction flask was purged with argon and stirred for 10-15 minutes, after which phosphorus pentachloride (210 mg, 0.00099 mol) was added and the reaction stirred until the solid phosphorous dissolved. 2-methyl-5-(4-trifluoromethylphenyl)-thiophene (54, 200 mg, 0.0008 mol) dissolved in 5 mL dichloromethane was slowly added to the stirring reaction. After the final addition, the reaction was left stirring under an atmosphere of argon for 4 hours. TLC analysis (5% ethyl acetate/hexane) indicated near disappearance of starting material and an emergence of two new spots at slower R_{f}. The reaction was poured into ice and stirred. After the ice melted, the organic phase was extracted with 30 mL dichloromethane, washed with brine (2×) and dried over MgSO₄ and filtered. The solvent was evaporated to half of its original volume and silica was added, then the solvent was removed under vacuum. The desired compound was isolated by flash chromatography with gradient solvent condition of 0 to 5% ethyl acetate/hexane over 18 minutes, then 5 to 20% ethyl acetate over 5 minutes and taken to the next step. ¹H NMR consistent with compound structure. Calculated molecular weight 322.32, MS (ESI) [M−H⁺]⁻=321.33.

### Step 3: Preparation of 2-methyl-5-(4-trifluoromethylphenyl)-thiophene-3-sulfinic acid sodium salt (56)

Into a round bottom flask, sodium sulfite (100 mg, 0.0008 mol) was dissolved in water (9 mL, 0.5 mol). The reaction flask was heated at 98° C. for 30 minutes until the solid fully dissolved. Sodium bicarbonate (33 mg, 0.00039 mol) and 2-methyl-5-(4-trifluoromethylphenyl)thiophene-2-sulfonyl chloride (55, 112 mg, 0.000329 mol) were added simultaneously to the reaction and the reaction heated overnight with a condenser attachment. After 16 hours, TLC analysis (20% ethyl acetate/hexane) indicated absence of starting material. The reaction was cooled to room temperature and the solvent was removed by lyophilization. The resulting solid was dissolved in 30 mL ethanol, the vessel refluxed for 30 minutes, and the mixture was hot filtered. The salt was collected and re-dissolved in ethanol and the above process repeated. The filtrates were collected and evaporated under reduced pressure to give the desired sulfinic acid salt. ¹H NMR (CD₃OD) consistent with compound structure. Calculated molecular weight 306.00, MS (ESI) [M−H⁺]⁻=305.01.

### Step 4: Preparation of {3-ethoxy-5-[2-methyl-5-(4-trifluoromethyl phenyl)thiophene-3-sulfonyl]-phenyl}-acetic acid methyl ester (57)

Into a flame dried vial, (3-ethoxy-5-trifluoromethanesulfonyloxy-phenyl)-acetic acid methyl ester (17, 102 mg, 0.000298 mol, prepared as in Step 2 of Scheme 15, Example 6), 2-methyl-5-(4-trifluoromethylphenyl)-thiophene-3-sulfinic acid sodium salt (56, 75 mg, 0.00023 mol), xanthphos (6 mg, 0.00001 mol), cesium carbonate (150 mg, 0.00046 mol), and tris(dibenzylideneacetone)dipalladium(0) (5 mg, 0.000005 mol) were combined in toluene (6 mL, 0.06 mol). The vial was purged with argon for 2-3 minutes and the reaction placed on an oil bath pre-heated at 117° C. for 5 hours. TLC analysis using 10% ethyl acetate/hexane showed the desired compound. The vial was cooled to room temperature and the solvent roto evaporated to dryness. The crude mixture was extracted with ethyl acetate (3×30 mL) and water (20 mL) and the organic layer was isolated, washed with brine, dried over MgSO₄ and filtered. The solvent was evaporated under reduced pressure. The resulting solid was re-dissolved in a minimal amount of ethyl acetate and this was placed onto a silica plate. The desired compound was isolated by plate chromatography eluting with 10% ethyl acetate/hexane solvent. ¹H NMR consistent with compound structure.

### Step 5: Preparation of {3-ethoxy-5-[2-methyl-5-(4-trifluoromethyl phenyl thiophene-3-sulfonyl]-phenyl}-acetic acid (P-0283)

The methyl ester 57 was dissolved in a 5 mL mixture of tetrahydrofuran/1N LiOH (4:1) and stirred vigorously overnight, after which TLC (20% ethyl acetate/hexane) indicated absence of starting material and a new spot around the baseline. The reaction was acidified by adding 1N HCl (pH 0-1 by pH paper), extracted with ethyl acetate (3 times the reaction volume) and dried over MgSO₄. The desired compound was isolated by flash chromatography using a gradient solvent condition of 0 to 3% methanol/dichloromethane over 25 minutes. ¹H NMR (CDCl₃) consistent with compound structure, purity >96%.

### Example 18

### Preparation of {3-Propoxy-5-[3-(4-trifluoromethoxyphenyl)-thiophene-2-sulfonyl]-phenyl}-acetic acid (P-0279)

Compound P-0279 was synthesized in five steps as shown in Scheme 27.

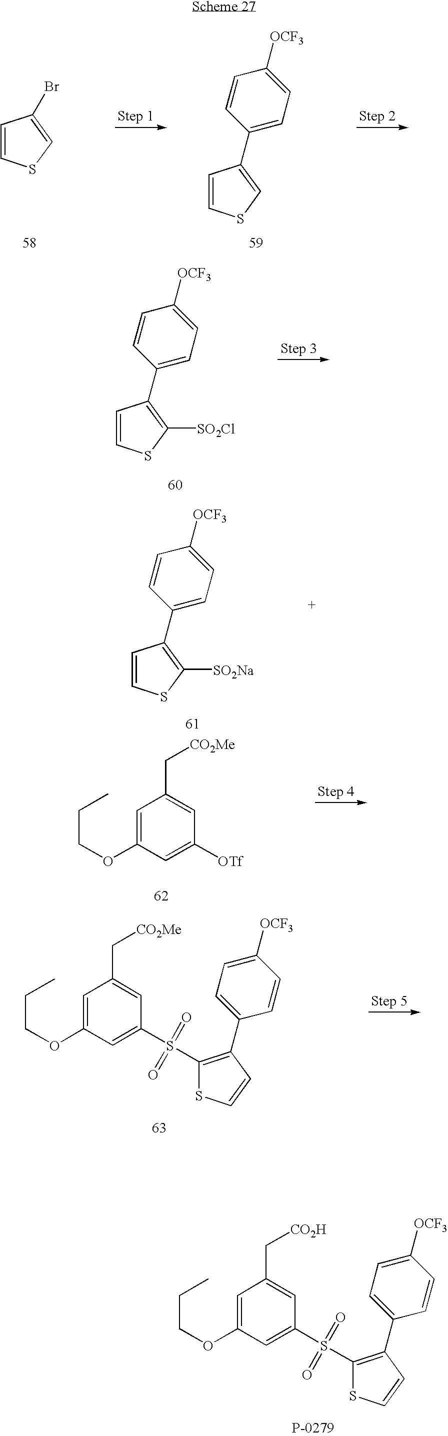

### Step 1: Preparation of 3-(4-trifluoromethoxyphenyl)-thiophene (59)

Into a 40 mL reaction vessel, 3-bromo-thiophene (58, 4.50E2 mg, 0.00276 mol), 4-trifluoromethoxyphenyl boronic acid (683 mg, 0.00332 mol), 1N K₂CO₃ (0.4 mL), tetra-n-butylammonium iodide (4 mg, 0.00001 mol) and tetrahydrofuran (8 mL, 0.1 mol) were combined. The mixture was stirred under an atmosphere of argon for 2-5 minutes, then tetrakis(triphenylphosphine)palladium(0) (8 mg, 0.000007 mol) was added. The vessel was placed on an oil bath preheated at 87° C. and stirred for 2 days. TLC analysis (hexane) showed the presence of starting material and two slower moving spots. The reaction was filtered and concentrated under reduced pressure. The crude reaction mixture was absorbed onto silica and the desired compound isolated by flash chromatography eluting with 100% hexane, which was used in the next step without further purification. ¹H NMR consistent with compound structure.

### Step 2: Preparation of 3-(4-trifluoromethoxyphenyl)thiophene-2-sulfonyl chloride (60)

Into a flame dried round bottom flask under an inert condition, chlorosulfonic acid (480 mg, 0.0042 mol) was dissolved in dichloromethane (5 mL, 0.08 mol). The flask was transferred into an ice bath under an argon flow and phosphorus pentachloride (340 mg, 0.0016 mol) added. The mixture was stirred until the solid dissolved. 3-(4-trifluoromethoxyphenyl)-thiophene (59, 328 mg, 0.00134 mol) was dissolved in 4 mL dichloromethane and added to the cold pentachloride-chlorosulfonic acid mixture. The reaction was stirred overnight under an inert atmosphere, after which TLC analysis (hexane) indicated absence of starting material, with new spots appearing with solvent condition of 20% ethyl acetate/hexane. The reaction mixture was poured into ice and extracted with dichloromethane (2×20 mL). The isolated organic was washed with brine (3×20 mL) and dried with MgSO₄. The crude mixture was filtered, the solvent was evaporated under reduced pressure, and the crude compound absorbed onto silica and purified by flash chromatography with a gradient of 0 to 25% ethyl acetate/hexane over 20 minutes. ¹H NMR consistent with compound structure with desired 2,3-substitution pattern.

### Step 3: Preparation of 3-(4-trifluoromethoxyphenyl)-thiophene-2-sulfinic acid sodium salt (61)

Into a round bottom flask, sodium sulfite (220 mg, 0.0017 mol) was dissolved in water (15 mL, 0.83 mol) and heated at 107° C. for 10-12 minutes. Solid gradually went into the solution. 3-(4-Trifluoromethoxyphenyl)thiophene-2-sulfonyl chloride (60, 223 mg, 0.000651 mol) and sodium bicarbonate (62 mg, 0.00074 mol) were mixed on weighing paper and the combined solids were added to the refluxing solution. After 4 hours, TLC analysis (20% ethyl acetate/hexane) indicated the presence of starting material. A nitrogen balloon was attached to the reaction flask and the reaction refluxed overnight, after which TLC indicated the absence of starting material. The reaction was cooled to room temperature and the solvent frozen using acetone-dry ice bath and the solvent was removed by lyophilization. After 16 hours, the solid salt was combined with 40 mL ethanol, refluxed for 40 minutes and filtered. The collected solid was re-dissolved in ethanol and the process repeated. The filtrates were combined and solvent was removed in vacuo to give the desired sulfinic acid salt. The white powder was carried on to the next step.

### Step 4: Preparation of {3-propoxy-5-[3-(4-trifluoromethoxyphenyl)-thiophene-2-sulfonyl-phenyl}-acetic acid methyl ester (63)

Into a flame dried round bottom flask under an inert condition, (3-propoxy-5-trifluoromethanesulfonyloxy-phenyl)-acetic acid methyl ester (62, 106 mg, 0.000298 mol, prepared as in Step 2 of Scheme 15, Example 6 by replacing iodoethane with 1-iodopropane in Step 1), 3-(4-trifluoromethoxyphenyl)-thiophene-2-sulfinic acid sodium salt (61, 221 mg, 0.000669 mol), cesium carbonate (295 mg, 0.000905 mol), xanthphos (10 mg, 0.00002 mol), tris(dibenzylideneacetone)dipalladium(0) (10 mg, 0.00001 mol), and toluene (15 mL, 0.14 mol) were combined. The reaction vessel was purged with argon for 5 minutes and heated at 117° C. for 5 hours, after which TLC (20% ethyl acetate/hexane) indicated the absence of starting material and multiple new spots. The solvent was evaporated under reduced pressure and the crude reaction mixture was introduced onto a prep silica plate. The desired compound was isolated by plate chromatography using 20% ethyl acetate/hexane. ¹H NMR consistent with compound structure.

### Step 5: Preparation of {3-propoxy-5-[3-(4-trifluoromethoxyphenyl)-thiophene-2-sulfonyl]-phenyl}-acetic acid (P-0279)

The methyl ester 63 was dissolved in a 5 mL mixture of tetrahydrofuran/1N LiOH (4:1) and stirred vigorously overnight, after which TLC (20% ethyl acetate/hexane) indicated the absence of starting material and a new spot around the baseline. The reaction was acidified by adding 1N HCl (pH 0-1 by pH paper), extracted with ethyl acetate (3 times the reaction volume) and dried over MgSO₄. The desired compound was isolated by flash chromatography using gradient solvent conditions of 0 to 3% methanol/dichloromethane over 25 minutes. ¹H NMR (CDCl₃) consistent with compound structure, purity >96%.

### Example 19

### Preparation of {3-ethoxy-5-[4-(4-trifluoromethylphenyl)thiophene-2-sulfonyl]-phenyl}-acetic acid (P-0278)

Compound P-0278 was synthesized in five steps as shown in Scheme 28.

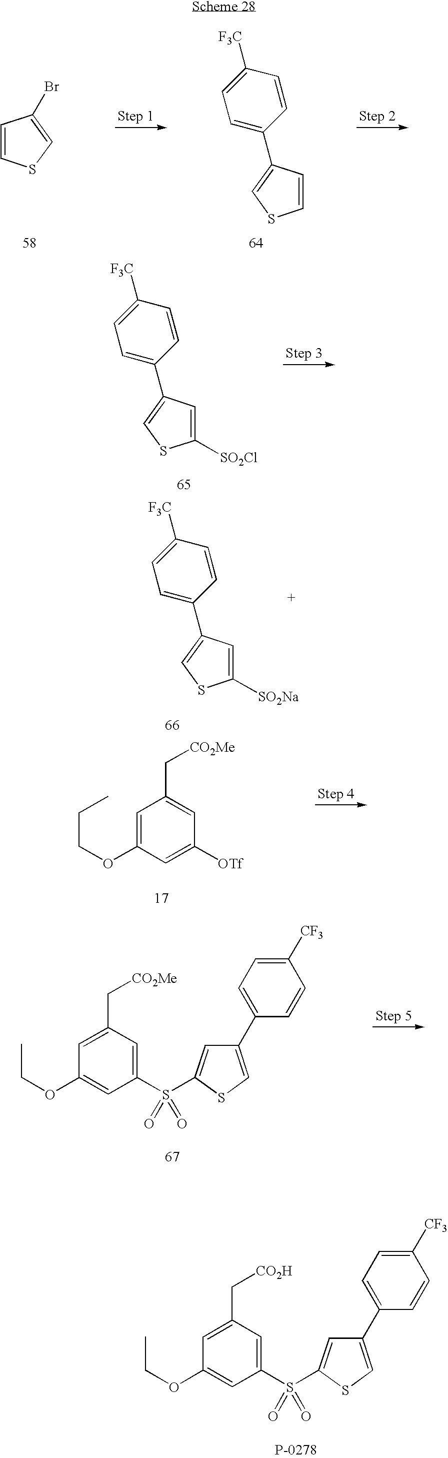

### Step 1: Preparation of 3-(4-trifluoromethylphenyl)-thiophene (64)

Into a 40 mL reaction vessel, 3-bromo-thiophene (58, 4.50E2 mg, 0.00276 mol), 4-(trifluoromethyl)phenylboronic acid (6.30E2 mg, 0.00332 mol), 1N K₂CO₃ (0.4 mL), tetra-n-butylammonium iodide (4 mg, 0.00001 mol) and tetrahydrofuran (8 mL, 0.1 mol) were combined. The mixture was stirred under an atmosphere of argon for 2-5 minutes, then tetrakis(triphenylphosphine)palladium(0) (8 mg, 0.000007 mol) was added. The vessel was placed on an oil bath preheated at 87° C. and stirred for 2 days, after which TLC analysis (hexane) showed the presence of starting material and two slower moving spots. The reaction was filtered and solvent concentrated down with silica. The desired compound was isolated by flash chromatography eluting with hexane and carried on to the next step. ¹H NMR consistent with compound structure.

### Step 2: Preparation of 4-(4-trifluoromethylphenyl)-thiophene-2-sulfonyl chloride (65)

Into a flame dried round bottom flask, chlorosulfonic acid (480 mg, 0.0042 mol) was dissolved in dichloromethane (8 mL, 0.1 mol) under an atmosphere of argon. The vessel was placed on an ice bath and stirred for 4-5 minutes. Phosphorus pentachloride (340 mg, 0.0016 mol) was slowly added over 2 minutes and the reaction stirred until the solid dissolved, after which 3-(4-trifluoromethylphenyl)-thiophene (64, 306 mg, 0.00134 mol) dissolved in 3 mL dichloromethane was added. The reaction was stirred overnight under a nitrogen balloon. After 16 hours, TLC analysis (hexane) showed the absence of starting material, while 20% ethyl acetate/hexane elution indicated three new spots. The reaction was slowly poured into a beaker filled with ice and stirred until the ice melted. This was extracted with 30 mL dichloromethane, which was subsequently washed twice with brine (10 mL), waiting for the emulsion layer to dissipate after addition of salt. The organic layer was collected and dried thoroughly with MgSO₄, which was then roto evaporated to half of its original volume. Silica was added to the mixture and the solvent removed. The desired compound was isolated by flash chromatography using a gradient of 0 to 25% ethyl acetate/hexane over 25 minutes, which was carried on to the next step. ¹H NMR consistent with compound structure.

### Step 3: Preparation of 4-(4-trifluoromethylphenyl)-thiophene-2-sulfinic acid sodium salt (66)

Into a round bottom flask, sodium sulfite (240 mg, 0.0019 mol) was dissolved in water. The reaction vessel was placed on an oil bath pre-heated at 102° C. and heated for 20-23 minutes. 4-(4-trifluoromethylphenyl)-thiophene-2-sulfonyl chloride (65, 250 mg, 0.00076 mol) and sodium bicarbonate (77 mg, 0.00092 mol) were mixed on weighing paper and slowly added to the reaction. The reaction was heated overnight, after which TLC (20% ethyl acetate/hexane) indicated the absence of starting material. The reaction was cooled to room temperature and the solvent frozen using acetone-dry ice bath. The solvent was removed via lyophilization. The crude white solid was combined with ethanol and refluxed for 20 minutes, then hot filtered and the salt was rinsed generously with hot ethanol. The filtrate was collected and evaporated under reduced pressure to give the desired sulfinic acid salt. ¹H NMR consistent with compound structure. Calculated molecular weight 291.98, MS (ESI) [M−H⁺]⁻=291.21.

### Step 4: Preparation of {3-ethoxy-5-[4-(4-trifluoromethylphenyl)thiophene-2-sulfonyl]-phenyl}-acetic acid methyl ester (67)

Into a flame dried scintillation vial, (3-ethoxy-5-trifluoromethanesulfonyloxy-phenyl)-acetic acid methyl ester (17, 102 mg, 0.000298 mol, prepared as in Step 2 of Scheme 15, Example 6), 4-(4-trifluoromethyl phenyl)-thiophene-2-sulfinic acid sodium salt (66, 112 mg, 0.000356 mol), and cesium carbonate (210 mg, 0.00064 mol) were dissolved in toluene (4 mL, 0.04 mol). The mixture was purged with argon for a few minutes, then xanthphos (5 mg, 0.000009 mol) and tris(dibenzylideneacetone)dipalladium(0) (5 mg, 0.000005 mol) were added. The vial was capped and the mixture heated at 117° C. for 5 hours, after which TLC analysis (20% ethyl acetate/hexane) indicated a trace of starting material and a new spot (fluorescent) running below the starting material. The reaction was cooled to room temperature and the solvent evaporated under reduced pressure. The crude mixture absorbed onto silica. The desired compound was isolated by flash chromatography using a gradient of 0 to 20% ethyl acetate/hexane over 25 minutes. ¹H NMR consistent with compound structure.

### Step 5: Preparation of {3-Ethoxy-5-[4-(4-trifluoromethylphenyl)thiophene-2-sulfonyl]-phenyl}-acetic acid (P-0278)

The methyl ester 67 was dissolved in a 4 mL mixture of tetrahydrofuran/1N LiOH (4:1) and stirred vigorously for 3 hours, after which TLC analysis (20% ethyl acetate/hexane) indicated the absence of starting material and a new spot around the baseline. The reaction was acidified by adding 1N HCl (pH 0-1 by pH paper), extracted with ethyl acetate (3 times the reaction volume) and dried over MgSO₄. The desired compound was isolated by flash chromatography with a gradient of 0 to 3% methanol/dichloromethane. ¹H NMR (CDCl₃) consistent with compound structure, purity >96%. Calculated molecular weight 470.49, MS (ESI) [M−H⁺]⁻=468.24.

### Example 20

### Synthesis of {3-ethoxy-5-[4-(4-trifluoromethyl-phenoxy)benzenesulfonyl]-phenyl}acetic acid (P-0029)

Compound P-0029 was synthesized in four steps as follows.

### Step 1: Preparation of methyl 2-(3-ethoxy-5-hydroxyphenyl)acetate

Into a 500 mL 3-necked flask, equipped with a thermometer, a stopper, a nitrogen inlet adapter, and a magnetic stir bar, was placed methyl 2-(3,5-dihydroxyphenyl)acetate (5.33 g, 29.3 mmol) and N,N-dimethylformamide (100 mL). The reaction mixture was placed under nitrogen and cooled to an internal temperature of −50° C. At this time sodium hydride (60% dispersion in mineral oil, 2.34 g, 58.5 mmol) was added in four portions over a period of 15 minutes, during which time the internal temperature increased to −22° C. The resulting slurry was stirred at room temperature for 40 minutes. The clear, green reaction mixture was once again cooled to an internal temperature of −50° C., and iodoethane (2.36 mL, 29.2 mmol) was added all at once. The reaction mixture was then placed in a −24° C. bath. Within 20 minutes the internal temperature increased from −57° C. to −24° C. The internal temperature was kept at −24° C. to −14° C. for 75 min, then allowed to warm to +11° C. over a period of 95 minutes. The reaction mixture was quenched with formic acid (15 mL) and stirred at room temperature for 20 minutes. The resulting slurry was filtered, rinsed sparingly with ethyl acetate, and concentrated under reduced pressure to give a viscous orange oil, which was loaded onto a Silica Gel plug. Elution with 20% ethyl acetate in hexanes, then 30% ethyl acetate in hexanes gave an oil, identified by ¹H NMR as methyl 2-(3-ethoxy-5-hydroxyphenyl)acetate (2.84 g, 46%). ¹H NMR (CDCl₃): δ6.38 (s, 1H), 6.33 (s, 1H), 6.29 (s, 1H), 3.97 (q, J=7 Hz, 2H), 3.68 (s, 3H), 3.50 (s, 2H), 1.37 (t, J=7 Hz, 3H).

### Step 2: Preparation of methyl 2-(3-ethoxy-5-(trifluoromethylsulfonyloxy)phenyl)acetate

Into a 1 L round bottom flask equipped with an addition funnel and nitrogen inlet adapter was added methyl 2-(3-ethoxy-5-hydroxyphenyl)acetate (2.1 g, 9.99 mmol) and dichloromethane (19.98 ml). The reaction mixture was cooled in a −78° C. bath under nitrogen. N,N-diisopropylethylamine (2.44 ml, 13.98 mmol) was added, followed by the dropwise addition of trifluoromethanesulfonic anhydride (2.02 ml, 11.99 mmol) in dichloromethane (10 mL) over a period of 6 minutes. The pale yellow slurry was stirred in the −78° C. bath. After 40 minutes the reaction mixture was poured into water (100 mL) and dichloromethane (100 mL) and extracted. The milky dichloromethane layer was loaded onto a Silica Gel plug and eluted with dichloromethane until 500 mL was collected. The dichloromethane layer was concentrated under reduced pressure to obtain 3.12 g (91%) of a colorless oil, identified by ¹H NMR as methyl 2-(3-ethoxy-5-(trifluoromethylsulfonyloxy)phenyl)acetate. ¹H NMR (DMSO-d6): δ6.94 (m, 1H), 6.92 (m, 2H), 4.02 (q, J=7 Hz, 2H), 3.72 (s, 2H), 3.58 (s, 3H), 1.28 (t, J=7 Hz, 3H).

### Step 3: Preparation of methyl 2-(3-ethoxy-5-(4-(4-(trifluoromethyl)phenoxy)phenylsulfonyl)phenyl)acetate

Methyl 2-(3-ethoxy-5-(trifluoromethylsulfonyloxy)phenyl)acetate (3.48 g, 10.17 mmol) was reacted in two portions as follows: Methyl 2-(3-ethoxy-5-(trifluoromethylsulfonyloxy)phenyl)acetate (1.74 g, 5.08 mmol), Cs₂CO₃ (2.49 g, 7.64 mmol), sodium 4-(4-(trifluoromethyl)phenoxy)benzenesulfinate dihydrate (2.09 g, 5.80 mmol), tris(dibenxylideneacetone)dipalladium(0) (0.465 g, 0.5 mmol), 4,5-bis(diphenylphosphino)-9,9-dimethylxanthene (0.588 g, 1.0 mmol), and dioxane (26 mL) were mixed in an 80 mL vessel and stirred well. Microwave irradiation in CEM (Matthews, N C) Discover (300 watt) was done at 160° C. with a 5 minute hold. The combined runs were poured onto the same Celite pad and rinsed 3-4 times with dichloromethane. Concentration under reduced pressure at 40° C. gave an orange oil (8.33 g), which was purified by chromatography on silica gel with 20% ethyl acetate in hexanes to yield 3.05 g (60.6%) of methyl 2-(3-ethoxy-5-(4-(4-(trifluoromethyl)phenoxy)phenylsulfonyl)phenyl)acetate. ¹H NMR (DMSO-d6): δ7.97 (d, J=9 Hz, 2H), 7.76 (d, J=8.5 Hz, 2H), 7.41 (br s, 1H), 7.28-7.26 (m, 3H), 7.21 (d, J=9 Hz, 2H), 7.11 (br s, 1H), 4.05 (q, J=7 Hz, 2H), 3.75 (s, 2H), 3.57 (s, 3H), 1.28 (t, J=7 Hz, 3H).

### Step 4: Preparation of {3-ethoxy-5-[4-(4-trifluoromethyl-phenoxy)benzenesulfonyl]-phenyl}acetic acid (P-0029)

Into a 2 L round bottomed flask was mixed methyl 2-(3-ethoxy-5-(4-(4-(trifluoromethyl)phenoxy)phenylsulfonyl)phenyl)acetate (29.9 g, 60.4 mmol) and tetrahydrofuran (201 ml). 1N potassium hydroxide (72.4 ml, 72.4 mmol) was added dropwise over 5 minutes, followed by the addition of methanol until the reaction mixture became homogeneous (˜75 mL). The solution was stirred at room temperature for 2 hours, then concentrated under reduced pressure until all traces of methanol were removed. The resulting pale brown solid was partitioned between 2N HCl (350 mL) and ethyl acetate (1.3 L), and extracted well. The ethyl acetate layer was separated and dried (Na₂SO₄). Concentration under reduced pressure gave a foam (26.76 g, 91%), which was recrystallized from 1:1 toluene:hexane. The resulting solid was dried in a vacuum oven at room temperature overnight to yield 22.5 g (84%) of {3-ethoxy-5-[4-(4-trifluoromethyl-phenoxy)benzenesulfonyl]-phenyl}acetic acid (P-0029). ¹H NMR (DMSO-d6): δ12.43 (br s, 1H), 7.97 (d, J=8.9 Hz, 2H), 7.75 (d, J=8.5 Hz, 2H), 7.4 (br s, 1H), 7.28-7.26 (m, 3H), 7.20 (d, J=8.9 Hz, 2H), 7.10 (br s, 1H), 4.05 (q, J=7 Hz, 2H), 3.63 (s, 2H), 1.28 (t, J=7 Hz, 3H).

### Example 21

### Expression and Purification of PPARs for Use in Biochemical and Cell Assays

Genetic Engineering

Plasmids encoding the Ligand-binding domains (LBDs) of PPARα, PPARγ, and PPARδ were engineered using common polymerase chain reaction (PCR) methods (pGal4-PPARα-LBD, pGal4-PPARγ-LBD, pGal4-PPARδ-LBD). The relevant DNA sequences and encoded protein sequences used in the assay are shown for each (see below). Complementary DNA cloned from various human tissues were purchased from Invitrogen, and these were used as substrates in the PCR reactions. Specific custom synthetic oligonucleotide primers (Invitrogen, see below) were designed to initiate the PCR product, and also to provide the appropriate restriction enzyme cleavage sites for ligation with the plasmids.

The plasmids used for ligation with the receptor-encoding inserts were either pET28 (Novagen) or a derivative of pET28, pET-BAM6, for expression using E. coli. In each of these cases the receptor LBD was engineered to include a Histidine tag for purification using metal affinity chromatography.

Protein Expression and Purification of PPAR's.

For protein expression, plasmids containing genes of interest were transformed into E. coli strain BL21 (DE3)RIL (Invitrogen) and transformants selected for growth on LB agar plates containing appropriate antibiotics. Single colonies were grown for 4 hrs at 37° C. in 200 ml LB media. For PPARα and PPARγ all protein expression was performed by large scale fermentation using a 30 L bioreactor. 400 ml of starter culture was added to 30 L TB culture and allowed to grow at 37° C. until an OD600 nm of 2-5 was obtained. The culture was cooled to 20° C. and 0.5 mM IPTG added, the culture was allowed to grow for a further 18 hrs.

For PPARδ protein expression, single colonies were grown for 4 hrs at 37° C. in 200 ml LB media. 16×1 L of fresh TB media in 2.8 L flasks were inoculated with 10 ml of starter culture and grown with constant shaking at 37° C. Once cultures reached an absorbance of 1.0 at 600 nm, an additive to improve the solubility of the PPARδ was added to the culture and 30 min later, 0.5 mM IPTG was added and cultures allowed to grow for a further 12 to 18 hrs at 20° C. Cells were harvested by centrifugation and pellets frozen at −80° C. until ready for lysis/purification.

For protein purification; all operations were carried out at 4° C. Frozen E. coli cell pellets were resuspended in lysis buffer and lysed using standard mechanical methods. Soluble proteins were purified via poly-Histidine tags using immobilized metal affinity purification (IMAC). For each of the PPAR's described all have been purified using a 3 step purification process utilizing IMAC, size exclusion chromatography and ion exchange chromatography. For PPARα the poly-Histidine tag was optionally removed using Thrombin (Calbiochem). In the case of PPARδ, during protein purification the solubility improving additive was present in order to maintain protein stability. During the final step of purification solubility improving additives were desalted away before concentration.

| Plasmid sequence and PCR primer information: | | | |
|---|---|---|---|
| PPARα (Nucleic acid SEQ ID NO:_{——}) (Protein SEQ ID NO:_{——}) | | | |
| P332. pET28 PPARA E199-Y468-X | | | |
| taatacgactcactataggggaattgt | | | |
| gagcggataacaattcccctctagaaataattttgtttaactttaagaaggagatatacc | | | |
| atgggcagcagccatcatcatcatcatcacagcagcggcctggtgccgcgcggcagccat | | | |
| M G S S H H H H H H S S G L V P R G S H | | | |
| atggaaactgcagatctcaaatctctggccaagagaatctacgaggcctacttgaagaac | | | |
| M E T A D L K S L A K R I Y E A Y L K N | | | |
| ttcaacatgaacaaggtcaaagcccgggtcatcctctcaggaaaggccagtaacaatcca | | | |
| F N M N K V K A R V I L S G K A S N N P | | | |
| cctttttgtcatacatgatatggagacactgtgtatggctgagaagacgctggtggccaag | | | |
| P F V I H D M E T L C M A E K T L V A K | | | |
| ctggtggccaatggcatccagaacaaggaggcggaggtccgcatctttcactgctgccag | | | |
| L V A N G I Q N K E A E V R I F H C C Q | | | |
| tgcacgtcagtggagaccgtcacggagctcacggaattcgccaaggccatcccaggcttc | | | |
| C T S V E T V T E L T E F A K A I P G F | | | |
| gcaaacttggacctgaacgatcaagtgacattgctaaaatacggagtttatgaggccata | | | |
| A N L D L N D Q V T L L K Y G V Y E A I | | | |
| ttcgccatgctgtcttctgtgatgaacaaagacgggatgctggtagcgtatggaaatggg | | | |
| F A M L S S V M N K D G M L V A Y G N G | | | |
| tttataactcgtgaattcctaaaaagcctaaggaaaccgttctgtgatatcatggaaccc | | | |
| F I T R E F L K S L R K P F C D I M E P | | | |
| aagtttgattttgccatgaagttcaatgcactggaactggatgacagtgatatctccctt | | | |
| K F D F A M K F N A L E L D D S D I S L | | | |
| tttgtggctgctatcatttgctgtggagatcgtcctggccttctaaacgtaggacacatt | | | |
| F V A A I I C C G D R P G L L N V G H I | | | |
| gaaaaaatgcaggagggtattgtacatgtgctcagactccacctgcagagcaaccacccg | | | |
| E K M Q E G I V H V L R L H L Q S N H P | | | |
| gacgatatctttctcttcccaaaacttcttcaaaaaatggcagacctccggcagctggtg | | | |
| D D I F L F P K L L Q K M A D L R Q L V | | | |
| acggagcatgcgcagctggtgcagatcatcaagaagacggagtcggatgctgcgctgcac | | | |
| T E H A Q L V Q I I K K T E S D A A L H | | | |
| ccgctactgcaggagatctacagggacatgtactgagtcgacaagcttgcggccgcactc | | | |
| P L L Q E I Y R D M Y - | | | |
| gagcaccaccaccaccaccactgagat | | | |
| PCR primers: | | | |
| PPARA | PPARA-S | GCTGACACATATGGAAACTGCAGATCTCAAATC | (SEQ ID NO:_{——}) |
| | PPARA-A | GTGACTGTCGACTCAGTACATGTCCCTGTAGA | (SEQ ID NO:_{——}) |
| PPARγ. (Nucleic acid SEQ ID NO:_{——}) (Protein SEQ ID NO:_{——}) | | | |
| P333. pET28 PPARG E205-Y475-X | | | |
| taatacgactcactataggggaattgt | | | |
| gagcggataacaattcccctctagaaataattttgtttaactttaagaaggagatatacc | | | |
| atgggcagcagccatcatcatcatcatcacagcagcggcctggtgccgcgcggcagccat | | | |
| M G S S H H H H H H S S G L V P R G S H | | | |
| atggagtccgctgacctccgggccctggcaaaacatttgtatgactcatacataaagtcc | | | |
| M E S A D L R A L A K H L Y D S Y I K S | | | |
| ttcccgctgaccaaagcaaaggcgagggcgatcttgacaggaaagacaacagacaaatca | | | |
| F P L T K A K A R A I L T G K T T D K S | | | |
| ccattcgttatctatgacatgaattccttaatgatgggagaagataaaatcaagttcaaa | | | |
| P F V I Y D M N S L M M G E D K I K F K | | | |
| cacatcacccccctgcaggagcagagcaaagaggtggccatccgcatctttcagggctgc | | | |
| H I T P L Q E Q S K E V A I R I F Q G C | | | |
| cagtttcgctccgtggaggctgtgcaggagatcacagagtatgccaaaagcattcctggt | | | |
| Q F R S V E A V Q E I T E Y A K S I P G | | | |
| tttgtaaatcttgacttgaacgaccaagtaactctcctcaaatatggagtccacgagatc | | | |
| F V N L D L N D Q V T L L K Y G V H E I | | | |
| atttacacaatgctggcctccttgatgaataaagatggggttctcatatccgagggccaa | | | |
| I Y T M L A S L M N K D G V L I S E G Q | | | |
| ggcttcatgacaagggagtttctaaagagcctgcgaaagccttttggtgactttatggag | | | |
| G F M T R E F L K S L R K P F G D F M E | | | |
| cccaagtttgagtttgctgtgaagttcaatgcactggaattagatgacagcgacttggca | | | |
| P K F E F A V K F N A L E L D D S D L A | | | |
| atatttattgctgtcattattctcagtggagaccgcccaggtttgctgaatgtgaagccc | | | |
| I F I A V I I L S G D R P G L L N V K P | | | |
| attgaagacattcaagacaacctgctacaagccctggagctccagctgaagctgaaccac | | | |
| I E D I Q D N L L Q A L E L Q L K L N H | | | |
| cctgagtcctcacagctgtttgccaagctgctccagaaaatgacagacctcagacagatt | | | |
| P E S S Q L F A K L L Q K M T D L R Q I | | | |
| gtcacggaacatgtgcagctactgcaggtgatcaagaagacggagacagacatgagtctt | | | |
| V T E H V Q L L Q V I K K T E T D M S L | | | |
| cacccgctcctgcaggagatctacaaggacttgtactaggtcgacaagcttgcggccgca | | | |
| H P L L Q E I Y K D L Y - | | | |
| ctcgagcaccaccaccaccaccactgagat | | | |
| PCR Primers: | | | |
| PPARG | PPARG-S | GCTCAGACATATGGAGTCCGCTGACCTCCGGGC | (SEQ ID NO:_{——}) |
| | PPARG-A | GTGACTGTCGACCTAGTACAAGTCCTTGTAGA | (SEQ ID NO:_{——}) |
| PPARδ: (Nucleic acid SEQ ID NO:_{——}) (Protein SEQ ID NO:_{——}) | | | |
| P1057. pET BAM6 PPARD G165-Y441-X | | | |
| taatacgactcactataggggaattgt | | | |
| gagcggataacaattcccctctagaaataattttgtttaactttaagaaggagatatacc | | | |
| atgaaaaaaggtcaccaccatcaccatcacggatcccagtacaacccacaggtggccgac | | | |
| M K K G H H H H H H G S Q Y N P Q V A D | | | |
| ctgaaggccttctccaagcacatctacaatgcctacctgaaaaacttcaacatgaccaaa | | | |
| L K A F S K H I Y N A Y L K N F N M T K | | | |
| aagaaggcccgcagcatcctcaccggcaaagccagccacacggcgccctttgtgatccac | | | |
| K K A R S I L T G K A S H T A P F V I H | | | |
| gacatcgagacattgtggcaggcagagaaggggctggtgtggaagcagttggtgaatggc | | | |
| D I E T L W Q A E K G L V W K Q L V N G | | | |
| ctgcctccctacaaggagatcagcgtgcacgtcttctaccgctgccagtgcaccacagtg | | | |
| L P P Y K E I S V H V F Y R C Q C T T V | | | |
| gagaccgtgcgggagctcactgagttcgccaagagcatccccagcttcagcagcctcttc | | | |
| E T V R E L T E F A K S I P S F S S L F | | | |
| ctcaacgaccaggttacccttctcaagtatggcgtgcacgaggccatcttcgccatgctg | | | |
| L N D Q V T L L K Y G V H E A I F A M L | | | |
| gcctctatcgtcaacaaggacgggctgctggtagccaacggcagtggctttgtcacccgt | | | |
| A S I V N K D G L L V A N G S G F V T R | | | |
| gagttcctgcgcagcctccgcaaacccttcagtgatatcattgagcctaagtttgaattt | | | |
| E F L R S L R K P F S D I I E P K F E F | | | |
| gctgtcaagttcaacgccctggaacttgatgacagtgacctggccctattcattgcggcc | | | |
| A V K F N A L E L D D S D L A L F I A A | | | |
| atcattctgtgtggagaccggccaggcctcatgaacgttccacgggtggaggctatccag | | | |
| I I L C G D R P G L M N V P R V E A I Q | | | |
| gacaccatcctgcgtgccctcgaattccacctgcaggccaaccaccctgatgcccagtac | | | |
| D T I L R A L E F H L Q A N H P D A Q Y | | | |
| ctcttccccaagctgctgcagaagatggctgacctgcggcaactggtcaccgagcacgcc | | | |
| L F P K L L Q K M A D L R Q L V T E H A | | | |
| cagatgatgcagcggatcaagaagaccgaaaccgagacctcgctgcaccctctgctccag | | | |
| Q M M Q R I K K T E T E T S L H P L L Q | | | |
| gagatctacaaggacatgtactaagtcgaccaccaccaccaccaccactgagatccggct | | | |
| E I Y K D M Y - | | | |
| ggccctactggccgaaaggaattcgaggccagcagggccaccgctgagcaataactagca | | | |
| taaccccttggggcctctaaacgggtcttgaggggttttttg | | | |
| PCR Primers: | | | |
| PPARD | PPARD-G165 | GTTGGATCCCAGTACAACCCACAGGTGGC | (SEQ ID NO:_{——}) |
| | PPARD-A | GTGACTGTCGACTTAGTACATGTCCTTGTAGA | (SEQ ID NO:_{——}) |

### Example 22

### Bio-Chemical Screening

The homogenous Alpha screen assay was used in the agonist mode to determine the ligand dependent interaction of the PPARs (α,δ,γ) with the coactivator Biotin-PGC-1 peptide (biotin-AHX-DGTPPPQEAEEPSLLKKLLLAPANT-CONH₂ (SEQ ID NO:______), supplied by Wyeth). All compounds tested were serially diluted 1:3 into DMSO for a total of 8 concentration points. Samples were prepared with His-tagged PPAR-LBD prepared per Example 21. Ni-chelate acceptor beads were added that bind to the his-tagged PPAR-LBD and streptavidin donor beads were added that bind to the biotin of the coactivator (Perkin-Elmer #6760619M) such that agonist activity correlates to signal from the donor and acceptor beads in close proximity. Each sample was prepared by mixing 1 μl of compound and 15 μl of 1.33× receptor/peptide mix, incubating for 15 minutes at room temperature, then adding 4 μl of 4× beads in assay buffer. The assay buffer was 50 mM HEPES, pH 7.5, 50 mM KCl, 1 mM DTT and 0.8% BSA. Final concentrations for each sample were 25 nM biotin-PGC-1 peptide, 20 nM PPARγ or 10 nM PPARα or δ, and each bead at 5 μg/ml, with compound added to the desired concentration resulting in final DMSO of 5%. WY-14643 (PPARα), farglitazar (PPARγ) and bezafibrate (PPARδ) were assayed as control samples. The samples were incubated for 1 hour in the dark at room temperature before taking the reading in the Fusion alpha or Alpha Quest reader. The signal vs. compound concentration was used to determine the EC₅₀. The data was expressed in μMol/L. The data points from the Fusion alpha instrument were transferred to Assay Explorer® (MDL) to generate a curve and calculate the inflection point of the curve as EC₅₀.

### Example 23

### Co-Transfection Assay

This assay serves to confirm the observed biochemical activity (Example 22) on the modulation of intended target molecule(s) at the cellular level. 293T cells (ATCC) were seeded at 1-2×10⁶ cells per well of a 6 well plate (Corning 3516) in 3 ml of growth medium (Dulbecco's eagle medium, Mediatech, with 10% FBS). These were incubated to 80-90% confluent and the medium was removed by aspirating. These cells were transfected with PPAR LBD and luciferase such that agonist results in activation of the luciferase. Measurement of luciferase activity of transfected cells treated with compounds directly correlates with agonist activity. To 100 μl of serum free growth medium was added 1 μg of pFR-Luc (Stratagene catalog number 219050), 6 μl Metafectene (Biontex, Inc.) and 1 mg of the pGal4-PPAR-LBD (α, γ or δ from Example 21). This was mixed by inverting, then incubated for 15-20 minutes at room temperature, and diluted with 900 μl of serum free growth medium. This was overlayed onto the 293T cells and incubated for 4-5 hours at 37° C. in CO₂ incubator. The transfection medium was removed by aspirating and growth medium was added and the cells incubated for 24 hours. The cells were then suspended in 5 ml of growth medium and diluted with an additional 15 ml of growth medium. For each test sample, 95 μl of the transfected cells were transferred per well of a 96 well culture plate. Compounds tested were diluted in DMSO to 200× the desired final concentration. This was diluted 10× with growth medium and 5 μl was added to the 95 μl of transfected cells. The plate was incubated for 24 hours 37° C. in CO₂ incubator. Luciferase reaction mixture was prepared by mixing 1 ml of lysis buffer, 1 ml of substrate in lysis buffer, and 3 ml of reaction buffer (Roche Diagnostics Luciferase assay kit #1814036). For each sample well, the growth medium was replaced with 50 ml of reaction mixture and the plate shaken for 15-20 minutes, and the luminescence was measured on a Victor2 V plate reader (Perkin Elmer). The signal vs. compound concentration was used to determine the EC₅₀.

Compounds having EC₅₀ of less than or equal to 1 μM in either the biochemical assay of Example 22 or this cell based assay for at least one of PPARα, PPARγ and PPARδ are shown in Table 14.

TABLE 14
Compounds of the invention having EC₅₀ of less than or equal to 1 μM
in at least one of PPARα, PPARγ or PPARδ activity assays.

P-0001, P-0002, P-0005, P-0009, P-0010, P-0011, P-0017, P-0018,
P-0019, P-0021, P-0025, P-0027, P-0029, P-0050, P-0056, P-0057,
P-0058, P-0059, P-0060, P-0061, P-0062, P-0064, P-0067, P-0068,
P-0073, P-0074, P-0075, P-0076, P-0077, P-0078, P-0080, P-0081,
P-0082, P-0087, P-0089, P-0090, P-0091, P-0092, P-0093, P-0094,
P-0095, P-0096, P-0097, P-0098, P-0099, P-0100, P-0101, P-0102,
P-0103, P-0104, P-0105, P-0106, P-0107, P-0108, P-0109, P-0110,
P-0111, P-0112, P-0113, P-0114, P-0115, P-0117, P-0118, P-0121,
P-0126, P-0127, P-0129, P-0132, P-0133, P-0134, P-0135, P-0136,
P-0137, P-0138, P-0139, P-0140, P-0150, P-0152, P-0155, P-0156,
P-0157, P-0158, P-0164, P-0165, P-0167, P-0174, P-0175, P-0178,
P-0180, P-0186, P-0187, P-0188, P-0190, P-0191, P-0193, P-0194,
P-0195, P-0196, P-0197, P-0198, P-0200, P-0201, P-0202, P-0205,
P-0208, P-0209, P-0210, P-0215, P-0217, P-0218, P-0220, P-0223,
P-0224, P-0225, P-0226, P-0227, P-0228, P-0229, P-0239, P-0244,
P-0247, P-0257, P-0258, P-0259, P-0260, P-0262, P-0266, P-0267,
P-0270, P-0271, P-0272, P-0273, P-0274, P-0275, P-0276, P-0277,
P-0280, P-0281, P-0282, P-0284, P-0285, P-0287, P-0288, P-0293,
P-0295

All patents and other references cited in the specification are indicative of the level of skill of those skilled in the art to which the invention pertains, and are incorporated by reference in their entireties, including any tables and figures, to the same extent as if each reference had been incorporated by reference in its entirety individually.

One skilled in the art would readily appreciate that the present invention is well adapted to obtain the ends and advantages mentioned, as well as those inherent therein. The methods, variances, and compositions described herein as presently representative of preferred embodiments are exemplary and are not intended as limitations on the scope of the invention. Changes therein and other uses will occur to those skilled in the art, which are encompassed within the spirit of the invention, and defined by the scope of the claims.

It will be readily apparent to one skilled in the art that varying substitutions and modifications may be made to the invention disclosed herein without departing from the scope and spirit of the invention. For example, variations can be made to provide additional compounds of Formula I and/or various methods of administration can be used. Thus, such additional embodiments are within the scope of the present invention and the following claims.

The invention illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed herein are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed herein may be resorted to by those skilled in the art, and that such modifications and variations are considered to be within the scope of this invention as defined by the appended claims.

In addition, where features or aspects of the invention are described in terms of Markush groups or other grouping of alternatives, those skilled in the art will recognize that the invention is also thereby described in terms of any individual member or subgroup of members of the Markush group or other group.

Also, unless indicated to the contrary, where various numerical values are provided for embodiments, additional embodiments are described by taking any 2 different values as the endpoints of a range. Such ranges are also within the scope of the described invention.

Thus, additional embodiments are within the scope of the invention and within the following claims.

PPAR Sequences

PPARA Accession No. NM_005036 (SEQ ID NO:_{——})
gcgccgcctc cttcggcgtt cgccccacgg accggcaggc ggcggaccgc ggcccaggct
gaagctcagg gccctgtctg ctctgtggac tcaacagttt gtggcaagac aagctcagaa
ctgagaagct gtcaccacag ttctggaggc tgggaagttc aagatcaaag tgccagcaga
ttcagtgtca tgtgaggacg tgcttcctgc ttcatagata agagtagctt ggagctcggc
ggcacaacca gcaccatctg gtcgcgatgg tggacacgga aagcccactc tgccccctct
ccccactcga ggccggcgat ctagagagcc cgttatctga agagttcctg caagaaatgg
gaaacatcca agagatttcg caatccatcg gcgaggatag ttctggaagc tttggcttta
cggaatacca gtatttagga agctgtcctg gctcagatgg ctcggtcatc acggacacgc
tttcaccagc ttcgagcccc tcctcggtga cttatcctgt ggtccccggc agcgtggacg
agtctcccag tggagcattg aacatcgaat gtagaatctg cggggacaag gcctcaggct
atcattacgg agtccacgcg tgtgaaggct gcaagggctt ctttcggcga acgattcgac
tcaagctggt gtatgacaag tgcgaccgca gctgcaagat ccagaaaaag aacagaaaca
aatgccagta ttgtcgattt cacaagtgcc tttctgtcgg gatgtcacac aacgcgattc
gttttggacg aatgccaaga tctgagaaag caaaactgaa agcagaaatt cttacctgtg
aacatgacat agaagattct gaaactgcag atctcaaatc tctggccaag agaatctacg
aggcctactt gaagaacttc aacatgaaca aggtcaaagc ccgggtcatc ctctcaggaa
aggccagtaa caatccacct tttgtcatac atgatatgga gacactgtgt atggctgaga
agacgctggt ggccaagctg gtggccaatg gcatccagaa caaggaggcg gaggtccgca
tctttcactg ctgccagtgc acgtcagtgg agaccgtcac ggagctcacg gaattcgcca
aggccatccc aggcttcgca aacttggacc tgaacgatca agtgacattg ctaaaatacg
gagtttatga ggccatattc gccatgctgt cttctgtgat gaacaaagac gggatgctgg
tagcgtatgg aaatgggttt ataactcgtg aattcctaaa aagcctaagg aaaccgttct
gtgatatcat ggaacccaag tttgattttg ccatgaagtt caatgcactg gaactggatg
acagtgatat ctcccttttt gtggctgcta tcatttgctg tggagatcgt cctggccttc
taaacgtagg acacattgaa aaaatgcagg agggtattgt acatgtgctc agactccacc
tgcagagcaa ccacccggac gatatctttc tcttcccaaa acttcttcaa aaaatggcag
acctccggca gctggtgacg gagcatgcgc agctggtgca gatcatcaag aagacggagt
cggatgctgc gctgcacccg ctactgcagg agatctacag ggacatgtac tgagttcctt
cagatcagcc acaccttttc caggagttct gaagctgaca gcactacaaa ggagacgggg
gagcagcacg attttgcaca aatatccacc actttaacct tagagcttgg acagtctgag
ctgtaggtaa ccggcatatt attccatatc tttgttttaa ccagtacttc taagagcata
gaactcaaat gctgggggta ggtggctaat ctcaggactg ggaagattac ggcgaattat
gctcaatggt ctgattttaa ctcacccgat gttaatcaat gcacattgct ttagatcaca
ttcgtgattt accatttaat taactggtaa cctcaaaatt cgtggcctgt cttcccattc
accccgcttt tgactattgt gctcctttat aattctgaaa actaatcagc actttttaac
aatgtttata atcctataag tctagatgta tccaaaggtg aagtatgtaa aaagcagcaa
aatatttatt tcaaagactt cacttctgtt tcctgaatct aaagaaagac aacatgctgc
tttttaatca taggatggag aattttaaag aactgtttgg gccaggcaca gtcgctcata
cttgtaatcc cagcactttg ggaggccgag gcgggtggat cacaaggtca gcagatcgag
accatcctgg ccaacatggt gaaaccctgt ctctactaaa aatacaaaaa ttagccgggt
gtggtggcac atgcctgtaa tcccagctac tcgggaagct gaggcaggag aattgcttga
accagggagt tggaggttgc agtgagctaa gactgcacca ctgcactcca gcctggtgac
agaacgagac tctgtcttaa aaacaaacaa acaaaaaaaa aatctgttag ataagctatc
aaaatgcagc tgttgttttg tttttggctc actgttttcg tggttgtaac taatatgtgg
aaaggcccat ttccaggttt gcgtagaaga gcccagaaaa cagagtctca agacccccgc
tctggactgt cataagctag cacccgtggt aagcgggacg agacaagctc ccgaagcccg
ccagcttcct gctccactca gctccgtcca gtcaacctga acccacccag tccagctgtc
tgtgggaatg gtggtgttct tagggacaga ctgacacctt acttgtcagt gttcctccgg
gccccatttg gcagctcccg tatcttttgt tatgttgctt ttaaagatat gatgttttat
tgttttaact cttggtgaca gtagatgctc tctggagcgc agacgaggca catgtgtctt
catagcctgg gctgggtggg agccagtcac cctgcggatc gagagagggg gtagagtctt
cttcaaatgg cagttttact tcaaatggca gatttcacaa gagttggtta ttttttacaa
tggtttaggt tgttaagtct cctttgtatg taaggtagtt ttttcaacat ctaaaatttt
tgttttagcc ttcaaaacca acttaccaac ctcagtccag ctgggaaggc agcgttgatt
atggtagttt gtcaagaata tatggacctg gaaacacttt ctctctctgt ccacctggta
gataaattgt cctgttgaga atttttagat ctggactgga actgccagga ccaccgcctc
cagggagtcg ctgggcacct ggaggtatcg tcgatgcctc tcccccatct ttagaaaatt
tggctcttct gaggtcatta ttattttaag aatgattagg attgataagg gtcccatgac
cagcattatg aaaatgcgag agtgggaagg acacagtgtg agacttccac tagaaaaaag
tgaaagttag ggttaggaca tcctttttta aaaattacaa atttagtccg ttttggtttt
tgtaatcagg ctaggcacag tggctcacac atggaatccc agcactttgg gaggccgagg
tgggaggatc acttgagccc aggagttcga gaccagccta ggcaacatag caagaccctg
tctgtacaca aaatttaaaa attagttcat cggggtggca cacatcagta gtcccagcta
ctctgcaggc tgaggtggga ggattgcttg aacccaggag gtcgaggctg cagtgagctg
tgatctcacc actgcattcc agcctgggtg acagagttag attccaccct ctcccacccc
ggcaaaaaaa aaaaaaaaag atgcaatcaa aggggctgtt ggccagcaat ggcagcagca
gcggcgggca gtctgcccaa gtgtcttagg aaccaaaagc aaataaaagt gtttccatat
atgccaccag ccaagtggcc atcctaattc agaaagaagc tagcctttga gtgtctgtca
tggtgcatcc gtttcagtat tatttcctaa aatgagaagc ccctgtgtca acaagatcca
ggggctggag cccaatgcca agcctgtgtt gtccccagcg accctgcagc tgctcgctct
gatgtaccct gtgccattca aggagatgtg gtccaggaaa gtgagcctca tggttttcag
agaagtcatt gttctgttta cattttcata aaacctgttt aaaatagctc cccgtctcag
gctttcagca gtaacagtga gctgactggc aagttcgatg ttagctcccg ggacactcag
cagcgatggt gagcattttg gtttccttaa ggcccagcaa gacttccagg gacatctctg
gtgaagccag aatggagaca cccgtgacct caggctgaaa gtcactcgac attggtctct
tgtgttgata gggaaggaaa tcaggcattc ctatttcttt aaataacaaa accactaatt
gccactcaat gctggaatat tttgggtcac ctaatcatag atttctcagg gcatcaatac
tcaaatatag gctgattatg ccccagttca aatgggaact attaacagag tgcatttctt
gcttgctggg tttcaacaga catcagccaa aagaacaaaa gagatgtcag gacagattcc
aggagtgtcg gagcacatgt gtggcacccg ctccctctgg cagcgaatgt aggaagtcgc
caaatttacc cactcttcaa caagtcattg tttaaacacg gtttttcatt ttctcaactt
ttaatagcaa aaagtgccaa agtcctcaga gacctaacag ccttggtcta ccgtgctgac
cagggtgaag gcacggcgag ggactcctcc cagacgtgcc tcttgtgtgc cagctggctg
tggctcggga gcagacgcag gcctctccat tgtccagggg agcctggcgg cgcatccctc
ctctcccacc tcctggcact tccagctggg tgtcccacat gttggattcc gtccccacca
cacttccaga gaccggagaa ctgtgcaggg cctaaggccg tttggatgaa ttgtcaaaac
aagatgcttc cagttacagc ggcaggagcg ggactgggag cacgggctga cggctgctgg
tgcctttctt cccacctcgc ttgcctgttt ccgcttgacc cttcctccag ctccgatgag
aagagtataa agcatcttcc taacgggtgt gtttgctata cgaacataat ggacgtgaag
tggggcagaa acccagaact cagcattcaa ggatgcccag gagagctgtc cctgttttaa
agagctgtgt tttgttttgt ttcgcattta gagagcagac aaggcaccct tctgctgcgc
tgatacgttt cttacactgg gccattttag acccccaggg aaacagcctt cctggagcgt
tgtctggagg ttccagggac agggcagcct cccagagccg agcaagagct caaggtacaa
atgagagatt tgctataccg tgagaagtca acaacttagc caccacttcc ccgcaatgga
ccatgtaaca aatacctcag caggccctgc aaaaggccat gctagagctg aggcgcacag
cctgtggcct ctgtagttag ggcaggtggg atggagactc cttgagtgca cacacctgag
cctgcccaca cacaggggag cagcatctcg tatgacgtct ggaaggaact tcggttgtgt
aaagggagcc ttgaagatac gtgcaaaagg tgctacccca atttggtgaa actgacattg
ggcacgtctt gggcttagga gaagcggccg atggtcccgg cctgcagtga caaacccccc
tccccgcacc gcccccagca ccccctctcc tcttcacctc ttcctgctgg ccacgaggaa
gccacttcct cagagagacc ctaccagatg cggatggaaa cagatgcacc aaagcaagcc
ctgatgaaac cgcgacttcc taaggtctgt ctcctctgaa cttgcacctg ggcctctctg
tgtttggttc caagcacttc ccacctcaaa ctcccatttt caaaccactg tatctctgcg
cacatctgct acttaccagc cgcatacatg atggagggtt ttttggtcct gatccagtgg
ccacacctgt ctttgaaatg tctcactgaa ctccagtttt aaaatagatt cattgcttca
acacagcaag cccaatgcac ccagctaaga ctggcttgac cgacagcctg gcctttggtg
gggggcttcc tggggcctgg ggaaagctgg ccaccttcaa cagctggtac ctcttcaaca
gtgtggcctt tcaaaatgca gatgccacca ggagaacatg cccacagctc accacctatg
gatgccatgg ctctgggcag ctttcaaagc aggttcctgt ggtctcctca gctgtttgag
ggggtaacag caaatcagcc tccattttaa aatgaaaaca ccagcctcca gatgtagggc
ctgctgggtg ttgctagccg ctggtcccca ggcacggtgc actttctcca cctcctgcag
cctccctgtt gtttctagac tcttgcacct ggtgagtgca aggataggtg acccaggggc
ctgcagcctt gtcctcagct cccatctcct ggactgccag cctcaccctc tgcagttagc
atggttggcc tgatgcaggg atcccgaggg attacttttt agaccttctt tcacattcag
aaaagtagta tagattcagg agaggcaaga aaattatgCt gtccatagaa gtcacccatg
aagactgatg ccaccacctg aaggctcatg attgttaaaa atgtccacgg gaacctctcg
tccacaggag gtttgtctca acacttccca tttttacggc attggcattg ccaagcatgg
ggaagtatct gctcttctca tgttaaaagt ggcccagctt ttcttaactc agtccaagct
gacttgttta gctgcactgg aatttcttac caaccaaata tttgcatcga gcaaaggggg
ctgtgtgcac ctccctaatg gcagcgatga tggctgctgt cattcaagcc catcttcaga
cgtcacagtc tggaagtgaa atgtccacaa acatctgtgg cagaaaaggc tatacggacc
acccagttgt gctgcagctt tacagagcaa ggaagggttg tggcaaataa atgattaacc
tgcctcgact gtgctgaggg caacaaaggc catctcacca aaggattatt cgatgccatt
aaatcatccc gtgaccttcc tgcttccgag tccatggcct ttgcccaggg catgtactcc
cctgagaggc cttctgccta gaaagatcta tgactgggtt ccaaagttga ggcctaggtt
tttgctggga tttagatatt ttcaggcacc attttgacag cattcaggaa aacggttatt
gaccccatag actagggtaa gaataaaggc aataaatttg gtctgactca gaatatagga
gatccatata tttctctgga aaccacagtg tacactaaaa tgtgaaattg aaggttttgt
taaaaagaaa aagataatga gcttcatgct ttgtttaatt acataatgat ttccattacg
ctatttctgt gaaatgcagc aggttcttaa acgttatttc agtggcatgg gctggaagct
tatcacaaaa agccatgtgt gtggccttat cagaacagaa agagacaggo tggtgcccaa
ggctgctgcc tgctccacct tttgccagct ctggacatct gaggacgtcc cggcagatct
ggaatggggc cctcaactga ccatttgctt ctcagaattt cagtttgaga catgagaggt
ataatcagtt acttttctcc ccccagagaa acccttttgt gaggggagag gagctatggt
atgtggttca gctgaaacac atacaactgc atccttttgg agtcctttgc caacaaaaac
agaccaacag accagatggt gtccatgttc aatatcatgt cttgatggac gcagctgatg
acctcaaata cttgagtggt ctcatggctg ttagatggat tatttgaaaa aaaaaaaaaa
aaaagagaga aaaaataatt gatttttaca tcagagatag caaactaaga cctggggagg
ggggtcagct tttattttat tttatttttt ttaagtttgc tagttgggtc aaatgtgagg
aggagggagt ctacctgcca cctcttctct tgcccctctt ctgcccacac atccagcatc
caaaatccat tcatttaatg aattgataaa gtgccgtgca aactggtgca caaacaggcc
cccagtccac gcagcctggc tcctaggaaa agtggtgacc gggcgtgggg gggcatgccg
cagccctggg acacagtcgg gcaccttccc cggaccccca ggccttggct gtgcctcaag
tcagagaggg tcagccttca ggccccggag acgagtgact ggccgatcat ttcacaataa
aatcactcac ttttggcaac ttcacttttt ttaaggcaca gtcagttcct tttctcatgt
acctcacaaa agatgaagac catgtagtac tctttttggt aaagttacag tgttcatgtt
aaatatcact tttttctaca ttgtgtggta aaaagaacta cgttaatagc tatatcttaa
atactgtgat ttgacttttt gaaaaatatc ctaatacaaa tattttacta acttacaatc
actcatttaa taagaaacat ttggattctt ttgaaatcag tgttaattga ctcatattct
taaaagcctg gctcttgacc ctattggaaa cacaaaggaa gctgaaatca aacatctaaa
atacactgcg tacacgtgtg cgtgcacaca cacacacaca cacacacaca cacagctctt
catttctcct gagccatgca gaatttactt tcaatgtgga aatctgttcc ctttaccaca
ctgtatatgc acagagcaca agagaggcta tctctagtca cttccaccag cgaggcctta
gactccgtat tagaggccac cgatttcata caacagtgtt tcgctaaaga cccttcacta
ttcttgttta gtaaatagct gtctgctctt cagggaactg ttacctatgg gttattacca
aagaacgctg gcaattggaa atgtcctgat ggaaattctt tgcacgtgcc ggttctctgg
catcctccag gtggcccaac ccaaagcaga aagcagaaac cacagacccc gtgagtctcc
ccataccttg tttccaataa cttggcaaaa cttcttggtg catattggtt acaccctctg
ggattcataa tgccattagg ctaaaaccct aagagagagg gttgacagaa acacacgcga
gaatgaggca gatcccagag caaggactgg gcccagactc tccacatgtg ctctactagt
gagtgcctta tactctcagt attttggggc ttacagcttc ttatttgtgc taaaaaggtg
cagttccaaa gtaggaactg ccacacaggc cccagcatcc tctctccaac ttcatacctc
tctcctggtg gggggagcgg gcatccagga cctccggaat caaggatgtg cagagaagag
cgaaagtaat ttttctagtc acatgaactg attggttcca ggcaattaga aaatggctat
aaaataacct taattttaaa aaaaaatctt gggtcttcgt tttcctatta ggagactgaa
ctgaccacat gtattgattt atatcctgaa tatatgggaa cttctgtgtt tgggatgtcc
tactgtaaga ctgatgaatg tacagagtta atttcagggt acagttttgc cttaatggtt
ttaaaaaata aactattttt taaaatttt
PPARA Accession No. NP_005027 (SEQ ID NO:_{——})
MVDTESPLCP LSPLEAGDLE SPLSEEFLQE MGNIQEISQS IGEDSSGSFG FTEYQYLGSC
PGSDGSVITD TLSPASSPSS VTYPVVPGSV DESPSGALNI ECRICGDKAS GYHYGVHACE
GCKGFFRRTI RLKLVYDKCD RSCKIQKKNR NKCQYCRFHK CLSVGMSHNA IRFGRMPRSE
KAKLKAEILT CEHDIEDSET ADLKSLAKRI YEAYLKNFNM NKVKARVILS GKASNNPPFV
IHDMETLCMA EKTLVAKLVA NGIQNKEAEV RIFHCCQCTS VETVTELTEF AKAIPGFANL
DLNDQVTLLK YGVYEAIFAM LSSVMNKDGM LVAYGNGFIT REFLKSLRKP FCDIMEPKFD
FAMKFNALEL DDSDISLFVA AIICCGDRPG LLNVGHIEKM QEGIVHVLRL HLQSNHPDDI
flfpkllqkm adlrqlvteh aqlvqiikkt esdaalhpll qeiyrdmy
PPARG Accession No. NM_015869 (SEQ ID NO:_{——})
actgatgtct tgactcatgg gtgtattcac aaattctgtt acttcaagtc tttttctttt
aacggattga tcttttgcta gatagagaca aaatatcagt gtgaattaca gcaaacccct
attccatgct gttatgggtg aaactctggg agattctcct attgacccag aaagcgattc
cttcactgat acactgtctg caaacatatc acaagaaatg accatggttg acacagagat
gccattctgg cccaccaact ttgggatcag ctccgtggat ctctccgtaa tggaagacca
ctcccactcc tttgatatca agcccttcac tactgttgac ttctccagca tttctactcc
acattacgaa gacattccat tcacaagaac agatccagtg gttgcagatt acaagtatga
cctgaaactt caagagtacc aaagtgcaat caaagtggag cctgcatctc caccttatta
ttctgagaag actcagctct acaataagcc tcatgaagag ccttccaact ccctcatggc
aattgaatgt cgtgtctgtg gagataaagc ttctggattt cactatggag ttcatgcttg
tgaaggatgc aagggtttct tccggagaac aatcagattg aagcttatct atgacagatg
tgatcttaac tgtcggatcc acaaaaaaag tagaaataaa tgtcagtact gtcggtttca
gaaatgcctt gcagtgggga tgtctcataa tgccatcagg tttgggcgga tgccacaggc
cgagaaggag aagctgttgg cggagatctc cagtgatatc gaccagctga atccagagtc
cgctgacctc cgggccctgg caaaacattt gtatgactca tacataaagt ccttcccgct
gaccaaagca aaggcgaggg cgatcttgac aggaaagaca acagacaaat caccattcgt
tatctatgac atgaattcct taatgatggg agaagataaa atcaagttca aacacatcac
ccccctgcag gagcagagca aagaggtggc catccgcatc tttcagggct gccagtttcg
ctccgtggag gctgtgcagg agatcacaga gtatgccaaa agcattcctg gttttgtaaa
tcttgacttg aacgaccaag taactctcct caaatatgga gtccacgaga tcatttacac
aatgctggcc tccttgatga ataaagatgg ggttctcata tccgagggcc aaggcttcat
gacaagggag tttctaaaga gcctgcgaaa gccttttggt gactttatgg agcccaagtt
tgagtttgct gtgaagttca atgcactgga attagatgac agcgacttgg caatatttat
tgctgtcatt attctcagtg gagaccgccc aggtttgctg aatgtgaagc ccattgaaga
cattcaagac aacctgctac aagccctgga gctccagctg aagctgaacc accctgagtc
ctcacagctg tttgccaagc tgctccagaa aatgacagac ctcagacaga ttgtcacgga
acacgtgcag ctactgcagg tgatcaagaa gacggagaca gacatgagtc ttcacccgct
cctgcaggag atctacaagg acttgtacta gcagagagtc ctgagccact gccaacattt
cccttcttcc agttgcacta ttctgaggga aaatctgaca cctaagaaat ttactgtgaa
aaagcatttt aaaaagaaaa ggttttagaa tatgatctat tttatgcata ttgtttataa
agacacattt acaatttact tttaatatta aaaattacca tattatgaaa aaaaaaaaaa
aaa
PPARG Accession No. NP_056953 (SEQ ID NO:_{——})
MGETLGDSPI DPESDSFTDT LSANISQEMT MVDTEMPFWP TNFGISSVDL SVMEDHSHSF
DIKPFTTVDF SSISTPHYED IPFTRTDPVV ADYKYDLKLQ EYQSAIKVEP ASPPYYSEKT
QLYNKPHEEP SNSLMAIECR VCGDKASGFH YGVHACEGCK GFFRRTIRLK LIYDRCDLNC
RIHKKSRNKC QYCRFQKCLA VGMSHNAIRF GRMPQAEKEK LLAEISSDID QLNPESADLR
ALAKHLYDSY IKSFPLTKAK ARAILTGKTT DKSPFVIYDM NSLMMGEDKI KFKHITPLQE
QSKEVAIRIF QGCQFRSVEA VQEITEYAKS IPGFVNLDLN DQVTLLKYGV HEIIYTMLAS
LMNKDGVLIS EGQGFMTREF LKSLRKPFGD FMEPKFEFAV KFNALELDDS DLAIFIAVII
LSGDRPGLLN VKPIEDIQDN LLQALELQLK LNHPESSQLF AKLLQKMTDL RQIVTEHVQL
LQVIKKTETD MSLHPLLQEI YKDLY
PPARD Accession No. NM_006238 (SEQ ID NO:_{——})
gcggagcgtg tgacgctgcg gccgccgcgg acctggggat taatgggaaa agttttggca
ggagcgggag aattctgcgg agcctgcggg acggcggcgg tggcgccgta ggcagccggg
acagtgttgt acagtgtttt gggcatgcac gtgatactca cacagtggct tctgctcacc
aacagatgaa gacagatgca ccaacgaggc tgatgggaac caccctgtag aggtccatct
gcgttcagac ccagacgatg ccagagctat gactgggcct gcaggtgtgg cgccgagggg
agatcagcca tggagcagcc acaggaggaa gcccctgagg tccgggaaga ggaggagaaa
gaggaagtgg cagaggcaga aggagcccca gagctcaatg ggggaccaca gcatgcactt
ccttccagca gctacacaga cctctcccgg agctcctcgc caccctcact gctggaccaa
ctgcagatgg gctgtgacgg ggcctcatgc ggcagcctca acatggagtg ccgggtgtgc
ggggacaagg catcgggctt ccactacggt gttcatgcat gtgaggggtg caagggcttc
ttccgtcgta cgatccgcat gaagctggag tacgagaagt gtgagcgcag ctgcaagatt
cagaagaaga accgcaacaa gtgccagtac tgccgcttcc agaagtgcct ggcactgggc
atgtcacaca acgctatccg ttttggtcgg atgccggagg ctgagaagag gaagctggtg
gcagggctga ctgcaaacga ggggagccag tacaacccac aggtggccga cctgaaggcc
ttctccaagc acatctacaa tgcctacctg aaaaacttca acatgaccaa aaagaaggcc
cgcagcatcc tcaccggcaa agccagccac acggcgccct ttgtgatcca cgacatcgag
acattgtggc aggcagagaa ggggctggtg tggaagcagt tggtgaatgg cctgcctccc
tacaaggaga tcagcgtgca cgtcttctac cgctgccagt gcaccacagt ggagaccgtg
cgggagctca ctgagttcgc caagagcatc cccagcttca gcagcctctt cctcaacgac
caggttaccc ttctcaagta tggcgtgcac gaggccatct tcgccatgct ggcctctatc
gtcaacaagg acgggctgct ggtagccaac ggcagtggct ttgtcacccg tgagttcctg
cgcagcctcc gcaaaccctt cagtgatatc attgagccta agtttgaatt tgctgtcaag
ttcaacgccc tggaacttga tgacagtgac ctggccctat tcattgcggc catcattctg
tgtggagacc ggccaggcct catgaacgtt ccacgggtgg aggctatcca ggacaccatc
ctgcgtgccc tcgaattcca cctgcaggcc aaccaccctg atgcccagta cctcttcccc
aagctgctgc agaagatggc tgacctgcgg caactggtca ccgagcacgc ccagatgatg
cagcggatca agaagaccga aaccgagacc tcgctgcacc ctctgctcca ggagatctac
aaggacatgt actaacggcg gcacccaggc ctccctgcag actccaatgg ggccagcact
ggaggggccc acccacatga cttttccatt gaccagccct tgagcacccg gcctggagca
gcagagtccc acgatcgccc tcagacacat gacacccacg gcctctggct ccctgtgccc
tctctcccgc ttcctccagc cagctctctt cctgtctttg ttgtctccct ctttctcagt
tcctctttct tttctaattc ctgttgctct gtttcttcct ttctgtaggt ttctctcttc
ccttctccct tgccctccct ttctctctcc accccccacg tctgtcctcc tttcttattc
tgtgagatgt tttgtattat ttcaccagca gcatagaaca ggacctctgc ttttgcacac
cttttcccca ggagcagaag agagtggggc ctgccctctg ccccatcatt gcacctgcag
gcttaggtcc tcacttctgt ctcctgtctt cagagcaaaa gacttgagcc atccaaagaa
acactaagct ctctgggcct gggttccagg gaaggctaag catggcctgg actgactgca
gccccctata gtcatggggt ccctgctgca aaggacagtg ggcaggaggc cccaggctga
gagccagatg cctccccaag actgtcattg cccctccgat gctgaggcca cccactgacc
caactgatcc tgctccagca gcacacctca gccccactga cacccagtgt ccttccatct
tcacactggt ttgccaggcc aatgttgctg atggccccct gcactggccg ctggacggca
ctctcccagc ttggaagtag gcagggttcc ctccaggtgg gcccccacct cactgaagag
gagcaagtct caagagaagg aggggggatt ggtggttgga ggaagcagca cacccaattc
tgcccctagg actcggggtc tgagtcctgg ggtcaggcca gggagagctc ggggcaggcc
ttccgccagc actcccactg cccccctgcc cagtagcagc cgcccacatt gtgtcagcat
ccagggccag ggcctggcct cacatccccc tgctcctttc tctagctggc tccacgggag
ttcaggcccc actccccctg aagctgcccc tccagcacac acacataagc actgaaatca
ctttacctgc aggctccatg cacctccctt ccctccctga ggcaggtgag aacccagaga
gaggggcctg caggtgagca ggcagggctg ggccaggtct ccggggaggc aggggtcctg
caggtcctgg tgggtcagcc cagcacctgc tcccagtggg agcttcccgg gataaactga
gcctgttcat tctgatgtcc atttgtccca atagctctac tgccctcccc ttccccttta
ctcagcccag ctggccacct agaagtctcc ctgcacagcc tctagtgtcc ggggaccttg
tgggaccagt cccacaccgc tggtccctgc cctcccctgc tcccaggttg aggtgcgctc
acctcagagc agggccaaag cacagctggg catgccatgt ctgagcggcg cagagccctc
caggcctgca ggggcaaggg gctggctgga gtctcagagc acagaggtag gagaactggg
gttcaagccc aggcttcctg ggtcctgcct ggtcctccct cccaaggagc cattctgtgt
gtgactctgg gtggaagtgc ccagcccctg cccctacggg cgctgcagcc tcccttccat
gccccaggat cactctctgc tggcaggatt cttcccgctc cccacctacc cagctgatgg
gggttggggt gcttcctttc aggccaaggc tatgaaggga cagctgctgg gacccacctc
cccctccccg gccacatgcc gcgtccctgc cccgacccgg gtctggtgct gaggatacag
ctcttctcag tgtctgaaca atctccaaaa ttgaaatgta tatttttgct aggagcccca
gcttcctgtg tttttaatat aaatagtgta cacagactga cgaaacttta aataaatggg
aattaaatat ttaa
PPARD Accession No. NP_006229 (SEQ ID NO:_{——})
MEQPQEEAPE VREEEEKEEV AEAEGAPELN GGPQHALPSS SYTDLSRSSS PPSLLDQLQM
GCDGASCGSL NMECRVCGDK ASGFHYGVHA CEGCKGFFRR TIRMKLEYEK CERSCKIQKK
NRNKCQYCRF QKCLALGMSH NAIRFGRMPE AEKRKLVAGL TANEGSQYNP QVADLKAFSK
HIYNAYLKNF NMTKKKARSI LTGKASHTAP FVIHDIETLW QAEKGLVWKQ LVNGLPPYKE
ISVHVFYRCQ CTTVETVREL TEFAKSIPSF SSLFLNDQVT LLKYGVHEAI FAMLASIVNK
DGLLVANGSG FVTREFLRSL RKPFSDIIEP KFEFAVKFNA LELDDSDLAL FIAAIILCGD
RPGLMNVPRV EAIQDTILRA LEFHLQANHP DAQYLFPKLL QKMADLRQLV TEHAQMMQRI
KKTETETSLH PLLQEIYKDM Y

What is claimed is:

1. A compound having the chemical structure 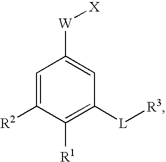
all salts, prodrugs, tautomers, and isomers thereof,
wherein:
X is selected from the group consisting of —C(O)OR¹⁶, —C(O)NR¹⁷R¹⁸, and a carboxylic acid isostere;
W is selected from the group consisting of a covalent bond, —NR⁵¹(CR⁴R⁵)₁₋₂—, —O—(CR⁴R⁵)₁₋₂—, —S—(CR⁴R⁵)₁₋₂—, —(CR⁴R⁵)₁₋₃—, and —CR⁶═CR⁷—;
R¹ and R² are independently selected from the group consisting of hydrogen, halogen, lower alkyl, lower alkenyl, lower alkynyl, —SR⁹, and —OR⁹, wherein lower alkyl, lower alkenyl and lower alkynyl are optionally substituted with one or more substituents selected from the group consisting of fluoro, —OH, —NH₂, lower alkoxy, fluoro substituted lower alkoxy, lower alkylthio, fluoro substituted lower alkylthio, cycloalkyl, heterocycloalkyl, aryl and heteroaryl, wherein cycloalkyl, heterocycloalkyl, aryl and heteroaryl are optionally substituted with one or more substituents selected from the group consisting of halogen, —OH, —NH₂, lower alkyl, fluoro substituted lower alkyl, lower alkenyl, fluoro substituted lower alkenyl, lower alkynyl, fluoro substituted lower alkynyl, lower alkoxy, fluoro substituted lower alkoxy, lower alkylthio, and fluoro substituted lower alkylthio;
R³ is selected from the group consisting of —[(CR⁴R⁵)ₘ—(Y)ₚ]ᵣ—R¹⁰ and —[(CR⁴R⁵)ₘ—(Y)ₚ]ᵣ—Ar₁-M-Ar₂;
L is selected from the group consisting of —O—, —S—, —NR⁵²—, —C(Z)-, —S(O)ₙ—, —C(Z)NR⁵²—, —NR⁵²C(Z)-, —NR⁵²(O)₂—, —S(O)₂NR⁵²—, —NR⁵²C(Z)NR⁵²— and —NR⁵²S(O)₂NR⁵²—;
Y is selected from the group consisting of —O—, —S—, —NR⁵³—, —C(Z)-, —S(O)ₙ—, —C(Z)NR⁵⁴—, —NR⁵⁴C(Z)-, —NR⁵⁴S(O)₂—, —S(O)₂NR⁵⁴—, —NR⁵⁴C(Z)NR⁵⁴—, and —NR⁵⁴S(O)₂NR⁵⁴—;
Ar₁ is selected from the group consisting of optionally substituted arylene and optionally substituted heteroarylene;
M is selected from the group consisting of a covalent bond, —CR¹⁹R²⁰—, —O—, —S—, —NR⁵³—, —C(Z)-, and —S(O)ₙ—;
Ar₂ is selected from the group consisting of optionally substituted aryl and optionally substituted heteroaryl;
R⁴ and R⁵ at each occurrence are independently selected from the group consisting of hydrogen, fluoro and lower alkyl, wherein lower alkyl is optionally substituted with one or more substituents selected from the group consisting of fluoro, —OH, —NH₂, lower alkoxy, fluoro substituted lower alkoxy, lower alkylthio, and fluoro substituted lower alkylthio; or
one R⁴ or R⁵ is selected from the group consisting of phenyl, 5-7 membered monocyclic heteroaryl, 3-7 membered monocyclic cycloalkyl, and 5-7 membered monocyclic heterocycloalkyl and any others of R⁴ and R⁵ are independently selected from the group consisting of hydrogen, fluoro and lower alkyl, wherein lower alkyl is optionally substituted with one or more substituents selected from the group consisting of fluoro, —OH, —NH₂, lower alkoxy, fluoro substituted lower alkoxy, lower alkylthio, and fluoro substituted lower alkylthio, and wherein phenyl, monocyclic heteroaryl, monocyclic cycloalkyl and monocyclic heterocycloalkyl are optionally substituted with one or more substituents selected from the group consisting of halogen, —OH, —NH₂, lower alkyl, fluoro substituted lower alkyl, lower alkoxy, fluoro substituted lower alkoxy, lower alkylthio, and fluoro substituted lower alkylthio; or
any two of R⁴ and R⁵ on the same or different carbons combine to form a 3-7 membered monocyclic cycloalkyl or 5-7 membered monocyclic heterocycloalkyl and any others of R⁴ and R⁵ are independently selected from the group consisting of hydrogen, fluoro and lower alkyl, wherein lower alkyl is optionally substituted with one or more substituents selected from the group consisting of fluoro, —OH, —NH₂, lower alkoxy, fluoro substituted lower alkoxy, lower alkylthio, and fluoro substituted lower alkylthio, and wherein the monocyclic cycloalkyl or monocyclic heterocycloalkyl are optionally substituted with one or more substituents selected from the group consisting of halogen, —OH, —NH₂, lower alkyl, fluoro substituted lower alkyl, lower alkoxy, fluoro substituted lower alkoxy, lower alkylthio, and fluoro substituted lower alkylthio;
R⁶ and R⁷ are independently hydrogen or lower alkyl, wherein lower alkyl is optionally substituted with one or more substituents selected from the group consisting of fluoro, —OH, —NH₂, lower alkoxy, fluoro substituted lower alkoxy, lower alkylthio, and fluoro substituted lower alkylthio; or
one of R⁶ and R⁷ is selected from the group consisting of phenyl, 5-7 membered monocyclic heteroaryl, 3-7 membered monocyclic cycloalkyl, and 5-7 membered monocyclic heterocycloalkyl and the other of R⁶ and R⁷ is hydrogen or lower alkyl, wherein lower alkyl is optionally substituted with one or more substituents selected from the group consisting of fluoro, —OH, —NH₂, lower alkoxy, fluoro substituted lower alkoxy, lower alkylthio, and fluoro substituted lower alkylthio, and wherein phenyl, monocyclic heteroaryl, monocyclic cycloalkyl and monocyclic heterocycloalkyl are optionally substituted with one or more substituents selected from the group consisting of halogen, —OH, —NH₂, lower alkyl, fluoro substituted lower alkyl, lower alkoxy, fluoro substituted lower alkoxy, lower alkylthio, and fluoro substituted lower alkylthio; or
R⁶ and R⁷ combine to form a 5-7 membered monocyclic cycloalkyl or 5-7 membered monocyclic heterocycloalkyl, wherein the monocyclic cycloalkyl or monocyclic heterocycloalkyl are optionally substituted with one or more substituents selected from the group consisting of halogen, —OH, —NH₂, lower alkyl, fluoro substituted lower alkyl, lower alkoxy, fluoro substituted lower alkoxy, lower alkylthio, and fluoro substituted lower alkylthio;
R⁹ at each occurrence is independently selected from the group consisting of lower alkyl, C₃₋₆ alkenyl, provided, however, that when R⁹ is C₃₋₆ alkenyl, no alkene carbon thereof is bound to the O of —OR⁹ or the S of —SR⁹, C₃₋₆ alkynyl, provided, however, that when R⁹ is C₃₋₆ alkynyl, no alkyne carbon thereof is bound to the O of —OR⁹ or the S of —SR⁹, cycloalkyl, heterocycloalkyl, aryl, and heteroaryl, wherein cycloalkyl, heterocycloalkyl, aryl and heteroaryl are optionally substituted with one or more substituents selected from the group consisting of halogen, —OH, —NH₂, lower alkyl, fluoro substituted lower alkyl, lower alkenyl, fluoro substituted lower alkenyl, lower alkynyl, fluoro substituted lower alkynyl, lower alkoxy, fluoro substituted lower alkoxy, lower alkylthio, and fluoro substituted lower alkylthio, and wherein lower alkyl, C₃₋₆ alkenyl and C₃₋₆ alkynyl are optionally substituted with one or more substituents selected from the group consisting of fluoro, —OH, —NH₂, lower alkoxy, fluoro substituted lower alkoxy, lower alkylthio, fluoro substituted lower alkylthio, cycloalkyl, heterocycloalkyl, aryl and heteroaryl, provided, however, that any substitution on the alkyl, C₃₋₆ alkenyl or C₃₋₆ alkynyl carbon bound to the O of —OR⁹ or the S of —SR⁹ is selected from the group consisting of fluoro, cycloalkyl, heterocycloalkyl, aryl and heteroaryl, wherein cycloalkyl, heterocycloalkyl, aryl, and heteroaryl substituents of alkyl, C₃₋₆ alkenyl and C₃₋₆ alkynyl are optionally substituted with one or more substituents selected from the group consisting of halogen, —OH, —NH₂, lower alkyl, fluoro substituted lower alkyl, lower alkenyl, fluoro substituted lower alkenyl, lower alkynyl, fluoro substituted lower alkynyl, lower alkoxy, fluoro substituted lower alkoxy, lower alkylthio, and fluoro substituted lower alkylthio;
R¹⁰ is selected from the group consisting of optionally substituted cycloalkyl, optionally substituted heterocycloalkyl, optionally substituted aryl, and optionally substituted heteroaryl;
R⁵¹ and R⁵² at each occurrence are independently selected from the group consisting of hydrogen, lower alkyl, phenyl, 5-7 membered monocyclic heteroaryl, 3-7 membered monocyclic cycloalkyl, and 5-7 membered monocyclic heterocycloalkyl, wherein lower alkyl is optionally substituted with one or more substituents selected from the group consisting of fluoro, —OH, —NH₂, lower alkoxy, fluoro substituted lower alkoxy, lower alkylthio, and fluoro substituted lower alkylthio, provided, however, that any substitution on the alkyl carbon bound to the N of —NR⁵¹— or —NR⁵²— is fluoro, and wherein phenyl, monocyclic heteroaryl, monocyclic cycloalkyl and monocyclic heterocycloalkyl are optionally substituted with one or more substituents selected from the group consisting of halogen, —OH, —NH₂, lower alkyl, fluoro substituted lower alkyl, lower alkoxy, fluoro substituted lower alkoxy, lower alkylthio, and fluoro substituted lower alkylthio;
R⁵³ at each occurrence is independently selected from the group consisting of hydrogen, lower alkyl, C₃₋₆ alkenyl, provided, however, that when R⁵³ is C₃₋₆ alkenyl, no alkene carbon thereof is bound to the N of —NR⁵³—, C₃₋₆ alkynyl, provided, however, that when R⁵³ is C₃₋₆ alkynyl, no alkyne carbon thereof is bound to the N of —NR⁵³—, cycloalkyl, heterocycloalkyl, aryl, heteroaryl, —C(Z)NR¹¹R¹², —S(O)₂NR¹¹R¹², —S(O)₂R¹³, —C(Z)R¹³, and —C(Z)OR¹⁵, wherein lower alkyl, C₃₋₆ alkenyl, and C₃₋₆ alkynyl, are optionally substituted with one or more substituents selected from the group consisting of fluoro, —OR²¹, —SR²¹, —NR²²R²³, cycloalkyl, heterocycloalkyl, aryl and heteroaryl, provided, however, that any substitution on the alkyl, C₃₋₆ alkenyl or C₃₋₆ alkynyl carbon bound to the N of any —NR⁵³— is selected from the group consisting of fluoro, cycloalkyl, heterocycloalkyl, aryl, and heteroaryl, and wherein any cycloalkyl, heterocycloalkyl, aryl or heteroaryl is optionally substituted with one or more substituents selected from the group consisting of halogen, —NO₂, —CN, —OR²¹, —SR²¹, —S(O)R²¹, —S(O)₂R²¹, —C(Z)R²¹, —C(Z)OR²¹, —NR²²R²³, —C(Z)NR²²R²³, —S(O)₂NR²²R²³, —C(NH)NR²²R²³, —NR²¹C(Z)R²¹, —NR²¹S(O)₂R²¹, —NR²¹C(Z)NR²²R²³, —NR²¹S(O)₂NR²²R²³, lower alkyl, lower alkenyl, and lower alkynyl, wherein the lower alkyl, lower alkenyl, and lower alkynyl optional substituents of cycloalkyl, heterocycloalkyl, aryl or heteroaryl are further optionally substituted with one or more substituents selected from the group consisting of fluoro, —OR²¹, —SR²¹, and —NR²²R²³;
R⁵⁴ at each occurrence is independently selected from the group consisting of hydrogen, lower alkyl, C₃₋₆ alkenyl, provided, however, that when R⁵⁴ is C₃₋₆ alkenyl, no alkene carbon thereof is bound to the N of any —NR⁵⁴—, C₃₋₆ alkynyl, provided, however, that when R⁵⁴ is C₃₋₆ alkynyl, no alkyne carbon thereof is bound to the N of any —NR⁵⁴—, cycloalkyl, heterocycloalkyl, aryl, and heteroaryl, wherein lower alkyl, C₃₋₆ alkenyl, and C₃₋₆ alkynyl, are optionally substituted with one or more substituents selected from the group consisting of fluoro, —OR²¹, —SR²¹, —NR²²R²³, cycloalkyl, heterocycloalkyl, aryl and heteroaryl, provided, however, that any substitution on the alkyl, C₃₋₆ alkenyl or C₃₋₆ alkynyl carbon bound to the N of any —NR⁵⁴— is selected from the group consisting of fluoro, cycloalkyl, heterocycloalkyl, aryl, and heteroaryl, and wherein any cycloalkyl, heterocycloalkyl, aryl or heteroaryl is optionally substituted with one or more substituents selected from the group consisting of halogen, —NO₂, —CN, —OR²¹, —SR²¹, —S(O)R²¹, —S(O)₂R²¹, —C(Z)R²¹, —C(Z)OR²¹, —NR²²R²³, —C(Z)NR²²R²³, —S(O)₂NR²²R²³, —C(NH)NR²²R²³, —NR²¹C(Z)R²¹, —NR²¹S(O)₂R²¹, —NR²¹C(Z)NR²²R²³, —NR²¹S(O)₂NR²²R²³, lower alkyl, lower alkenyl, and lower alkynyl, wherein the lower alkyl, lower alkenyl, and lower alkynyl optional substituents of cycloalkyl, heterocycloalkyl, aryl or heteroaryl are further optionally substituted with one or more substituents selected from the group consisting of fluoro, —OR²¹, —SR²¹, and —NR²²R²³;
R¹¹ and R¹² at each occurrence are independently selected from the group consisting of hydrogen, lower alkyl, C₃₋₆ alkenyl, provided, however, that when R¹¹ and/or R¹² is C₃₋₆ alkenyl, no alkene carbon thereof is bound to the N of any —C(Z)NR¹¹R¹² or —S(O)₂NR¹¹R¹², C₃₋₆ alkynyl, provided, however, that when R¹¹ and/or R¹² is C₃₋₆ alkynyl, no alkyne carbon thereof is bound to the N of any —C(Z)NR¹¹R¹² or —S(O)₂NR¹¹R¹², cycloalkyl, heterocycloalkyl, aryl, and heteroaryl, wherein lower alkyl, C₃₋₆ alkenyl, and C₃₋₆ alkynyl, are optionally substituted with one or more substituents selected from the group consisting of fluoro, —OR²¹, —SR²¹, —NR²²R²³, cycloalkyl, heterocycloalkyl, aryl and heteroaryl, provided, however, that any substitution on the alkyl, C₃₋₆ alkenyl or C₃₋₆ alkynyl carbon bound to the N of any —C(Z)NR¹¹R¹² or —S(O)₂NR¹¹R¹² is selected from the group consisting of fluoro, cycloalkyl, heterocycloalkyl, aryl, and heteroaryl, and wherein any cycloalkyl, heterocycloalkyl, aryl or heteroaryl is optionally substituted with one or more substituents selected from the group consisting of halogen, —NO₂, —CN, —OR²¹, —SR²¹, —S(O)R²¹, —S(O)₂R²¹, —C(Z)R²¹, —C(Z)OR²¹, —NR²²R²³, —C(Z)NR²²R²³, —S(O)₂NR²²R²³, —C(NH)NR²²R²³, —NR²¹C(Z)R²¹, —NR²¹S(O)R²¹, —NR²¹C(Z)NR²²R²³, —NR²¹S(O)₂NR²²R²³, lower alkyl, lower alkenyl, and lower alkynyl, wherein the lower alkyl, lower alkenyl, and lower alkynyl optional substituents of cycloalkyl, heterocycloalkyl, aryl or heteroaryl are further optionally substituted with one or more substituents selected from the group consisting of fluoro, —OR²¹, —SR²¹, and —NR²²R²³; or
R¹¹ and R¹² together with the nitrogen to which they are attached form a 5-7 membered monocyclic heterocycloalkyl or a 5 or 7 membered monocyclic nitrogen containing heteroaryl, wherein the monocyclic heterocycloalkyl or monocyclic nitrogen containing heteroaryl is optionally substituted with one or more substituents selected from the group consisting of halogen, —OH, —NH₂, —NO₂, —CN, lower alkyl, fluoro substituted lower alkyl, lower alkoxy, fluoro substituted lower alkoxy, lower alkylthio, fluoro substituted lower alkylthio, mono-alkylamino, di-alkylamino, and cycloalkylamino;
R¹³ at each occurrence is independently selected from the group consisting of lower alkyl, C₃₋₆ alkenyl, provided, however, that when R¹³ is C₃₋₆ alkenyl, no alkene carbon thereof is bound to C(Z) of —C(Z)R¹³, or S(O)₂ of —S(O)₂R¹³, C₃₋₆ alkynyl, provided, however, that when R¹³ is C₃₋₆ alkynyl, no alkyne carbon thereof is bound to C(Z) of —C(Z)R¹³, or S(O)₂ of —S(O)₂R¹³, cycloalkyl, heterocycloalkyl, aryl, and heteroaryl, wherein lower alkyl, C₃₋₆ alkenyl, and C₃₋₆ alkynyl, are optionally substituted with one or more substituents selected from the group consisting of fluoro, —OR²¹, —SR²¹, —NR²²R²³, cycloalkyl, heterocycloalkyl, aryl and heteroaryl, and wherein any cycloalkyl, heterocycloalkyl, aryl or heteroaryl is optionally substituted with one or more substituents selected from the group consisting of halogen, —NO₂, —CN, —OR²¹, —SR²¹, —S(O)R²¹, —S(O)₂R²¹, —C(Z)R²¹, —C(Z)OR²¹, —NR²²R²³, —C(Z)NR²²R²³, —S(O)₂NR²²R²³, —C(NH)NR²²R²³, —NR²¹C(Z)R²¹, —NR²¹S(O)₂R²¹, —NR²¹C(Z)NR²²R²³, —NR²¹S(O)₂NR²²R²³, lower alkyl, lower alkenyl, and lower alkynyl, wherein the lower alkyl, lower alkenyl, and lower alkynyl, optional substituents of cycloalkyl, heterocycloalkyl, aryl or heteroaryl are further optionally substituted with one or more substituents selected from the group consisting of fluoro, —OR²¹, —SR²¹, and —NR²²R²³;
R¹⁵ at each occurrence is independently selected from the group consisting of hydrogen, lower alkyl, C₃₋₆ alkenyl, provided, however, that when R¹⁵ is C₃₋₆ alkenyl, no alkene carbon thereof is bound to O of OR¹⁵, C₃₋₆ alkynyl, provided, however, that when R¹⁵ is C₃₋₆ alkynyl, no alkyne carbon thereof is bound to O of OR¹⁵, cycloalkyl, heterocycloalkyl, aryl, and heteroaryl, wherein lower alkyl, C₃₋₆ alkenyl, and C₃₋₆ alkynyl, are optionally substituted with one or more substituents selected from the group consisting of fluoro, —OR²¹, —SR²¹, —NR²²R²³, cycloalkyl, heterocycloalkyl, aryl and heteroaryl, provided, however, that any substitution on the alkyl, C₃₋₆ alkenyl or C₃₋₆ alkynyl carbon bound to the O of any OR¹⁵ is selected from the group consisting of fluoro, cycloalkyl, heterocycloalkyl, aryl, and heteroaryl, and wherein any cycloalkyl, heterocycloalkyl, aryl or heteroaryl is optionally substituted with one or more substituents selected from the group consisting of halogen, —NO₂, —CN, —OR²¹, —SR²¹, —S(O)R²¹, —S(O)₂R²¹, —C(Z)R²¹, —C(Z)OR²¹, —NR²²R²³, —C(Z)NR²²R²³, —S(O)₂NR²²R²³, —C(NH)NR²²R²³, —NR²¹C(Z)R²¹, —NR²¹S(O)₂R²¹, —NR²¹C(Z)NR²²R²³, —NR²¹S(O)NR²²R²³, lower alkyl, lower alkenyl, and lower alkynyl, wherein the lower alkyl, lower alkenyl, and lower alkynyl optional substituents of cycloalkyl, heterocycloalkyl, aryl or heteroaryl are further optionally substituted with one or more substituents selected from the group consisting of fluoro, —OR²¹, —SR²¹, and —NR²²R²³;
R¹⁶ is selected from the group consisting of hydrogen, lower alkyl, phenyl, 5-7 membered monocyclic heteroaryl, 3-7 membered monocyclic cycloalkyl, and 5-7 membered monocyclic heterocycloalkyl, wherein phenyl, monocyclic heteroaryl, monocyclic cycloalkyl and monocyclic heterocycloalkyl are optionally substituted with one or more substituents selected from the group consisting of halogen, —OH, —NH₂, lower alkyl, fluoro substituted lower alkyl, lower alkoxy, fluoro substituted lower alkoxy, lower alkylthio, and fluoro substituted lower alkylthio, and wherein lower alkyl is optionally substituted with one or more substituents selected from the group consisting of fluoro, —OH, —NH₂, lower alkoxy, fluoro substituted lower alkoxy, lower alkylthio and fluoro substituted lower alkylthio, provided, however, that when R¹⁶ is lower alkyl, any substitution on the alkyl carbon bound to the O of OR¹⁶ is fluoro;
R¹⁷ and R¹⁸ are independently selected from the group consisting of hydrogen, lower alkyl, phenyl, 5-7 membered monocyclic heteroaryl, 3-7 membered monocyclic cycloalkyl, and 5-7 membered monocyclic heterocycloalkyl, wherein phenyl, monocyclic heteroaryl, monocyclic cycloalkyl and monocyclic heterocycloalkyl are optionally substituted with one or more substituents selected from the group consisting of halogen, —OH, —NH₂, lower alkyl, fluoro substituted lower alkyl, lower alkoxy, fluoro substituted lower alkoxy, lower alkylthio, and fluoro substituted lower alkylthio, and wherein lower alkyl is optionally substituted with one or more substituents selected from the group consisting of fluoro, —OH, —NH₂, lower alkoxy, fluoro substituted lower alkoxy, lower alkylthio and fluoro substituted lower alkylthio, provided, however, that when R¹⁷ and/or R¹⁸ is lower alkyl, any substitution on the alkyl carbon bound to the N of NR¹⁷R¹⁸ is fluoro; or
R¹⁷ and R¹⁸ together with the nitrogen to which they are attached form a 5-7 membered monocyclic heterocycloalkyl or a 5 or 7 membered nitrogen containing monocyclic heteroaryl, wherein the monocyclic heterocycloalkyl or monocyclic nitrogen containing heteroaryl is optionally substituted with one or more substituents selected from the group consisting of halogen, —OH, —NH₂, lower alkyl, fluoro substituted lower alkyl, lower alkoxy, fluoro substituted lower alkoxy, lower alkylthio, and fluoro substituted lower alkylthio;
R¹⁹ and R²⁰ are independently selected from the group consisting of hydrogen, lower alkyl, lower alkenyl, lower alkynyl, cycloalkyl, heterocycloalkyl, aryl, and heteroaryl, wherein lower alkyl, lower alkenyl, and lower alkynyl, are optionally substituted with one or more substituents selected from the group consisting of fluoro, —OR²¹, —SR²¹, —NR²²R²³, cycloalkyl, heterocycloalkyl, aryl and heteroaryl, and wherein any cycloalkyl, heterocycloalkyl, aryl or heteroaryl is optionally substituted with one or more substituents selected from the group consisting of halogen, —NO₂, —CN, —OR²¹, —SR²¹, —S(O)R²¹, —S(O)₂R²¹, —C(Z)R²¹, —C(Z)OR²¹, —NR²²R²³, —C(Z)NR²²R²³, —S(O)₂NR²²R²³, —C(NH)NR²²R²³, —NR²¹C(Z)R²¹, —NR²¹S(O)₂R²¹, —NR²¹C(Z)NR²²R²³, —NR²¹S(O)₂NR²²R²³, lower alkyl, lower alkenyl, and lower alkynyl, wherein the lower alkyl, lower alkenyl, and lower alkynyl optional substituents of cycloalkyl, heterocycloalkyl, aryl or heteroaryl are further optionally substituted with substituents selected from the group consisting of fluoro, —OR²¹, —SR²¹, and —NR²²R²³; or
R¹⁹ and R²⁰ combine to form a 3-7 membered monocyclic cycloalkyl or 5-7 membered monocyclic heterocycloalkyl, wherein the monocyclic cycloalkyl or monocyclic heterocycloalkyl are optionally substituted with one or more substituents selected from the group consisting of halogen, —OH, —NH₂, lower alkyl, fluoro substituted lower alkyl, lower alkoxy, fluoro substituted lower alkoxy, lower alkylthio, and fluoro substituted lower alkylthio;
R²¹, R²², and R²³ at each occurrence are independently hydrogen or lower alkyl optionally substituted with one or more substituents selected from the group consisting of fluoro, lower alkoxy, fluoro substituted lower alkoxy, lower alkylthio, fluoro substituted lower alkylthio, mono-alkylamino, di-alkylamino, and cycloalkylamino provided, however, that any substitution on the lower alkyl carbon bound to O, S, or N of any of OR²¹, SR²¹, NR²¹, NR²² or NR²³ is fluoro, and further provided, however, that R²¹ bound to S, S(O), S(O)₂ or C(Z) is not hydrogen; or
R²² and R²³ together with the nitrogen to which they are attached form a 5-7 membered monocyclic heterocycloalkyl or a 5 or 7 membered monocyclic nitrogen containing heteroaryl, wherein the monocyclic heterocycloalkyl or monocyclic nitrogen containing heteroaryl is optionally substituted with one or more substituents selected from the group consisting of halogen, —OH, —NH₂, —NO₂, —CN, lower alkyl, fluoro substituted lower alkyl, lower alkoxy, fluoro substituted lower alkoxy, lower alkylthio, fluoro substituted lower alkylthio, mono-alkylamino, di-alkylamino, and cycloalkylamino;
Z is O or S;
m is 1, 2, 3, or 4;
n is 1 or 2;
p is 0 or 1, provided, however, that when p is 1, m is 1, and L is —O—, —S—, —NR⁵²—, —C(Z)NR⁵²—, —S(O)₂NR⁵²—, —NR⁵²C(Z)NR⁵²—, or NR⁵²S(O)₂NR⁵²—, then Y is not —O—, —S—, —NR⁵³—, —NR⁵⁴C(Z)-, —NR⁵⁴S(O)₂—, —NR⁵⁴C(Z)NR⁵⁴—, or NR⁵⁴S(O)₂NR⁵⁴—; and
r is 0 or 1.

2. The compound according to claim 1, wherein L is —S(O)₂— and R³ is R¹⁰, wherein R¹⁰ is optionally substituted phenyl.

3. The compound according to claim 2, wherein R¹⁰ is phenyl optionally substituted with one or more substituents selected from the group consisting of fluoro, —OH, —NH₂, lower alkyl, fluoro substituted lower alkyl, lower alkoxy, and fluoro substituted lower alkoxy.

4. The compound according to claim 1, wherein L is —O— and R³ is R¹⁰, wherein R¹⁰ is optionally substituted phenyl.

5. The compound according to claim 4, wherein R¹⁰ is phenyl optionally substituted with one or more substituents selected from the group consisting of fluoro, —OH, —NH₂, lower alkyl, fluoro substituted lower alkyl, lower alkoxy, and fluoro substituted lower alkoxy.

6. The compound according to any of claims 1-5, wherein at least one of R¹ and R² is —OR⁹.

7. The compound according to claim 6, wherein one of R¹ and R² is —OR⁹ and the other of R¹ and R² is hydrogen or halo.

8. The compound according to claim 7, wherein R² is —OR⁹ and R¹ is hydrogen.

9. The compound according to claim 8, wherein R⁹ is lower alkyl optionally substituted with one or more substituents selected from the group consisting of fluoro, lower alkoxy, fluoro substituted lower alkoxy, lower alkylthio, fluoro substituted lower alkylthio, cycloalkyl, and fluoro substituted cycloalkyl.

10. The compound according to claim 9, wherein X is C(O)OR¹⁶.

11. The compound according to claim 10, wherein R¹⁶ is H.

12. The compound according to claim 11, wherein W is —(CR⁴R⁵)₁₋₃—.

13. The compound according to claim 12, wherein W is —CH₂— or —CH₂CH₂—.

14. The compound according to claim 13, wherein R⁹ is lower alkyl optionally substituted with one or more substituents selected from the group consisting of fluoro, —OH, lower alkoxy, and lower alkylthio.

15. The compound according to claim 1 having the chemical structure 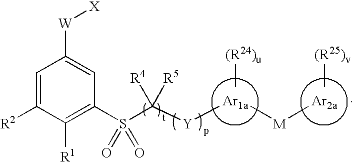
all salts, prodrugs, tautomers, and isomers thereof,
wherein:
X is selected from the group consisting of —C(O)OR¹⁶, —C(O)NR¹⁷R¹⁸, and a carboxylic acid isostere;
W is selected from the group consisting of a covalent bond, —NR⁵¹(CR⁴R⁵)₁₋₂—, —O—(CR⁴R⁵)₁₋₂—, —S—(CR⁴R⁵)₁₋₂—, —(CR⁴R⁵)₁₋₃—, and —CR⁶═CR⁷—;
Y is selected from the group consisting of —O—, —S—, —NR⁵³—, —C(Z)-, —S(O)ₙ—, —C(Z)NR⁵⁴—, —NR⁵⁴C(Z)-, —NR⁵⁴S(O)₂—, —S(O)₂NR⁵⁴—, —NR⁵⁴C(Z)NR⁵⁴—, and —NR⁵⁴S(O)₂NR⁵⁴—;
M is selected from the group consisting of a covalent bond, —CR¹⁹R²⁰—, —O—, —S—, —NR⁵³—, —C(Z)-, and —S(O)ₙ—;
Ar₁ₐ is selected from the group consisting of arylene and heteroarylene;
Ar₂ₐ is selected from the group consisting of aryl and heteroaryl;
R¹ and R² are independently selected from the group consisting of hydrogen, halogen, lower alkyl, lower alkenyl, lower alkynyl, —SR⁹, and —OR⁹, wherein lower alkyl, lower alkenyl and lower alkynyl are optionally substituted with one or more substituents selected from the group consisting of fluoro, —OH, —NH₂, lower alkoxy, fluoro substituted lower alkoxy, lower alkylthio, fluoro substituted lower alkylthio, cycloalkyl, heterocycloalkyl, aryl and heteroaryl, wherein cycloalkyl, heterocycloalkyl, aryl and heteroaryl are optionally substituted with one or more substituents selected from the group consisting of halogen, —OH, —NH₂, lower alkyl, fluoro substituted lower alkyl, lower alkenyl, fluoro substituted lower alkenyl, lower alkynyl, fluoro substituted lower alkynyl, lower alkoxy, fluoro substituted lower alkoxy, lower alkylthio, and fluoro substituted lower alkylthio;
R⁴ and R⁵ at each occurrence are independently selected from the group consisting of hydrogen, fluoro and lower alkyl, wherein lower alkyl is optionally substituted with one or more substituents selected from the group consisting of fluoro, —OH, —NH₂, lower alkoxy, fluoro substituted lower alkoxy, lower alkylthio, and fluoro substituted lower alkylthio; or
one R⁴ or R⁵ is selected from the group consisting of phenyl, 5-7 membered monocyclic heteroaryl, 3-7 membered monocyclic cycloalkyl, and 5-7 membered monocyclic heterocycloalkyl and any others of R⁴ and R⁵ are independently selected from the group consisting of hydrogen, fluoro and lower alkyl, wherein lower alkyl is optionally substituted with one or more substituents selected from the group consisting of fluoro, —OH, —NH₂, lower alkoxy, fluoro substituted lower alkoxy, lower alkylthio, and fluoro substituted lower alkylthio, and wherein phenyl, monocyclic heteroaryl, monocyclic cycloalkyl and monocyclic heterocycloalkyl are optionally substituted with one or more substituents selected from the group consisting of halogen, —OH, —NH₂, lower alkyl, fluoro substituted lower alkyl, lower alkoxy, fluoro substituted lower alkoxy, lower alkylthio, and fluoro substituted lower alkylthio; or
any two of R⁴ and R⁵ on the same or different carbons combine to form a 3-7 membered monocyclic cycloalkyl or 5-7 membered monocyclic heterocycloalkyl and any others of R⁴ and R⁵ are independently selected from the group consisting of hydrogen, fluoro and lower alkyl, wherein lower alkyl is optionally substituted with one or more substituents selected from the group consisting of fluoro, —OH, —NH₂, lower alkoxy, fluoro substituted lower alkoxy, lower alkylthio, and fluoro substituted lower alkylthio, and wherein the monocyclic cycloalkyl or monocyclic heterocycloalkyl are optionally substituted with one or more substituents selected from the group consisting of halogen, —OH, —NH₂, lower alkyl, fluoro substituted lower alkyl, lower alkoxy, fluoro substituted lower alkoxy, lower alkylthio, and fluoro substituted lower alkylthio;
R⁶ and R⁷ are independently hydrogen or lower alkyl, wherein lower alkyl is optionally substituted with one or more substituents selected from the group consisting of fluoro, —OH, —NH₂, lower alkoxy, fluoro substituted lower alkoxy, lower alkylthio, and fluoro substituted lower alkylthio; or
one of R⁶ and R⁷ is selected from the group consisting of phenyl, 5-7 membered monocyclic heteroaryl, 3-7 membered monocyclic cycloalkyl, and 5-7 membered monocyclic heterocycloalkyl and the other of R⁶ and R⁷ is hydrogen or lower alkyl, wherein lower alkyl is optionally substituted with one or more substituents selected from the group consisting of fluoro, —OH, —NH₂, lower alkoxy, fluoro substituted lower alkoxy, lower alkylthio, and fluoro substituted lower alkylthio, and wherein phenyl, monocyclic heteroaryl, monocyclic cycloalkyl and monocyclic heterocycloalkyl are optionally substituted with one or more substituents selected from the group consisting of halogen, —OH, —NH₂, lower alkyl, fluoro substituted lower alkyl, lower alkoxy, fluoro substituted lower alkoxy, lower alkylthio, and fluoro substituted lower alkylthio; or
R⁶ and R⁷ combine to form a 5-7 membered monocyclic cycloalkyl or 5-7 membered monocyclic heterocycloalkyl, wherein the monocyclic cycloalkyl or monocyclic heterocycloalkyl are optionally substituted with one or more substituents selected from the group consisting of halogen, —OH, —NH₂, lower alkyl, fluoro substituted lower alkyl, lower alkoxy, fluoro substituted lower alkoxy, lower alkylthio, and fluoro substituted lower alkylthio;
R⁹ at each occurrence is independently selected from the group consisting of lower alkyl, C₃₋₆ alkenyl, provided, however, that when R⁹ is C₃₋₆ alkenyl, no alkene carbon thereof is bound to the O of —OR⁹ or the S of —SR⁹, C₃₋₆ alkynyl, provided, however, that when R⁹ is C₃₋₆ alkynyl, no alkyne carbon thereof is bound to the O of —OR⁹ or the S of —SR⁹, cycloalkyl, heterocycloalkyl, aryl, and heteroaryl, wherein cycloalkyl, heterocycloalkyl, aryl and heteroaryl are optionally substituted with one or more substituents selected from the group consisting of halogen, —OH, —NH₂, lower alkyl, fluoro substituted lower alkyl, lower alkenyl, fluoro substituted lower alkenyl, lower alkynyl, fluoro substituted lower alkynyl, lower alkoxy, fluoro substituted lower alkoxy, lower alkylthio, and fluoro substituted lower alkylthio, and wherein lower alkyl, C₃₋₆ alkenyl and C₃₋₆ alkynyl are optionally substituted with one or more substituents selected from the group consisting of fluoro, —OH, —NH₂, lower alkoxy, fluoro substituted lower alkoxy, lower alkylthio, fluoro substituted lower alkylthio, cycloalkyl, heterocycloalkyl, aryl and heteroaryl, provided, however, that any substitution on the alkyl, C₃₋₆ alkenyl or C₃₋₆ alkynyl carbon bound to the O of —OR⁹ or the S of —SR⁹ is selected from the group consisting of fluoro, cycloalkyl, heterocycloalkyl, aryl and heteroaryl, wherein cycloalkyl, heterocycloalkyl, aryl, and heteroaryl substituents of alkyl, C₃₋₆ alkenyl and C₃₋₆ alkynyl are optionally substituted with one or more substituents selected from the group consisting of halogen, —OH, —NH₂, lower alkyl, fluoro substituted lower alkyl, lower alkenyl, fluoro substituted lower alkenyl, lower alkynyl, fluoro substituted lower alkynyl, lower alkoxy, fluoro substituted lower alkoxy, lower alkylthio, and fluoro substituted lower alkylthio;
R⁵¹ is selected from the group consisting of hydrogen, lower alkyl, phenyl, 5-7 membered monocyclic heteroaryl, 3-7 membered monocyclic cycloalkyl, and 5-7 membered monocyclic heterocycloalkyl, wherein lower alkyl is optionally substituted with one or more substituents selected from the group consisting of fluoro, —OH, —NH₂, lower alkoxy, fluoro substituted lower alkoxy, lower alkylthio, and fluoro substituted lower alkylthio, provided, however, that any substitution on the alkyl carbon bound to the N of —NR⁵¹— is fluoro, and wherein phenyl, monocyclic heteroaryl, monocyclic cycloalkyl and monocyclic heterocycloalkyl are optionally substituted with one or more substituents selected from the group consisting of halogen, —OH, —NH₂, lower alkyl, fluoro substituted lower alkyl, lower alkoxy, fluoro substituted lower alkoxy, lower alkylthio, and fluoro substituted lower alkylthio;
R⁵³ at each occurrence is independently selected from the group consisting of hydrogen, lower alkyl, C₃₋₆ alkenyl, provided, however, that when R⁵³ is C₃₋₆ alkenyl, no alkene carbon thereof is bound to the N of —NR⁵³—, C₃₋₆ alkynyl, provided, however, that when R⁵³ is C₃₋₆ alkynyl, no alkyne carbon thereof is bound to the N of —NR⁵³—, cycloalkyl, heterocycloalkyl, aryl, heteroaryl, —C(Z)NR¹¹R¹², —S(O)₂NR¹¹R¹², —S(O)₂R¹³, —C(Z)R¹³, and —C(Z)OR¹⁵, wherein lower alkyl, C₃₋₆ alkenyl, and C₃₋₆ alkynyl, are optionally substituted with one or more substituents selected from the group consisting of fluoro, —OR²¹, —SR²¹, —NR²²R²³, cycloalkyl, heterocycloalkyl, aryl and heteroaryl, provided, however, that any substitution on the alkyl, C₃₋₆ alkenyl or C₃₋₆ alkynyl carbon bound to the N of any —NR⁵³— is selected from the group consisting of fluoro, cycloalkyl, heterocycloalkyl, aryl, and heteroaryl, and wherein any cycloalkyl, heterocycloalkyl, aryl or heteroaryl is optionally substituted with one or more substituents selected from the group consisting of halogen, —NO₂, —CN, —OR²¹, —SR²¹, —S(O)R²¹, —S(O)₂R²¹, —C(Z)R²¹, —C(Z)OR²¹, —NR²²R²³, —C(Z)NR²²R²³, —S(O)₂NR²²R²³, —C(NH)NR²²R²³, —NR²¹C(Z)R²¹, —NR²¹S(O)R²¹, —NR²¹C(Z)NR²²R²³, —NR²¹S(O)₂NR²²R²³, lower alkyl, lower alkenyl, and lower alkynyl, wherein the lower alkyl, lower alkenyl, and lower alkynyl optional substituents of cycloalkyl, heterocycloalkyl, aryl or heteroaryl are further optionally substituted with one or more substituents selected from the group consisting of fluoro, —OR²¹, —SR²¹, and —NR²²R²³;
R⁵⁴ at each occurrence is independently selected from the group consisting of hydrogen, lower alkyl, C₃₋₆ alkenyl, provided, however, that when R⁵⁴ is C₃₋₆ alkenyl, no alkene carbon thereof is bound to the N of any —NR⁵⁴—, C₃₋₆ alkynyl, provided, however, that when R⁵⁴ is C₃₋₆ alkynyl, no alkyne carbon thereof is bound to the N of any —NR⁵⁴—, cycloalkyl, heterocycloalkyl, aryl, and heteroaryl, wherein lower alkyl, C₃₋₆ alkenyl, and C₃₋₆ alkynyl, are optionally substituted with one or more substituents selected from the group consisting of fluoro, —OR²¹, —SR²¹, —NR²²R²³, cycloalkyl, heterocycloalkyl, aryl and heteroaryl, provided, however, that any substitution on the alkyl, C₃₋₆ alkenyl or C₃₋₆ alkynyl carbon bound to the N of any —NR⁵⁴— is selected from the group consisting of fluoro, cycloalkyl, heterocycloalkyl, aryl, and heteroaryl, and wherein any cycloalkyl, heterocycloalkyl, aryl or heteroaryl is optionally substituted with one or more substituents selected from the group consisting of halogen, —NO₂, —CN, —OR²¹, —SR²¹, —S(O)R²¹, —S(O)₂R²¹, —C(Z)R²¹, —C(Z)OR²¹, —NR²²R²³, —C(Z)NR²²R²³, —S(O)₂NR²²R²³, —C(NH)NR²²R²³, —NR²¹C(Z)R²¹, —NR²¹S(O)R²¹, —NR²¹C(Z)NR²²R²³, —NR²¹S(O)₂NR²²R²³, lower alkyl, lower alkenyl, and lower alkynyl, wherein the lower alkyl, lower alkenyl, and lower alkynyl optional substituents of cycloalkyl, heterocycloalkyl, aryl or heteroaryl are further optionally substituted with one or more substituents selected from the group consisting of fluoro, —OR²¹, —SR²¹, and —NR²²R²³;
R¹¹ and R¹² at each occurrence are independently selected from the group consisting of hydrogen, lower alkyl, C₃₋₆ alkenyl, provided, however, that when R¹¹ and/or R¹² is C₃₋₆ alkenyl, no alkene carbon thereof is bound to the N of any —C(Z)NR¹¹R¹² or —S(O)₂NR¹¹R¹², C₃₋₆ alkynyl, provided, however, that when R¹¹ and/or R¹² is C₃₋₆ alkynyl, no alkyne carbon thereof is bound to the N of any —C(Z)NR¹¹R¹² or —S(O)₂NR¹¹R¹², cycloalkyl, heterocycloalkyl, aryl, and heteroaryl, wherein lower alkyl, C₃₋₆ alkenyl, and C₃₋₆ alkynyl, are optionally substituted with one or more substituents selected from the group consisting of fluoro, —OR²¹, —SR²¹, —NR²²R²³, cycloalkyl, heterocycloalkyl, aryl and heteroaryl, provided, however, that any substitution on the alkyl, C₃₋₆ alkenyl or C₃₋₆ alkynyl carbon bound to the N of any —C(Z)NR¹¹R¹² or —S(O)₂NR¹¹R¹² is selected from the group consisting of fluoro, cycloalkyl, heterocycloalkyl, aryl, and heteroaryl, and wherein any cycloalkyl, heterocycloalkyl, aryl or heteroaryl is optionally substituted with one or more substituents selected from the group consisting of halogen, —NO₂, —CN, —OR²¹, —SR²¹, —S(O)R²¹, —S(O)₂R²¹, —C(Z)R¹², —C(Z)OR²¹, —NR²²R²³, —C(Z)NR²²R²³, —S(O)₂NR²²R²³, —C(NH)NR²²R²³, —NR²¹C(Z)R²¹, —NR²¹S(O)R²¹, —NR²¹C(Z)NR²²R²³, —NR²¹S(O)₂NR²²R²³, lower alkyl, lower alkenyl, and lower alkynyl, wherein the lower alkyl, lower alkenyl, and lower alkynyl optional substituents of cycloalkyl, heterocycloalkyl, aryl or heteroaryl are further optionally substituted with one or more substituents selected from the group consisting of fluoro, —OR²¹, —SR²¹, and —NR²²R²³; or
R¹¹ and R¹² together with the nitrogen to which they are attached form a 5-7 membered monocyclic heterocycloalkyl or a 5 or 7 membered monocyclic nitrogen containing heteroaryl, wherein the monocyclic heterocycloalkyl or monocyclic nitrogen containing heteroaryl is optionally substituted with one or more substituents selected from the group consisting of halogen, —OH, —NH₂, —NO₂, —CN, lower alkyl, fluoro substituted lower alkyl, lower alkoxy, fluoro substituted lower alkoxy, lower alkylthio, fluoro substituted lower alkylthio, mono-alkylamino, di-alkylamino, and cycloalkylamino;
R¹³ at each occurrence is independently selected from the group consisting of lower alkyl, C₃₋₆ alkenyl, provided, however, that when R¹³ is C₃₋₆ alkenyl, no alkene carbon thereof is bound to C(Z) of —C(Z)R¹³, or S(O)₂ of —S(O)₂R¹³, C₃₋₆ alkynyl, provided, however, that when R¹³ is C₃₋₆ alkynyl, no alkyne carbon thereof is bound to C(Z) of —C(Z)R¹³, or S(O)₂ of —S(O)₂R¹³, cycloalkyl, heterocycloalkyl, aryl, and heteroaryl, wherein lower alkyl, C₃₋₆ alkenyl, and C₃₋₆ alkynyl, are optionally substituted with one or more substituents selected from the group consisting of fluoro, —OR²¹, —SR²¹, —NR²²R²³, cycloalkyl, heterocycloalkyl, aryl and heteroaryl, and wherein any cycloalkyl, heterocycloalkyl, aryl or heteroaryl is optionally substituted with one or more substituents selected from the group consisting of halogen, —NO₂, —CN, —OR²¹, —SR²¹, —S(O)R²¹, —S(O)₂R²¹, —C(Z)R²¹, —C(Z)OR²¹, —NR²²R²³, —C(Z)NR²²R²³, —S(O)₂NR²²R²³, —C(NH)NR²²R²³, —NR²¹C(Z)R²¹, —NR²¹S(O)₂R²¹, —NR²¹C(Z)NR²²R²³, —NR²¹S(O)₂NR²²R²³, lower alkyl, lower alkenyl, and lower alkynyl, wherein the lower alkyl, lower alkenyl, and lower alkynyl, optional substituents of cycloalkyl, heterocycloalkyl, aryl or heteroaryl are further optionally substituted with one or more substituents selected from the group consisting of fluoro, —OR²¹, —SR²¹, and —NR²²R²³;
R¹⁵ at each occurrence is independently selected from the group consisting of hydrogen, lower alkyl, C₃₋₆ alkenyl, provided, however, that when R¹⁵ is C₃₋₆ alkenyl, no alkene carbon thereof is bound to O of OR¹⁵, C₃₋₆ alkynyl, provided, however, that when R¹⁵ is C₃₋₆ alkynyl, no alkyne carbon thereof is bound to O of OR¹⁵, cycloalkyl, heterocycloalkyl, aryl, and heteroaryl, wherein lower alkyl, C₃₋₆ alkenyl, and C₃₋₆ alkynyl, are optionally substituted with one or more substituents selected from the group consisting of fluoro, —OR²¹, —SR²¹, —NR²²R²³, cycloalkyl, heterocycloalkyl, aryl and heteroaryl, provided, however, that any substitution on the alkyl, C₃₋₆ alkenyl or C₃₋₆ alkynyl carbon bound to the O of any OR¹⁵ is selected from the group consisting of fluoro, cycloalkyl, heterocycloalkyl, aryl, and heteroaryl, and wherein any cycloalkyl, heterocycloalkyl, aryl or heteroaryl is optionally substituted with one or more substituents selected from the group consisting of halogen, —NO₂, —CN, —OR²¹, —SR²¹, —S(O)R²¹, —S(O)₂R²¹, —C(Z)R²¹, —C(Z)OR²¹, —NR²²R²³, —C(Z)NR²²R²³, —S(O)₂NR²²R²³, —C(NH)NR²²R²³, —NR²¹C(Z)R²¹, —NR²¹S(O)₂R²¹, —NR²¹C(Z)NR²²R²³, —NR²¹S(O)₂NR²²R²³, lower alkyl, lower alkenyl, and lower alkynyl, wherein the lower alkyl, lower alkenyl, and lower alkynyl optional substituents of cycloalkyl, heterocycloalkyl, aryl or heteroaryl are further optionally substituted with one or more substituents selected from the group consisting of fluoro, —OR²¹, —SR²¹, and —NR²²R²³;
R¹⁶ is selected from the group consisting of hydrogen, lower alkyl, phenyl, 5-7 membered monocyclic heteroaryl, 3-7 membered monocyclic cycloalkyl, and 5-7 membered monocyclic heterocycloalkyl, wherein phenyl, monocyclic heteroaryl, monocyclic cycloalkyl and monocyclic heterocycloalkyl are optionally substituted with one or more substituents selected from the group consisting of halogen, —OH, —NH₂, lower alkyl, fluoro substituted lower alkyl, lower alkoxy, fluoro substituted lower alkoxy, lower alkylthio, and fluoro substituted lower alkylthio, and wherein lower alkyl is optionally substituted with one or more substituents selected from the group consisting of fluoro, —OH, —NH₂, lower alkoxy, fluoro substituted lower alkoxy, lower alkylthio and fluoro substituted lower alkylthio, provided, however, that when R¹⁶ is lower alkyl, any substitution on the alkyl carbon bound to the O of OR¹⁶ is fluoro;
R¹⁷ and R¹⁸ are independently selected from the group consisting of hydrogen, lower alkyl, phenyl, 5-7 membered monocyclic heteroaryl, 3-7 membered monocyclic cycloalkyl, and 5-7 membered monocyclic heterocycloalkyl, wherein phenyl, monocyclic heteroaryl, monocyclic cycloalkyl and monocyclic heterocycloalkyl are optionally substituted with one or more substituents selected from the group consisting of halogen, —OH, —NH₂, lower alkyl, fluoro substituted lower alkyl, lower alkoxy, fluoro substituted lower alkoxy, lower alkylthio, and fluoro substituted lower alkylthio, and wherein lower alkyl is optionally substituted with one or more substituents selected from the group consisting of fluoro, —OH, —NH₂, lower alkoxy, fluoro substituted lower alkoxy, lower alkylthio and fluoro substituted lower alkylthio, provided, however, that when R¹⁷ and/or R¹⁸ is lower alkyl, any substitution on the alkyl carbon bound to the N of NR¹⁷R¹⁸ is fluoro; or
R¹⁷ and R¹⁸ together with the nitrogen to which they are attached form a 5-7 membered monocyclic heterocycloalkyl or a 5 or 7 membered nitrogen containing monocyclic heteroaryl, wherein the monocyclic heterocycloalkyl or monocyclic nitrogen containing heteroaryl is optionally substituted with one or more substituents selected from the group consisting of halogen, —OH, —NH₂, lower alkyl, fluoro substituted lower alkyl, lower alkoxy, fluoro substituted lower alkoxy, lower alkylthio, and fluoro substituted lower alkylthio;
R¹⁹ and R²⁰ are independently selected from the group consisting of hydrogen, lower alkyl, lower alkenyl, lower alkynyl, cycloalkyl, heterocycloalkyl, aryl, and heteroaryl, wherein lower alkyl, lower alkenyl, and lower alkynyl, are optionally substituted with one or more substituents selected from the group consisting of fluoro, —OR²¹, —SR²¹, —NR²²R²³, cycloalkyl, heterocycloalkyl, aryl and heteroaryl, and wherein any cycloalkyl, heterocycloalkyl, aryl or heteroaryl is optionally substituted with one or more substituents selected from the group consisting of halogen, —NO₂, —CN, —OR²¹, —SR²¹, —S(O)R²¹, —S(O)₂R²¹, —C(Z)R²¹, —C(Z)OR²¹, —NR²²R²³, —C(Z)NR²²R²³, —S(O)₂NR²²R²³, —C(NH)NR²²R²³, —NR²¹C(Z)R²¹, —NR²¹S(O)₂R²¹, —NR²¹C(Z)NR²²R²³, —NR²¹S(O)₂NR²²R²³, lower alkyl, lower alkenyl, and lower alkynyl, wherein the lower alkyl, lower alkenyl, and lower alkynyl optional substituents of cycloalkyl, heterocycloalkyl, aryl or heteroaryl are further optionally substituted with substituents selected from the group consisting of fluoro, —OR²¹, —SR²¹, and —NR²²R²³; or
R¹⁹ and R²⁰ combine to form a 3-7 membered monocyclic cycloalkyl or 5-7 membered monocyclic heterocycloalkyl, wherein the monocyclic cycloalkyl or monocyclic heterocycloalkyl are optionally substituted with one or more substituents selected from the group consisting of halogen, —OH, —NH₂, lower alkyl, fluoro substituted lower alkyl, lower alkoxy, fluoro substituted lower alkoxy, lower alkylthio, and fluoro substituted lower alkylthio;
R²¹, R²², and R²³ at each occurrence are independently hydrogen or lower alkyl optionally substituted with one or more substituents selected from the group consisting of fluoro, lower alkoxy, fluoro substituted lower alkoxy, lower alkylthio, fluoro substituted lower alkylthio, mono-alkylamino, di-alkylamino, and cycloalkylamino provided, however, that any substitution on the lower alkyl carbon bound to O, S, or N of any of OR²¹, SR²¹, NR²¹, NR²² or NR²³ is fluoro, and further provided, however, that R²¹ bound to S, S(O), S(O)₂ or C(Z) is not hydrogen; or
R²² and R²³ together with the nitrogen to which they are attached form a 5-7 membered monocyclic heterocycloalkyl or a 5 or 7 membered monocyclic nitrogen containing heteroaryl, wherein the monocyclic heterocycloalkyl or monocyclic nitrogen containing heteroaryl is optionally substituted with one or more substituents selected from the group consisting of halogen, —OH, —NH₂, —NO₂, —CN, lower alkyl, fluoro substituted lower alkyl, lower alkoxy, fluoro substituted lower alkoxy, lower alkylthio, fluoro substituted lower alkylthio, mono-alkylamino, di-alkylamino, and cycloalkylamino;
R²⁴ at each occurrence is independently selected from the group consisting of halogen, lower alkyl, lower alkenyl, lower alkynyl, —NO₂, —CN, —OR²⁶, —SR²⁶, —OC(O)R²⁶, —OC(S)R²⁶, —C(O)R²⁶, —C(S)R²⁶, —C(O)OR²⁶, —C(S)OR²⁶, —S(O)R²⁶, —S(O)R²⁶, —C(O)NR²⁷R²⁸, —C(S)NR²⁷R²⁸, —S(O)₂NR²⁷R²⁸, —C(NH)NR²⁷R²⁸, —NR²⁶C(O)R²⁶, —NR²⁶C(S)R²⁶, —NR²⁶S(O)₂R²⁶, NR²⁶C(O)NR²⁷R²⁸, NR²⁶C(S)NR²⁷R²⁸, —NR²⁶S(O)₂NR²⁷R²⁸, and —NR²⁷R²⁸, wherein lower alkyl is optionally substituted with one or more substituents selected from the group consisting of fluoro, —OR³⁶, —SR³⁶, and —NR³⁷R³⁸, and wherein lower alkenyl and lower alkynyl are optionally substituted with one or more substituents selected from the group consisting of fluoro, —OR³⁶, —SR³⁶, —NR³⁷R³⁸, and —R³⁵;
R²⁵ at each occurrence is independently selected from the group consisting of halogen, lower alkyl, lower alkenyl, lower alkynyl, cycloalkyl, heterocycloalkyl, aryl, heteroaryl, —NO₂, —CN, —OR²⁹, —SR²⁹, —OC(O)R²⁹, —OC(S)R²⁹, —C(O)R²⁹, —C(S)R²⁹, —C(O)OR²⁹, —C(S)OR²⁹, —S(O)R²⁹, —S(O)₂R²⁹, —C(O)NR²⁹R²⁹, —C(S)NR²⁹R²⁹, —S(O)₂NR²⁹R²⁹, —C(NH)NR³⁰R³¹, —NR²⁹C(O)R²⁹, —NR²⁹C(S)R²⁹, —NR²⁹R²⁹, —NR²⁹C(O)NR²⁹R²⁹, —NR²⁹C(S)NR²⁹R²⁹, —NR²⁹S(O)₂NR²⁹R²⁹, and —NR²⁹R²⁹, wherein lower alkyl is optionally substituted with one or more substituents selected from the group consisting of fluoro, —OR³⁶, —SR³⁶—NR³⁷R³⁸, and —R³², and wherein lower alkenyl and lower alkynyl are optionally substituted with one or more substituents selected from the group consisting of fluoro, —OR³⁶, —SR³⁶, —NR³⁷R³⁸, —R³⁵ and —R³², and wherein cycloalkyl, heterocycloalkyl, aryl, and heteroaryl are optionally substituted with one or more substituents selected from the group consisting of halogen, —NO₂, —CN, —OR³⁶, —SR³⁶, —NR³⁷R³⁸, —R³⁵, —R³³, and —R³⁴;
R²⁶, R²⁷ and R²⁸ at each occurrence are independently selected from the group consisting of hydrogen, lower alkyl, C₃₋₆ alkenyl, provided, however, that no alkene carbon thereof is bound to any O, S, N, C(O), C(S), S(O) or S(O)₂ of R²⁴, and C₃₋₆ alkynyl, provided, however, that no alkyne carbon thereof is bound to any O, S, N, C(O), C(S), S(O) or S(O)₂ of R²⁴, wherein lower alkyl is optionally substituted with one or more substituents selected from the group consisting of fluoro, —OR³⁶, —SR³⁶, and —NR³⁷R³⁸, and wherein lower alkenyl and lower alkynyl are optionally substituted with one or more substituents selected from the group consisting of fluoro, —OR³⁶, —SR³⁶, —NR³⁷R³⁸, and —R³⁵, further provided, however, that R²⁶ bound to S, C(O), C(S), S(O), or S(O)₂ is not hydrogen, or
R²⁷ and R²⁸ combine with the nitrogen to which they are attached to form cycloalkylamino;
R²⁹, R³⁰ and R³¹ at each occurrence are independently selected from the group consisting of hydrogen, lower alkyl, C₃₋₆ alkenyl, provided, however, that no alkene carbon thereof is bound to any O, S, N, C(O), C(S), S(O) or S(O)₂ of R²⁵, C₃₋₆ alkynyl, provided, however, that no alkyne carbon thereof is bound to any O, S, N, C(O), C(S), S(O) or S(O)₂ of R²⁵, cycloalkyl, heterocycloalkyl, aryl and heteroaryl, or
R³⁰ and R³¹ combine with the nitrogen to which they are attached to form a 5-7 membered heterocycloalkyl or a 5 or 7 membered nitrogen containing heteroaryl, wherein lower alkyl is optionally substituted with one or more substituents selected from the group consisting of fluoro, —OR³⁶, —SR³⁶, —NR³⁷R³⁸ and R³², wherein lower alkenyl and lower alkynyl are optionally substituted with one or more substituents selected from the group consisting of fluoro, —OR³⁶, —SR³⁶, —NR³⁷R³⁸, —R³⁵ and —R³², and wherein cycloalkyl, heterocycloalkyl, aryl, heteroaryl, 5-7 membered heterocycloalkyl, and 5 or 7 membered nitrogen containing heteroaryl are optionally substituted with one or more substituents selected from the group consisting of halogen, —NO₂, —CN, —OH, —NH₂, —OR³⁶, —SR³⁶, —NHR³⁶, —NR³⁷R³⁸, —R³³, —R³⁴, and —R³⁵, further provided, however, that R²⁹ bound to S, C(O), C(S), S(O), or S(O)₂ is not hydrogen;
R³² at each occurrence is independently selected from the group consisting of cycloalkyl, heterocycloalkyl, aryl and heteroaryl, wherein cycloalkyl, heterocycloalkyl, aryl and heteroaryl are optionally substituted with one or more substituents selected from the group consisting of halogen, —NO₂, —CN, —OR³⁶, —SR³⁶, —NR³⁷R³⁸, —R³³, —R³⁴, and —R³⁵;
R³³ at each occurrence is independently lower alkenyl optionally substituted with one or more substituents selected from the group consisting of fluoro, —OR³⁶, —SR³⁶, —NR³⁷R³⁸ and —R³⁵;
R³⁴ at each occurrence is independently lower alkynyl optionally substituted with one or more substituents selected from the group consisting of fluoro, —OR³⁶, —SR³⁶, —NR³⁷R³⁸ and R³⁵;
R³⁵ at each occurrence is independently lower alkyl optionally substituted with one or more substituents selected from the group consisting of fluoro, —OR³⁶, —SR³⁶, and —NR³⁷R³⁸;
R³⁶, R³⁷ and R³⁸ at each occurrence is independently hydrogen or lower alkyl optionally substituted with one or more substituents selected from the group consisting of fluoro, lower alkoxy, fluoro substituted lower alkoxy, lower alkylthio, fluoro substituted lower alkylthio, mono-alkylamino, di-alkylamino, and cycloalkylamino, or —NR³⁷R³⁸ is cycloalkylamino, provided, however, that any substitution on the lower alkyl carbon bound to the O, S, or N of any of OR³⁶, SR⁶, NR³⁶, NR³⁷ or NR³⁸ is fluoro, and further provided, however, that R³⁶ bound to S is not hydrogen;
Z is O or S;
n is 1 or 2;
u is 0, 1, 2, 3 or 4;
v is 0, 1, 2, 3, 4, or 5;
p is 0 or 1; and
t is 0, 1, 2, 3 or 4, provided, however, that when t=0, then p=0.

16. The compound according to claim 15, wherein at least one of R¹ and R² is —OR⁹.

17. The compound according to claim 16, wherein one of R¹ and R² is —OR⁹ and the other of R¹ and R² is hydrogen or halo.

18. The compound according to claim 17, wherein R² is —OR⁹ and R¹ is hydrogen.

19. The compound according to claim 18, wherein R⁹ is lower alkyl optionally substituted with one or more substituents selected from the group consisting of fluoro, lower alkoxy, fluoro substituted lower alkoxy, lower alkylthio, fluoro substituted lower alkylthio, cycloalkyl, and fluoro substituted cycloalkyl.

20. The compound according to claim 1 having the chemical structure

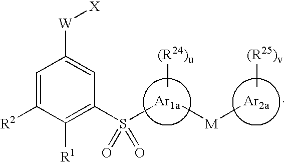

21. The compound according to claim 20, wherein at least one of R¹ and R² is —OR⁹.

22. The compound according to claim 21, wherein one of R¹ and R² is —OR⁹ and the other of R¹ and R² is hydrogen or halo.

23. The compound according to claim 22, wherein R² is —OR⁹ and R¹ is hydrogen.

24. The compound according to claim 23, wherein R⁹ is lower alkyl optionally substituted with one or more substituents selected from the group consisting of fluoro, lower alkoxy, fluoro substituted lower alkoxy, lower alkylthio, fluoro substituted lower alkylthio, cycloalkyl, and fluoro substituted cycloalkyl.

25. The compound according to any of claims 20-24, wherein Ar₁ₐ is phenyl and M is bound to Ar₁ₐ para to the S(O)₂.

26. The compound according to claim 25, wherein Ar₂ₐ is phenyl.

27. The compound according to claim 26, wherein v is 1 and R²⁵ is bound para to M.

28. The compound according to claim 27, wherein M is a covalent bond or —O—.

29. The compound according to claim 28, wherein X is C(O)OR¹⁶.

30. The compound according to claim 29, wherein R¹⁶ is H.

31. The compound according to claim 30, wherein W is —(CR⁴R⁵)₁₋₃—.

32. The compound according to claim 31, wherein W is —CH₂— or —CH₂CH₂—.

33. The compound according to claim 24, wherein R⁹ is lower alkyl optionally substituted with one or more substituents selected from the group consisting of fluoro, lower alkoxy, and lower alkylthio, Ar₁ₐ is phenyl, M is a covalent bond or —O— and is bound to Ar₁ₐ para to the S(O)₂, u is 0, v is 1, Ar₂ₐ is phenyl, W is —CH₂—, X is —COOH, and R²⁵ is selected from the group consisting of halogen, lower alkyl, lower alkoxy, and lower alkylthio, wherein lower alkyl, lower alkoxy, and lower alkylthio are optionally substituted with one or more substituents selected from the group consisting of fluoro, lower alkoxy, fluoro substituted lower alkoxy, lower alkylthio, and fluoro substituted lower alkylthio.

34. The compound according to claim 33, wherein R⁹ is lower alkyl, M is —O—, and R²⁵ is optionally fluoro substituted lower alkyl or optionally fluoro substituted lower alkoxy.

35. The compound according to claim 34, wherein R²⁵ is bound to Ar₂ₐ para to M.

36. The compound according to claim 34, wherein R²⁵ is bound to Ar₂ₐ meta to M.

37. The compound according to claim 24, wherein R⁹ is lower alkyl optionally substituted with one or more substituents selected from the group consisting of fluoro, lower alkoxy, and lower alkylthio, Ar₁ₐ is phenyl, M is a covalent bond or —O— and is bound to Ar₁ₐ meta to the —S(O)₂—, u is 0, v is 1, Ar₂ₐ is phenyl, W is —CH₂—, X is —COOH, and R²⁵ is selected from the group consisting of halogen, lower alkyl, lower alkoxy, and lower alkylthio, wherein lower alkyl, lower alkoxy, and lower alkylthio are optionally substituted with one or more substituents selected from the group consisting of fluoro, lower alkoxy, fluoro substituted lower alkoxy, lower alkylthio, and fluoro substituted lower alkylthio.

38. The compound according to claim 37, wherein R⁹ is lower alkyl and R²⁵ is optionally fluoro substituted lower alkyl or optionally fluoro substituted lower alkoxy.

39. The compound according to claim 38, wherein R²⁵ is bound to Ar₂ₐ para to M.

40. The compound according to claim 38, wherein R²⁵ is bound to Ar₂ₐ meta to M.

41. The compound according to claim 1 having the chemical structure 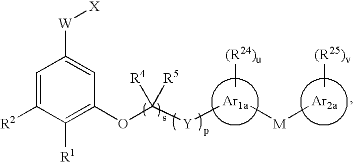
all salts, prodrugs, tautomers, and isomers thereof,
wherein:
X is selected from the group consisting of —C(O)OR¹⁶, —C(O)NR¹⁷R¹⁸, and a carboxylic acid isostere;
W is selected from the group consisting of a covalent bond, —NR⁵¹(CR⁴R⁵)₁₋₂—, —O—(CR⁴R⁵)₁₋₂—, —S—(CR⁴R⁵)₁₋₂—, —(CR⁴R⁵)₁₋₃—, and —CR⁶═CR⁷—;
Y is selected from the group consisting of —O—, —S—, —NR⁵³—, —C(Z)-, —S(O)ₙ—, —C(Z)NR⁵⁴—, —NR⁵⁴C(Z)-, —NR⁵⁴S(O)₂—, —S(O)₂NR⁵⁴—, —NR⁵⁴C(Z)NR⁵⁴—, and —NR⁵⁴S(O)₂NR⁵⁴—;
M is selected from the group consisting of a covalent bond, —CR¹⁹R²⁰—, —O—, —S—, —NR⁵³—, —C(Z)-, and —S(O)ₙ—;
Ar₁ₐ is selected from the group consisting of arylene and heteroarylene;
Ar₂ₐ is selected from the group consisting of aryl and heteroaryl;
R¹ and R² are independently selected from the group consisting of hydrogen, halogen, lower alkyl, lower alkenyl, lower alkynyl, —SR⁹, and —OR⁹, wherein lower alkyl, lower alkenyl and lower alkynyl are optionally substituted with one or more substituents selected from the group consisting of fluoro, —OH, —NH₂, lower alkoxy, fluoro substituted lower alkoxy, lower alkylthio, fluoro substituted lower alkylthio, cycloalkyl, heterocycloalkyl, aryl and heteroaryl, wherein cycloalkyl, heterocycloalkyl, aryl and heteroaryl are optionally substituted with one or more substituents selected from the group consisting of halogen, —OH, —NH₂, lower alkyl, fluoro substituted lower alkyl, lower alkenyl, fluoro substituted lower alkenyl, lower alkynyl, fluoro substituted lower alkynyl, lower alkoxy, fluoro substituted lower alkoxy, lower alkylthio, and fluoro substituted lower alkylthio;
R⁴ and R⁵ at each occurrence are independently selected from the group consisting of hydrogen, fluoro and lower alkyl, wherein lower alkyl is optionally substituted with one or more substituents selected from the group consisting of fluoro, —OH, —NH₂, lower alkoxy, fluoro substituted lower alkoxy, lower alkylthio, and fluoro substituted lower alkylthio; or
one R⁴ or R⁵ is selected from the group consisting of phenyl, 5-7 membered monocyclic heteroaryl, 3-7 membered monocyclic cycloalkyl, and 5-7 membered monocyclic heterocycloalkyl and any others of R⁴ and R⁵ are independently selected from the group consisting of hydrogen, fluoro and lower alkyl, wherein lower alkyl is optionally substituted with one or more substituents selected from the group consisting of fluoro, —OH, —NH₂, lower alkoxy, fluoro substituted lower alkoxy, lower alkylthio, and fluoro substituted lower alkylthio, and wherein phenyl, monocyclic heteroaryl, monocyclic cycloalkyl and monocyclic heterocycloalkyl are optionally substituted with one or more substituents selected from the group consisting of halogen, —OH, —NH₂, lower alkyl, fluoro substituted lower alkyl, lower alkoxy, fluoro substituted lower alkoxy, lower alkylthio, and fluoro substituted lower alkylthio; or
any two of R⁴ and R⁵ on the same or different carbons combine to form a 3-7 membered monocyclic cycloalkyl or 5-7 membered monocyclic heterocycloalkyl and any others of R⁴ and R⁵ are independently selected from the group consisting of hydrogen, fluoro and lower alkyl, wherein lower alkyl is optionally substituted with one or more substituents selected from the group consisting of fluoro, —OH, —NH₂, lower alkoxy, fluoro substituted lower alkoxy, lower alkylthio, and fluoro substituted lower alkylthio, and wherein the monocyclic cycloalkyl or monocyclic heterocycloalkyl are optionally substituted with one or more substituents selected from the group consisting of halogen, —OH, —NH₂, lower alkyl, fluoro substituted lower alkyl, lower alkoxy, fluoro substituted lower alkoxy, lower alkylthio, and fluoro substituted lower alkylthio;
R⁶ and R⁷ are independently hydrogen or lower alkyl, wherein lower alkyl is optionally substituted with one or more substituents selected from the group consisting of fluoro, —OH, —NH₂, lower alkoxy, fluoro substituted lower alkoxy, lower alkylthio, and fluoro substituted lower alkylthio; or
one of R⁶ and R⁷ is selected from the group consisting of phenyl, 5-7 membered monocyclic heteroaryl, 3-7 membered monocyclic cycloalkyl, and 5-7 membered monocyclic heterocycloalkyl and the other of R⁶ and R⁷ is hydrogen or lower alkyl, wherein lower alkyl is optionally substituted with one or more substituents selected from the group consisting of fluoro, —OH, —NH₂, lower alkoxy, fluoro substituted lower alkoxy, lower alkylthio, and fluoro substituted lower alkylthio, and wherein phenyl, monocyclic heteroaryl, monocyclic cycloalkyl and monocyclic heterocycloalkyl are optionally substituted with one or more substituents selected from the group consisting of halogen, —OH, —NH₂, lower alkyl, fluoro substituted lower alkyl, lower alkoxy, fluoro substituted lower alkoxy, lower alkylthio, and fluoro substituted lower alkylthio; or
R⁶ and R⁷ combine to form a 5-7 membered monocyclic cycloalkyl or 5-7 membered monocyclic heterocycloalkyl, wherein the monocyclic cycloalkyl or monocyclic heterocycloalkyl are optionally substituted with one or more substituents selected from the group consisting of halogen, —OH, —NH₂, lower alkyl, fluoro substituted lower alkyl, lower alkoxy, fluoro substituted lower alkoxy, lower alkylthio, and fluoro substituted lower alkylthio;
R⁹ at each occurrence is independently selected from the group consisting of lower alkyl, C₃₋₆ alkenyl, provided, however, that when R⁹ is C₃₋₆ alkenyl, no alkene carbon thereof is bound to the O of —OR⁹ or the S of —SR⁹, C₃₋₆ alkynyl, provided, however, that when R⁹ is C₃₋₆ alkynyl, no alkyne carbon thereof is bound to the O of —OR⁹ or the S of —SR⁹, cycloalkyl, heterocycloalkyl, aryl, and heteroaryl, wherein cycloalkyl, heterocycloalkyl, aryl and heteroaryl are optionally substituted with one or more substituents selected from the group consisting of halogen, —OH, —NH₂, lower alkyl, fluoro substituted lower alkyl, lower alkenyl, fluoro substituted lower alkenyl, lower alkynyl, fluoro substituted lower alkynyl, lower alkoxy, fluoro substituted lower alkoxy, lower alkylthio, and fluoro substituted lower alkylthio, and wherein lower alkyl, C₃₋₆ alkenyl and C₃₋₆ alkynyl are optionally substituted with one or more substituents selected from the group consisting of fluoro, —OH, —NH₂, lower alkoxy, fluoro substituted lower alkoxy, lower alkylthio, fluoro substituted lower alkylthio, cycloalkyl, heterocycloalkyl, aryl and heteroaryl, provided, however, that any substitution on the alkyl, C₃₋₆ alkenyl or C₃₋₆ alkynyl carbon bound to the O of —OR⁹ or the S of —SR⁹ is selected from the group consisting of fluoro, cycloalkyl, heterocycloalkyl, aryl and heteroaryl, wherein cycloalkyl, heterocycloalkyl, aryl, and heteroaryl substituents of alkyl, C₃₋₆ alkenyl and C₃₋₆ alkynyl are optionally substituted with one or more substituents selected from the group consisting of halogen, —OH, —NH₂, lower alkyl, fluoro substituted lower alkyl, lower alkenyl, fluoro substituted lower alkenyl, lower alkynyl, fluoro substituted lower alkynyl, lower alkoxy, fluoro substituted lower alkoxy, lower alkylthio, and fluoro substituted lower alkylthio;
R⁵¹ is selected from the group consisting of hydrogen, lower alkyl, phenyl, 5-7 membered monocyclic heteroaryl, 3-7 membered monocyclic cycloalkyl, and 5-7 membered monocyclic heterocycloalkyl, wherein lower alkyl is optionally substituted with one or more substituents selected from the group consisting of fluoro, —OH, —NH₂, lower alkoxy, fluoro substituted lower alkoxy, lower alkylthio, and fluoro substituted lower alkylthio, provided, however, that any substitution on the alkyl carbon bound to the N of —NR⁵¹— is fluoro, and wherein phenyl, monocyclic heteroaryl, monocyclic cycloalkyl and monocyclic heterocycloalkyl are optionally substituted with one or more substituents selected from the group consisting of halogen, —OH, —NH₂, lower alkyl, fluoro substituted lower alkyl, lower alkoxy, fluoro substituted lower alkoxy, lower alkylthio, and fluoro substituted lower alkylthio;
R⁵³ at each occurrence is independently selected from the group consisting of hydrogen, lower alkyl, C₃₋₆ alkenyl, provided, however, that when R⁵³ is C₃₋₆ alkenyl, no alkene carbon thereof is bound to the N of —NR⁵³—, C₃₋₆ alkynyl, provided, however, that when R⁵³ is C₃₋₆ alkynyl, no alkyne carbon thereof is bound to the N of —NR⁵³—, cycloalkyl, heterocycloalkyl, aryl, heteroaryl, —C(Z)NR¹¹R¹², —S(O)₂NR¹¹R¹², —S(O)₂R¹³, —C(Z)R¹³, and —C(Z)OR¹⁵, wherein lower alkyl, C₃₋₆ alkenyl, and C₃₋₆ alkynyl, are optionally substituted with one or more substituents selected from the group consisting of fluoro, —OR²¹, —SR²¹, —NR²²R²³, cycloalkyl, heterocycloalkyl, aryl and heteroaryl, provided, however, that any substitution on the alkyl, C₃₋₆ alkenyl or C₃₋₆ alkynyl carbon bound to the N of any —NR⁵³— is selected from the group consisting of fluoro, cycloalkyl, heterocycloalkyl, aryl, and heteroaryl, and wherein any cycloalkyl, heterocycloalkyl, aryl or heteroaryl is optionally substituted with one or more substituents selected from the group consisting of halogen, —NO₂, —CN, —OR²¹, —SR²¹, —S(O)R²¹, —S(O)₂R²¹, —C(Z)R²¹, —C(Z)OR²¹—NR²²R²³, —C(Z)NR²²R²³, —S(O)₂NR²²R²³, —C(NH)NR²²R²³, —NR²¹C(Z)R²¹, —NR²¹S(O)R²¹, —NR²¹C(Z)NR²²R²³, —NR²¹S(O)₂NR²²R²³, lower alkyl, lower alkenyl, and lower alkynyl, wherein the lower alkyl, lower alkenyl, and lower alkynyl optional substituents of cycloalkyl, heterocycloalkyl, aryl or heteroaryl are further optionally substituted with one or more substituents selected from the group consisting of fluoro, —OR²¹, —SR²¹, and —NR²²R²³;
R⁵⁴ at each occurrence is independently selected from the group consisting of hydrogen, lower alkyl, C₃₋₆ alkenyl, provided, however, that when R⁵⁴ is C₃₋₆ alkenyl, no alkene carbon thereof is bound to the N of any —NR⁵⁴—, C₃₋₆ alkynyl, provided, however, that when R⁵⁴ is C₃₋₆ alkynyl, no alkyne carbon thereof is bound to the N of any —NR⁵⁴—, cycloalkyl, heterocycloalkyl, aryl, and heteroaryl, wherein lower alkyl, C₃₋₆ alkenyl, and C₃₋₆ alkynyl, are optionally substituted with one or more substituents selected from the group consisting of fluoro, —OR²¹, —SR²¹, —NR²²R²³, cycloalkyl, heterocycloalkyl, aryl and heteroaryl, provided, however, that any substitution on the alkyl, C₃₋₆ alkenyl or C₃₋₆ alkynyl carbon bound to the N of any —NR⁵⁴— is selected from the group consisting of fluoro, cycloalkyl, heterocycloalkyl, aryl, and heteroaryl, and wherein any cycloalkyl, heterocycloalkyl, aryl or heteroaryl is optionally substituted with one or more substituents selected from the group consisting of halogen, —NO₂, —CN, —OR²¹, —SR²¹, —S(O)R²¹, —S(O)₂R²¹, —C(Z)R²¹, —C(Z)OR²¹, —NR²²R²³, —C(Z)NR²²R²³, —S(O)₂NR²²R²³, —C(NH)NR²²R²³, —NR²¹C(Z)R²¹, —NR²¹S(O)₂R²¹, —NR²¹C(Z)NR²²R²³, —NR²¹S(O)₂NR²²R²³, lower alkyl, lower alkenyl, and lower alkynyl, wherein the lower alkyl, lower alkenyl, and lower alkynyl optional substituents of cycloalkyl, heterocycloalkyl, aryl or heteroaryl are further optionally substituted with one or more substituents selected from the group consisting of fluoro, —OR²¹, —SR²¹, and —NR²²R²³;
R¹¹ and R¹² at each occurrence are independently selected from the group consisting of hydrogen, lower alkyl, C₃₋₆ alkenyl, provided, however, that when R¹¹ and/or R¹² is C₃₋₆ alkenyl, no alkene carbon thereof is bound to the N of any —C(Z)NR¹¹R¹² or —S(O)₂NR¹¹R¹², C₃₋₆ alkynyl, provided, however, that when R¹¹ and/or R¹² is C₃₋₆ alkynyl, no alkyne carbon thereof is bound to the N of any —C(Z)NR¹¹R¹² or —S(O)₂NR¹¹R¹², cycloalkyl, heterocycloalkyl, aryl, and heteroaryl, wherein lower alkyl, C₃₋₆ alkenyl, and C₃₋₆ alkynyl, are optionally substituted with one or more substituents selected from the group consisting of fluoro, —OR²¹, —SR²¹, —NR²²R²³, cycloalkyl, heterocycloalkyl, aryl and heteroaryl, provided, however, that any substitution on the alkyl, C₃₋₆ alkenyl or C₃₋₆ alkynyl carbon bound to the N of any —C(Z)NR¹¹R¹² or —S(O)₂NR¹¹R¹² is selected from the group consisting of fluoro, cycloalkyl, heterocycloalkyl, aryl, and heteroaryl, and wherein any cycloalkyl, heterocycloalkyl, aryl or heteroaryl is optionally substituted with one or more substituents selected from the group consisting of halogen, —NO₂, —CN, —OR²¹, —SR²¹, —S(O)R²¹, —S(O)₂R²¹, —C(Z)R²¹, —C(Z)OR²¹, —NR²²R²³, —C(Z)NR²²R²³, —S(O)₂NR²²R²³, —C(NH)NR²²R²³, —NR²¹C(Z)R²¹, —NR²¹S(O)R²¹, —NR²¹C(Z)NR²¹R²², —NR²¹S(O)₂NR²²R²³, lower alkyl, lower alkenyl, and lower alkynyl, wherein the lower alkyl, lower alkenyl, and lower alkynyl optional substituents of cycloalkyl, heterocycloalkyl, aryl or heteroaryl are further optionally substituted with one or more substituents selected from the group consisting of fluoro, —OR²¹, —SR²¹, and —NR²²R²³; or
R¹¹ and R¹² together with the nitrogen to which they are attached form a 5-7 membered monocyclic heterocycloalkyl or a 5 or 7 membered monocyclic nitrogen containing heteroaryl, wherein the monocyclic heterocycloalkyl or monocyclic nitrogen containing heteroaryl is optionally substituted with one or more substituents selected from the group consisting of halogen, —OH, —NH₂, —NO₂, —CN, lower alkyl, fluoro substituted lower alkyl, lower alkoxy, fluoro substituted lower alkoxy, lower alkylthio, fluoro substituted lower alkylthio, mono-alkylamino, di-alkylamino, and cycloalkylamino;
R¹³ at each occurrence is independently selected from the group consisting of lower alkyl, C₃₋₆ alkenyl, provided, however, that when R¹³ is C₃₋₆ alkenyl, no alkene carbon thereof is bound to C(Z) of —C(Z)R¹³, or S(O)₂ of —S(O)₂R¹³, C₃₋₆ alkynyl, provided, however, that when R¹³ is C₃₋₆ alkynyl, no alkyne carbon thereof is bound to C(Z) of —C(Z)R¹³, or S(O)₂ of —S(O)₂R¹³, cycloalkyl, heterocycloalkyl, aryl, and heteroaryl, wherein lower alkyl, C₃₋₆ alkenyl, and C₃₋₆ alkynyl, are optionally substituted with one or more substituents selected from the group consisting of fluoro, —OR²¹, —SR²¹, —NR²²R²³, cycloalkyl, heterocycloalkyl, aryl and heteroaryl, and wherein any cycloalkyl, heterocycloalkyl, aryl or heteroaryl is optionally substituted with one or more substituents selected from the group consisting of halogen, —NO₂, —CN, —OR²¹, —SR²¹, —S(O)R²¹, —S(O)₂R²¹, —C(Z)R²¹, —C(Z)OR²¹—NR²²R²³, —C(Z)NR²²R²³, —S(O)₂NR²²R²³, —C(NH)NR²²R²³, —NR²¹C(Z)R²¹, —NR²¹S(O)₂R²¹, —NR²¹C(Z)NR²²R²³, —NR²¹S(O)₂NR²²R²³, lower alkyl, lower alkenyl, and lower alkynyl, wherein the lower alkyl, lower alkenyl, and lower alkynyl, optional substituents of cycloalkyl, heterocycloalkyl, aryl or heteroaryl are further optionally substituted with one or more substituents selected from the group consisting of fluoro, —OR²¹, —SR²¹, and —NR²²R²³;
R¹⁵ at each occurrence is independently selected from the group consisting of hydrogen, lower alkyl, C₃₋₆ alkenyl, provided, however, that when R¹⁵ is C₃₋₆ alkenyl, no alkene carbon thereof is bound to O of OR¹⁵, C₃₋₆ alkynyl, provided, however, that when R¹⁵ is C₃₋₆ alkynyl, no alkyne carbon thereof is bound to O of OR¹⁵, cycloalkyl, heterocycloalkyl, aryl, and heteroaryl, wherein lower alkyl, C₃₋₆ alkenyl, and C₃₋₆ alkynyl, are optionally substituted with one or more substituents selected from the group consisting of fluoro, —OR²¹, —SR²¹, —NR²²R²³, cycloalkyl, heterocycloalkyl, aryl and heteroaryl, provided, however, that any substitution on the alkyl, C₃₋₆ alkenyl or C₃₋₆ alkynyl carbon bound to the O of any OR¹⁵ is selected from the group consisting of fluoro, cycloalkyl, heterocycloalkyl, aryl, and heteroaryl, and wherein any cycloalkyl, heterocycloalkyl, aryl or heteroaryl is optionally substituted with one or more substituents selected from the group consisting of halogen, —NO₂, —CN, —OR²¹, —SR²¹, —S(O)R²¹, —S(O)₂R²¹, —C(Z)R²¹, —C(Z)OR²¹, —NR²²R²³, —C(Z)NR²²R²³, —S(O)₂NR²²R²³, —C(NH)NR²²R²³, —NR²¹C(Z)R²¹, —NR²¹S(O)₂R²¹, —NR²¹C(Z)NR²²R²³, —NR²¹S(O)₂NR²²R²³, lower alkyl, lower alkenyl, and lower alkynyl, wherein the lower alkyl, lower alkenyl, and lower alkynyl optional substituents of cycloalkyl, heterocycloalkyl, aryl or heteroaryl are further optionally substituted with one or more substituents selected from the group consisting of fluoro, —OR²¹, —SR²¹, and —NR²²R²³;
R¹⁶ is selected from the group consisting of hydrogen, lower alkyl, phenyl, 5-7 membered monocyclic heteroaryl, 3-7 membered monocyclic cycloalkyl, and 5-7 membered monocyclic heterocycloalkyl, wherein phenyl, monocyclic heteroaryl, monocyclic cycloalkyl and monocyclic heterocycloalkyl are optionally substituted with one or more substituents selected from the group consisting of halogen, —OH, —NH₂, lower alkyl, fluoro substituted lower alkyl, lower alkoxy, fluoro substituted lower alkoxy, lower alkylthio, and fluoro substituted lower alkylthio, and wherein lower alkyl is optionally substituted with one or more substituents selected from the group consisting of fluoro, —OH, —NH₂, lower alkoxy, fluoro substituted lower alkoxy, lower alkylthio and fluoro substituted lower alkylthio, provided, however, that when R¹⁶ is lower alkyl, any substitution on the alkyl carbon bound to the O of OR¹⁶ is fluoro;
R¹⁷ and R¹⁸ are independently selected from the group consisting of hydrogen, lower alkyl, phenyl, 5-7 membered monocyclic heteroaryl, 3-7 membered monocyclic cycloalkyl, and 5-7 membered monocyclic heterocycloalkyl, wherein phenyl, monocyclic heteroaryl, monocyclic cycloalkyl and monocyclic heterocycloalkyl are optionally substituted with one or more substituents selected from the group consisting of halogen, —OH, —NH₂, lower alkyl, fluoro substituted lower alkyl, lower alkoxy, fluoro substituted lower alkoxy, lower alkylthio, and fluoro substituted lower alkylthio, and wherein lower alkyl is optionally substituted with one or more substituents selected from the group consisting of fluoro, —OH, —NH₂, lower alkoxy, fluoro substituted lower alkoxy, lower alkylthio and fluoro substituted lower alkylthio, provided, however, that when R¹⁷ and/or R¹⁸ is lower alkyl, any substitution on the alkyl carbon bound to the N of NR¹⁷R¹⁸ is fluoro; or
R¹⁷ and R¹⁸ together with the nitrogen to which they are attached form a 5-7 membered monocyclic heterocycloalkyl or a 5 or 7 membered nitrogen containing monocyclic heteroaryl, wherein the monocyclic heterocycloalkyl or monocyclic nitrogen containing heteroaryl is optionally substituted with one or more substituents selected from the group consisting of halogen, —OH, —NH₂, lower alkyl, fluoro substituted lower alkyl, lower alkoxy, fluoro substituted lower alkoxy, lower alkylthio, and fluoro substituted lower alkylthio;
R¹⁹ and R²⁰ are independently selected from the group consisting of hydrogen, lower alkyl, lower alkenyl, lower alkynyl, cycloalkyl, heterocycloalkyl, aryl, and heteroaryl, wherein lower alkyl, lower alkenyl, and lower alkynyl, are optionally substituted with one or more substituents selected from the group consisting of fluoro, —OR²¹, —SR²¹, —NR²²R²³, cycloalkyl, heterocycloalkyl, aryl and heteroaryl, and wherein any cycloalkyl, heterocycloalkyl, aryl or heteroaryl is optionally substituted with one or more substituents selected from the group consisting of halogen, —NO₂, —CN, —OR²¹, —SR²¹, —S(O)R²¹, —S(O)₂R²¹, —C(Z)R²¹, —C(Z)OR²¹, —NR²²R²³, —C(Z)NR²²R²³, —S(O)₂NR²²R²³, —C(NH)NR²²R²³, —NR²¹C(Z)R²¹, —NR²¹S(O)₂R²¹, —NR²¹C(Z)NR²²R²³, —NR²¹S(O)₂NR²²R²³, lower alkyl, lower alkenyl, and lower alkynyl, wherein the lower alkyl, lower alkenyl, and lower alkynyl optional substituents of cycloalkyl, heterocycloalkyl, aryl or heteroaryl are further optionally substituted with substituents selected from the group consisting of fluoro, —OR²¹, —SR²¹, and —NR²²R²³; or
R¹⁹ and R²⁰ combine to form a 3-7 membered monocyclic cycloalkyl or 5-7 membered monocyclic heterocycloalkyl, wherein the monocyclic cycloalkyl or monocyclic heterocycloalkyl are optionally substituted with one or more substituents selected from the group consisting of halogen, —OH, —NH₂, lower alkyl, fluoro substituted lower alkyl, lower alkoxy, fluoro substituted lower alkoxy, lower alkylthio, and fluoro substituted lower alkylthio;
R²¹, R²², and R²³ at each occurrence are independently hydrogen or lower alkyl optionally substituted with one or more substituents selected from the group consisting of fluoro, lower alkoxy, fluoro substituted lower alkoxy, lower alkylthio, fluoro substituted lower alkylthio, mono-alkylamino, di-alkylamino, and cycloalkylamino provided, however, that any substitution on the lower alkyl carbon bound to O, S, or N of any of OR²¹, SR²¹, NR²¹, NR²² or NR²³ is fluoro, and further provided, however, that R²¹ bound to S, S(O), S(O)₂ or C(Z) is not hydrogen; or
R²² and R²³ together with the nitrogen to which they are attached form a 5-7 membered monocyclic heterocycloalkyl or a 5 or 7 membered monocyclic nitrogen containing heteroaryl, wherein the monocyclic heterocycloalkyl or monocyclic nitrogen containing heteroaryl is optionally substituted with one or more substituents selected from the group consisting of halogen, —OH, —NH₂, —NO₂, —CN, lower alkyl, fluoro substituted lower alkyl, lower alkoxy, fluoro substituted lower alkoxy, lower alkylthio, fluoro substituted lower alkylthio, mono-alkylamino, di-alkylamino, and cycloalkylamino;
R²⁴ at each occurrence is independently selected from the group consisting of halogen, lower alkyl, lower alkenyl, lower alkynyl, —NO₂, —CN, —OR²⁶, —SR²⁶, —OC(O)R²⁶, —OC(S)R²⁶, —C(O)R²⁶, —C(S)R²⁶, —C(O)OR²⁶—C(S)OR²⁶, —S(O)R²⁶, —S(O)R²⁶, —C(O)NR²⁷R²⁸, —C(S)NR²⁷R²⁸, —S(O)₂NR²⁷R²⁸, —C(NH)NR²⁷R²⁸, —NR²⁶C(O)R²⁶, —NR²⁶C(S)R²⁶, —NR²⁶S(O)₂R²⁶, NR²⁶C(O)NR²⁷R²⁸, NR²⁶C(S)NR²⁷R²⁸, —NR²⁶S(O)₂NR²⁷R²⁸, and —NR²⁷R²⁸, wherein lower alkyl is optionally substituted with one or more substituents selected from the group consisting of fluoro, —OR³⁶, —SR³⁶, and —NR³⁷R³⁸, and wherein lower alkenyl and lower alkynyl are optionally substituted with one or more substituents selected from the group consisting of fluoro, —R³⁶, —SR³⁶, —NR³⁷R³⁸ and —R³⁵;
R²⁵ at each occurrence is independently selected from the group consisting of halogen, lower alkyl, lower alkenyl, lower alkynyl, cycloalkyl, heterocycloalkyl, aryl, heteroaryl, —NO₂, —CN, —OR²⁹, —SR²⁹, —OC(O)R²⁹, —OC(S)R²⁹, —C(O)R²⁹, —C(S)R²⁹, —C(O)OR²⁹, —C(S)OR²⁹, —S(O)R²⁹, —S(O)₂R²⁹, —C(O)NR²⁹R²⁹, —C(S)NR²⁹R²⁹, —S(O)₂NR²⁹R²⁹, —C(NH)NR³⁰R³¹, —NR²⁹C(O)R²⁹, —NR²⁹C(S)R²⁹, —NR²⁹S(O)₂R²⁹, —NR²⁹C(O)NR²⁹R²⁹, —NR²⁹C(S)NR²⁹R²⁹, —NR²⁹S(O)₂NR²⁹R²⁹, and —NR²⁹R²⁹, wherein lower alkyl is optionally substituted with one or more substituents selected from the group consisting of fluoro, —OR³⁶, —SR³⁶—NR³⁷R³⁸, and R³², and wherein lower alkenyl and lower alkynyl are optionally substituted with one or more substituents selected from the group consisting of fluoro, —OR³⁶, —SR³⁶, —NR³⁷R³⁸, —R³⁵ and —R³², and wherein cycloalkyl, heterocycloalkyl, aryl, and heteroaryl are optionally substituted with one or more substituents selected from the group consisting of halogen, —NO₂, —CN, —OR³⁶, —SR³⁶, —NR³⁷R³⁸, —R³⁵, —R³³, and —R³⁴;
R²⁶, R²⁷ and R²⁸ at each occurrence are independently selected from the group consisting of hydrogen, lower alkyl, C₃₋₆ alkenyl, provided, however, that no alkene carbon thereof is bound to any O, S, N, C(O), C(S), S(O) or S(O)₂ of R²⁴, and C₃₋₆ alkynyl, provided, however, that no alkyne carbon thereof is bound to any O, S, N, C(O), C(S), S(O) or S(O)₂ of R²⁴, wherein lower alkyl is optionally substituted with one or more substituents selected from the group consisting of fluoro, —OR³⁶, —SR³⁶, and —NR³⁷R³⁸, and wherein lower alkenyl and lower alkynyl are optionally substituted with one or more substituents selected from the group consisting of fluoro, —OR³⁶, —SR³⁶, —NR³⁷R³⁸, and —R³⁵, further provided, however, that R²⁶ bound to S, C(O), C(S), S(O), or S(O)₂ is not hydrogen, or
R²⁷ and R²⁸ combine with the nitrogen to which they are attached to form cycloalkylamino;
R²⁹, R³⁰ and R³¹ at each occurrence are independently selected from the group consisting of hydrogen, lower alkyl, C₃₋₆ alkenyl, provided, however, that no alkene carbon thereof is bound to any O, S, N, C(O), C(S), S(O) or S(O)₂ of R²⁵, C₃₋₆ alkynyl, provided, however, that no alkyne carbon thereof is bound to any O, S, N, C(O), C(S), S(O) or S(O)₂ of R²⁵, cycloalkyl, heterocycloalkyl, aryl and heteroaryl, or
R³⁰ and R³¹ combine with the nitrogen to which they are attached to form a 5-7 membered heterocycloalkyl or a 5 or 7 membered nitrogen containing heteroaryl, wherein lower alkyl is optionally substituted with one or more substituents selected from the group consisting of fluoro, —OR³⁶, —SR³⁶—NR³⁷R³⁸, and —R³² wherein lower alkenyl and lower alkynyl are optionally substituted with one or more substituents selected from the group consisting of fluoro, —OR³⁶, —SR³⁶, —NR³⁷R³⁸, —R³⁵ and —R³², and wherein cycloalkyl, heterocycloalkyl, aryl, heteroaryl, 5-7 membered heterocycloalkyl, and 5 or 7 membered nitrogen containing heteroaryl are optionally substituted with one or more substituents selected from the group consisting of halogen, —NO₂, —CN, —OH, —NH₂, —OR³⁶, —SR³⁶, —NHR³⁶, —NR³⁷R³⁸, —R³³, —R³⁴, and —R³⁵, further provided, however, that R²⁹ bound to S, C(O), C(S), S(O), or S(O)₂ is not hydrogen;
R³² at each occurrence is independently selected from the group consisting of cycloalkyl, heterocycloalkyl, aryl and heteroaryl, wherein cycloalkyl, heterocycloalkyl, aryl and heteroaryl are optionally substituted with one or more substituents selected from the group consisting of halogen, —NO₂, —CN, —OR³⁶, —SR³⁶, —NR³⁷R³⁸, —R³³, —R³⁴, and —R³⁵;
R³³ at each occurrence is independently lower alkenyl optionally substituted with one or more substituents selected from the group consisting of fluoro, —OR³⁶, —SR³⁶, —NR³⁷R³⁸ and —R³⁵;
R³⁴ at each occurrence is independently lower alkynyl optionally substituted with one or more substituents selected from the group consisting of fluoro, —OR³⁶, —SR³⁶, —NR³⁷R³⁸ and —R³⁵;
R³⁵ at each occurrence is independently lower alkyl optionally substituted with one or more substituents selected from the group consisting of fluoro, —OR³⁶, —SR³⁶, and —NR³⁷R³⁸;
R³⁶, R³⁷ and R³⁸ at each occurrence is independently hydrogen or lower alkyl optionally substituted with one or more substituents selected from the group consisting of fluoro, lower alkoxy, fluoro substituted lower alkoxy, lower alkylthio, fluoro substituted lower alkylthio, mono-alkylamino, di-alkylamino, and cycloalkylamino, or —NR³⁷R³⁸ is cycloalkylamino, provided, however, that any substitution on the lower alkyl carbon bound to the O, S, or N of any of OR³⁶, SR³⁶, NR³⁶, NR³⁷ or NR³⁸ is fluoro, and further provided, however, that R³⁶ bound to S is not hydrogen;
Z is O or S;
n is 1 or 2;
u is 0, 1, 2, 3 or 4;
v is 0, 1, 2, 3, 4, or 5;
p is 0 or 1; and
s is 0, 1, 2, 3 or 4, provided, however, that when s=0, then p=0 and when s is 1, 2, 3, or 4 and p=0, then Ar₁ₐ is not pyrazolyl, imidazolyl, isoxazolyl, oxazolyl, thiazolyl, or isothiazolyl, and when s=0, p=0, and Ar₂ₐ is phenyl, 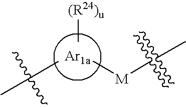
is not 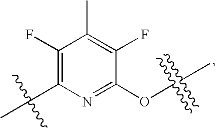
wherein 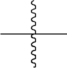
indicates the attachment point to O and 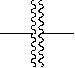
indicates the attachment point to Ar₂ₐ.

42. The compound according to claim 41, wherein at least one of R¹ and R² is —OR⁹.

43. The compound according to claim 42, wherein one of R¹ and R² is —OR⁹ and the other of R¹ and R² is hydrogen or halo.

44. The compound according to claim 43, wherein R² is —OR⁹ and R¹ is hydrogen.

45. The compound according to claim 44, wherein R⁹ is lower alkyl optionally substituted with one or more substituents selected from the group consisting of fluoro, lower alkoxy, fluoro substituted lower alkoxy, lower alkylthio, fluoro substituted lower alkylthio, cycloalkyl, and fluoro substituted cycloalkyl.

46. The compound according to claim 41 having the chemical structure

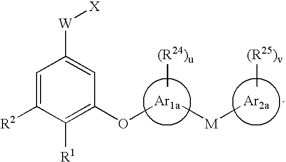

47. The compound according to claim 46, wherein at least one of R¹ and R² is —OR⁹.

48. The compound according to claim 47, wherein one of R¹ and R² is —OR⁹ and the other of R¹ and R² is hydrogen or halo.

49. The compound according to claim 48, wherein R² is —OR⁹ and R¹ is hydrogen.

50. The compound according to claim 49, wherein R⁹ is lower alkyl optionally substituted with one or more substituents selected from the group consisting of fluoro, lower alkoxy, fluoro substituted lower alkoxy, lower alkylthio, fluoro substituted lower alkylthio, cycloalkyl, and fluoro substituted cycloalkyl.

51. The compound according to any of claims 46-50, wherein Ar₁ₐ is phenyl and M is bound to Ar₁ₐ para to the S(O)₂.

52. The compound according to claim 51, wherein Ar₂ₐ is phenyl.

53. The compound according to claim 52, wherein v is 1 and R²⁵ is bound para to M.

54. The compound according to claim 53, wherein M is a covalent bond or —O—.

55. The compound according to claim 54, wherein X is C(O)OR¹⁶.

56. The compound according to claim 55, wherein R¹⁶ is H.

57. The compound according to claim 56, wherein W is —(CR⁴R⁵)₁₋₃—.

58. The compound according to claim 57, wherein W is —CH₂— or —CH₂CH₂—.

59. The compound according to claim 50, wherein R⁹ is lower alkyl optionally substituted with one or more substituents selected from the group consisting of fluoro, lower alkoxy, and lower alkylthio, Ar₁ₐ is phenyl, M is a covalent bond or —O— and is bound to Ar₁ₐ para to the —O—, u is 0, v is 1, Ar₂ₐ is phenyl, W is —CH₂—, X is —COOH, and R²⁵ is selected from the group consisting of halogen, lower alkyl, lower alkoxy, and lower alkylthio, wherein lower alkyl, lower alkoxy, and lower alkylthio are optionally substituted with one or more substituents selected from the group consisting of fluoro, lower alkoxy, fluoro substituted lower alkoxy, lower alkylthio, and fluoro substituted lower alkylthio.

60. The compound according to claim 59, wherein R⁹ is lower alkyl, M is —O—, and R²⁵ is optionally fluoro substituted lower alkyl or optionally fluoro substituted lower alkoxy.

61. The compound according to claim 60, wherein R²⁵ is bound to Ar₂ₐ para to M.

62. The compound according to claim 60, wherein R²⁵ is bound to Ar₂ₐ meta to M.

63. The compound according to claim 50, wherein R⁹ is lower alkyl optionally substituted with one or more substituents selected from the group consisting of fluoro, lower alkoxy, and lower alkylthio, Ar₁ₐ is phenyl, M is a covalent bond or —O— and is bound to Ar₁ₐ meta to the —O—, u is 0, v is 1, Ar₂ₐ is phenyl, W is —CH₂—, X is —COOH, and R²⁵ is selected from the group consisting of halogen, lower alkyl, lower alkoxy, and lower alkylthio, wherein lower alkyl, lower alkoxy, and lower alkylthio are optionally substituted with one or more substituents selected from the group consisting of fluoro, lower alkoxy, fluoro substituted lower alkoxy, lower alkylthio, and fluoro substituted lower alkylthio.

64. The compound according to claim 63, wherein R⁹ is lower alkyl and R²⁵ is optionally fluoro substituted lower alkyl or optionally fluoro substituted lower alkoxy.

65. The compound according to claim 64, wherein R²⁵ is bound to Ar₂ₐ para to M.

66. The compound according to claim 64, wherein R²⁵ is bound to Ar₂ₐ meta to M.

67. The compound according to claim 1, wherein said compound is selected from the group consisting of:
{3-Butoxy-5-[4-(4-trifluoromethoxy-phenoxy)-benzenesulfonyl]-phenyl}-acetic acid,
{3-Methoxy-5-[4-(4-trifluoromethoxy-phenoxy)-benzenesulfonyl]-phenyl}-acetic acid,
{3-(2-Methoxy-ethoxy)-5-[4-(4-trifluoromethyl-phenoxy)-benzenesulfonyl]-phenyl}-acetic acid,
{3-(2-Methoxy-ethoxy)-5-[4-(4-trifluoromethoxy-phenoxy)-benzenesulfonyl]-phenyl}-acetic acid,
{3-Methoxy-5-[4-(4-trifluoromethyl-phenoxy)-benzenesulfonyl]-phenyl}-acetic acid,
{3-Benzyloxy-5-[4-(4-trifluoromethyl-phenoxy)-benzenesulfonyl]-phenyl}-acetic acid,
{3-Butoxy-5-[4-(4-trifluoromethyl-phenoxy)-benzenesulfonyl]-phenyl}-acetic acid,
{3-Ethoxy-5-[4-(4-trifluoromethoxy-phenoxy)-benzenesulfonyl]-phenyl}-acetic acid,
{3-Propoxy-5-[4-(4-trifluoromethoxy-phenoxy)-benzenesulfonyl]-phenyl}-acetic acid,
{3-Propoxy-5-[3-(4-trifluoromethyl-phenoxy)-benzenesulfonyl]-phenyl}-acetic acid,
[3-Ethoxy-5-(4′-trifluoromethyl-biphenyl-3-sulfonyl)phenyl]-acetic acid,
{3-Ethoxy-5-[4-(4-trifluoromethoxy-phenoxy)-benzenesulfonyl]-phenyl}-acetic acid methyl ester,
{3-Ethoxy-5-[4-(4-trifluoromethyl-phenoxy)-benzenesulfonyl]-phenyl}-acetic acid,
[3-Ethoxy-5-(4′-trifluoromethoxy-biphenyl-3-sulfonyl)-phenyl]-acetic acid,
3-{3-Propoxy-5-[4-(4-trifluoromethyl-phenoxy)-benzenesulfonyl]-phenyl}-propionic acid,
3-{3-Ethoxy-5-[4-(4-trifluoromethyl-phenoxy)-benzenesulfonyl]-phenyl}-propionic acid, and
3-{3-Propoxy-5-[4-(4-trifluoromethoxy-phenoxy)-benzenesulfonyl]-phenyl}-propionic acid.

68. The compound according to claim 1, wherein said compound is selected from the group consisting of:
[3-Butoxy-5-(4-trifluoromethyl-benzenesulfonyl)-phenyl]-acetic acid,
[3-Butoxy-5-(4-methoxy-benzenesulfonyl)-phenyl]-acetic acid,
[3-Butoxy-5-(4-trifluoromethoxy-benzenesulfonyl)-phenyl]-acetic acid, and
[3-butoxy-5-(3-methoxy-benzenesulfonyl)-phenyl]-acetic acid.

69. A composition comprising:
a pharmaceutically acceptable carrier; and
a compound according to any of claims 1, 15, 20, 41 or 46.

70. A method for treating a subject suffering from or at risk of a disease or condition for which PPAR modulation provides a therapeutic benefit, comprising administering to said subject an effective amount of a composition according to claim 69.

71. The method according to claim 70, wherein said compound is approved for administration to a human.

72. The method according to claim 70, wherein said disease or condition is a PPAR-mediated disease or condition.

73. The method according to claim 70, wherein said disease or condition is selected from the group consisting of obesity, overweight condition, bulimia, anorexia nervosa, hyperlipidemia, dyslipidemia, hypoalphalipoproteinemia, hypertriglyceridemia, and hypercholesterolemia, low HDL, Metabolic Syndrome, Type II diabetes mellitus, Type I diabetes, hyperinsulinemia, impaired glucose tolerance, insulin resistance, a diabetic complication of neuropathy, nephropathy, retinopathy, diabetic foot ulcer or cataracts, hypertension, coronary heart disease, heart failure, congestive heart failure, atherosclerosis, arteriosclerosis, stroke, cerebrovascular disease, myocardial infarction, peripheral vascular disease, vitiligo, uveitis, pemphigus foliaceus, inclusion body myositis, polymyositis, dermatomyositis, scleroderma, Grave's disease, Hashimoto's disease, chronic graft versus host disease, rheumatoid arthritis, inflammatory bowel syndrome, Crohn's disease, systemic lupus erythematosis, Sjogren's Syndrome, multiple sclerosis, asthma, chronic obstructive pulmonary disease, polycystic kidney disease, polycystic ovary syndrome, pancreatitis, nephritis, hepatitis, eczema, psoriasis, dermatitis, impaired wound healing, Alzheimer's disease, Parkinson's disease, amyotrophic lateral sclerosis, spinal cord injury, acute disseminated encephalomyelitis, Guillain-Barre syndrome, thrombosis, infarction of the large or small intestine, renal insufficiency, erectile dysfunction, urinary incontinence, neurogenic bladder, ophthalmic inflammation, macular degeneration, pathologic neovascularization, HCV infection, HIV infection, Helicobacter pylori infection, neuropathic or inflammatory pain, infertility, and cancer.

74. A kit comprising a pharmaceutical composition according to claim 69.

75. The kit according to claim 74, further comprising a written indication that said composition is approved for administering to a human.

76. The kit according to claim 75, wherein said composition is approved for a medical indication selected from the group consisting of obesity, overweight condition, bulimia, anorexia nervosa, hyperlipidemia, dyslipidemia, hypoalphalipoproteinemia, hypertriglyceridemia, and hypercholesterolemia, low HDL, Metabolic Syndrome, Type II diabetes mellitus, Type I diabetes, hyperinsulinemia, impaired glucose tolerance, insulin resistance, a diabetic complication of neuropathy, nephropathy, retinopathy, diabetic foot ulcer or cataracts, hypertension, coronary heart disease, heart failure, congestive heart failure, atherosclerosis, arteriosclerosis, stroke, cerebrovascular disease, myocardial infarction, peripheral vascular disease, vitiligo, uveitis, pemphigus foliaceus, inclusion body myositis, polymyositis, dermatomyositis, scleroderma, Grave's disease, Hashimoto's disease, chronic graft versus host disease, rheumatoid arthritis, inflammatory bowel syndrome, Crohn's disease, systemic lupus erythematosis, Sjogren's Syndrome, multiple sclerosis, asthma, chronic obstructive pulmonary disease, polycystic kidney disease, polycystic ovary syndrome, pancreatitis, nephritis, hepatitis, eczema, psoriasis, dermatitis, impaired wound healing, Alzheimer's disease, Parkinson's disease, amyotrophic lateral sclerosis, spinal cord injury, acute disseminated encephalomyelitis, Guillain-Barre syndrome, thrombosis, infarction of the large or small intestine, renal insufficiency, erectile dysfunction, urinary incontinence, neurogenic bladder, ophthalmic inflammation, macular degeneration, pathologic neovascularization, HCV infection, HIV infection, Helicobacter pylori infection, neuropathic or inflammatory pain, infertility, and cancer.


SEQUENCE LISTING (19 sequences)

SEQ ID NO: 1 - DNA, 1014 bp, Artificial Sequence
  Description of Artificial Sequence Synthetic construct
taatacgactcactataggggaattgtgagcggataacaattcccctctagaaataattttgtttaactttaagaaggagatataccatgggcagcagccatcatcatcatcatcacagcagcggcctggtgccgcgcggcagccatatggaaactgcagatctcaaatctctggccaagagaatctacgaggcctacttgaagaacttcaacatgaacaaggtcaaagcccgggtcatcctctcaggaaaggccagtaacaatccaccttttgtcatacatgatatggagacactgtgtatggctgagaagacgctggtggccaagctggtggccaatggcatccagaacaaggaggcggaggtccgcatctttcactgctgccagtgcacgtcagtggagaccgtcacggagctcacggaattcgccaaggccatcccaggcttcgcaaacttggacctgaacgatcaagtgacattgctaaaatacggagtttatgaggccatattcgccatgctgtcttctgtgatgaacaaagacgggatgctggtagcgtatggaaatgggtttataactcgtgaattcctaaaaagcctaaggaaaccgttctgtgatatcatggaacccaagtttgattttgccatgaagttcaatgcactggaactggatgacagtgatatctccctttttgtggctgctatcatttgctgtggagatcgtcctggccttctaaacgtaggacacattgaaaaaatgcaggagggtattgtacatgtgctcagactccacctgcagagcaaccacccggacgatatctttctcttcccaaaacttcttcaaaaaatggcagacctccggcagctggtgacggagcatgcgcagctggtgcagatcatcaagaagacggagtcggatgctgcgctgcacccgctactgcaggagatctacagggacatgtactgagtcgacaagcttgcggccgcactcgagcaccaccaccaccaccactgagat

SEQ ID NO: 2 - protein, 291 aa, Artificial Sequence
  Description of Artificial Sequence Synthetic construct
MGSSHHHHHHSSGLVPRGSHMETADLKSLAKRIYEAYLKNFNMNKVKARVILSGKASNNPPFVIHDMETLCMAEKTLVAKLVANGIQNKEAEVRIFHCCQCTSVETVTELTEFAKAIPGFANLDLNDQVTLLKYGVYEAIFAMLSSVMNKDGMLVAYGNGFITREFLKSLRKPFCDIMEPKFDFAMKFNALELDDSDISLFVAAIICCGDRPGLLNVGHIEKMQEGIVHVLRLHLQSNHPDDIFLFPKLLQKMADLRQLVTEHAQLVQIIKKTESDAALHPLLQEIYRDMY

SEQ ID NO: 3 - DNA, 33 bp, Artificial Sequence
  Description of Artificial Sequence Synthetic primer
gctgacacatatggaaactgcagatctcaaatc

SEQ ID NO: 4 - DNA, 32 bp, Artificial Sequence
  Description of Artificial Sequence Synthetic primer
gtgactgtcgactcagtacatgtccctgtaga

SEQ ID NO: 5 - DNA, 1017 bp, Artificial Sequence
  Description of Artificial Sequence Synthetic construct
taatacgactcactataggggaattgtgagcggataacaattcccctctagaaataattttgtttaactttaagaaggagatataccatgggcagcagccatcatcatcatcatcacagcagcggcctggtgccgcgcggcagccatatggagtccgctgacctccgggccctggcaaaacatttgtatgactcatacataaagtccttcccgctgaccaaagcaaaggcgagggcgatcttgacaggaaagacaacagacaaatcaccattcgttatctatgacatgaattccttaatgatgggagaagataaaatcaagttcaaacacatcacccccctgcaggagcagagcaaagaggtggccatccgcatctttcagggctgccagtttcgctccgtggaggctgtgcaggagatcacagagtatgccaaaagcattcctggttttgtaaatcttgacttgaacgaccaagtaactctcctcaaatatggagtccacgagatcatttacacaatgctggcctccttgatgaataaagatggggttctcatatccgagggccaaggcttcatgacaagggagtttctaaagagcctgcgaaagccttttggtgactttatggagcccaagtttgagtttgctgtgaagttcaatgcactggaattagatgacagcgacttggcaatatttattgctgtcattattctcagtggagaccgcccaggtttgctgaatgtgaagcccattgaagacattcaagacaacctgctacaagccctggagctccagctgaagctgaaccaccctgagtcctcacagctgtttgccaagctgctccagaaaatgacagacctcagacagattgtcacggaacatgtgcagctactgcaggtgatcaagaagacggagacagacatgagtcttcacccgctcctgcaggagatctacaaggacttgtactaggtcgacaagcttgcggccgcactcgagcaccaccaccaccaccactgagat

SEQ ID NO: 6 - protein, 292 aa, Artificial Sequence
  Description of Artificial Sequence Synthetic construct
MGSSHHHHHHSSGLVPRGSHMESADLRALAKHLYDSYIKSFPLTKAKARAILTGKTTDKSPFVIYDMNSLMMGEDKIKFKHITPLQEQSKEVAIRIFQGCQFRSVEAVQEITEYAKSIPGFVNLDLNDQVTLLKYGVHEIIYTMLASLMNKDGVLISEGQGFMTREFLKSLRKPFGDFMEPKFEFAVKFNALELDDSDLAIFIAVIILSGDRPGLLNVKPIEDIQDNLLQALELQLKLNHPESSQLFAKLLQKMTDLRQIVTEHVQLLQVIKKTETDMSLHPLLQEIYKDLY

SEQ ID NO: 7 - DNA, 33 bp, Artificial Sequence
  Description of Artificial Sequence Synthetic primer
gctcagacatatggagtccgctgacctccgggc

SEQ ID NO: 8 - DNA, 32 bp, Artificial Sequence
  Description of Artificial Sequence Synthetic primer
gtgactgtcgacctagtacaagtccttgtaga

SEQ ID NO: 9 - DNA, 1089 bp, Artificial Sequence
  Description of Artificial Sequence Synthetic construct
taatacgactcactataggggaattgtgagcggataacaattcccctctagaaataattttgtttaactttaagaaggagatataccatgaaaaaaggtcaccaccatcaccatcacggatcccagtacaacccacaggtggccgacctgaaggccttctccaagcacatctacaatgcctacctgaaaaacttcaacatgaccaaaaagaaggcccgcagcatcctcaccggcaaagccagccacacggcgccctttgtgatccacgacatcgagacattgtggcaggcagagaaggggctggtgtggaagcagttggtgaatggcctgcctccctacaaggagatcagcgtgcacgtcttctaccgctgccagtgcaccacagtggagaccgtgcgggagctcactgagttcgccaagagcatccccagcttcagcagcctcttcctcaacgaccaggttacccttctcaagtatggcgtgcacgaggccatcttcgccatgctggcctctatcgtcaacaaggacgggctgctggtagccaacggcagtggctttgtcacccgtgagttcctgcgcagcctccgcaaacccttcagtgatatcattgagcctaagtttgaatttgctgtcaagttcaacgccctggaacttgatgacagtgacctggccctattcattgcggccatcattctgtgtggagaccggccaggcctcatgaacgttccacgggtggaggctatccaggacaccatcctgcgtgccctcgaattccacctgcaggccaaccaccctgatgcccagtacctcttccccaagctgctgcagaagatggctgacctgcggcaactggtcaccgagcacgcccagatgatgcagcggatcaagaagaccgaaaccgagacctcgctgcaccctctgctccaggagatctacaaggacatgtactaagtcgaccaccaccaccaccaccactgagatccggctggccctactggccgaaaggaattcgaggccagcagggccaccgctgagcaataactagcataaccccttggggcctctaaacgggtcttgaggggttttttg

SEQ ID NO: 10 - protein, 287 aa, Artificial Sequence
  Description of Artificial Sequence Synthetic construct
MKKGHHHHHHGSQYNPQVADLKAFSKHIYNAYLKNFNMTKKKARSILTGKASHTAPFVIHDIETLWQAEKGLVWKQLVNGLPPYKEISVHVFYRCQCTTVETVRELTEFAKSIPSFSSLFLNDQVTLLKYGVHEAIFAMLASIVNKDGLLVANGSGFVTREFLRSLRKPFSDIIEPKFEFAVKFNALELDDSDLALFIAAIILCGDRPGLMNVPRVEAIQDTILRALEFHLQANHPDAQYLFPKLLQKMADLRQLVTEHAQMMQRIKKTETETSLHPLLQEIYKDMY

SEQ ID NO: 11 - DNA, 29 bp, Artificial Sequence
  Description of Artificial Sequence Synthetic primer
gttggatcccagtacaacccacaggtggc

SEQ ID NO: 12 - DNA, 32 bp, Artificial Sequence
  Description of Artificial Sequence Synthetic primer
gtgactgtcgacttagtacatgtccttgtaga

SEQ ID NO: 13 - protein, 25 aa, Artificial Sequence
  Description of Artificial Sequence Synthetic peptide
DGTPPPQEAEEPSLLKKLLLAPANT

SEQ ID NO: 14 - DNA, 10049 bp, Homo sapiens
gcgccgcctccttcggcgttcgccccacggaccggcaggcggcggaccgcggcccaggctgaagctcagggccctgtctgctctgtggactcaacagtttgtggcaagacaagctcagaactgagaagctgtcaccacagttctggaggctgggaagttcaagatcaaagtgccagcagattcagtgtcatgtgaggacgtgcttcctgcttcatagataagagtagcttggagctcggcggcacaaccagcaccatctggtcgcgatggtggacacggaaagcccactctgccccctctccccactcgaggccggcgatctagagagcccgttatctgaagagttcctgcaagaaatgggaaacatccaagagatttcgcaatccatcggcgaggatagttctggaagctttggctttacggaataccagtatttaggaagctgtcctggctcagatggctcggtcatcacggacacgctttcaccagcttcgagcccctcctcggtgacttatcctgtggtccccggcagcgtggacgagtctcccagtggagcattgaacatcgaatgtagaatctgcggggacaaggcctcaggctatcattacggagtccacgcgtgtgaaggctgcaagggcttctttcggcgaacgattcgactcaagctggtgtatgacaagtgcgaccgcagctgcaagatccagaaaaagaacagaaacaaatgccagtattgtcgatttcacaagtgcctttctgtcgggatgtcacacaacgcgattcgttttggacgaatgccaagatctgagaaagcaaaactgaaagcagaaattcttacctgtgaacatgacatagaagattctgaaactgcagatctcaaatctctggccaagagaatctacgaggcctacttgaagaacttcaacatgaacaaggtcaaagcccgggtcatcctctcaggaaaggccagtaacaatccaccttttgtcatacatgatatggagacactgtgtatggctgagaagacgctggtggccaagctggtggccaatggcatccagaacaaggaggcggaggtccgcatctttcactgctgccagtgcacgtcagtggagaccgtcacggagctcacggaattcgccaaggccatcccaggcttcgcaaacttggacctgaacgatcaagtgacattgctaaaatacggagtttatgaggccatattcgccatgctgtcttctgtgatgaacaaagacgggatgctggtagcgtatggaaatgggtttataactcgtgaattcctaaaaagcctaaggaaaccgttctgtgatatcatggaacccaagtttgattttgccatgaagttcaatgcactggaactggatgacagtgatatctccctttttgtggctgctatcatttgctgtggagatcgtcctggccttctaaacgtaggacacattgaaaaaatgcaggagggtattgtacatgtgctcagactccacctgcagagcaaccacccggacgatatctttctcttcccaaaacttcttcaaaaaatggcagacctccggcagctggtgacggagcatgcgcagctggtgcagatcatcaagaagacggagtcggatgctgcgctgcacccgctactgcaggagatctacagggacatgtactgagttccttcagatcagccacaccttttccaggagttctgaagctgacagcactacaaaggagacgggggagcagcacgattttgcacaaatatccaccactttaaccttagagcttggacagtctgagctgtaggtaaccggcatattattccatatctttgttttaaccagtacttctaagagcatagaactcaaatgctgggggtaggtggctaatctcaggactgggaagattacggcgaattatgctcaatggtctgattttaactcacccgatgttaatcaatgcacattgctttagatcacattcgtgatttaccatttaattaactggtaacctcaaaattcgtggcctgtcttcccattcaccccgcttttgactattgtgctcctttataattctgaaaactaatcagcactttttaacaatgtttataatcctataagtctagatgtatccaaaggtgaagtatgtaaaaagcagcaaaatatttatttcaaagacttcacttctgtttcctgaatctaaagaaagacaacatgctgctttttaatcataggatggagaattttaaagaactgtttgggccaggcacagtcgctcatacttgtaatcccagcactttgggaggccgaggcgggtggatcacaaggtcagcagatcgagaccatcctggccaacatggtgaaaccctgtctctactaaaaatacaaaaattagccgggtgtggtggcacatgcctgtaatcccagctactcgggaagctgaggcaggagaattgcttgaaccagggagttggaggttgcagtgagctaagactgcaccactgcactccagcctggtgacagaacgagactctgtcttaaaaacaaacaaacaaaaaaaaaatctgttagataagctatcaaaatgcagctgttgttttgtttttggctcactgttttcgtggttgtaactaatatgtggaaaggcccatttccaggtttgcgtagaagagcccagaaaacagagtctcaagacccccgctctggactgtcataagctagcacccgtggtaagcgggacgagacaagctcccgaagcccgccagcttcctgctccactcagctccgtccagtcaacctgaacccacccagtccagctgtctgtgggaatggtggtgttcttagggacagactgacaccttacttgtcagtgttcctccgggccccatttggcagctcccgtatcttttgttatgttgcttttaaagatatgatgttttattgttttaactcttggtgacagtagatgctctctggagcgcagacgaggcacatgtgtcttcatagcctgggctgggtgggagccagtcaccctgcggatcgagagagggggtagagtcttcttcaaatggcagttttacttcaaatggcagatttcacaagagttggttattttttacaatggtttaggttgttaagtctcctttgtatgtaaggtagttttttcaacatctaaaatttttgttttagccttcaaaaccaacttaccaacctcagtccagctgggaaggcagcgttgattatggtagtttgtcaagaatatatggacctggaaacactttctctctctgtccacctggtagataaattgtcctgttgagaatttttagatctggactggaactgccaggaccaccgcctccagggagtcgctgggcacctggaggtatcgtcgatgcctctcccccatctttagaaaatttggctcttctgaggtcattattattttaagaatgattaggattgataagggtcccatgaccagcattatgaaaatgcgagagtgggaaggacacagtgtgagacttccactagaaaaaagtgaaagttagggttaggacatccttttttaaaaattacaaatttagtccgttttggtttttgtaatcaggctaggcacagtggctcacacatggaatcccagcactttgggaggccgaggtgggaggatcacttgagcccaggagttcgagaccagcctaggcaacatagcaagaccctgtctgtacacaaaatttaaaaattagttcatcggggtggcacacatcagtagtcccagctactctgcaggctgaggtgggaggattgcttgaacccaggaggtcgaggctgcagtgagctgtgatctcaccactgcattccagcctgggtgacagagttagattccaccctctcccaccccggcaaaaaaaaaaaaaaaagatgcaatcaaaggggctgttggccagcaatggcagcagcagcggcgggcagtctgcccaagtgtcttaggaaccaaaagcaaataaaagtgtttccatatatgccaccagccaagtggccatcctaattcagaaagaagctagcctttgagtgtctgtcatggtgcatccgtttcagtattatttcctaaaatgagaagcccctgtgtcaacaagatccaggggctggagcccaatgccaagcctgtgttgtccccagcgaccctgcagctgctcgctctgatgtaccctgtgccattcaaggagatgtggtccaggaaagtgagcctcatggttttcagagaagtcattgttctgtttacattttcataaaacctgtttaaaatagctccccgtctcaggctttcagcagtaacagtgagctgactggcaagttcgatgttagctcccgggacactcagcagcgatggtgagcattttggtttccttaaggcccagcaagacttccagggacatctctggtgaagccagaatggagacacccgtgacctcaggctgaaagtcactcgacattggtctcttgtgttgatagggaaggaaatcaggcattcctatttctttaaataacaaaaccactaattgccactcaatgctggaatattttgggtcacctaatcatagatttctcagggcatcaatactcaaatataggctgattatgccccagttcaaatgggaactattaacagagtgcatttcttgcttgctgggtttcaacagacatcagccaaaagaacaaaagagatgtcaggacagattccaggagtgtcggagcacatgtgtggcacccgctccctctggcagcgaatgtaggaagtcgccaaatttacccactcttcaacaagtcattgtttaaacacggtttttcattttctcaacttttaatagcaaaaagtgccaaagtcctcagagacctaacagccttggtctaccgtgctgaccagggtgaaggcacggcgagggactcctcccagacgtgcctcttgtgtgccagctggctgtggctcgggagcagacgcaggcctctccattgtccaggggagcctggcggcgcatccctcctctcccacctcctggcacttccagctgggtgtcccacatgttggattccgtccccaccacacttccagagaccggagaactgtgcagggcctaaggccgtttggatgaattgtcaaaacaagatgcttccagttacagcggcaggagcgggactgggagcacgggctgacggctgctggtgcctttcttcccacctcgcttgcctgtttccgcttgacccttcctccagctccgatgagaagagtataaagcatcttcctaacgggtgtgtttgctatacgaacataatggacgtgaagtggggcagaaacccagaactcagcattcaaggatgcccaggagagctgtccctgttttaaagagctgtgttttgttttgtttcgcatttagagagcagacaaggcacccttctgctgcgctgatacgtttcttacactgggccattttagacccccagggaaacagccttcctggagcgttgtctggaggttccagggacagggcagcctcccagagccgagcaagagctcaaggtacaaatgagagatttgctataccgtgagaagtcaacaacttagccaccacttccccgcaatggaccatgtaacaaatacctcagcaggccctgcaaaaggccatgctagagctgaggcgcacagcctgtggcctctgtagttagggcaggtgggatggagactccttgagtgcacacacctgagcctgcccacacacaggggagcagcatctcgtatgacgtctggaaggaacttcggttgtgtaaagggagccttgaagatacgtgcaaaaggtgctaccccaatttggtgaaactgacattgggcacgtcttgggcttaggagaagcggccgatggtcccggcctgcagtgacaaacccccctccccgcaccgcccccagcaccccctctcctcttcacctcttcctgctggccacgaggaagccacttcctcagagagaccctaccagatgcggatggaaacagatgcaccaaagcaagccctgatgaaaccgcgacttcctaaggtctgtctcctctgaacttgcacctgggcctctctgtgtttggttccaagcacttcccacctcaaactcccattttcaaaccactgtatctctgcgcacatctgctacttaccagccgcatacatgatggagggttttttggtcctgatccagtggccacacctgtctttgaaatgtctcactgaactccagttttaaaatagattcattgcttcaacacagcaagcccaatgcacccagctaagactggcttgaccgacagcctggcctttggtggggggcttcctggggcctggggaaagctggccaccttcaacagctggtacctcttcaacagtgtggcctttcaaaatgcagatgccaccaggagaacatgcccacagctcaccacctatggatgccatggctctgggcagctttcaaagcaggttcctgtggtctcctcagctgtttgagggggtaacagcaaatcagcctccattttaaaatgaaaacaccagcctccagatgtagggcctgctgggtgttgctagccgctggtccccaggcacggtgcactttctccacctcctgcagcctccctgttgtttctagactcttgcacctggtgagtgcaaggataggtgacccaggggcctgcagccttgtcctcagctcccatctcctggactgccagcctcaccctctgcagttagcatggttggcctgatgcagggatcccgagggattactttttagaccttctttcacattcagaaaagtagtatagattcaggagaggcaagaaaattatgctgtccatagaagtcacccatgaagactgatgccaccacctgaaggctcatgattgttaaaaatgtccacgggaacctctcgtccacaggaggtttgtctcaacacttcccatttttacggcattggcattgccaagcatggggaagtatctgctcttctcatgttaaaagtggcccagcttttcttaactcagtccaagctgacttgtttagctgcactggaatttcttaccaaccaaatatttgcatcgagcaaagggggctgtgtgcacctccctaatggcagcgatgatggctgctgtcattcaagcccatcttcagacgtcacagtctggaagtgaaatgtccacaaacatctgtggcagaaaaggctatacggaccacccagttgtgctgcagctttacagagcaaggaagggttgtggcaaataaatgattaacctgcctcgactgtgctgagggcaacaaaggccatctcaccaaaggattattcgatgccattaaatcatcccgtgaccttcctgcttccgagtccatggcctttgcccagggcatgtactcccctgagaggccttctgcctagaaagatctatgactgggttccaaagttgaggcctaggtttttgctgggatttagatattttcaggcaccattttgacagcattcaggaaaacggttattgaccccatagactagggtaagaataaaggcaataaatttggtctgactcagaatataggagatccatatatttctctggaaaccacagtgtacactaaaatgtgaaattgaaggttttgttaaaaagaaaaagataatgagcttcatgctttgtttaattacataatgatttccattacgctatttctgtgaaatgcagcaggttcttaaacgttatttcagtggcatgggctggaagcttatcacaaaaagccatgtgtgtggccttatcagaacagaaagagacaggctggtgcccaaggctgctgcctgctccaccttttgccagctctggacatctgaggacgtcccggcagatctggaatggggccctcaactgaccatttgcttctcagaatttcagtttgagacatgagaggtataatcagttacttttctccccccagagaaacccttttgtgaggggagaggagctatggtatgtggttcagctgaaacacatacaactgcatccttttggagtcctttgccaacaaaaacagaccaacagaccagatggtgtccatgttcaatatcatgtcttgatggacgcagctgatgacctcaaatacttgagtggtctcatggctgttagatggattatttgaaaaaaaaaaaaaaaaaagagagaaaaaataattgatttttacatcagagatagcaaactaagacctggggaggggggtcagcttttattttattttattttttttaagtttgctagttgggtcaaatgtgaggaggagggagtctacctgccacctcttctcttgcccctcttctgcccacacatccagcatccaaaatccattcatttaatgaattgataaagtgccgtgcaaactggtgcacaaacaggcccccagtccacgcagcctggctcctaggaaaagtggtgaccgggcgtgggggggcatgccgcagccctgggacacagtcgggcaccttccccggacccccaggccttggctgtgcctcaagtcagagagggtcagccttcaggccccggagacgagtgactggccgatcatttcacaataaaatcactcacttttggcaacttcactttttttaaggcacagtcagttccttttctcatgtacctcacaaaagatgaagaccatgtagtactctttttggtaaagttacagtgttcatgttaaatatcacttttttctacattgtgtggtaaaaagaactacgttaatagctatatcttaaatactgtgatttgactttttgaaaaatatcctaatacaaatattttactaacttacaatcactcatttaataagaaacatttggattcttttgaaatcagtgttaattgactcatattcttaaaagcctggctcttgaccctattggaaacacaaaggaagctgaaatcaaacatctaaaatacactgcgtacacgtgtgcgtgcacacacacacacacacacacacacacacagctcttcatttctcctgagccatgcagaatttactttcaatgtggaaatctgttccctttaccacactgtatatgcacagagcacaagagaggctatctctagtcacttccaccagcgaggccttagactccgtattagaggccaccgatttcatacaacagtgtttcgctaaagacccttcactattcttgtttagtaaatagctgtctgctcttcagggaactgttacctatgggttattaccaaagaacgctggcaattggaaatgtcctgatggaaattctttgcacgtgccggttctctggcatcctccaggtggcccaacccaaagcagaaagcagaaaccacagaccccgtgagtctccccataccttgtttccaataacttggcaaaacttcttggtgcatattggttacaccctctgggattcataatgccattaggctaaaaccctaagagagagggttgacagaaacacacgcgagaatgaggcagatcccagagcaaggactgggcccagactctccacatgtgctctactagtgagtgccttatactctcagtattttggggcttacagcttcttatttgtgctaaaaaggtgcagttccaaagtaggaactgccacacaggccccagcatcctctctccaacttcatacctctctcctggtggggggagcgggcatccaggacctccggaatcaaggatgtgcagagaagagcgaaagtaatttttctagtcacatgaactgattggttccaggcaattagaaaatggctataaaataaccttaattttaaaaaaaaatcttgggtcttcgttttcctattaggagactgaactgaccacatgtattgatttatatcctgaatatatgggaacttctgtgtttgggatgtcctactgtaagactgatgaatgtacagagttaatttcagggtacagttttgccttaatggttttaaaaaataaactattttttaaaatttt

SEQ ID NO: 15 - protein, 468 aa, Homo sapiens
MVDTESPLCPLSPLEAGDLESPLSEEFLQEMGNIQEISQSIGEDSSGSFGFTEYQYLGSCPGSDGSVITDTLSPASSPSSVTYPVVPGSVDESPSGALNIECRICGDKASGYHYGVHACEGCKGFFRRTIRLKLVYDKCDRSCKIQKKNRNKCQYCRFHKCLSVGMSHNAIRFGRMPRSEKAKLKAEILTCEHDIEDSETADLKSLAKRIYEAYLKNFNMNKVKARVILSGKASNNPPFVIHDMETLCMAEKTLVAKLVANGIQNKEAEVRIFHCCQCTSVETVTELTEFAKAIPGFANLDLNDQVTLLKYGVYEAIFAMLSSVMNKDGMLVAYGNGFITREFLKSLRKPFCDIMEPKFDFAMKFNALELDDSDISLFVAAIICCGDRPGLLNVGHIEKMQEGIVHVLRLHLQSNHPDDIFLFPKLLQKMADLRQLVTEHAQLVQIIKKTESDAALHPLLQEIYRDMY

SEQ ID NO: 16 - DNA, 1863 bp, Homo sapiens
actgatgtcttgactcatgggtgtattcacaaattctgttacttcaagtctttttcttttaacggattgatcttttgctagatagagacaaaatatcagtgtgaattacagcaaacccctattccatgctgttatgggtgaaactctgggagattctcctattgacccagaaagcgattccttcactgatacactgtctgcaaacatatcacaagaaatgaccatggttgacacagagatgccattctggcccaccaactttgggatcagctccgtggatctctccgtaatggaagaccactcccactcctttgatatcaagcccttcactactgttgacttctccagcatttctactccacattacgaagacattccattcacaagaacagatccagtggttgcagattacaagtatgacctgaaacttcaagagtaccaaagtgcaatcaaagtggagcctgcatctccaccttattattctgagaagactcagctctacaataagcctcatgaagagccttccaactccctcatggcaattgaatgtcgtgtctgtggagataaagcttctggatttcactatggagttcatgcttgtgaaggatgcaagggtttcttccggagaacaatcagattgaagcttatctatgacagatgtgatcttaactgtcggatccacaaaaaaagtagaaataaatgtcagtactgtcggtttcagaaatgccttgcagtggggatgtctcataatgccatcaggtttgggcggatgccacaggccgagaaggagaagctgttggcggagatctccagtgatatcgaccagctgaatccagagtccgctgacctccgggccctggcaaaacatttgtatgactcatacataaagtccttcccgctgaccaaagcaaaggcgagggcgatcttgacaggaaagacaacagacaaatcaccattcgttatctatgacatgaattccttaatgatgggagaagataaaatcaagttcaaacacatcacccccctgcaggagcagagcaaagaggtggccatccgcatctttcagggctgccagtttcgctccgtggaggctgtgcaggagatcacagagtatgccaaaagcattcctggttttgtaaatcttgacttgaacgaccaagtaactctcctcaaatatggagtccacgagatcatttacacaatgctggcctccttgatgaataaagatggggttctcatatccgagggccaaggcttcatgacaagggagtttctaaagagcctgcgaaagccttttggtgactttatggagcccaagtttgagtttgctgtgaagttcaatgcactggaattagatgacagcgacttggcaatatttattgctgtcattattctcagtggagaccgcccaggtttgctgaatgtgaagcccattgaagacattcaagacaacctgctacaagccctggagctccagctgaagctgaaccaccctgagtcctcacagctgtttgccaagctgctccagaaaatgacagacctcagacagattgtcacggaacacgtgcagctactgcaggtgatcaagaagacggagacagacatgagtcttcacccgctcctgcaggagatctacaaggacttgtactagcagagagtcctgagccactgccaacatttcccttcttccagttgcactattctgagggaaaatctgacacctaagaaatttactgtgaaaaagcattttaaaaagaaaaggttttagaatatgatctattttatgcatattgtttataaagacacatttacaatttacttttaatattaaaaattaccatattatgaaaaaaaaaaaaaaaa

SEQ ID NO: 17 - protein, 505 aa, Homo sapiens
MGETLGDSPIDPESDSFTDTLSANISQEMTMVDTEMPFWPTNFGISSVDLSVMEDHSHSFDIKPFTTVDFSSISTPHYEDIPFTRTDPVVADYKYDLKLQEYQSAIKVEPASPPYYSEKTQLYNKPHEEPSNSLMAIECRVCGDKASGFHYGVHACEGCKGFFRRTIRLKLIYDRCDLNCRIHKKSRNKCQYCRFQKCLAVGMSHNAIRFGRMPQAEKEKLLAEISSDIDQLNPESADLRALAKHLYDSYIKSFPLTKAKARAILTGKTTDKSPFVIYDMNSLMMGEDKIKFKHITPLQEQSKEVAIRIFQGCQFRSVEAVQEITEYAKSIPGFVNLDLNDQVTLLKYGVHEIIYTMLASLMNKDGVLISEGQGFMTREFLKSLRKPFGDFMEPKFEFAVKFNALELDDSDLAIFIAVIILSGDRPGLLNVKPIEDIQDNLLQALELQLKLNHPESSQLFAKLLQKMTDLRQIVTEHVQLLQVIKKTETDMSLHPLLQEIYKDLY

SEQ ID NO: 18 - DNA, 3734 bp, Homo sapiens
gcggagcgtgtgacgctgcggccgccgcggacctggggattaatgggaaaagttttggcaggagcgggagaattctgcggagcctgcgggacggcggcggtggcgccgtaggcagccgggacagtgttgtacagtgttttgggcatgcacgtgatactcacacagtggcttctgctcaccaacagatgaagacagatgcaccaacgaggctgatgggaaccaccctgtagaggtccatctgcgttcagacccagacgatgccagagctatgactgggcctgcaggtgtggcgccgaggggagatcagccatggagcagccacaggaggaagcccctgaggtccgggaagaggaggagaaagaggaagtggcagaggcagaaggagccccagagctcaatgggggaccacagcatgcacttccttccagcagctacacagacctctcccggagctcctcgccaccctcactgctggaccaactgcagatgggctgtgacggggcctcatgcggcagcctcaacatggagtgccgggtgtgcggggacaaggcatcgggcttccactacggtgttcatgcatgtgaggggtgcaagggcttcttccgtcgtacgatccgcatgaagctggagtacgagaagtgtgagcgcagctgcaagattcagaagaagaaccgcaacaagtgccagtactgccgcttccagaagtgcctggcactgggcatgtcacacaacgctatccgttttggtcggatgccggaggctgagaagaggaagctggtggcagggctgactgcaaacgaggggagccagtacaacccacaggtggccgacctgaaggccttctccaagcacatctacaatgcctacctgaaaaacttcaacatgaccaaaaagaaggcccgcagcatcctcaccggcaaagccagccacacggcgccctttgtgatccacgacatcgagacattgtggcaggcagagaaggggctggtgtggaagcagttggtgaatggcctgcctccctacaaggagatcagcgtgcacgtcttctaccgctgccagtgcaccacagtggagaccgtgcgggagctcactgagttcgccaagagcatccccagcttcagcagcctcttcctcaacgaccaggttacccttctcaagtatggcgtgcacgaggccatcttcgccatgctggcctctatcgtcaacaaggacgggctgctggtagccaacggcagtggctttgtcacccgtgagttcctgcgcagcctccgcaaacccttcagtgatatcattgagcctaagtttgaatttgctgtcaagttcaacgccctggaacttgatgacagtgacctggccctattcattgcggccatcattctgtgtggagaccggccaggcctcatgaacgttccacgggtggaggctatccaggacaccatcctgcgtgccctcgaattccacctgcaggccaaccaccctgatgcccagtacctcttccccaagctgctgcagaagatggctgacctgcggcaactggtcaccgagcacgcccagatgatgcagcggatcaagaagaccgaaaccgagacctcgctgcaccctctgctccaggagatctacaaggacatgtactaacggcggcacccaggcctccctgcagactccaatggggccagcactggaggggcccacccacatgacttttccattgaccagcccttgagcacccggcctggagcagcagagtcccacgatcgccctcagacacatgacacccacggcctctggctccctgtgccctctctcccgcttcctccagccagctctcttcctgtctttgttgtctccctctttctcagttcctctttcttttctaattcctgttgctctgtttcttcctttctgtaggtttctctcttcccttctcccttgccctccctttctctctccaccccccacgtctgtcctcctttcttattctgtgagatgttttgtattatttcaccagcagcatagaacaggacctctgcttttgcacaccttttccccaggagcagaagagagtggggcctgccctctgccccatcattgcacctgcaggcttaggtcctcacttctgtctcctgtcttcagagcaaaagacttgagccatccaaagaaacactaagctctctgggcctgggttccagggaaggctaagcatggcctggactgactgcagccccctatagtcatggggtccctgctgcaaaggacagtgggcaggaggccccaggctgagagccagatgcctccccaagactgtcattgcccctccgatgctgaggccacccactgacccaactgatcctgctccagcagcacacctcagccccactgacacccagtgtccttccatcttcacactggtttgccaggccaatgttgctgatggccccctgcactggccgctggacggcactctcccagcttggaagtaggcagggttccctccaggtgggcccccacctcactgaagaggagcaagtctcaagagaaggaggggggattggtggttggaggaagcagcacacccaattctgcccctaggactcggggtctgagtcctggggtcaggccagggagagctcggggcaggccttccgccagcactcccactgcccccctgcccagtagcagccgcccacattgtgtcagcatccagggccagggcctggcctcacatccccctgctcctttctctagctggctccacgggagttcaggccccactccccctgaagctgcccctccagcacacacacataagcactgaaatcactttacctgcaggctccatgcacctcccttccctccctgaggcaggtgagaacccagagagaggggcctgcaggtgagcaggcagggctgggccaggtctccggggaggcaggggtcctgcaggtcctggtgggtcagcccagcacctgctcccagtgggagcttcccgggataaactgagcctgttcattctgatgtccatttgtcccaatagctctactgccctccccttcccctttactcagcccagctggccacctagaagtctccctgcacagcctctagtgtccggggaccttgtgggaccagtcccacaccgctggtccctgccctcccctgctcccaggttgaggtgcgctcacctcagagcagggccaaagcacagctgggcatgccatgtctgagcggcgcagagccctccaggcctgcaggggcaaggggctggctggagtctcagagcacagaggtaggagaactggggttcaagcccaggcttcctgggtcctgcctggtcctccctcccaaggagccattctgtgtgtgactctgggtggaagtgcccagcccctgcccctacgggcgctgcagcctcccttccatgccccaggatcactctctgctggcaggattcttcccgctccccacctacccagctgatgggggttggggtgcttcctttcaggccaaggctatgaagggacagctgctgggacccacctccccctccccggccacatgccgcgtccctgccccgacccgggtctggtgctgaggatacagctcttctcagtgtctgaacaatctccaaaattgaaatgtatatttttgctaggagccccagcttcctgtgtttttaatataaatagtgtacacagactgacgaaactttaaataaatgggaattaaatatttaa

SEQ ID NO: 19 - protein, 441 aa, Homo sapiens
MEQPQEEAPEVREEEEKEEVAEAEGAPELNGGPQHALPSSSYTDLSRSSSPPSLLDQLQMGCDGASCGSLNMECRVCGDKASGFHYGVHACEGCKGFFRRTIRMKLEYEKCERSCKIQKKNRNKCQYCRFQKCLALGMSHNAIRFGRMPEAEKRKLVAGLTANEGSQYNPQVADLKAFSKHIYNAYLKNFNMTKKKARSILTGKASHTAPFVIHDIETLWQAEKGLVWKQLVNGLPPYKEISVHVFYRCQCTTVETVRELTEFAKSIPSFSSLFLNDQVTLLKYGVHEAIFAMLASIVNKDGLLVANGSGFVTREFLRSLRKPFSDIIEPKFEFAVKFNALELDDSDLALFIAAIILCGDRPGLMNVPRVEAIQDTILRALEFHLQANHPDAQYLFPKLLQKMADLRQLVTEHAQMMQRIKKTETETSLHPLLQEIYKDMY